(19) United States
(12) Patent Application Publication
(10) Pub. No.: US 2022/0251577 A1
(43) Pub. Date: Aug. 11, 2022

(54) ENDONUCLEASE-RESISTANT MESSENGER RNA AND USES THEREOF

(71) Applicant: ModernaTX, Inc., Cambridge, MA (US)

(72) Inventors: Alicia BICKNELL, Cambridge, MA (US); Vladimir PRESNYAK, Manchester, NH (US); Caroline KÖHRER, Cambridge, MA (US)

(21) Appl. No.: 17/618,776

(22) PCT Filed: Jun. 24, 2020
(86) PCT No.: PCT/US2020/039228

§ 371 Date: Dec. 13, 2021

Related U.S. Application Data
(60) Provisional application No. 62/865,813, filed on Jun. 24, 2019.

(51) Int. Cl. C12N 15/67; A61K 31/7105

(52) U.S. Cl. CPC C12N 15/67; A61K 31/7105

(57) ABSTRACT

The present disclosure provides messenger RNAs (mRNAs) with alterations which provide increased endonuclease resistance to the mRNA and methods and uses thereof.

### RELATED APPLICATIONS

This application claims the benefit of U.S. Provisional Application Ser. No. 62/865,813, filed on Jun. 24, 2019, which is hereby incorporated by reference in its entirety.

### BACKGROUND

Administration of an exogenous mRNA (e.g., a synthetic or in vitro-transcribed mRNA) that structurally resembles natural mRNA can result in the controlled production of therapeutic proteins or peptides via the endogenous and constitutively-active translation machinery (e.g. ribosomes) that exists within a patient's own cells. In recent years, the development and use of mRNA as a therapeutic agent has demonstrated potential for treatment of numerous diseases and for the development of novel approaches in regenerative medicine, cancer, rare diseases and vaccination (Stanton et al (2017) Messenger RNA as a Novel Therapeutic Approach. In: Garnder A. (eds) RNA Therapeutics. Topics in Medicinal Chemistry, vol 27 Springer, Cham; Sabnis et al. (2018) Mol Ther 26:1509-1519; Hassett et al. (2019) Mol Ther Nucleic Acids 15:P1-11

The utility of mRNA as a therapeutic can be limited by poor stability and susceptibility to degradation in vivo, resulting in reduced production of the encoded therapeutic polypeptide. In eukaryotic cells, the degradation of mRNA is an essential determinant in the regulation of gene expression. Ribonucleases (e.g., endonucleases and exonucleases) represent a class of enzymes that mediate the degradation of RNA polynucleotides (e.g., mRNA) into smaller components. While cells degrade mRNA to regulate the amount of proteins that can be translated from each mRNA molecule, cells also naturally modify mRNA molecules in a way that increases the stability of the mRNA molecule, thereby increasing the protein output under specific conditions and at certain times.

In order for this class of drugs to achieve optimal therapeutic effect, control and regulation of mRNA translation and mRNA stability would be desirable. The development of methods that overcome degradation challenges associated with the administration of exogenous mRNA should allow for reduced dose levels and/or frequency. Therefore, compositions comprising novel endonuclease resistant sequence motifs, and methods of detecting and removing endonuclease sensitive motifs would be of great benefit.

### SUMMARY OF THE DISCLOSURE

Improving mRNA stability in vivo is a desirable feature for mRNA therapeutics. The present invention is based, at least in part, on the discovery of endonuclease sensitive sequence motifs located immediately upstream of an endonuclease cleavage site in mRNA molecules. The presence of these endonuclease sensitive sequence motifs in an mRNA for therapeutic expression has been shown to correlate with the susceptibility of the mRNA to degradation. Further, it has been shown that a decrease in or removal of these motifs (e.g., by substitution of one or more nucleotides present in the motifs) from the mRNA (e.g., from the 3′ UTR) increases the stability (e.g., half-life) of the mRNA in cells, presumably owing to an increase in the resistance of the mRNA to endonuclease activity in the cytoplasm. While in vitro transcribed mRNA can be introduced into cells (e.g., by transfection) and undergo translation by the endogenous translation machinery of a cell, the introduced mRNA is also subject to degradation by endogenous exonuclease and endonuclease activity. Moreover, mRNA can be degraded even before it reaches a cell; RNA is unstable in some bodily fluids, particularly in human serum. Thus, natural, unmodified mRNA can be degraded between the time it is administered to a subject and the time it enters a cell. Within a cell, a natural mRNA decays with a half-life of between 30 minutes and several days.

Accordingly, the present disclosure is generally directed to therapeutic mRNA molecules having improved stability and methods of improving or increasing mRNA stability, by increasing the nuclease stability of the mRNA, while maintaining or increasing the ability of the mRNA to function as a template for translation of a polypeptide (e.g., a therapeutically relevant protein), thereby increasing the efficiency of an mRNA as a drug. In some aspects, the disclosure provides methods for reducing the endonuclease sensitivity of an mRNA molecule, methods for generating an endonuclease-resistant mRNA from an endonuclease-susceptible mRNA and compositions comprising the stabilized and/or endonuclease resistant mRNA. The present disclosure is also directed to methods of treating a disease or disorder (e.g., cancer, autoimmune disease, infectious disease, metabolic disease) using a stabilized and/or endonuclease-resistant mRNA generated by the methods described herein to express a therapeutically relevant protein within a cell or a subject.

In some aspects, the disclosure provides a method of increasing stability of an mRNA, the method comprising: providing an mRNA comprising at least one endonuclease sensitive sequence motif, wherein the endonuclease sensitive sequence motif comprises the nucleotide sequence WGA, wherein W=adenine (A) or uracil (U); and altering the at least one endonuclease sensitive sequence motif, thereby increasing stability of the mRNA.

In some aspects, the disclosure provides a method of increasing resistance and/or decreasing susceptibility of an mRNA to endonuclease activity, wherein the mRNA comprises at least one endonuclease sensitive sequence motif, wherein the endonuclease sensitive sequence motif comprises the nucleotide sequence WGA, wherein W=adenine (A) or uracil (U), the method comprising altering the at least one endonuclease sensitive sequence motif, thereby increasing resistance and/or decreasing susceptibility of the mRNA to endonuclease activity.

In some aspects, the disclosure provides a method of increasing the half-life of an mRNA, wherein the mRNA comprises at least one endonuclease sensitive sequence motif, wherein the endonuclease sensitive sequence motif comprises the nucleotide sequence WGA, wherein W=adenine (A) or uracil (U), the method comprising altering the at least one endonuclease sensitive sequence motif, thereby increasing the half-life of the mRNA.

In some aspects, the disclosure provides a method of increasing stability of an mRNA, increasing resistance and/or decreasing susceptibility of an mRNA to endonuclease activity, and/or increasing the half-life of an mRNA, wherein the mRNA comprises at least one endonuclease sensitive sequence motif, wherein the endonuclease sensitive sequence motif comprises the nucleotide sequence WGA, wherein W=adenine (A) or uracil (U), the method comprising altering the at least one endonuclease sensitive sequence motif, thereby increasing the stability of an mRNA, increasing resistance and/or decreasing susceptibility of an mRNA to endonuclease activity, and/or increasing the half-life of an mRNA. In some aspects, the mRNA comprises a 5′ untranslated region (5′ UTR), an open reading frame (ORF) encoding a polypeptide, and a 3′ UTR, wherein the 5′ UTR, ORF and/or 3′ UTR comprise at least one endonuclease sensitive sequence motif. In some aspects, the 5′ UTR comprises at least one endonuclease sensitive sequence motif. In some aspects, the ORF comprises at least one endonuclease sensitive sequence motif. In some aspects, the 3′UTR comprises at least one endonuclease sensitive sequence motif. In some aspects, the 5′UTR and ORF each comprise at least one endonuclease sensitive sequence motif, and wherein the at least one endonuclease sensitive sequence motif is the same or is different. In some aspects, the 5′UTR and 3′UTR each comprise at least one endonuclease sensitive sequence motif, and wherein the at least one endonuclease sensitive sequence motif is the same or is different. In some aspects, the ORF and the 3′UTR each comprise at least one endonuclease sensitive sequence motif, and wherein the at least one endonuclease sensitive sequence motif is the same or is different. In some aspects, the 5′UTR, ORF and 3′UTR each comprise at least one endonuclease sensitive sequence motif, and wherein the at least one endonuclease sensitive sequence motif is the same or is different.

In any of the foregoing aspects, the endonuclease sensitive sequence motif is about 3-4, about 4-6, about 6-10, about 3, 4, 5, 6, 7, 8, 9, or 10 nucleotides in length. In some aspects, the endonuclease sensitive sequence motif is 3 nucleotides in length.

In any of the foregoing aspects, the disclosure provides a method of increasing stability of an mRNA, increasing resistance and/or decreasing susceptibility of an mRNA to endonuclease activity, and/or increasing the half-life of an mRNA, the method comprising altering the at least one endonuclease sensitive sequence motif of an mRNA, wherein altering the at least one endonuclease sensitive sequence motif comprises a substitution or chemical modification of at least one nucleotide comprising the endonuclease sensitive sequence motif. In some aspects, altering the at least one endonuclease sensitive sequence motif comprises inserting one or more nucleotides into the motif, deleting one or more nucleotides from the motif, substituting one or more nucleotides comprising the motif, or a combination thereof.

In any of the foregoing aspects, the disclosure provides a method of increasing stability of an mRNA, increasing resistance and/or decreasing susceptibility of an mRNA to endonuclease activity, and/or increasing the half-life of an mRNA, wherein the mRNA comprises at least one endonuclease sensitive sequence motif comprising the nucleotide sequence WGA, wherein W=adenine (A) or uracil (U), the method comprising altering the at least one endonuclease sensitive sequence motif, thereby increasing the stability of an mRNA, increasing resistance and/or decreasing susceptibility of an mRNA to endonuclease activity, and/or increasing the half-life of an mRNA. In some aspects, the method comprising altering the at least one endonuclease sensitive sequence motif, wherein W=adenine (A), and wherein W is substituted with cytosine (C), guanine (G), or uracil (U). In some aspects, the method comprising altering the at least one endonuclease sensitive sequence motif, wherein W=uracil (U), and wherein W is substituted with cytosine (C), guanine (G), or adenine (A). In some aspects, the method comprising altering the at least one endonuclease sensitive sequence motif, wherein W=adenine (A), wherein W is preceded by an AG, and wherein AG is substituted with GC. In some aspects, the method comprising altering the at least one endonuclease sensitive sequence motif, wherein W is substituted with cytosine (C), guanine (G), or uracil (U). In some aspects, the method comprising altering the at least one endonuclease sensitive sequence motif, wherein W=adenine (A), wherein W is substituted with guanine (G), wherein W is preceded by CG, and wherein the C is substituted with A. In some aspects, the method comprising altering the at least one endonuclease sensitive sequence motif, wherein W=adenine (A), wherein W is substituted with cytosine (C) or uracil (U), wherein W is preceded by uracil (U) and cytosine (C), and wherein UC is substituted with AG. In some aspects, the method comprising altering the at least one endonuclease sensitive sequence motif, wherein W=adenine (A), wherein W is substituted with cytosine (C), guanine (G), or uracil (U), wherein W is preceded by a UU, and wherein the first U comprising the UU is substituted with C. In some aspects, the method comprising altering the at least one endonuclease sensitive sequence motif, wherein W=uracil (U), wherein W is substituted with cytosine (C), guanine (G), or adenine (A), wherein W is preceded by an AG, and wherein the AG is substituted with UC. In some aspects, the method comprising altering the at least one endonuclease sensitive sequence motif, wherein W=uracil (U), wherein W is substituted with adenine (A) or guanine (G), wherein W is preceded by a CU, and wherein the C is substituted with U. In some aspects, the method comprising altering the at least one endonuclease sensitive sequence motif, wherein W is substituted with cytosine (C), wherein W is preceded by a UC, and wherein the UC is substituted with AG. In some aspects, the method comprising altering the at least one endonuclease sensitive sequence motif, wherein W=adenine (A), and wherein the adenine (A) following the guanine (G) of the sequence motif is substituted with guanine (G).

In any of the foregoing aspects, the disclosure provides a method of increasing stability of an mRNA, increasing resistance and/or decreasing susceptibility of an mRNA to endonuclease activity, and/or increasing the half-life of an mRNA, wherein the mRNA comprises at least one endonuclease sensitive sequence motif in the 3′UTR, the method comprising altering the at least one endonuclease sensitive sequence motif, thereby increasing the stability of an mRNA, increasing resistance and/or decreasing susceptibility of an mRNA to endonuclease activity, and/or increasing the half-life of an mRNA. In some aspects, the 3′UTR comprises at least one endonuclease sensitive sequence motif, wherein the sequence motif comprises the stop codon UGA, wherein the G is substituted with A, thereby forming the stop codon UAA. In some aspects, the 3′UTR comprises at least one endonuclease sensitive sequence motif, wherein the sequence motif comprises the stop codon UGA, wherein the GA is substituted with an AG, thereby forming the stop codon UAG.

In any of the foregoing aspects, the disclosure provides a method of increasing stability of an mRNA, increasing resistance and/or decreasing susceptibility of an mRNA to endonuclease activity, and/or increasing the half-life of an mRNA, wherein the mRNA comprises at least one endonuclease sensitive sequence motif in the ORF, the method comprising altering the at least one endonuclease sensitive sequence motif, thereby increasing the stability of an mRNA, increasing resistance and/or decreasing susceptibility of an mRNA to endonuclease activity, and/or increasing the half-life of an mRNA. In some aspects, the at least one endonuclease sensitive sequence motif comprises the nucleotide sequence AGA, wherein the endonuclease sensitive sequence motif is a codon comprising the ORF, wherein the codon encodes the amino acid arginine, and wherein altering the at least one sequence motif results in the formation of a degenerate codon encoding arginine, and wherein the codon and the degenerate codon are different. In some aspects, the degenerate codon is selected from the group consisting of: AGG, CGU, CGC, CGA, and CGG.

In any of the foregoing aspects, the disclosure provides a method of increasing stability of an mRNA, increasing resistance and/or decreasing susceptibility of an mRNA to endonuclease activity, and/or increasing the half-life of an mRNA, wherein the mRNA comprises at least one endonuclease sensitive sequence motif in the ORF, the method comprising altering the at least one endonuclease sensitive sequence motif, thereby increasing the stability of an mRNA, increasing resistance and/or decreasing susceptibility of an mRNA to endonuclease activity, and/or increasing the half-life of an mRNA. In some aspects, the at least one endonuclease sensitive sequence motif comprises the nucleotide sequence AGA, wherein the 5′ A is the 3^{rd} position (wobble position) of a codon comprising the ORF, wherein the codon encodes an amino acid selected from the group consisting of: lysine, threonine, arginine, isoleucine, glutamine, proline, leucine, glutamic acid, alanine, glycine, valine, and serine; wherein altering the at least one endonuclease sensitive sequence motif results in the formation of a degenerate codon encoding the amino acid, wherein the codon and the degenerate codon are different. In some aspects, the at least one endonuclease sensitive sequence motif comprises the nucleotide sequence UGA, wherein the U is the 3^{rd} position (wobble position) of a codon comprising the ORF, wherein the codon encodes an amino acid selected from the group consisting of: asparagine, threonine, serine, isoleucine, histidine, proline arginine, leucine, aspartic acid, alanine, glycine, valine, tyrosine, cysteine, and phenylalanine, wherein altering the at least one endonuclease sensitive sequence motif results in the formation of a degenerate codon encoding the amino acid, and wherein the codon and the degenerate codon are different. In some aspects, the at least one endonuclease sensitive sequence motif comprises the nucleotide sequence AGA, wherein the G is the 3^{rd} position (wobble position) of a codon comprising the ORF, wherein the codon encodes an amino acid selected from the group consisting of: lysine, glutamine, and glutamic acid, wherein altering the at least one endonuclease sensitive sequence motif results in the formation of degenerate codon encoding the amino acid, and wherein the codon and the degenerate codon are different. In some aspects, the at least one endonuclease sensitive sequence motif comprises the nucleotide sequence UGA, wherein the G is the 3^{rd} position (wobble position) of a codon comprising the ORF, wherein the codon encodes an amino acid selected from the group consisting of: leucine and valine, wherein altering the at least one endonuclease sensitive sequence motif results in the formation of a degenerate codon encoding the amino acid, and wherein the codon and the degenerate codon are different.

In any of the foregoing aspects, the disclosure provides a method of increasing stability of an mRNA, increasing resistance and/or decreasing susceptibility of an mRNA to endonuclease activity, and/or increasing the half-life of an mRNA, wherein the mRNA comprises at least one endonuclease sensitive sequence motif, the method comprising altering the at least one endonuclease sensitive sequence motif, thereby increasing the stability of an mRNA, increasing resistance and/or decreasing susceptibility of an mRNA to endonuclease activity, and/or increasing the half-life of an mRNA. In some aspects, altering the at least one endonuclease sensitive sequence motif increases stability of the mRNA. In some aspects, altering the at least one endonuclease sensitive sequence motif increases the half-life of the mRNA.

In any of the foregoing aspects, the disclosure provides a method of increasing stability of an mRNA, increasing resistance and/or decreasing susceptibility of an mRNA to endonuclease activity, and/or increasing the half-life of an mRNA, wherein the mRNA comprises at least one endonuclease sensitive sequence motif, the method comprising altering the at least one endonuclease sensitive sequence motif, and wherein increased resistance of the mRNA to endonuclease activity is determined relative to an unaltered mRNA, wherein the unaltered mRNA contains at least one additional endonuclease sensitive sequence motif relative to the altered mRNA.

In some aspects, the disclosure provides an mRNA produced by a method of increasing stability of an mRNA, increasing resistance and/or decreasing susceptibility of an mRNA to endonuclease activity, and/or increasing the half-life of an mRNA, wherein the mRNA comprises at least one endonuclease sensitive sequence motif, the method comprising altering the at least one endonuclease sensitive sequence motif, thereby increasing the stability of an mRNA, increasing resistance and/or decreasing susceptibility of an mRNA to endonuclease activity, and/or increasing the half-life of an mRNA.

In some aspects, the disclosure provides an mRNA comprising: a 5′ UTR, an ORF encoding a polypeptide, and a 3′ UTR, wherein the 3′ UTR comprises the nucleotide sequence of SEQ ID NO: 1, comprising from 5′ to 3′ a first endonuclease sensitive sequence motif (UGA1), a second endonuclease sensitive sequence motif (UGA2), and a third endonuclease sensitive sequence motif (UGA3), wherein at least one of UGA1, UGA2 and/or UGA3 is altered by deletion, substitution or insertion. In some aspects, UGA1 is altered by deletion. In some aspects, UGA2 is altered by a substitution. In some aspects, UGA3 is altered by a substitution. In some aspects, UGA2 is altered by substitution of G with cytosine (C), or adenine (A). In some aspects, UGA2 is altered by substitution of G with cytosine (C). In some aspects, UGA3 is altered by substitution of G with cytosine (C), or adenine (A). In some aspects, UGA3 is altered by substitution of G with cytosine (C). In some aspects, UGA2 and UGA3 are altered by substitution of G with cytosine (C), or adenine (A). In some aspects, UGA2 and UGA3 are altered by substitution of G with cytosine (C). In some aspects, the ORF encodes a polypeptide of interest. In some aspects, the polypeptide of interest is a therapeutic polypeptide. In some aspects, the 3′ UTR comprises a poly-A region. In some aspects, the 3′ UTR further comprises a terminal 3′-stabilizing region comprising 1 to 500 nucleosides. In some aspects, the 3′-stabilizing region comprises a plurality of alternative nucleosides. In some aspects, the alternative nucleoside is L-adenosine. In some aspects, the 3′-stabilizing region is conjugated to the 3′ UTR by a linker.

In some aspects, the disclosure provides an mRNA comprising: a 5′ UTR, an ORF encoding a polypeptide, and a 3′ UTR comprising the nucleotide sequence of SEQ ID NO: 2. In some aspects, the ORF encodes a polypeptide of interest. In some aspects, the polypeptide of interest is a therapeutic polypeptide. In some aspects, the 3′ UTR comprises a poly-A region. In some aspects, the 3′ UTR further comprises a terminal 3′-stabilizing region comprising 1 to 500 nucleosides. In some aspects, the 3′-stabilizing region comprises a plurality of alternative nucleosides. In some aspects, the alternative nucleoside is L-adenosine. In some aspects, the 3′-stabilizing region is conjugated to the 3′ UTR by a linker.

In any of the foregoing aspects, the disclosure provides a lipid nanoparticle comprising an mRNA altered by a method of the disclosure for increased stability and/or potency. In some aspects, a pharmaceutical composition comprises the mRNA or the lipid nanoparticle comprising the mRNA, and a pharmaceutically acceptable carrier, diluent or excipient.

In any of the foregoing aspects, the disclosure provides a lipid nanoparticle comprising an mRNA altered by a method of the disclosure for increased stability and/or potency, and an optional pharmaceutically acceptable carrier, diluent, or excipient, or a pharmaceutical composition comprising the lipid nanoparticle, is for use in treating or delaying progression of a disease or disorder in a subject, wherein the treating or delaying progression of the disease or disorder comprises administration of the lipid nanoparticle, and an optional pharmaceutically acceptable carrier, diluent, or excipient, or the pharmaceutical composition.

In any of the foregoing aspects, the disclosure provides a lipid nanoparticle comprising an mRNA altered by a method of the disclosure for increased stability and/or potency, and an optional pharmaceutically acceptable carrier, diluent, or excipient, or a pharmaceutical composition comprising the lipid nanoparticle, is for use in the manufacture of a medicament for treating or delaying progression of a disease or disorder in a subject, wherein the medicament comprises the lipid nanoparticle, and an optional pharmaceutically acceptable carrier, or the pharmaceutical composition and wherein the treatment comprises administration of the medicament.

In any of the foregoing aspects, the disclosure provides a kit comprising a container comprising an mRNA altered by a method of the disclosure for increased stability and/or potency, and an optional pharmaceutically acceptable carrier, a lipid nanoparticle comprising the mRNA and an optional pharmaceutically acceptable carrier, or a pharmaceutical composition comprising the mRNA or the lipid nanoparticle comprising the mRNA, and an optional pharmaceutically acceptable carrier, and a package insert comprising instructions for administration of the mRNA, the lipid nanoparticle, or the pharmaceutical composition for treating or delaying progression of a disease or disorder in a subject.

In some aspects, the disclosure provides a method of treating or delaying progression of a disease or disorder in a subject in need thereof, the method comprising administering an mRNA altered by a method of the disclosure for increased stability and/or potency, and an optional pharmaceutically acceptable carrier, a lipid nanoparticle comprising the mRNA and an optional pharmaceutically acceptable carrier, or a pharmaceutical composition comprising the mRNA or the lipid nanoparticle comprising the mRNA, and an optional pharmaceutically acceptable carrier, thereby treating or delaying progression of the disease or disorder in the subject.

### BRIEF DESCRIPTION OF THE DRAWINGS

FIG. 1 provides a schematic depicting a proposed mechanism of endonuclease-mediated mRNA degradation.

FIG. 2 provides a schematic depicting the experimental workflow of the 5PSeq RNA sequencing method for sequencing 5′ phosphorylated (5′P) mRNA decay intermediates to determine the locations of endonuclease cleavage on mRNA. Mapping 5′P data to the transcriptome identifies the nucleotide sequence of the mRNA that surrounds endonuclease cleavage sites.

FIG. 3A provides an RNASeq and 5PSeq assembly graph showing RNASeq and 5PSeq read counts for a representative gene (HSD3B7 gene encoding 3 beta-hydroxysteroid dehydrogenase type 7) from mouse liver and mouse AML12 cell transcriptome sequencing data. FIG. 3B provides a sequence logo depicting a consensus sequence motif located upstream of endonuclease cleavage sites found in the transcriptomes of mouse liver and mouse AML12 cells, as determined by 5PSeq.

FIG. 4A provides a 5PSeq assembly graph of 5PSeq RNA sequence data showing read counts corresponding to each position on an in vitro transcribed mRNA encoding GFP, following its transfection into AML12 cells. The four most abundant 5P species are indicated with black arrows. FIG. 4B provides a schematic depicting the GFP construct used in FIG. 4A. Sequences surrounding the most abundant 5P species are indicated. 3 of 4 sequences conform to endonuclease sensitive motif identified in transcriptomic data (FIG. 3B).

FIG. 5A provides a schematic depicting the steps of the canonical 5′ exonuclease-mediated mRNA degradation pathway. FIGS. 5B and 5C show a 5PSeq assembly graph of 5PSeq RNA sequence data showing that read counts corresponding to 5′ phosphorylated mRNA degradation products are similar for an mRNA encoding GFP having a hydrolysable 5′ cap (Cap 1; FIG. 5B) or a non-hydrolyzable 5′ cap (Cap 2; FIG. 5C).

FIG. 6A provides a graph depicting the eGFP fluorescent signal from cells transfected with an eGFP-encoding mRNA containing a 5′ cap that allows translation of the mRNA (Cap 1 from FIG. 5B) and with an eGFP-encoding mRNA containing a 5′ cap that blocks translation of the mRNA (PglycS NoMe). FIGS. 6B and 6C show a 5PSeq assembly graph of 5PSeq RNA sequence data showing that read counts corresponding to 5′ phosphorylated mRNA degradation products are similar for an mRNA encoding GFP having a 5′ cap that allows translation (Cap 1; FIG. 6B) or a 5′ cap that blocks translation (PglycS NoMe; FIG. 6C)

FIG. 7A provides a schematic depicting an endonuclease sensitive GFP-degron-encoding mRNA construct with an A100 poly(A) tail and a series of mRNA construct variants derived from the endonuclease sensitive GFP-degron-encoding mRNA construct, indicating the mRNA components (5′ UTR, ORF, 3′ UTR) that have been substituted to remove endonuclease sensitive sequence motifs. (Endo Sensitive=no substitution of endonuclease sensitive sequence motifs (grey); Variant 1=substitution of sequences in 3′ UTR (white with black outline); Variant 2,3,4=substitution of sequences in 5′ UTR (grey with black outline), ORF (dark grey, black, and white), and 3′ UTR (white with black outline); Variant 5=substitution of sequences in 5′ UTR (grey with black outline); Variant 6 and Variant 7=substitution of sequences in ORF (grey with white text and white with black text). FIG. 7B provides a graph depicting the GFP-degron fluorescent signal over time in mouse AML12 cells electroporated with the GFP-degron-encoding mRNA constructs, as indicated. FIG. 7C provides a graph depicting the functional half-life of the mRNA constructs following electroporation into AML12 cells, as indicated.

FIG. 8A provides a schematic depicting an endonuclease sensitive GFP-degron-encoding mRNA construct with an 3′stabilizing region attached to the A100 poly(A) tail (e.g., LA10 tail), and a series of mRNA construct variants derived from the endonuclease sensitive GFP-degron-encoding mRNA construct indicating the mRNA components (5′ UTR, ORF, 3′ UTR) that have been substituted to remove endonuclease sensitive sequence motifs. (Endo Sensitive=no substitution of endonuclease sensitive sequence motifs (grey); Variant 1=substitution of 3′ UTR (white with black outline); Variant 2, Variant 3, Variant 4=substitution of endonuclease sensitive sequences in 5′ UTR (grey with black outline), ORF (dark grey, black, and white), and 3′ UTR (white with black outline); Variant 5=substitution of endonuclease sensitive sequence in 5′ UTR (grey with black outline); Variant 6 and Variant 7=substitutions of endonuclease sensitive sequences in the ORF (grey with white text and white with black text). FIG. 8B provides a graph depicting the GFP-degron fluorescent signal over time in mouse AML12 cells electroporated with the GFP-degron-encoding mRNA constructs, as indicated. FIG. 8C provides a graph depicting the functional half-life of the mRNA constructs following electroporation into AML12 cells, as indicated.

### DETAILED DESCRIPTION

Without being bound by theory, it is believed that the number, position, or both the number and position of the endonuclease sensitive sequence motifs described herein affects the susceptibility of mRNA molecules to nucleases and that altering the number, position, or both the number and position of such endonuclease sensitive sequence motifs increases or improves the stability of the mRNA, by, for example, increasing half-life, increasing potency, decreasing susceptibility to endonucleases and/or increasing resistance of the mRNA to endonucleases. Further, altering endonuclease sensitive sequence motifs in an mRNA provides a means to control the level of resistance of the mRNA to nucleases and thereby control the expression of the mRNA in a cell.

Accordingly, the disclosure provides methods for identifying and altering endonuclease sensitive sequence motifs in an mRNA, methods for generating an mRNA having increased or improved stability, methods for generating an mRNA having increased or improved half-life, methods for generating an mRNA having increased or improved potency, methods for generating an mRNA having decreased susceptibility to endonucleases, and methods for generating an endonuclease-resistant mRNA from an endonuclease-susceptible mRNA comprising at least one or more endonuclease sensitive sequence motifs, wherein the methods generally comprise altering the number and/or position of the endonuclease sensitive sequence motifs in the mRNA. The disclosure also provides methods of using the resulting mRNA molecules in therapy.

Methods of Increasing Stability of mRNA

The disclosure generally provides methods of increasing or improving stability of an mRNA by increasing or improving resistance of the mRNA to endonuclease activity. In some embodiments, the methods provided by the disclosure relate to increasing or improving endonuclease resistance, increasing or improving stability, increasing or improving half-life, and/or decreasing susceptibility of an mRNA to endonucleases. In some embodiments, the methods provided by the disclosure relate to increasing the potency of an mRNA resulting from increasing the endonuclease resistance of the mRNA.

In some aspects, the disclosure provides a method of increasing stability of an mRNA, comprising:

(i) providing an mRNA comprising at least one endonuclease sensitive sequence motif, wherein the endonuclease sensitive sequence motif comprises the nucleotide sequence WGA, wherein W=adenine (A) or uracil (U); and

(ii) altering the at least one endonuclease sensitive sequence motif, such that stability of the mRNA is increased.

In some embodiments, the disclosure provides a method of increasing the resistance or decreasing susceptibility of an mRNA to endonuclease activity, wherein the mRNA comprises at least one endonuclease sensitive sequence motif, wherein the endonuclease sensitive sequence motif comprises the nucleotide sequence WGA, wherein W=adenine (A) or uracil (U), the method comprising altering the at least one endonuclease sensitive sequence motif, thereby increasing the resistance or decreasing susceptibility of the mRNA to endonuclease activity.

In some embodiments, the disclosure provides a method of increasing the half-life of an mRNA, wherein the mRNA comprises at least one endonuclease sensitive sequence motif, wherein the endonuclease sensitive sequence motif comprises the nucleotide sequence WGA, wherein W=adenine (A) or uracil (U), the method comprising altering the at least one endonuclease sensitive sequence motif, thereby increasing the half-life of the mRNA.

In some embodiments, altering (e.g., by substitution, insertion or deletion) the at least one endonuclease sensitive sequence motif increases or improves stability of the mRNA. In some embodiments, an increase or improvement in stability of the mRNA is determined by reference to an unaltered mRNA counterpart. In some embodiments, the stability of an mRNA is increased by at least about 5%, 10%, 20%, 30%, 40%, 50%, 60%, 70%, 80%, 90%, 100%, 105%, 110%, 115%, 120%, 125%, 130%, 135%, 140%, 150%, 160%, 170%, 175%, 180%, 190%, 200%, 250%, 300%, 350%, 400%, 450%, 500%, 600%, 700%, 800%, 900%, 1,000% or more relative to an unaltered mRNA counterpart. In some embodiments, the stability of the mRNA is increased by at least 1-fold, 2-fold, 3-fold, 4-fold, 5-fold, 6-fold, 7-fold, 8-fold, 9-fold, 10-fold or more relative to an unaltered mRNA counterpart.

In some embodiments, altering (e.g., by substitution, insertion or deletion) the at least one endonuclease sensitive sequence motif increases the half-life of the mRNA. In some embodiments, an increase in the half-life of the mRNA is determined by reference to an unaltered mRNA counterpart. In some embodiments, the half-life of an mRNA is increased by at least about 5%, 10%, 20%, 30%, 40%, 50%, 60%, 70%, 80%, 90%, 100%, 105%, 110%, 115%, 120%, 125%, 130%, 135%, 140%, 150%, 160%, 170%, 175%, 180%, 190%, 200%, 250%, 300%, 350%, 400%, 450%, 500%, 600%, 700%, 800%, 900%, 1,000% or more relative to an unaltered mRNA counterpart. In some embodiments, the half-life of the mRNA is increased by at least 1-fold, 2-fold, 3-fold, 4-fold, 5-fold, 6-fold, 7-fold, 8-fold, 9-fold, 10-fold or more relative to an unaltered mRNA counterpart.

In some embodiments, altering (e.g., by substitution, insertion or deletion) the at least one endonuclease sensitive sequence motif increases resistance of the mRNA to endonuclease activity. In some embodiments, an increase in resistance of the mRNA to endonuclease activity is determined by reference to an unaltered mRNA counterpart. In some embodiments, the endonuclease resistance of the mRNA is increased by at least about 5%, 10%, 20%, 30%, 40%, 50%, 60%, 70%, 80%, 90%, 100%, 105%, 110%, 115%, 120%, 125%, 130%, 135%, 140%, 150%, 160%, 170%, 175%, 180%, 190%, 200%, 250%, 300%, 350%, 400%, 450%, 500%, 600%, 700%, 800%, 900%, 1,000% or more relative to an unaltered mRNA counterpart. In some embodiments, the resistance of the mRNA to endonuclease activity is increased by at least 1-fold, 2-fold, 3-fold, 4-fold, 5-fold, 6-fold, 7-fold, 8-fold, 9-fold, 10-fold or more relative to an unaltered mRNA counterpart.

In some embodiments, altering (e.g., by substitution, insertion or deletion) the at least one endonuclease sensitive sequence motif decreases susceptibility of the mRNA to endonuclease activity. In some embodiments, a decrease in susceptibly of the mRNA to endonuclease acidity is determined by reference to an unaltered mRNA counterpart. In some embodiments, susceptibility of the mRNA to endonuclease activity is decreased by at least about 5%, 10%, 20%, 30%, 40%, 50%, 60%, 70%, 80%, 90%, 100%, 105%, 110%, 115%, 120%, 125%, 130%, 135%, 140%, 150%, 160%, 170%, 175%, 180%, 190%, 200%, 250%, 300%, 350%, 400%, 450%, 500%, 600%, 700%, 800%, 900%, 1,000% or more relative to an unaltered mRNA counterpart. In some embodiments, the susceptibly of the mRNA to endonuclease activity is decreased by at least 1-fold, 2-fold, 3-fold, 4-fold, 5-fold, 6-fold, 7-fold, 8-fold, 9-fold, 10-fold or more relative to an unaltered mRNA counterpart.

In some embodiments, the unaltered mRNA counterpart contains at least one or more additional endonuclease sensitive sequence motifs relative to the stabilized mRNA (e.g., 1, 2, 3, 4, 5, 6, 7, 8, 9, 10, 11, 12, 13, 14, 15, 16, 17, 18, 19, 20 or more endonuclease sensitive sequence motifs). In some embodiments, the unaltered mRNA counterpart contains at least one additional endonuclease sensitive sequence motif relative to the stabilized, therapeutic mRNA. In some embodiments, the unaltered mRNA counterpart contains at least two additional endonuclease sensitive sequence motifs relative to the stabilized mRNA. In some embodiments, the unaltered mRNA counterpart contains at least three additional endonuclease sensitive sequence motifs relative to the stabilized mRNA. In some embodiments, the unaltered mRNA counterpart contains at least four additional endonuclease sensitive sequence motifs relative to the stabilized mRNA. In some embodiments, the unaltered mRNA counterpart contains at least five additional endonuclease sensitive sequence motifs relative to the stabilized mRNA. In some embodiments, the unaltered mRNA counterpart contains at least six additional endonuclease sensitive sequence motifs relative to the stabilized mRNA. In some embodiments, the unaltered mRNA counterpart contains at least seven additional endonuclease sensitive sequence motifs relative to the stabilized mRNA. In some embodiments, the unaltered mRNA counterpart contains at least eight additional endonuclease sensitive sequence motifs relative to the stabilized mRNA. In some embodiments, the unaltered mRNA counterpart contains at least nine additional endonuclease sensitive sequence motifs relative to the stabilized mRNA. In some embodiments, the unaltered mRNA counterpart contains at least ten additional endonuclease sensitive sequence motifs relative to the stabilized mRNA.

In some embodiments, the mRNA comprises at least one or more endonuclease sensitive sequence motifs (e.g., 2, 3, 4, 5, 6, 7, 8, 9, 10, 11, 12, 13, 14, 15, 16, 17, 18, 19, 20 or more motifs). In some embodiments, the mRNA comprises more than one endonuclease sensitive sequence motif and the nucleotide sequence of each of the endonuclease sensitive sequence motifs is the same (i.e., identical). In some embodiments, the mRNA comprises more than one endonuclease sensitive sequence motif and the nucleotide sequence of each of the endonuclease sensitive sequence motifs is different. In some embodiments, the mRNA comprises more than one endonuclease sensitive sequence motif (e.g., 3 motifs), and one or more of the nucleotide sequences of the endonuclease sensitive sequence motifs (e.g. 2 motifs) are the same (i.e., identical). In some embodiments, the mRNA comprises more than one endonuclease sensitive sequence motif (e.g., 3 motifs), and one or more of the nucleotide sequences of the endonuclease sensitive sequence motifs (e.g. 2 motifs) are different.

In some embodiments, a non-coding region, a coding region, or a combination thereof, present in the starting, unaltered mRNA comprises at least one endonuclease sensitive sequence motif. In some embodiments, the mRNA comprises a 5′ untranslated region (5′ UTR), an open reading frame (ORF) encoding a polypeptide, and a 3′ UTR, wherein one or more of the 5′ UTR, ORF and/or 3′UTR comprises at least one endonuclease sensitive sequence motif. In one embodiment, at least one of the 5′ UTR, ORF and/or 3′ UTR comprise at least one endonuclease sensitive sequence motif. In one embodiment, the 5′ UTR comprises at least one endonuclease sensitive sequence motif. In one embodiment, the ORF comprises at least one endonuclease sensitive sequence motif. In one embodiment, the 3′ UTR comprises at least one endonuclease sensitive sequence motif. In one embodiment, the 5′ UTR, ORF and 3′ UTR each comprise at least one endonuclease sensitive sequence motif.

In some embodiments, the 5′UTR and ORF each comprise at least one endonuclease sensitive sequence motif, wherein the at least one endonuclease sensitive sequence motif is the same or is different. In some embodiments, the 5′UTR and 3′UTR each comprise at least one endonuclease sensitive sequence motif, wherein the at least one endonuclease sensitive sequence motif is the same or is different. In some embodiments, the ORF and the 3′UTR each comprise at least one endonuclease sensitive sequence motif, wherein the at least one endonuclease sensitive sequence motif is the same or is different. In some embodiments, the 5′UTR, ORF and 3′UTR each comprise at least one endonuclease sensitive sequence motif, wherein the at least one endonuclease sensitive sequence motif is the same or is different.

In some embodiments, the mRNA comprises at least one endonuclease sensitive sequence motif in a 5′UTR, ORF, and/or 3′UTR, wherein the endonuclease sensitive sequence motif is WGA, wherein W=adenine (A). In some embodiments, mRNA comprises at least one endonuclease sensitive sequence motif in a 5′UTR, ORF, and/or 3′UTR, wherein the endonuclease sensitive sequence motif is WGA, wherein W=uracil (U). In some embodiments, mRNA comprises at least one endonuclease sensitive sequence motif in a 5′UTR, ORF, and/or 3′UTR, wherein the endonuclease sensitive sequence motif is AGA or UGA. In some embodiments, the mRNA comprises at least one endonuclease sensitive sequence motif in a 5′UTR, ORF, and/or 3′UTR, wherein the endonuclease sensitive sequence motif is AGA. In some embodiments, the mRNA comprises at least one endonuclease sensitive sequence motif in a 5′UTR, ORF, and/or 3′UTR, wherein the endonuclease sensitive sequence motif is UGA.

In some embodiments, altering the at least one endonuclease sensitive sequence motif comprises introducing a substitution, insertion, deletion or chemical modification of at least one nucleotide comprising the endonuclease sensitive sequence motif. In some embodiments, altering the at least one endonuclease sensitive sequence motif comprises substitution of one or more nucleotides of the endonuclease sensitive motif with one or more different nucleotides, deleting one or more nucleotides from the endonuclease sensitive motif, replacing all of the nucleotides of the endonuclease sensitive motif with different nucleotides, deleting all of the nucleotides of the endonuclease sequence motif or a combination thereof (e.g., when there are two or more endonuclease sensitive sequence motifs in the mRNA) to alter, delete or replace one or more endonuclease sensitive motifs in the mRNA. In one embodiment, the at least one endonuclease sensitive sequence motif is altered by substitution of one or more nucleotides of the endonuclease sensitive sequence motif. In one embodiment, the at least one endonuclease sensitive sequence motif is altered by deletion of one or more nucleotides of the endonuclease sensitive sequence motif. In one embodiment, the at least one endonuclease sensitive sequence motif is altered by replacement of all of nucleotides of the endonuclease sensitive sequence motif with alternative nucleotides. In one embodiment, the at least one endonuclease sensitive sequence motif is altered by deletion of all of the nucleotides of the endonuclease sensitive sequence motif. In one embodiment, altering the at least one endonuclease sensitive sequence motif comprises chemically modifying at least one nucleotide of the endonuclease sensitive sequence motif.

In one embodiment, the disclosure provides an mRNA comprising at least one endonuclease sensitive sequence motif, wherein the endonuclease sensitive sequence motif is WGA, wherein W=adenine (A), and wherein W is substituted with a cytosine (C), guanine (G), or uracil (U). In one embodiment, the mRNA comprises at least one endonuclease sensitive sequence motif in the 5′UTR, wherein the endonuclease sensitive sequence motif is WGA, wherein W=adenine (A), wherein W is substituted with a cytosine (C), guanine (G), or uracil (U). In one embodiment, the mRNA comprises at least one endonuclease sensitive sequence motif in the ORF, wherein the endonuclease sensitive sequence motif is WGA, wherein W=adenine (A), wherein W is substituted with a cytosine (C), guanine (G), or uracil (U). In one embodiment, the mRNA comprises at least one endonuclease sensitive sequence motif in the 3′UTR, wherein the endonuclease sensitive sequence motif is WGA, wherein W=adenine (A), wherein W is substituted with a cytosine (C), guanine (G), or uracil (U). In one embodiment, the mRNA comprises at least one endonuclease sensitive sequence motif in each of the 5′UTR and the ORF, wherein the endonuclease sensitive sequence motif is WGA, wherein W=adenine (A), wherein W is substituted with a cytosine (C), guanine (G), or uracil (U). In one embodiment, the mRNA comprises at least one endonuclease sensitive sequence motif in each of the ORF and 3′UTR, wherein the endonuclease sensitive sequence motif is WGA, wherein W=adenine (A), wherein W is substituted with a cytosine (C), guanine (G), or uracil (U). In one embodiment, the mRNA comprises at least one endonuclease sensitive sequence motif in each of the 5′UTR, ORF and 3′UTR, wherein the endonuclease sensitive sequence motif is WGA, wherein W=adenine (A), wherein W is substituted with a cytosine (C), guanine (G), or uracil (U). In one embodiment, substitution of nucleotide in the endonuclease sensitive sequence motif increases or improves the stability of the mRNA, increases or improves mRNA half-life, increases or improves mRNA potency, decreases or reduces susceptibility of the mRNA to endonuclease activity, and/or increases or improves resistance of the mRNA to endonuclease activity.

In one embodiment, the disclosure provides an mRNA comprising at least one endonuclease sensitive sequence motif, wherein the endonuclease sensitive sequence motif is WGA, wherein W=uracil (U), and wherein W is substituted with a cytosine (C), guanine (G), or adenine (A). In one embodiment, the mRNA comprises at least one endonuclease sensitive sequence motif in the 5′UTR, wherein the endonuclease sensitive sequence motif is WGA, wherein W=uracil (U), and wherein W is substituted with a cytosine (C), guanine (G), or adenine (A). In one embodiment, the mRNA comprises at least one endonuclease sensitive sequence motif in the ORF, wherein the endonuclease sensitive sequence motif is WGA, wherein W=uracil (U), and wherein W is substituted with a cytosine (C), guanine (G), or adenine (A). In one embodiment, the mRNA comprises at least one endonuclease sensitive sequence motif in the 3′UTR, wherein the endonuclease sensitive sequence motif is WGA, wherein W=uracil (U), and wherein W is substituted with a cytosine (C), guanine (G), or adenine (A). In one embodiment, the mRNA comprises at least one endonuclease sensitive sequence motif in each of the 5′UTR and ORF, wherein the endonuclease sensitive sequence motif is WGA, wherein W=uracil (U), and wherein W is substituted with a cytosine (C), guanine (G), or adenine (A). In one embodiment, the mRNA comprises at least one endonuclease sensitive sequence motif in the each of the ORF and 3′ UTR, wherein the endonuclease sensitive sequence motif is WGA, wherein W=uracil (U), and wherein W is substituted with a cytosine (C), guanine (G), or adenine (A). In one embodiment, the mRNA comprises at least one endonuclease sensitive sequence motif in each of the 5′UTR, ORF and 3′UTR, wherein the endonuclease sensitive sequence motif is WGA, wherein W=uracil (U), and wherein W is substituted with a cytosine (C), guanine (G), or adenine (A). In one embodiment, substitution of nucleotide in the endonuclease sensitive sequence motif increases or improves the stability of the mRNA, increases or improves mRNA half-life, increases or improves mRNA potency, decreases or reduces susceptibility of the mRNA to endonuclease activity, and/or increases or improves resistance of the mRNA to endonuclease activity.

In some embodiments, the disclosure provides an mRNA comprising at least one endonuclease sensitive sequence motif, wherein the at least one endonuclease sensitive sequence motif comprises the nucleotide sequence WGA, wherein W=adenine (A), and wherein W is substituted with a cytosine (C), guanine (G), or uracil (U). In some embodiments, an mRNA of the disclosure comprises at least one endonuclease sensitive sequence motif in the ORF, wherein the at least one endonuclease sensitive sequence motif comprises the nucleotide sequence WGA, wherein W=adenine (A), and wherein W is substituted with a cytosine (C), guanine (G), or uracil (U). In some embodiments, an mRNA of the disclosure comprises at least one endonuclease sensitive sequence motif in the ORF comprising the nucleotide sequence AGA, wherein the nucleotide sequence AGA is a codon encoding Arg in the first reading frame of the ORF, and wherein the nucleotide sequence AGA is substituted with a degenerate codon that encodes Arg (e.g., CGT, CGC, CGA, or CGG). In some embodiments, an mRNA of the disclosure comprises at least one endonuclease sensitive sequence motif in the ORF comprising the nucleotide sequence AGA, wherein the nucleotide sequence AGA is not a codon in the first reading frame of the ORF, and wherein the nucleotide sequence AGA is altered by substitution according to Table 1, thereby increasing or improving the mIRNA stability, increasing or improving mRNA half-life, and/or increasing or improving mRNA resistance to endonuclease activity.

TABLE 1
Altering an endonuclease sensitive sequence motif
comprising the nucleotide sequence AGA

| | | Encoded |
|---|---|---|
| Unaltered | | Amino |
| Sequence | Altered Sequences | Acid |
| AAA\|GAN | AAG\|GAN | Lys-X |
| ACA\|GAN | ACC\|GAN, ACU\|GAN, ACG\|GAN | Thr-X |
| AGA\|GAN | AGG\|GAN, CGC\|GAN, CCG\|GAN | Arg-X |
| AUA\|GAN | AUC\|GAN | Ile-X |
| CAA\|GAN | CAG\|GAN | Gln-X |
| CCA\|GAN | CCC\|GAN, CCG\|GAN | Pro-X |
| CGA\|GAN | AGG\|GAN, CGC\|GAN, CGG\|GAN | Arg-X |
| CUA\|GAN | CUG\|GAN, CUC\|GAN | Leu-X |
| GAA\|GAN | GAG\|GAN | Glu-X |
| GCA\|GAN | GCC\|GAN, GCG\|GAN | Ala-X |
| GGA\|GAN | GGC\|GAN, GGG\|GAN | Gly-X |
| GUA\|GAN | GUG\|GAN, GUC\|GAN | Val-X |
| UAA\|GAN | UAG\|GAN | Stop-X |
| UCA\|GAN | AGC\|GAN, UCC\|GAN, UCG\|GAN | Ser-X |
| UGA\|GAN | UAG\|GAN | Stop-X |
| UUA\|GAN | CUG\|GAN, UUG\|GAN, CUC\|GAN | Leu-X |
| AAG\|ANN | AAA\|ANN | Lys-Y |
| CAG\|ANN | CAA\|ANN | Gln-Y |
| GAG\|ANN | GAA\|ANN | Glu-Y |
| UAG\|ANN | UAA\|ANN | Stop-Y |

(endonuclease sensitive sequence motif before altering by substitution shown in underline; following altering by substitution shown in bold; N = A, U, G, C; X = Asp, Glu; Y = Ile, Met, Thr, Asn, Lys, Ser, Arg)

In some embodiments, the disclosure provides an mIRNA comprising at least one endonuclease sensitive sequence motif, wherein the at least one endonuclease sensitive sequence motif comprises the nucleotide sequence WGA, wherein W=uracil (U), and wherein W is substituted with a cytosine (C), guanine (G), or adenine (A). In some embodiments, an mRNA of the disclosure comprises at least one endonuclease sensitive sequence motif in the ORF, wherein the at least one endonuclease sensitive sequence motif comprises the nucleotide sequence WGA, wherein W=uracil (U), and wherein W is substituted with a cytosine (C), guanine (G), or adenine (A). In some embodiments, an mRNA of the disclosure comprises at least one endonuclease sensitive sequence motif in the ORF comprising the nucleotide sequence UGA, wherein the nucleotide sequence UGA is not a codon in the first reading frame of the ORF, and wherein the nucleotide sequence UGA is altered by substitution according to Table 2, thereby increasing or improving the miRNA stability, increasing or improving miRNA half-life, and/or increasing or improving miRNA resistance to endonuclease activity.

TABLE 2
Altering an endonuclease sensitive sequence motif
comprising the nucleotide sequence UGA

| | | Encoded |
|---|---|---|
| Unaltered | Altered | Amino |
| Sequence | Sequences | Acid |
| AAU\|GAN | AAC\|GAN | Asn-X, |
| ACU\|GAN | ACC\|GAN, ACG\|GAN | Thr-X |
| AGU\|GAN | AGC\|GAN, UCC\|GAN, UCG\|GAN | Ser-X |
| AUU\|GAN | AUC\|GAN | Ile-X |
| CAU\|GAN | CAC\|GAN | His-X |
| CCU\|GAN | CCC\|GAN, CCG\|GAN | Pro-X |
| CGU\|GAN | AGG\|GAN, CGC\|GAN, CGG\|GAN | Arg-X |
| CUU\|GAN | CUG\|GAN, CUC\|GAN, UUG\|GAN | Leu-X |
| GAU\|GAN | GAC\|GAN | Asp-X |
| GCU\|GAN | GCC\|GAN, GCG\|GAN | Ala-X |
| GGU\|GAN | GGC\|GAN, GGG\|GAN | Gly-X |
| GUU\|GAN | GUG\|GAN, GUC\|GAN | Val-X |
| UAU\|GAN | UAC\|GAN | Tyr-X |
| UCU\|GAN | AGC\|GAN, UCC\|GAN, UCG\|GAN | Ser-X |
| UGU\|GAN | UGC\|GAN | Cys-X |
| UUU\|GAN | UUC\|GAN | Phe-X |
| CUG\|ANN | CUC\|ANN, CUU\|ANN, CUA\|ANN, | Leu-Y |
| | UUAIANN | |
| GUG\|ANN | GUC\|ANN, GUU\|ANN, GUA\|ANN | Val-Y |
| UUG\|ANN | CUC\|ANN, UUA\|ANN, CUU\|ANN, | Leu-Y |
| | CUA\|ANN | |

(endonuclease sensitive sequence motif before altering by substitution shown in underline; following altering by substitution shown in bold; N = A, U, G, C; X = Asp, Glu; Y = Ile, Met, Thr, Asn, Lys, Ser, Arg)

In some embodiments, an mRNA of the disclosure comprises at least one endonuclease sensitive sequence motif in the 3′UTR comprising the nucleotide sequence UGA, wherein the nucleotide sequence UGA is positioned at the 5′end of the 3′UTR and is a stop codon in the first reading frame of the ORF, and wherein the nucleotide sequence UGA is substituted with a degenerate codon that is a stop codon (e.g., UAA, UAG). In some embodiments, an mIRNA of the disclosure comprises at least one endonuclease sensitive sequence motif in the 3′UTR comprising the nucleotide sequence UGA, wherein the nucleotide sequence UGA is positioned at the 5′end of the 3′UTR and is a first stop codon in the first reading frame of the ORF, and wherein the nucleotide sequence UGA is altered by deletion.

In one embodiment the disclosure provides an mRNA comprising at least one endonuclease sensitive sequence motif, wherein the at least one endonuclease sensitive sequence motif comprises WGA, wherein W=adenine (A), wherein W is preceded by an AG, and wherein the AG is substituted with a GC. In some embodiments, the disclosure provides an mRNA comprising at least one endonuclease sensitive sequence motif, wherein the at least one endonuclease sensitive sequence motif is WGA, wherein W=adenine (A), wherein W is altered by substitution with a cytosine (C), guanine (G), or uracil (U), wherein W is preceded by an AG, and wherein the AG is substituted with a GC. In some embodiments, the disclosure provides an mRNA comprising at least one endonuclease sensitive sequence motif, wherein the at least one endonuclease sensitive sequence motif is WGA, wherein W=adenine (A), wherein W is substituted with a guanine (G), wherein W is preceded by a CG, and wherein the C is substituted with an A. In some embodiments, the disclosure provides an mRNA comprising at least one endonuclease sensitive sequence motif, wherein the at least one endonuclease sensitive sequence motif is WGA, wherein W=adenine (A), wherein W is substituted with a cytosine (C) or uracil (U), wherein W is preceded by a UC, and wherein the UC is substituted with an AG. In some embodiments, the disclosure provides an mRNA comprising at least one endonuclease sensitive sequence motif, wherein the at least one endonuclease sensitive sequence motif is WGA, wherein W=adenine (A), wherein W is substituted with a cytosine (C), guanine (G), or uracil (U), wherein W is preceded by a UU, and wherein the first U comprising the UU is substituted with a C.

In some embodiments, the disclosure provides an mRNA comprising at least one endonuclease sensitive sequence motif, wherein the at least one endonuclease sensitive sequence motif is WGA, wherein W=uracil (U), wherein W is substituted with a cytosine (C), guanine (G), or adenine (A), wherein W is preceded by an AG, and wherein the AG is substituted with a UC. In some embodiments, the disclosure provides an mRNA comprising at least one endonuclease sensitive sequence motif, wherein the at least one endonuclease sensitive sequence motif is WGA, wherein W=uracil (U), wherein W is substituted with an adenine (A) or guanine (G), wherein W is preceded by a CU, and wherein the C is substituted with a U. In some embodiments, the disclosure provides an mRNA comprising at least one endonuclease sensitive sequence motif, wherein the at least one endonuclease sensitive sequence motif is WGA, wherein W=uracil (U), wherein W is substituted with a cytosine (C), wherein W is preceded by a UC, and wherein the UC is substituted with an AG.

In some embodiments, the disclosure provides an mRNA comprising at least one endonuclease sensitive sequence motif, wherein the at least one endonuclease sensitive sequence motif is WGA, wherein W=adenine (A), and wherein the adenine (A) following the guanine (G) of the endonuclease sensitive sequence motif is substituted with a guanine (G).

In some embodiments, the disclosure provides an mRNA comprising at least one endonuclease sensitive sequence motif, wherein the at least one endonuclease sensitive sequence motif comprises a stop codon UGA, wherein the G is substituted with A, thereby forming the stop codon UAA. In some embodiments, the disclosure provides an mRNA comprising at least one endonuclease sensitive sequence motif, wherein the at least one endonuclease sensitive sequence motif comprises the stop codon UGA, wherein the GA is mutated by substitution to an AG, thereby forming the stop codon UAG.

In some embodiments, the disclosure provides an mRNA comprising at least one endonuclease sensitive sequence motif, wherein the endonuclease sensitive sequence motif is about 3, about 3-4, about 4-6, about 6-10, about 3, 4, 5, 6, 7, 8, 9, or 10 nucleotides in length. In some embodiments, the endonuclease sensitive sequence motif is 3 nucleotides in length. In some embodiments, the endonuclease sensitive sequence motif is 4 nucleotides in length.

In some embodiments, the disclosure provides an mRNA comprising at least one endonuclease sensitive sequence motif, wherein the at least one endonuclease sensitive sequence motif comprises the nucleotide sequence AGA, wherein the endonuclease sensitive sequence motif is present in the ORF, wherein the motif is present in a codon encoding the amino acid arginine, and wherein altering the at least one endonuclease sensitive sequence motif results in the formation of a degenerate codon encoding arginine, wherein the codon and the degenerate codon are different. In some embodiments, the degenerate codon is selected from the group consisting of: AGG, CGU, CGC, CGA, and CGG. In some embodiments, the disclosure provides an mRNA comprising at least one endonuclease sensitive sequence motif, wherein the at least one endonuclease sensitive sequence motif comprises the nucleotide sequence AGA, wherein the endonuclease sensitive sequence motif is present in the ORF, wherein the motif is present in a codon encoding the amino acid arginine, and wherein the endonuclease sensitive sequence motif is altered to a degenerate codon is selected from the group consisting of: AGG, CGU, CGC, CGA, and CGG.

In some embodiments, the disclosure provides an mRNA comprising at least one endonuclease sensitive sequence motif, wherein the at least one endonuclease sensitive sequence motif comprises the nucleotide sequence AGA, wherein the 5′ A is the 3rd position (wobble position) of a codon comprising the ORF, wherein the codon encodes an amino acid selected from the group consisting of: lysine, threonine, arginine, isoleucine, glutamine, proline, leucine, glutamic acid, alanine, glycine, valine, and serine; wherein altering the at least one endonuclease sensitive sequence motif results in the formation of a degenerate codon encoding the selected amino acid, wherein the codon and the degenerate codon are different.

In some embodiments, the disclosure provides an mRNA comprising at least one endonuclease sensitive sequence motif, wherein the at least one endonuclease sensitive sequence motif comprises the nucleotide sequence UGA, wherein the U is the 3rd position (wobble position) of a codon comprising the ORF, wherein the codon encodes an amino acid selected from the group consisting of: asparagine, threonine, serine, isoleucine, histidine, proline arginine, leucine, aspartic acid, alanine, glycine, valine, tyrosine, cysteine, and phenylalanine, wherein altering the at least one endonuclease sensitive sequence motif results in the formation of a degenerate codon encoding the selected amino acid, wherein the codon and the degenerate codon are different.

In some embodiments, the disclosure provides an mRNA comprising at least one endonuclease sensitive sequence motif, wherein the at least one endonuclease sensitive sequence motif comprises the nucleotide sequence AGA, wherein the G is the 3^{rd} position (wobble position) of a codon comprising the ORF, wherein the codon encodes an amino acid selected from the group consisting of: lysine, glutamine, and glutamic acid, wherein altering the at least one endonuclease sensitive sequence motif results in the formation of degenerate codon encoding the selected amino acid, wherein the codon and the degenerate codon are different.

In some embodiments, the disclosure provides an mRNA comprising at least one endonuclease sensitive sequence motif, wherein the at least one endonuclease sensitive sequence motif comprises the nucleotide sequence UGA, wherein the G is the 3rd position (wobble position) of a codon comprising the ORF, wherein the codon encodes an amino acid selected from the group consisting of: leucine and valine, wherein altering the at least one endonuclease sensitive sequence motif results in the formation of a degenerate codon encoding the selected amino acid, wherein the codon and the degenerate codon are different.

In some embodiments, the disclosure provides an mRNA comprising at least one endonuclease sensitive sequence motif (e.g., two, three, four, five, six, seven, eight, nine, ten, 10-20, 20-30, 30-40, 40-50, 50-60, 60-70, 70-80, 80-90, 90-100, 100-250, 250-500, 500-1000 or more endonuclease sensitive sites in the mRNA (e.g., 5′UTR, ORF and/or 3′UTR)) wherein about 5-10%, 10-20%, 20-30%, 30-40%, 40-50%, 50-60%, 60-70%, 70-80%, 80-90%, 90-100% or about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99% or 100% of the endonuclease sensitive sequence motifs present in the unaltered mRNA are altered by substitution or deletion of one or more nucleotides present in the endonuclease sensitive sequence motifs.

In some embodiments, the disclosure provides an mRNA comprising at least one endonuclease sensitive sequence motif (e.g., two, three, four, five, six, seven, eight, nine, ten, 10-20, 20-30, 30-40, 40-50, 50-60, 60-70, 70-80, 80-90, 90-100, 100-250, 250-500, 500-1000 or more endonuclease sensitive sites in the mRNA (e.g., 5′UTR, ORF and/or 3′UTR)) wherein about 5-10%, 10-20%, 20-30%, 30-40%, 40-50%, 50-60%, 60-70%, 70-80%, 80-90%, 90-100% or about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99% or 100% of the endonuclease sensitive sequence motifs present in the unaltered mRNA are altered by substitution of one or more nucleotides present in the endonuclease sensitive sequence motifs.

In some embodiments, the disclosure provides an mRNA comprising at least one endonuclease sensitive sequence motif (e.g., two, three, four, five, six, seven, eight, nine, ten, 10-20, 20-30, 30-40, 40-50, 50-60, 60-70, 70-80, 80-90, 90-100, 100-250, 250-500, 500-1000 or more endonuclease sensitive sites in the mRNA (e.g., 5′UTR, ORF and/or 3′UTR)) wherein about 5-10%, 10-20%, 20-30%, 30-40%, 40-50%, 50-60%, 60-70%, 70-80%, 80-90%, 90-100% or about 5%, about 10%, about 20%, about 30%, about 40%, about 50%, about 60%, about 70%, about 80%, about 85%, about 90%, about 91%, about 92%, about 93%, about 94%, about 95%, about 96%, about 97%, about 98%, about 99% or 100% of the endonuclease sensitive sequence motifs present in the unaltered mRNA are altered by deletion of one or more nucleotides present in the endonuclease sensitive sequence motifs.

In some embodiments, the disclosure provides an mRNA comprising at least one endonuclease sensitive sequence motif, wherein all or substantially all, or some of the endonuclease sensitive sequence motifs (e.g., 2, 3, 4, 5, 6, 7, 8, 9, 10, 11, 12, 13, 14, 15, 16, 17, 18, 19, 20 or more motifs) present in the mRNA are altered by substitution or deletion of one or more nucleotides present in the endonuclease sensitive sequence motifs. In some embodiments, the number, location, or both, of endonuclease sensitive sequence motifs that are altered is selected to control the endonuclease resistance, stability, half-life, and/or potency of the resulting stabilized therapeutic mRNA molecule and, thus, the amount of protein translated over time.

Methods to Characterize Stabilized mRNA

In some aspects, the disclosure provides methods of generating a stabilized mRNA (e.g., by increasing or improving half-life, increasing or improving potency, increasing or improving resistance to endonuclease activity, decreasing or reducing susceptibility to endonuclease activity), wherein the method comprises identifying at least one endonuclease sensitive sequence motif in an endonuclease-susceptible mRNA and altering the at least one endonuclease sensitive sequence motif such that stability of the mRNA is improved or increased relative to an unaltered mRNA.

Methods to identify endonuclease sensitive sequence motifs in nucleic acid sequences, such as mRNA, are known to one skilled in the art. For example, in some embodiments, sequence analysis of an mRNA sequence is used to identify the presence, number, and position of an endonuclease sensitive sequence motif as described herein. In some embodiments, identification of intrinsic features (e.g., a endonuclease sensitive sequence motif) of a nucleotide sequence is accomplished using software to query or search the nucleotide sequence for the feature.

In some embodiments, a method of identifying an endonuclease sensitive sequence motif in a nucleic acid sequence comprises profiling genome-wide abundance of mRNA degradation intermediates. mRNA degradation products that are generated by endonuclease cleavage comprise a 5′ RNA product and a 3′ RNA product, wherein the 3′ RNA product comprises a terminal 5′ monophosphate (e.g., a 5′ phosphorylated or 5′P mRNA decay intermediate). In some embodiments, a method of profiling mRNA degradation intermediates comprises measuring the abundance of 3′ RNA products with a 5′ monophosphate. Exemplary methods include 5PSeq as described below and by Pelechano, et al (2016) Nat. Protoc. 11:359-376, which is incorporated by reference herein.

Briefly, 5PSeq comprises selective ligation of a synthetic, oligonucleotide-containing RNA adaptor molecule to the 5′P mRNA decay intermediate, wherein the 5′ monophosphate is necessary for ligation between the RNA adaptor molecule and the 5′P mRNA decay intermediate. In some embodiments, ligated 5′P mRNA degradation products are enriched by removal of 5′P non-mRNA molecules. Methods of enrichment are known in the art, including selection of mRNA comprising a poly adenylated tail or depletion of ribosomal RNA.

Upon enrichment, a sequencing library is generated wherein the ligated 5′P mRNA is subjected to reverse transcription (RT) followed by polymerase chain reaction (PCR) to generate a library of complementary DNA (cDNA), wherein the PCR uses a forward primer that anneals to the sequence in the 5′P RNA adaptor and a reverse primer that anneals to sequence generated by the RT primer. Given that only 5′P mRNA degradation products comprise an RNA adaptor molecule, use of a forward primer that anneals the RNA adaptor molecule yields cDNA only generated from mRNA comprising a 5′ monophosphate. The cDNA library is sequenced using a method of high throughput sequencing known in the art. Non-limiting examples of methods of high throughput sequencing include clonal amplification-based sequencing platforms (e.g., Illumina) and single molecule sequencing platforms (e.g., single-molecule real-time sequencing). In some embodiments, alignment of sequence reads generated by 5PSeq allows identification of endonucleolytic cleavage sites. In some embodiments, alignment of sequence reads generated by 5PSeq relative to sequencing reads of genomic RNA is used to identify sites of endonuclease-mediated cleavage in endogenous RNA transcripts. In some embodiments, alignment of sequence reads generated by 5PSeq relative to sequencing reads of a delivered mRNA is used to identify sites of endonuclease-mediated cleavage in a therapeutic mRNA of the disclosure. In some embodiments, evaluation of the sequence surrounding a site of endonuclease-mediated cleavage is used to determine an endonuclease sensitive sequence motif.

In some embodiments, the disclosure provides an mRNA comprising at least one endonuclease sensitive sequence motif, wherein the endonuclease sensitive site motif is altered by substitution, deletion or chemical modification, such that the resulting mRNA has increased stability relative to an unaltered mRNA. In some embodiments, increased stability is determined by measuring the half-life of an mRNA altered to be endonuclease resistant relative to an unaltered mRNA, wherein increased mRNA half-life is indicative of increased stability. Methods of measuring mRNA half-life are known in the art. In some embodiments, an mRNA of the disclosure is altered by substitution, deletion or chemical modification of endonuclease sensitive sequence motifs in the 5′UTR, the 3′UTR, or a combination thereof. In some embodiments, the half-life of an mRNA altered in the 5′UTR, the 3′UTR, or a combination thereof is measured by use of an ORF that encodes a reporter protein. The half-life of an mRNA with an ORF that encodes a reporter protein is determined by measuring the expression of the reporter protein in cells contacted with the mRNA. The abundance of signal of the expressed reporter protein quantified at regular intervals over time provides a measure of the mRNA half-life. In some embodiments, a reporter protein is a fluorescent protein, wherein expression of the reporter protein is quantified by measuring the mean fluorescence intensity of contacted cells at regular intervals over time. In some embodiments, a reporter protein is a bioluminescent protein, wherein expression of the reporter protein is quantified by measuring the bioluminescent signal of contacted cells at regular intervals over time. In some embodiments, a reporter protein is an enzyme, wherein expression of the reporter protein is quantified by measuring the level of an enzymatic product present in contacted cells at regular intervals over time. In some embodiments, a reporter protein is recognized by a specific antibody, wherein expression of the reporter protein over time is determined by quantitative immunoblotting using an antibody specific to the reporter protein. Analysis of expression of a reporter protein at regular intervals over time is used to determine the half-life of an mRNA encoding the reporter protein.

In some embodiments, an mRNA of the disclosure is altered to increase stability by substitution, deletion or chemical modification of endonuclease sensitive sequence motifs in the 5′UTR, the 3′UTR, the ORF, or a combination thereof. In some embodiments, the half-life is measured by a method of RNA quantification. In some embodiments, an altered mRNA is contacted with cells and the quantity of the mRNA in contacted cells is measured by a method of RNA quantification at regular intervals over time. Methods of RNA quantification are known in the art. Non-limiting examples of RNA quantification include northern analysis, nuclease protection assays, in situ hybridization, quantitative real time PCR (RT-PCR), branched DNA assay, and RNA Sequencing. In some embodiments, analysis of the quantity of mRNA in contacted cells at regular intervals over time is used to determine the half-life of the mRNA.

In some embodiments, an mRNA of the disclosure that is altered to increase endonuclease resistance and/or decrease endonuclease susceptibility has increased potency relative to an unaltered mRNA. In some embodiments, increased potency is determined by measuring the level of protein translated from an mRNA altered to be endonuclease resistant compared to an unaltered mRNA, wherein an increased level of translated protein is used to indicated increased potency. In some embodiments, an mRNA altered to be increase endonuclease resistance and/or decrease endonuclease susceptibility results in an increased level of translated protein when contacted with cells when compared to an unaltered mRNA. In some embodiments, an mRNA of the disclosure is altered to increase endonuclease resistance and/or decrease endonuclease susceptibility by substitution, deletion or chemical modification of endonuclease sensitive sequence motifs in the 5′UTR, the 3′UTR, or a combination thereof. In some embodiments, the level of translated protein produced from an mRNA altered in the 5′UTR, the 3′UTR, or a combination thereof is measured by using an ORF that encodes a reporter protein.

The level of protein translated from an mRNA comprising an ORF that encodes a reporter protein is determined by measuring the expression of the reporter protein in cells contacted with the mRNA at regular intervals over time. Analysis of the area under the curve (AUC) of signal of the expressed reporter protein over time provides a measure of the total reporter protein translated from an mRNA in contacted cells. In some embodiments, a reporter protein is a fluorescent protein, wherein the AUC of cellular mean fluorescence intensity over time provides a measure of total reporter protein translated from an mRNA. In some embodiments, a reporter protein is a bioluminescent protein, wherein the AUC of cellular bioluminescent signal over time provides a measure of total reporter protein translated from an mRNA. In some embodiments, a reporter protein is an enzyme, wherein the AUC of cellular enzymatic product produced over time provides a measure of total reporter protein translated from an mRNA. In some embodiments, a reporter protein is recognized by a specific antibody, wherein expression of the reporter protein is measured by quantitate immunoblotting using an antibody specific to the reporter protein. The AUC of cellular reporter protein produced over time as measured by quantitate immunoblot provides a measure of total reporter protein translated from an mRNA.

In some embodiments, an mRNA of the disclosure is altered to increase endonuclease resistance and/or decrease endonuclease susceptibility by substitution, deletion or chemical modification of endonuclease sensitive sequence motifs in the 5′UTR, the 3′UTR, the ORF, or a combination thereof. In some embodiments, the potency of an mRNA altered in the 5′UTR, the 3′UTR, the ORF, or a combination thereof is determined by measuring the quantity of protein translated from the mRNA using a method of protein quantification. Methods of quantifying translated protein in a cell are known in the art. Non-limiting examples of methods to quantifying cellular proteins translated from an mRNA provided to the cell include high-performance liquid chromatography (HPLC), liquid chromatography-mass spectrometry (LC/MS), matrix assisted laser desorption/ionization time of flight (MALDI-TOF) mass spectrometry, enzyme-linked immunosorbent assay (ELISA), protein immunoprecipitation, and quantitative immunoblotting. In some embodiments, analysis of the AUC of quantity of translated protein in contacted cells over time is used to determine the total level of protein translated from an mRNA. In some embodiments, the total level of translated protein of an mRNA encoding the protein and altered to be endonuclease resistant is compared to an unaltered mRNA counterpart (e.g., a starting mRNA prior to altering). In some embodiments, an mRNA altered to be endonuclease resistant produces a higher level of total translated protein in contacted cells when compared to an unaltered mRNA counterpart.

Exemplary Stabilized Therapeutic mRNAs

In some aspects, the disclosure provides a therapeutic, stabilized mRNA with increased or improved resistance to endonuclease activity and/or decreased or reduced susceptibility to endonuclease activity as described herein. In some embodiments, a therapeutic, stabilized mRNA is provided by alteration of one or more endonuclease sensitive sequence motifs from an unaltered mRNA comprising a 5′UTR, an ORF encoding a polypeptide, and a 3′UTR. In some embodiments, a therapeutic, stabilized mRNA is produced by altering one or more endonuclease sensitive sequence motifs (e.g., WGA, wherein W=adenine (A) or uracil (U)) in the 5′UTR, the ORF, and/or the 3′UTR of an unaltered mRNA.

In some embodiments, the 5′UTR of an unaltered mRNA comprises one or more endonuclease sensitive sequence motifs (e.g., 1, 2, 3, 4, 5, 6, 7, 8, 9, 10, 11, 12, 13, 14, 15, 16, 17, 18, 19, or 20 motifs comprising the nucleotide sequence UGA or AGA) that are altered by substitution or deletion to generate a stabilized 5′UTR with increased or improved endonuclease resistance and or decreased or reduced endonuclease susceptibility. In some embodiments, the 5′UTR of an unaltered mRNA comprises one endonuclease sensitive sequence motifs that is altered by substitution or deletion. In some embodiments, the 5′UTR of an unaltered mRNA comprises two endonuclease sensitive sequence motifs that are altered by substitution or deletion, wherein the endonuclease sensitive motifs are the same or different. In some embodiments, the 5′UTR of an unaltered mRNA comprises three endonuclease sensitive sequence motifs that are altered by substitution or deletion, wherein the endonuclease sensitive motifs are the same or different. In some embodiments, the 5′UTR of an unaltered mRNA comprises four endonuclease sensitive sequence motifs that are altered by substitution or deletion, wherein the endonuclease sensitive motifs are the same or different. In some embodiments, the 5′UTR of an unaltered mRNA comprises five endonuclease sensitive sequence motifs that are altered by substitution or deletion, wherein the endonuclease sensitive motifs are the same or different.

In some embodiments, a 5′UTR of an unaltered mRNA comprises a nucleotide sequence set forth by SEQ ID NO: 3, wherein at least one endonuclease sensitive sequence motifs comprising the nucleotide sequence AGA is altered by substitution or deletion to generate a stabilized mRNA with increased or improved endonuclease resistance and/or decreased or reduced endonuclease susceptibility. In some embodiments, altering the at least one endonuclease sensitive sequence motifs comprising the nucleotide sequence AGA comprises deletion of one or more nucleotides. In some embodiments, altering the at least one endonuclease sensitive sequence motifs comprising the nucleotide sequence AGA comprises substitution of one or more nucleotides. In some embodiments, altering the at least one endonuclease sensitive sequence motifs comprising the nucleotide sequence AGA comprises substitution of the first adenine, the second guanine, the third adenine, or any combination thereof. In some embodiments, altering the at least one endonuclease sensitive sequence motifs comprising the nucleotide sequence AGA comprises substitution of the first adenine (A) with a cytosine (C) or guanine (G). In some embodiments, altering the at least one endonuclease sensitive sequence motifs comprising the nucleotide sequence AGA comprises substitution of the second guanine (G) with a cytosine (C), adenine (A) or uracil (U). In some embodiments, altering the at least one endonuclease sensitive sequence motifs comprising the nucleotide sequence AGA comprises substitution of the third adenine (A) with a cytosine (C), uracil (U) or guanine (G).

In some embodiments, a 5′UTR comprising the nucleotide sequence set forth by SEQ ID NO: 3 comprises one endonuclease sensitive sequence motif (e.g., comprising the nucleotide sequence AGA) that is altered by substitution or deletion. In some embodiments, a 5′UTR comprising the nucleotide sequence set forth by SEQ ID NO: 3 comprises one endonuclease sensitive sequence motif (e.g., comprising the nucleotide sequence AGA) that are altered by substitution or deletion. In some embodiments, a 5′UTR comprising the nucleotide sequence set forth by SEQ ID NO: 3 comprises two endonuclease sensitive sequence motifs (e.g., comprising the nucleotide sequence AGA) that are altered by substitution or deletion. In some embodiments, a 5′UTR comprising the nucleotide sequence set forth by SEQ ID NO: 3 comprises three endonuclease sensitive sequence motifs (e.g., comprising the nucleotide sequence AGA) that are altered by substitution or deletion. In some embodiments, a 5′UTR comprising the nucleotide sequence set forth by SEQ ID NO: 3 comprises four endonuclease sensitive sequence motifs (e.g., comprising the nucleotide sequence AGA) that are altered by substitution or deletion. In some embodiments, a 5′UTR comprising the nucleotide sequence set forth by SEQ ID NO: 3 comprises five endonuclease sensitive sequence motifs (e.g., comprising the nucleotide sequence AGA) that are altered by substitution or deletion. In some embodiments, a 5′UTR comprising the nucleotide sequence set forth by SEQ ID NO: 3 comprises six endonuclease sensitive sequence motifs (e.g., comprising the nucleotide sequence AGA) that are altered by substitution or deletion.

In some embodiments, a 5′UTR of an unaltered mRNA comprises a nucleotide sequence set forth by SEQ ID NO: 3, wherein the 5′UTR comprises a first endonuclease sensitive sequence motif comprising the nucleotide sequence AGA that is altered by substitution or deletion. In some embodiments, a 5′UTR of an unaltered mRNA comprises a nucleotide sequence set forth by SEQ ID NO: 3, wherein the 5′UTR comprises a second endonuclease sensitive sequence motif comprising the nucleotide sequence AGA that is altered by substitution or deletion. In some embodiments, a 5′UTR of an unaltered mRNA comprises a nucleotide sequence set forth by SEQ ID NO: 3, wherein the 5′UTR comprises a third endonuclease sensitive sequence motif comprising the nucleotide sequence AGA that is altered by substitution or deletion. In some embodiments, a 5′UTR of an unaltered mRNA comprises a nucleotide sequence set forth by SEQ ID NO: 3, wherein the 5′UTR comprises a fourth endonuclease sensitive sequence motif comprising the nucleotide sequence AGA that is altered by substitution or deletion. In some embodiments, a 5′UTR of an unaltered mRNA comprises a nucleotide sequence set forth by SEQ ID NO: 3, wherein the 5′UTR comprises a fifth endonuclease sensitive sequence motif comprising the nucleotide sequence AGA that is altered by substitution or deletion. In some embodiments, a 5′UTR of an unaltered mRNA comprises a nucleotide sequence set forth by SEQ ID NO: 3, wherein the 5′UTR comprises a sixth endonuclease sensitive sequence motif comprising the nucleotide sequence AGA that is altered by substitution or deletion.

In some embodiments, a 5′UTR of an unaltered mRNA comprises a nucleotide sequence set forth by SEQ ID NO: 3, wherein the 5′UTR comprises at least one endonuclease sensitive sequence motifs comprising the nucleotide sequence UGA that are altered by substitution or deletion to generate a stabilized mRNA with increased or improved endonuclease resistance and/or decreased or reduced endonuclease susceptibility. In some embodiments, a 5′UTR of an unaltered mRNA comprises a nucleotide sequence set forth by SEQ ID NO: 3, wherein the 5′UTR comprises at least two endonuclease sensitive sequence motifs comprising the nucleotide sequence UGA that are altered by substitution or deletion to generate a stabilized mRNA with increased or improved endonuclease resistance and/or decreased or reduced endonuclease susceptibility. In some embodiments, a 5′UTR of an unaltered mRNA comprises a nucleotide sequence set forth by SEQ ID NO: 3, wherein the 5′UTR comprises at least three endonuclease sensitive sequence motifs comprising the nucleotide sequence UGA that are altered by substitution or deletion to generate a stabilized mRNA with increased or improved endonuclease resistance and/or decreased or reduced endonuclease susceptibility. In some embodiments, a 5′UTR of an unaltered mRNA comprises a nucleotide sequence set forth by SEQ ID NO: 3, wherein the 5′UTR comprises at least four endonuclease sensitive sequence motifs comprising the nucleotide sequence UGA that are altered by substitution or deletion to generate a stabilized mRNA with increased or improved endonuclease resistance and/or decreased or reduced endonuclease susceptibility. In some embodiments, a 5′UTR of an unaltered mRNA comprises a nucleotide sequence set forth by SEQ ID NO: 3, wherein the 5′UTR comprises at least five endonuclease sensitive sequence motifs comprising the nucleotide sequence UGA that are altered by substitution or deletion to generate a stabilized mRNA with increased or improved endonuclease resistance and/or decreased or reduced endonuclease susceptibility. In some embodiments, a 5′UTR of an unaltered mRNA comprises a nucleotide sequence set forth by SEQ ID NO: 3, wherein the 5′UTR comprises at least six endonuclease sensitive sequence motifs comprising the nucleotide sequence UGA that are altered by substitution or deletion to generate a stabilized mRNA with increased or improved endonuclease resistance and/or decreased or reduced endonuclease susceptibility.

In some embodiments, a 5′UTR of an unaltered mRNA comprises a nucleotide sequence set forth by SEQ ID NO: 3, wherein the 5′UTR comprises at least six endonuclease sensitive sequence motifs comprising the nucleotide sequence AGA that are altered by substitution or deletion to generate a stabilized mRNA with increased or improved endonuclease resistance and/or decreased or reduced endonuclease susceptibility. An exemplary 5′UTR wherein at six endonuclease sensitive sequence motifs comprising the nucleotide sequence AGA are altered by substitution with the nucleotide sequence ACA comprises the nucleotide sequence set forth by SEQ ID NO: 4 listed in Table 3.

TABLE 3
Altered Endonuclease Sensitive Sequence Motifs
in Exemplary 5′ UTRs

| | | SEQ |
|---|---|---|
| | | ID |
| 5′ UTR | Sequence | NO: |
| V1.1 5′UTR | GGGAAAUAAGAGAGAAAAGAAGAGUAAGAAGAA | 3 |
| | AUAUAAGAGCCACC | |
| V1.1 5′UTR | GGGAAAUAACACACAAAACAACAGUAACAACAA | 4 |
| (no AGA) | AUAUAACAGCCACC | |

In one embodiment, the ORF of an unaltered mRNA comprises one or more endonuclease sensitive sequence motifs (e.g., 1, 2, 3, 4, 5, 6, 7, 8, 9, 10, 11, 12, 13, 14, 15, 16, 17, 18, 19, or 20 motifs comprising the nucleotide sequence UGA or AGA) that are altered by substitution or deletion to generate a stabilized ORF with increased or improved endonuclease resistance and/or decreased or reduced endonuclease susceptibility. In some embodiments, the ORF of an unaltered mRNA comprises one endonuclease sensitive sequence motifs that is altered by substitution or deletion. In some embodiments, the ORF of an unaltered mRNA comprises two endonuclease sensitive sequence motifs that are altered by substitution or deletion, wherein the endonuclease sensitive motifs are the same or different. In some embodiments, the ORF of an unaltered mRNA comprises three endonuclease sensitive sequence motifs that are altered by substitution or deletion, wherein the endonuclease sensitive motifs are the same or different. In some embodiments, the ORF of an unaltered mRNA comprises four endonuclease sensitive sequence motifs that are altered by substitution or deletion, wherein the endonuclease sensitive motifs are the same or different. In some embodiments, the ORF of an unaltered mRNA comprises five endonuclease sensitive sequence motifs that are altered by substitution or deletion, wherein the endonuclease sensitive motifs are the same or different.

In some embodiments, the 3′UTR of an unaltered mRNA comprises one or more endonuclease sensitive sequence motifs (e.g., 1, 2, 3, 4, 5, 6, 7, 8, 9, 10, 11, 12, 13, 14, 15, 16, 17, 18, 19, or 20 motifs comprising the nucleotide sequence UGA or AGA) that are altered by substitution or deletion to generate a stabilized 3′UTR with increased or improved endonuclease resistance and/or decreased or reduced endonuclease susceptibility. In some embodiments, the 3′UTR of an unaltered mRNA comprises one endonuclease sensitive sequence motifs that is altered by substitution or deletion. In some embodiments, the 3′UTR of an unaltered mRNA comprises two endonuclease sensitive sequence motifs that are altered by substitution or deletion, wherein the endonuclease sensitive motifs are the same or different. In some embodiments, the 3′UTR of an unaltered mRNA comprises three endonuclease sensitive sequence motifs that are altered by substitution or deletion, wherein the endonuclease sensitive motifs are the same or different. In some embodiments, the 3′UTR of an unaltered mRNA comprises four endonuclease sensitive sequence motifs that are altered by substitution or deletion, wherein the endonuclease sensitive motifs are the same or different. In some embodiments, the 3′UTR of an unaltered mRNA comprises five endonuclease sensitive sequence motifs that are altered by substitution or deletion, wherein the endonuclease sensitive motifs are the same or different.

In some embodiments, a 3′UTR of an unaltered mRNA comprises a nucleotide sequence set forth by SEQ ID NO: 1, wherein at least one endonuclease sensitive sequence motifs comprising the nucleotide sequence UGA is altered by substitution or deletion to generate a stabilized mRNA with increased or improved endonuclease resistance and/or decreased or reduced endonuclease susceptibility. In some embodiments, altering the at least one endonuclease sensitive sequence motifs comprising the nucleotide sequence UGA comprises deletion of one or more nucleotides. In some embodiments, altering the at least one endonuclease sensitive sequence motifs comprising the nucleotide sequence UGA comprises substitution of one or more nucleotides. In some embodiments, altering the at least one endonuclease sensitive sequence motifs comprising the nucleotide sequence UGA comprises substitution of the first uracil, the second guanine, the third adenine, or any combination thereof. In some embodiments, altering the at least one endonuclease sensitive sequence motifs comprising the nucleotide sequence UGA comprises substitution of the first uracil (U) with a cytosine (C) or guanine (G). In some embodiments, altering the at least one endonuclease sensitive sequence motifs comprising the nucleotide sequence UGA comprises substitution of the second guanine (G) with a cytosine (C), adenine (A) or uracil (U). In some embodiments, altering the at least one endonuclease sensitive sequence motifs comprising the nucleotide sequence UGA comprises substitution of the third adenine (A) with a cytosine (C), uracil (U) or guanine (G).

In some embodiments, a 3′UTR comprising the nucleotide sequence set forth by SEQ ID NO: 1 comprises one endonuclease sensitive sequence motif (e.g., comprising the nucleotide sequence UGA) that is altered by substitution or deletion. In some embodiments, a 3′UTR comprising the nucleotide sequence set forth by SEQ ID NO: 1 comprises two endonuclease sensitive sequence motifs (e.g., comprising the nucleotide sequence UGA) that are altered by substitution or deletion. In some embodiments, a 3′UTR comprising the nucleotide sequence set forth by SEQ ID NO: 1 comprises three endonuclease sensitive sequence motifs (e.g., comprising the nucleotide sequence UGA) that are altered by substitution or deletion.

In some embodiments, a 3′UTR of an unaltered mRNA comprises a nucleotide sequence set forth by SEQ ID NO: 1, wherein the 3′UTR comprises a first endonuclease sensitive sequence motif comprising the nucleotide sequence UGA that is a stop codon, and wherein the first endonuclease sensitive sequence motif is altered by deletion to generate a stabilized mRNA with increased or improved endonuclease resistance and/or decreased or reduced endonuclease susceptibility. Exemplary 3′UTRs altered by deletion of a first endonuclease sensitive sequence motif, wherein the endonuclease sensitive sequence motif is a stop codon comprising the nucleotide sequence UGA, comprise nucleotide sequences set forth by SEQ ID NO: 5, SEQ ID NO: 6, SEQ ID NO: 7, and SEQ ID NO: 2.

In some embodiments, a 3′UTR of an unaltered mRNA comprises a nucleotide sequence set forth by SEQ ID NO: 1, wherein the 3′UTR comprises at least two endonuclease sensitive sequence motifs comprising the nucleotide sequence UGA that are altered by substitution or deletion to generate a stabilized mRNA with increased or improved endonuclease resistance and/or decreased or reduced endonuclease susceptibility. Exemplary 3′UTRs wherein at least two endonuclease sensitive sequence motifs (e.g., UGA) are altered by substitution or deletion comprise nucleotide sequences set forth by SEQ ID NO: 6 and SEQ ID NO: 7.

In some embodiments, a 3′UTR of an unaltered mRNA comprises a nucleotide sequence set forth by SEQ ID NO: 1, wherein the 3′UTR comprises at least three endonuclease sensitive sequence motifs comprising the nucleotide sequence UGA that are altered by substitution or deletion to generate a stabilized mRNA with increased or improved endonuclease resistance and/or decreased or reduced endonuclease susceptibility. An exemplary 3′UTR wherein at least three endonuclease sensitive sequence motifs (e.g., UGA) are altered by substitution or deletion comprises the nucleotide sequence set forth by SEQ ID NO: 2.

In some aspects, the disclosure provides a stabilized, therapeutic mRNA comprising a 5′ UTR, an ORF encoding a polypeptide, and a 3′ UTR, wherein the 3′ UTR comprises the nucleotide sequence of SEQ ID NO: 5.

In some aspects, the disclosure provides a stabilized, therapeutic mRNA comprising a 5′ UTR, an ORF encoding a polypeptide, and a 3′ UTR, wherein the 3′ UTR comprises the nucleotide sequence of SEQ ID NO: 6.

In some aspects, the disclosure provides a stabilized, therapeutic mRNA comprising a 5′ UTR, an ORF encoding a polypeptide, and a 3′ UTR, wherein the 3′ UTR comprises the nucleotide sequence of SEQ ID NO: 7.

In some aspects, the disclosure provides a stabilized, therapeutic mRNA comprising a 5′ UTR, an ORF encoding a polypeptide, and a 3′ UTR, wherein the 3′ UTR comprises the nucleotide sequence of SEQ ID NO: 2.

In some embodiments, the ORF encodes a polypeptide of interest described herein. In some embodiments, the polypeptide of interest is a therapeutic polypeptide described herein.

TABLE 4
Altered Endonuclease Sensitive Sequence Motifs in Exemplary 3′ UTRs

| | | SEQ ID |
|---|---|---|
| 3′ UTR | Sequence | NO: |
| v1.1 3′UTR | UGAUAAUAGGCUGGAGCCUCGGUGGCCUAGCUUCUUGCC | 1 |
| (+UGA1, +UGA2, | CCUUGGGCCUCCCCCCAGCCCCUCCUCCCCUUCCUGCAC | |
| +UGA3) | CCGUACCCCCGUGGUCUUUGAAUAAAGUCUGAGUGGGCG | |
| | GC | |
| v1.1 3′ UTR | UAAUAGGCUGGAGCCUCGGUGGCCUAGCUUCUUGCCCCU | 5 |
| (−UGA1, +UGA2, | UGGGCCUCCCCCCAGCCCCUCCUCCCCUUCCUGCACCCG | |
| +UGA3) | UACCCCCGUGGUCUUUGAAUAAAGUCUGAGUGGGCGGC | |
| v1.1 3′ UTR | UAAUAGGCUGGAGCCUCGGUGGCCUAGCUUCUUGCCCCU | 6 |
| (−UGA1, −UGA2, | UGGGCCUCCCCCCAGCCCCUCCUCCCCUUCCUGCACCCG | |
| +UGA3) | UACCCCCGUGGUCUUUCAAUAAAGUCUGAGUGGGCGGC | |
| v1.1 3′ UTR | UAAUAGGCUGGAGCCUCGGUGGCCUAGCUUCUUGCCCCU | 7 |
| (−UGA1, +UGA2, | UGGGCCUCCCCCCAGCCCCUCCUCCCCUUCCUGCACCCG | |
| −UGA3) | UACCCCCGUGGUCUUUGAAUAAAGUCUCAGUGGGCGGC | |
| v1.1 3′ UTR | UAAUAGGCUGGAGCCUCGGUGGCCUAGCUUCUUGCCCCU | 2 |
| (−UGA1, −UGA2, | UGGGCCUCCCCCCAGCCCCUCCUCCCCUUCCUGCACCCG | |
| −UGA3) | UACCCCCGUGGUCUUUCAAUAAAGUCUCAGUGGGCGGC | |

(endonuclease sensitive sequence motifs before altering by substitution or deletion are shown underlined, after altering by substitution shown in bold)

mRNA Construct Components

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′UTR), a 3′ untranslated region (3′UTR), and/or a coding region (e.g., an open reading frame). In some embodiments, an mRNA provided by the disclosure comprises a 5′ UTR comprising a nucleotide sequence selected from the group consisting of: SEQ ID NO: 3 (V1.0 5′UTR), SEQ ID NO: 8 (V1.1 5′UTR), or any 5′ UTR referred to by sequence in Table 5. In some embodiments, an mRNA provided by the disclosure comprises a 5′ UTR comprising a nucleotide sequence that is at least 50%, at least 60%, at least 70%, at least 80%, at least 85%, at least 90%, at least 95%, at least 98%, at least 99%, or 100% identical to a nucleotide sequence selected from the group consisting of: SEQ ID NO: 3 (V1.0 5′UTR), SEQ ID NO: 8 (V1.1 5′UTR), or any 5′ UTR referred to by sequence in Table 5.

In some embodiments, an mRNA of the disclosure comprises a 5′UTR wherein endonuclease sensitive sequence motifs in the 5′UTR is altered to increase or improve endonuclease resistance and/or decrease or reduce endonuclease susceptibility. In some embodiments, the 5′UTR prior to altering comprises a nucleotide sequence selected from a group consisting of: SEQ ID NO: 3 (V1.0 5′UTR), SEQ ID NO: 8 (V1.1 5′UTR), or any 5′ UTR referred to by sequence in Table 5. In some embodiments, the 5′UTR prior to altering comprises a nucleotide sequence that is at least 50%, at least 60%, at least 70%, at least 80%, at least 85%, at least 90%, at least 95%, at least 98%, at least 99%, or 100% identical to a nucleotide sequence selected from a group consisting of: SEQ ID NO: 3 (V1.0 5′UTR), SEQ ID NO: 8 (V1.1 5′UTR), or any 5′ UTR referred to by sequence in Table 5.

In some embodiments, an mRNA provided by the disclosure comprises a 3′UTR comprising a nucleotide sequence selected from a group consisting of: SEQ ID NO: 1 (v1.1 3′UTR) or any 3′UTR referred to by sequence in Table 6. In some embodiments, an mRNA provided by the disclosure comprises a 3′ UTR comprising a nucleotide sequence that is at least 50%, at least 60%, at least 70%, at least 80%, at least 85%, at least 90%, at least 95%, at least 98%, at least 99%, or 100% identical to a nucleotide sequence selected from the group consisting of: SEQ ID NO: 1 (v1.1 3′UTR) or any 3′UTR referred to by sequence in Table 6.

In some embodiments, an mRNA provided by the disclosure comprises a 3′UTR comprising at least one endonuclease sensitive sequence motif, wherein the 3′UTR is altered to increase or improve endonuclease resistance and/or decrease or reduce endonuclease susceptibility. In some embodiments, the 3′UTR prior to altering comprises a nucleotide sequence selected from a group consisting of: SEQ ID NO: 1 (v1.1 3′UTR) or any 3′UTR referred to by sequence in Table 6. In some embodiments, the 3′UTR prior to altering comprises a nucleotide sequence that is at least 50%, at least 60%, at least 70%, at least 80%, at least 85%, at least 90%, at least 95%, at least 98%, at least 99%, or 100% identical to a nucleotide sequence selected from the group consisting of: SEQ ID NO: 1 (v1.1 3′UTR) or any 3′UTR referred to by sequence in Table 6.

An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m₂^{7,O3′}GpppG, m₂^{7,O3′}GppppG, m₂^{7,O2′}GppppG, m⁷Gpppm⁷G, m⁷3′dGpppG, m₂^{7,O3′}GpppG, m₂^{7,O3′}GppppG, and m₂^{7,O2′}GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

### 5′ UTR and Translation Initiation

In certain embodiments, the polynucleotide (e.g., mRNA) encoding a polypeptide of the present disclosure comprises a 5′ UTR and/or a translation initiation sequence. Natural 5′ UTRs comprise sequences involved in translation initiation. For example, Kozak sequences comprise natural 5′ UTRs and are commonly known to be involved in the process by which the ribosome initiates translation of many genes. 5′ UTRs also have been known to form secondary structures which are involved in elongation factor binding.

By engineering the features typically found in abundantly expressed genes of specific target organs, one can enhance the stability and protein production of the polynucleotides of the disclosure. For example, introduction of 5′ UTR of mRNA known to be upregulated in cancers, such as c-myc, could be used to enhance expression of a nucleic acid molecule, such as a polynucleotide, in cancer cells. Untranslated regions useful in the design and manufacture of polynucleotides include, but are not limited, to those disclosed in International Patent Publication No. WO 2014/164253 (see also US20160022840).

Shown in Table 5 is a listing of exemplary 5′ UTRs. Variants of 5′ UTRs can be utilized wherein one or more nucleotides are added or removed to the termini, including A, U, C or G.

TABLE 5
Exemplary 5′-UTRs

| | | SEQ |
|---|---|---|
| 5′ UTR | | ID |
| Identifier | Sequence | NO. |
| V1.0 5′ UTR | GGGAAAUAAGAGAGAAAAGAAGAGUAAG | 3 |
| | AAGAAAUAUAAGAGCCACC | |
| V1.0 5′ UTR-A | AGGAAAUAAGAGAGAAAAGAAGAGUAAG | 9 |
| | AAGAAAUAUAAGAGCCACC | |
| V1.0 5′UTR- | UAAGAGAGAAAAGAAGAGUAAGAAGAAA | 10 |
| minus leader | UAUAAGAGCCACC | |
| V1.1 5′UTR | GGGAAAUAAGAGAGAAAAGAAGAGUAAG | 8 |
| | AAGAAAUAUAAGACCCCGGCGCCGCCAC | |
| | C | |
| V1.1 5′UTR-A | AGGAAAUAAGAGAGAAAAGAAGAGUAAG | 11 |
| | AAGAAAUAUAAGACCCCGGCGCCGCCAC | |
| | C | |
| V1.1 5′UTR- | UAAGAGAGAAAAGAAGAGUAAGAAGAAA | 12 |
| minus leader | UAUAAGACCCCGGCGCCGCCACC | |
| 5UTR-001 | GGGAGAUCAGAGAGAAAAGAAGAGUAAG | 13 |
| | AAGAAAUAUAAGAGCCACC | |
| 5′UTR-002 | GGAAUAAAAGUCUCAACACAACAUAUAC | 14 |
| | AAAACAAACGAAUCUCAAGCAAUCAAGC | |
| | AUUCUACUUCUAUUGCAGCAAUUUAAAU | |
| | CAUUUCUUUUAAAGCAAAAGCAAUUUUC | |
| | UGAAAAUUUUCACCAUUUACGAACGAUA | |
| | GCAAC | |
| 5′UTR-003 | GGGAGACAAGCUUGGCAUUCCGGUACUG | 15 |
| | UUGGUAAAGCCACC | |
| 5′UTR-004 | GGGAAUUAACAGAGAAAAGAAGAGUAAG | 16 |
| | AAGAAAUAUAAGAGCCACC | |
| 5′UTR-005 | GGGAAAUUAGACAGAAAAGAAGAGUAAG | 17 |
| | AAGAAAUAUAAGAGCCACC | |
| 5′UTR-006 | GGGAAAUAAGAGAGUAAAGAACAGUAAG | 18 |
| | AAGAAAUAUAAGAGCCACC | |
| 5′UTR-007 | GGGAAAAAAGAGAGAAAAGAAGACUAAG | 19 |
| | AAGAAAUAUAAGAGCCACC | |
| 5′UTR-008 | GGGAAAUAAGAGAGAAAAGAAGAGUAAG | 20 |
| | AAGAUAUAUAAGAGCCACC | |
| 5′UTR-009 | GGGAAAUAAGAGACAAAACAAGAGUAAG | 21 |
| | AAGAAAUAUAAGAGCCACC | |
| 5′UTR-010 | GGGAAAUUAGAGAGUAAAGAACAGUAAG | 22 |
| | UAGAAUUAAAAGAGCCACC | |
| 5′UTR-011 | GGGAAAUAAGAGAGAAUAGAAGAGUAAG | 23 |
| | AAGAAAUAUAAGAGCCACC | |
| 5′UTR-012 | GGGAAAUAAGAGAGAAAAGAAGAGUAAG | 24 |
| | AAGAAAAUUAAGAGCCACC | |
| 5′UTR-013 | GGGAAAUAAGAGAGAAAAGAAGAGUAAG | 25 |
| | AAGAAAUUUAAGAGCCACC | |
| 5′UTR-014 | UCAAGCUUUUGGACCCUCGUACAGAAGC | 26 |
| | UAAUACGACUCACUAUAGGGAAAUAAGA | |
| | GAGAAAAGAAGAGUAAGAAGAAAUAUAA | |
| | GAGCCACC | |
| 5′UTR-015 | GGACAGAUCGCCUGGAGACGCCAUCCAC | 27 |
| | GCUGUUUUGACCUCCAUAGAAGACACCG | |
| | GGACCGAUCCAGCCUCCGCGGCCGGGAAC | |
| | GGUGCAUUGGAACGCGGAUUCCCCGUGC | |
| | CAAGAGUGACUCACCGUCCUUGACACG | |
| 5′UTR-016 | GGCGCUGCCUACGGAGGUGGCAGCCAUC | 28 |
| | UCCUUCUCGGCAUC | |
| S065 core | CCUCAUAUCCAGGCUCAAGAAUAGAGCU | 29 |
| | CAGUGUUUUGUUGUUUAAUCAUUCCGAC | |
| | GUGUUUUGCGAUAUUCGCGCAAAGCAGC | |
| | CAGUCGCGCGCUUGCUUUUAAGUAGAGU | |
| | UGUUUUUCCACCCGUUUGCCAGGCAUCU | |
| | UUAAUUUAACAUAUUUUUAUUUUUCAGG | |
| | CUAACCUA | |
| S065 | GGGAGACCUCAUAUCCAGGCUCAAGAAU | 30 |
| | AGAGCUCAGUGUUUUGUUGUUUAAUCAU | |
| | UCCGACGUGUUUUGCGAUAUUCGCGCAA | |
| | AGCAGCCAGUCGCGCGCUUGCUUUUAAG | |
| | UAGAGUUGUUUUUCCACCCGUUUGCCAG | |
| | GCAUCUUUAAUUUAACAUAUUUUUAUUU | |
| | UUCAGGCUAACCUAAAGCAGAGAA | |
| combo3_S065 | GGGAGACCUCAUAUCCAGGCUCAAGAAU | 31 |
| (S065 | AGAGCUCAGUGUUUUGUUGUUUAAUCAU | |
| ExtKozak) | UCCGACGUGUUUUGCGAUAUUCGCGCAA | |
| | AGCAGCCAGUCGCGCGCUUGCUUUUAAG | |
| | UAGAGUUGUUUUUCCACCCGUUUGCCAG | |
| | GCAUCUUUAAUUUAACAUAUUUUUAUUU | |
| | UUCAGGCUAACCUACGCCGCCACC | |

Other non-UTR sequences can also be used as regions or subregions within the polynucleotides. For example, introns or portions of introns sequences can be incorporated into regions of the polynucleotides. Incorporation of intronic sequences can increase protein production as well as polynucleotide levels.

Combinations of features can be included in flanking regions and can be contained within other features. For example, the ORF can be flanked by a 5′ UTR which can contain a strong Kozak translational initiation signal and/or a 3′ UTR which can include an oligo(dT) sequence for templated addition of a poly-A tail. A 5′ UTR can comprise a first polynucleotide fragment and a second polynucleotide fragment from the same and/or different genes such as the 5′ UTRs described in U.S. Patent Application Publication No. 2010-0293625.

These UTRs or portions thereof can be placed in the same orientation as in the transcript from which they were selected or can be altered in orientation or location. Hence a 5′ or 3′ UTR can be inverted, shortened, lengthened, made with one or more other 5′ UTRs or 3′ UTRs.

In some embodiments, the UTR sequences can be changed in some way in relation to a reference sequence. For example, a 3′ or 5′ UTR can be altered relative to a wild type or native UTR by the change in orientation or location as taught above or can be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ or 3′ UTR can be used. As used herein, a “double” UTR is one in which two copies of the same UTR are encoded either in series or substantially in series. For example, a double beta-globin 3′ UTR can be used as described in U.S. Patent Application Publication No. 2010-0129877.

In some embodiments, flanking regions can be heterologous. In some embodiments, the 5′ untranslated region can be derived from a different species than the 3′ untranslated region. The untranslated region can also include translation enhancer elements (TEE). As a non-limiting example, the TEE can include those described in U.S. Patent Application Publication No. 2009-0226470.

In some embodiments, the mRNAs provided by the disclosure comprise a 5′ UTR comprising a T7 leader sequence at the 5′ end of the 5′ UTR. In some embodiments, the mRNA of the disclosure comprises a 5′ UTR comprising a T7 leader sequence comprising the sequence GGGAGA at the 5′ end of the 5′ UTR. In some embodiments, the mRNA of the disclosure comprises a 5′ UTR comprising a T7 leader sequence comprising the sequence GGGAAA at the 5′ end of the 5′ UTR. In some embodiments, the mRNA comprises a 5′ UTR which does not comprise a T7 leader sequence at the 5′ end of the 5′ UTR. In another aspect, the disclosure provides an mRNA comprising a 5′ UTR, wherein the nucleotide sequence of the 5′ UTR comprises any one of the nucleotide sequences set forth in Table 5.

### 3′ UTR and the AU Rich Elements

In certain embodiments, the polynucleotide (e.g., mRNA) encoding a polypeptide further comprises a 3′UTR. 3′UTR is the section of mRNA that immediately follows the translation termination codon and often contains regulatory regions that post-transcriptionally influence gene expression. Regulatory regions within the 3′UTR can influence polyadenylation, translation efficiency, localization, and stability of the mRNA. In one embodiment, the 3′UTR useful for the disclosure comprises a binding site for regulatory proteins or microRNAs. In some embodiments, the 3′-UTR has a silencer region, which binds to repressor proteins and inhibits the expression of the mRNA. In other embodiments, the 3′UTR comprises an AU-rich element. Proteins bind AREs to affect the stability or decay rate of transcripts in a localized manner or affect translation initiation. In other embodiments, the 3′UTR comprises the sequence AAUAAA that directs addition of several hundred adenine residues called the poly(A) tail to the end of the mRNA transcript.

Table 6 shows a listing of 3′-untranslated regions useful for the mRNAs encoding a polypeptide. Variants of 3′ UTRs can be utilized wherein one or more nucleotides are added or removed to the termini, including A, U, C or G.

TABLE 6
Exemplary 3′-Untranslated Regions

| 3′ UTR | Name/ | | |
|---|---|---|---|
| Identifier | Description | Sequence | SEQ ID NO. |
| Downstream | 3′ UTR | UAAUAGGCUGGAGCCUCGGUGGCCUAGC | 5 |
| UTR | (−UGA1 | UUCUUGCCCCUUGGGCCUCCCCCCAGCCC | |
| | +UGA2 | CUCCUCCCCUUCCUGCACCCGUACCCCCG | |
| | +UGA3) | UGGUCUUUGAAUAAAGUCUGAGUGGGCG | |
| | | GC | |
| Downstream | v1.1 3′ UTR | UGAUAAUAGGCUGGAGCCUCGGUGGCCU | 1 |
| UTR | (+UGA1, | AGCUUCUUGCCCCUUGGGCCUCCCCCCAG | |
| | +UGA2, | CCCCUCCUCCCCUUCCUGCACCCGUACCC | |
| | +UGA3) | CCGUGGUCUUUGAAUAAAGUCUGAGUGG | |
| | | GCGGC | |
| 3′ UTR-001 | Creatine | GCGCCUGCCCACCUGCCACCGACUGCUGG | 32 |
| | Kinase | AACCCAGCCAGUGGGAGGGCCUGGCCCAC | |
| | | CAGAGUCCUGCUCCCUCACUCCUCGCCCC | |
| | | GCCCCCUGUCCCAGAGUCCCACCUGGGGG | |
| | | CUCUCUCCACCCUUCUCAGAGUUCCAGUU | |
| | | UCAACCAGAGUUCCAACCAAUGGGCUCCA | |
| | | UCCUCUGGAUUCUGGCCAAUGAAAUAUC | |
| | | UCCCUGGCAGGGUCCUCUUCUUUUCCCAG | |
| | | AGCUCCACCCCAACCAGGAGCUCUAGUUA | |
| | | AUGGAGAGCUCCCAGCACACUCGGAGCU | |
| | | UGUGCUUUGUCUCCACGCAAAGCGAUAA | |
| | | AUAAAAGCAUUGGUGGCCUUUGGUCUUU | |
| | | GAAUAAAGCCUGAGUAGGAAGUCUAGA | |
| 3′ UTR-002 | Myoglobin | GCCCCUGCCGCUCCCACCCCCACCCAUCU | 33 |
| | | GGGCCCCGGGUUCAAGAGAGAGCGGGGU | |
| | | CUGAUCUCGUGUAGCCAUAUAGAGUUUG | |
| | | CUUCUGAGUGUCUGCUUUGUUUAGUAGA | |
| | | GGUGGGCAGGAGGAGCUGAGGGGCUGGG | |
| | | GCUGGGGUGUUGAAGUUGGCUUUGCAUG | |
| | | CCCAGCGAUGCGCCUCCCUGUGGGAUGUC | |
| | | AUCACCCUGGGAACCGGGAGUGGCCCUU | |
| | | GGCUCACUGUGUUCUGCAUGGUUUGGAU | |
| | | CUGAAUUAAUUGUCCUUUCUUCUAAAUC | |
| | | CCAACCGAACUUCUUCCAACCUCCAAACU | |
| | | GGCUGUAACCCCAAAUCCAAGCCAUUAAC | |
| | | UACACCUGACAGUAGCAAUUGUCUGAUU | |
| | | AAUCACUGGCCCCUUGAAGACAGCAGAA | |
| | | UGUCCCUUUGCAAUGAGGAGGAGAUCUG | |
| | | GGCUGGGCGGGCCAGCUGGGGAAGCAUU | |
| | | UGACUAUCUGGAACUUGUGUGUGCCUCC | |
| | | UCAGGUAUGGCAGUGACUCACCUGGUUU | |
| | | UAAUAAAACAACCUGCAACAUCUCAUGG | |
| | | UCUUUGAAUAAAGCCUGAGUAGGAAGUC | |
| | | UAGA | |
| 3′ UTR-003 | α-actin | ACACACUCCACCUCCAGCACGCGACUUCU | 34 |
| | | CAGGACGACGAAUCUUCUCAAUGGGGGG | |
| | | GCGGCUGAGCUCCAGCCACCCCGCAGUCA | |
| | | CUUUCUUUGUAACAACUUCCGUUGCUGC | |
| | | CAUCGUAAACUGACACAGUGUUUAUAAC | |
| | | GUGUACAUACAUUAACUUAUUACCUCAU | |
| | | UUUGUUAUUUUUCGAAACAAAGCCCUGU | |
| | | GGAAGAAAAUGGAAAACUUGAAGAAGCA | |
| | | UUAAAGUCAUUCUGUUAAGCUGCGUAAA | |
| | | UGGUCUUUGAAUAAAGCCUGAGUAGGAA | |
| | | GUCUAGA | |
| 3′ UTR-004 | Albumin | CAUCACAUUUAAAAGCAUCUCAGCCUACC | 35 |
| | | AUGAGAAUAAGAGAAAGAAAAUGAAGAU | |
| | | CAAAAGCUUAUUCAUCUGUUUUUCUUUU | |
| | | UCGUUGGUGUAAAGCCAACACCCUGUCU | |
| | | AAAAAACAUAAAUUUCUUUAAUCAUUUU | |
| | | GCCUCUUUUCUCUGUGCUUCAAUUAAUA | |
| | | AAAAAUGGAAAGAAUCUAAUAGAGUGGU | |
| | | ACAGCACUGUUAUUUUUCAAAGAUGUGU | |
| | | UGCUAUCCUGAAAAUUCUGUAGGUUCUG | |
| | | UGGAAGUUCCAGUGUUCUCUCUUAUUCC | |
| | | ACUUCGGUAGAGGAUUUCUAGUUUCUUG | |
| | | UGGGCUAAUUAAAUAAAUCAUUAAUACU | |
| | | CUUCUAAUGGUCUUUGAAUAAAGCCUGA | |
| | | GUAGGAAGUCUAGA | |
| 3′ UTR-005 | α-globin | GCUGCCUUCUGCGGGGCUUGCCUUCUGGC | 36 |
| | | CAUGCCCUUCUUCUCUCCCUUGCACCUGU | |
| | | ACCUCUUGGUCUUUGAAUAAAGCCUGAG | |
| | | UAGGAAGGCGGCCGCUCGAGCAUGCAUC | |
| | | UAGA | |
| 3′ UTR-006 | G-CSF | GCCAAGCCCUCCCCAUCCCAUGUAUUUAU | 37 |
| | | CUCUAUUUAAUAUUUAUGUCUAUUUAAG | |
| | | CCUCAUAUUUAAAGACAGGGAAGAGCAG | |
| | | AACGGAGCCCCAGGCCUCUGUGUCCUUCC | |
| | | CUGCAUUUCUGAGUUUCAUUCUCCUGCC | |
| | | UGUAGCAGUGAGAAAAAGCUCCUGUCCU | |
| | | CCCAUCCCCUGGACUGGGAGGUAGAUAG | |
| | | GUAAAUACCAAGUAUUUAUUACUAUGAC | |
| | | UGCUCCCCAGCCCUGGCUCUGCAAUGGGC | |
| | | ACUGGGAUGAGCCGCUGUGAGCCCCUGG | |
| | | UCCUGAGGGUCCCCACCUGGGACCCUUGA | |
| | | GAGUAUCAGGUCUCCCACGUGGGAGACA | |
| | | AGAAAUCCCUGUUUAAUAUUUAAACAGC | |
| | | AGUGUUCCCCAUCUGGGUCCUUGCACCCC | |
| | | UCACUCUGGCCUCAGCCGACUGCACAGCG | |
| | | GCCCCUGCAUCCCCUUGGCUGUGAGGCCC | |
| | | CUGGACAAGCAGAGGUGGCCAGAGCUGG | |
| | | GAGGCAUGGCCCUGGGGUCCCACGAAUU | |
| | | UGCUGGGGAAUCUCGUUUUUCUUCUUAA | |
| | | GACUUUUGGGACAUGGUUUGACUCCCGA | |
| | | ACAUCACCGACGCGUCUCCUGUUUUUCUG | |
| | | GGUGGCCUCGGGACACCUGCCCUGCCCCC | |
| | | ACGAGGGUCAGGACUGUGACUCUUUUUA | |
| | | GGGCCAGGCAGGUGCCUGGACAUUUGCC | |
| | | UUGCUGGACGGGGACUGGGGAUGUGGGA | |
| | | GGGAGCAGACAGGAGGAAUCAUGUCAGG | |
| | | CCUGUGUGUGAAAGGAAGCUCCACUGUC | |
| | | ACCCUCCACCUCUUCACCCCCCACUCACC | |
| | | AGUGUCCCCUCCACUGUCACAUUGUAACU | |
| | | GAACUUCAGGAUAAUAAAGUGUUUGCCU | |
| | | CCAUGGUCUUUGAAUAAAGCCUGAGUAG | |
| | | GAAGGCGGCCGCUCGAGCAUGCAUCUAG | |
| | | A | |
| 3′ UTR-007 | Col1a2; | ACUCAAUCUAAAUUAAAAAAGAAAGAAA | 38 |
| | collagen, type | UUUGAAAAAACUUUCUCUUUGCCAUUUC | |
| | I, alpha 2 | UUCUUCUUCUUUUUUAACUGAAAGCUGA | |
| | | AUCCUUCCAUUUCUUCUGCACAUCUACUU | |
| | | GCUUAAAUUGUGGGCAAAAGAGAAAAAG | |
| | | AAGGAUUGAUCAGAGCAUUGUGCAAUAC | |
| | | AGUUUCAUUAACUCCUUCCCCCGCUCCCC | |
| | | CAAAAAUUUGAAUUUUUUUUUCAACACU | |
| | | CUUACACCUGUUAUGGAAAAUGUCAACC | |
| | | UUUGUAAGAAAACCAAAAUAAAAAUUGA | |
| | | AAAAUAAAAACCAUAAACAUUUGCACCA | |
| | | CUUGUGGCUUUUGAAUAUCUUCCACAGA | |
| | | GGGAAGUUUAAAACCCAAACUUCCAAAG | |
| | | GUUUAAACUACCUCAAAACACUUUCCCA | |
| | | UGAGUGUGAUCCACAUUGUUAGGUGCUG | |
| | | ACCUAGACAGAGAUGAACUGAGGUCCUU | |
| | | GUUUUGUUUUGUUCAUAAUACAAAGGUG | |
| | | CUAAUUAAUAGUAUUUCAGAUACUUGAA | |
| | | GAAUGUUGAUGGUGCUAGAAGAAUUUGA | |
| | | GAAGAAAUACUCCUGUAUUGAGUUGUAU | |
| | | CGUGUGGUGUAUUUUUUAAAAAAUUUGA | |
| | | UUUAGCAUUCAUAUUUUCCAUCUUAUUC | |
| | | CCAAUUAAAAGUAUGCAGAUUAUUUGCC | |
| | | CAAAUCUUCUUCAGAUUCAGCAUUUGUU | |
| | | CUUUGCCAGUCUCAUUUUCAUCUUCUUCC | |
| | | AUGGUUCCACAGAAGCUUUGUUUCUUGG | |
| | | GCAAGCAGAAAAAUUAAAUUGUACCUAU | |
| | | UUUGUAUAUGUGAGAUGUUUAAAUAAAU | |
| | | UGUGAAAAAAAUGAAAUAAAGCAUGUUU | |
| | | GGUUUUCCAAAAGAACAUAU | |
| 3′ UTR-008 | Col6a2; | CGCCGCCGCCCGGGCCCCGCAGUCGAGGG | 39 |
| | collagen, type | UCGUGAGCCCACCCCGUCCAUGGUGCUAA | |
| | VI, alpha 2 | GCGGGCCCGGGUCCCACACGGCCAGCACC | |
| | | GCUGCUCACUCGGACGACGCCCUGGGCCU | |
| | | GCACCUCUCCAGCUCCUCCCACGGGGUCC | |
| | | CCGUAGCCCCGGCCCCCGCCCAGCCCCAG | |
| | | GUCUCCCCAGGCCCUCCGCAGGCUGCCCG | |
| | | GCCUCCCUCCCCCUGCAGCCAUCCCAAGG | |
| | | CUCCUGACCUACCUGGCCCCUGAGCUCUG | |
| | | GAGCAAGCCCUGACCCAAUAAAGGCUUU | |
| | | GAACCCAU | |
| 3′ UTR-009 | RPN1; | GGGGCUAGAGCCCUCUCCGCACAGCGUGG | 40 |
| | ribophorin I | AGACGGGGCAAGGAGGGGGGUUAUUAGG | |
| | | AUUGGUGGUUUUGUUUUGCUUUGUUUAA | |
| | | AGCCGUGGGAAAAUGGCACAACUUUACC | |
| | | UCUGUGGGAGAUGCAACACUGAGAGCCA | |
| | | AGGGGUGGGAGUUGGGAUAAUUUUUAUA | |
| | | UAAAAGAAGUUUUUCCACUUUGAAUUGC | |
| | | UAAAAGUGGCAUUUUUCCUAUGUGCAGU | |
| | | CACUCCUCUCAUUUCUAAAAUAGGGACG | |
| | | UGGCCAGGCACGGUGGCUCAUGCCUGUA | |
| | | AUCCCAGCACUUUGGGAGGCCGAGGCAG | |
| | | GCGGCUCACGAGGUCAGGAGAUCGAGAC | |
| | | UAUCCUGGCUAACACGGUAAAACCCUGU | |
| | | CUCUACUAAAAGUACAAAAAAUUAGCUG | |
| | | GGCGUGGUGGUGGGCACCUGUAGUCCCA | |
| | | GCUACUCGGGAGGCUGAGGCAGGAGAAA | |
| | | GGCAUGAAUCCAAGAGGCAGAGCUUGCA | |
| | | GUGAGCUGAGAUCACGCCAUUGCACUCC | |
| | | AGCCUGGGCAACAGUGUUAAGACUCUGU | |
| | | CUCAAAUAUAAAUAAAUAAAUAAAUAAA | |
| | | UAAAUAAAUAAAUAAAAAUAAAGCGAGA | |
| | | UGUUGCCCUCAAA | |
| 3′ UTR-010 | LRP1; low | GGCCCUGCCCCGUCGGACUGCCCCCAGAA | 41 |
| | density | AGCCUCCUGCCCCCUGCCAGUGAAGUCCU | |
| | lipoprotein | UCAGUGAGCCCCUCCCCAGCCAGCCCUUC | |
| | receptor- | CCUGGCCCCGCCGGAUGUAUAAAUGUAA | |
| | related protein | AAAUGAAGGAAUUACAUUUUAUAUGUGA | |
| | 1 | GCGAGCAAGCCGGCAAGCGAGCACAGUA | |
| | | UUAUUUCUCCAUCCCCUCCCUGCCUGCUC | |
| | | CUUGGCACCCCCAUGCUGCCUUCAGGGAG | |
| | | ACAGGCAGGGAGGGCUUGGGGCUGCACC | |
| | | UCCUACCCUCCCACCAGAACGCACCCCAC | |
| | | UGGGAGAGCUGGUGGUGCAGCCUUCCCC | |
| | | UCCCUGUAUAAGACACUUUGCCAAGGCU | |
| | | CUCCCCUCUCGCCCCAUCCCUGCUUGCCC | |
| | | GCUCCCACAGCUUCCUGAGGGCUAAUUCU | |
| | | GGGAAGGGAGAGUUCUUUGCUGCCCCUG | |
| | | UCUGGAAGACGUGGCUCUGGGUGAGGUA | |
| | | GGCGGGAAAGGAUGGAGUGUUUUAGUUC | |
| | | UUGGGGGAGGCCACCCCAAACCCCAGCCC | |
| | | CAACUCCAGGGGCACCUAUGAGAUGGCC | |
| | | AUGCUCAACCCCCCUCCCAGACAGGCCCU | |
| | | CCCUGUCUCCAGGGCCCCCACCGAGGUUC | |
| | | CCAGGGCUGGAGACUUCCUCUGGUAAAC | |
| | | AUUCCUCCAGCCUCCCCUCCCCUGGGGAC | |
| | | GCCAAGGAGGUGGGCCACACCCAGGAAG | |
| | | GGAAAGCGGGCAGCCCCGUUUUGGGGAC | |
| | | GUGAACGUUUUAAUAAUUUUUGCUGAAU | |
| | | UCCUUUACAACUAAAUAACACAGAUAUU | |
| | | GUUAUAAAUAAAAUUGU | |
| 3′ UTR-011 | Nnt1; | AUAUUAAGGAUCAAGCUGUUAGCUAAUA | 42 |
| | cardiotrophin- | AUGCCACCUCUGCAGUUUUGGGAACAGG | |
| | like cytokine | CAAAUAAAGUAUCAGUAUACAUGGUGAU | |
| | factor 1 | GUACAUCUGUAGCAAAGCUCUUGGAGAA | |
| | | AAUGAAGACUGAAGAAAGCAAAGCAAAA | |
| | | ACUGUAUAGAGAGAUUUUUCAAAAGCAG | |
| | | UAAUCCCUCAAUUUUAAAAAAGGAUUGA | |
| | | AAAUUCUAAAUGUCUUUCUGUGCAUAUU | |
| | | UUUUGUGUUAGGAAUCAAAAGUAUUUUA | |
| | | UAAAAGGAGAAAGAACAGCCUCAUUUUA | |
| | | GAUGUAGUCCUGUUGGAUUUUUUAUGCC | |
| | | UCCUCAGUAACCAGAAAUGUUUUAAAAA | |
| | | ACUAAGUGUUUAGGAUUUCAAGACAACA | |
| | | UUAUACAUGGCUCUGAAAUAUCUGACAC | |
| | | AAUGUAAACAUUGCAGGCACCUGCAUUU | |
| | | UAUGUUUUUUUUUUCAACAAAUGUGACU | |
| | | AAUUUGAAACUUUUAUGAACUUCUGAGC | |
| | | UGUCCCCUUGCAAUUCAACCGCAGUUUG | |
| | | AAUUAAUCAUAUCAAAUCAGUUUUAAUU | |
| | | UUUUAAAUUGUACUUCAGAGUCUAUAUU | |
| | | UCAAGGGCACAUUUUCUCACUACUAUUU | |
| | | UAAUACAUUAAAGGACUAAAUAAUCUUU | |
| | | CAGAGAUGCUGGAAACAAAUCAUUUGCU | |
| | | UUAUAUGUUUCAUUAGAAUACCAAUGAA | |
| | | ACAUACAACUUGAAAAUUAGUAAUAGUA | |
| | | UUUUUGAAGAUCCCAUUUCUAAUUGGAG | |
| | | AUCUCUUUAAUUUCGAUCAACUUAUAAU | |
| | | GUGUAGUACUAUAUUAAGUGCACUUGAG | |
| | | UGGAAUUCAACAUUUGACUAAUAAAAUG | |
| | | AGUUCAUCAUGUUGGCAAGUGAUGUGGC | |
| | | AAUUAUCUCUGGUGACAAAAGAGUAAAA | |
| | | UCAAAUAUUUCUGCCUGUUACAAAUAUC | |
| | | AAGGAAGACCUGCUACUAUGAAAUAGAU | |
| | | GACAUUAAUCUGUCUUCACUGUUUAUAA | |
| | | UACGGAUGGAUUUUUUUUCAAAUCAGUG | |
| | | UGUGUUUUGAGGUCUUAUGUAAUUGAUG | |
| | | ACAUUUGAGAGAAAUGGUGGCUUUUUUU | |
| | | AGCUACCUCUUUGUUCAUUUAAGCACCA | |
| | | GUAAAGAUCAUGUCUUUUUAUAGAAGUG | |
| | | UAGAUUUUCUUUGUGACUUUGCUAUCGU | |
| | | GCCUAAAGCUCUAAAUAUAGGUGAAUGU | |
| | | GUGAUGAAUACUCAGAUUAUUUGUCUCU | |
| | | CUAUAUAAUUAGUUUGGUACUAAGUUUC | |
| | | UCAAAAAAUUAUUAACACAUGAAAGACA | |
| | | AUCUCUAAACCAGAAAAAGAAGUAGUAC | |
| | | AAAUUUUGUUACUGUAAUGCUCGCGUUU | |
| | | AGUGAGUUUAAAACACACAGUAUCUUUU | |
| | | GGUUUUAUAAUCAGUUUCUAUUUUGCUG | |
| | | UGCCUGAGAUUAAGAUCUGUGUAUGUGU | |
| | | GUGUGUGUGUGUGUGCGUUUGUGUGUUA | |
| | | AAGCAGAAAAGACUUUUUUAAAAGUUUU | |
| | | AAGUGAUAAAUGCAAUUUGUUAAUUGAU | |
| | | CUUAGAUCACUAGUAAACUCAGGGCUGA | |
| | | AUUAUACCAUGUAUAUUCUAUUAGAAGA | |
| | | AAGUAAACACCAUCUUUAUUCCUGCCCU | |
| | | UUUUCUUCUCUCAAAGUAGUUGUAGUUA | |
| | | UAUCUAGAAAGAAGCAAUUUUGAUUUCU | |
| | | UGAAAAGGUAGUUCCUGCACUCAGUUUA | |
| | | AACUAAAAAUAAUCAUACUUGGAUUUUA | |
| | | UUUAUUUUUGUCAUAGUAAAAAUUUUAA | |
| | | UUUAUAUAUAUUUUUAUUUAGUAUUAUC | |
| | | UUAUUCUUUGCUAUUUGCCAAUCCUUUG | |
| | | UCAUCAAUUGUGUUAAAUGAAUUGAAAA | |
| | | UUCAUGCCCUGUUCAUUUUAUUUUACUU | |
| | | UAUUGGUUAGGAUAUUUAAAGGAUUUUU | |
| | | GUAUAUAUAAUUUCUUAAAUUAAUAUUC | |
| | | CAAAAGGUUAGUGGACUUAGAUUAUAAA | |
| | | UUAUGGCAAAAAUCUAAAAACAACAAAA | |
| | | AUGAUUUUUAUACAUUCUAUUUCAUUAU | |
| | | UCCUCUUUUUCCAAUAAGUCAUACAAUU | |
| | | GGUAGAUAUGACUUAUUUUAUUUUUGUA | |
| | | UUAUUCACUAUAUCUUUAUGAUAUUUAA | |
| | | GUAUAAAUAAUUAAAAAAAUUUAUUGUA | |
| | | CCUUAUAGUCUGUCACCAAAAAAAAAAA | |
| | | AUUAUCUGUAGGUAGUGAAAUGCUAAUG | |
| | | UUGAUUUGUCUUUAAGGGCUUGUUAACU | |
| | | AUCCUUUAUUUUCUCAUUUGUCUUAAAU | |
| | | UAGGAGUUUGUGUUUAAAUUACUCAUCU | |
| | | AAGCAAAAAAUGUAUAUAAAUCCCAUUA | |
| | | CUGGGUAUAUACCCAAAGGAUUAUAAAU | |
| | | CAUGCUGCUAUAAAGACACAUGCACACG | |
| | | UAUGUUUAUUGCAGCACUAUUCACAAUA | |
| | | GCAAAGACUUGGAACCAACCCAAAUGUC | |
| | | CAUCAAUGAUAGACUUGAUUAAGAAAAU | |
| | | GUGCACAUAUACACCAUGGAAUACUAUG | |
| | | CAGCCAUAAAAAAGGAUGAGUUCAUGUC | |
| | | CUUUGUAGGGACAUGGAUAAAGCUGGAA | |
| | | ACCAUCAUUCUGAGCAAACUAUUGCAAG | |
| | | GACAGAAAACCAAACACUGCAUGUUCUC | |
| | | ACUCAUAGGUGGGAAUUGAACAAUGAGA | |
| | | ACACUUGGACACAAGGUGGGGAACACCA | |
| | | CACACCAGGGCCUGUCAUGGGGUGGGGG | |
| | | GAGUGGGGAGGGAUAGCAUUAGGAGAUA | |
| | | UACCUAAUGUAAAUGAUGAGUUAAUGGG | |
| | | UGCAGCACACCAACAUGGCACAUGUAUA | |
| | | CAUAUGUAGCAAACCUGCACGUUGUGCA | |
| | | CAUGUACCCUAGAACUUAAAGUAUAAUU | |
| | | AAAAAAAAAAAGAAAACAGAAGCUAUUU | |
| | | AUAAAGAAGUUAUUUGCUGAAAUAAAUG | |
| | | UGAUCUUUCCCAUUAAAAAAAUAAAGAA | |
| | | AUUUUGGGGUAAAAAAACACAAUAUAUU | |
| | | GUAUUCUUGAAAAAUUCUAAGAGAGUGG | |
| | | AUGUGAAGUGUUCUCACCACAAAAGUGA | |
| | | UAACUAAUUGAGGUAAUGCACAUAUUAA | |
| | | UUAGAAAGAUUUUGUCAUUCCACAAUGU | |
| | | AUAUAUACUUAAAAAUAUGUUAUACACA | |
| | | AUAAAUACAUACAUUAAAAAAUAAGUAA | |
| | | AUGUA | |
| 3′ UTR-012 | Col6a1; | CCCACCCUGCACGCCGGCACCAAACCCUG | 43 |
| | collagen, type | UCCUCCCACCCCUCCCCACUCAUCACUAA | |
| | VI, alpha 1 | ACAGAGUAAAAUGUGAUGCGAAUUUUCC | |
| | | CGACCAACCUGAUUCGCUAGAUUUUUUU | |
| | | UAAGGAAAAGCUUGGAAAGCCAGGACAC | |
| | | AACGCUGCUGCCUGCUUUGUGCAGGGUC | |
| | | CUCCGGGGCUCAGCCCUGAGUUGGCAUCA | |
| | | CCUGCGCAGGGCCCUCUGGGGCUCAGCCC | |
| | | UGAGCUAGUGUCACCUGCACAGGGCCCUC | |
| | | UGAGGCUCAGCCCUGAGCUGGCGUCACCU | |
| | | GUGCAGGGCCCUCUGGGGCUCAGCCCUGA | |
| | | GCUGGCCUCACCUGGGUUCCCCACCCCGG | |
| | | GCUCUCCUGCCCUGCCCUCCUGCCCGCCC | |
| | | UCCCUCCUGCCUGCGCAGCUCCUUCCCUA | |
| | | GGCACCUCUGUGCUGCAUCCCACCAGCCU | |
| | | GAGCAAGACGCCCUCUCGGGGCCUGUGCC | |
| | | GCACUAGCCUCCCUCUCCUCUGUCCCCAU | |
| | | AGCUGGUUUUUCCCACCAAUCCUCACCUA | |
| | | ACAGUUACUUUACAAUUAAACUCAAAGC | |
| | | AAGCUCUUCUCCUCAGCUUGGGGCAGCCA | |
| | | UUGGCCUCUGUCUCGUUUUGGGAAACCA | |
| | | AGGUCAGGAGGCCGUUGCAGACAUAAAU | |
| | | CUCGGCGACUCGGCCCCGUCUCCUGAGGG | |
| | | UCCUGCUGGUGACCGGCCUGGACCUUGGC | |
| | | CCUACAGCCCUGGAGGCCGCUGCUGACCA | |
| | | GCACUGACCCCGACCUCAGAGAGUACUCG | |
| | | CAGGGGCGCUGGCUGCACUCAAGACCCUC | |
| | | GAGAUUAACGGUGCUAACCCCGUCUGCU | |
| | | CCUCCCUCCCGCAGAGACUGGGGCCUGGA | |
| | | CUGGACAUGAGAGCCCCUUGGUGCCACA | |
| | | GAGGGCUGUGUCUUACUAGAAACAACGC | |
| | | AAACCUCUCCUUCCUCAGAAUAGUGAUG | |
| | | UGUUCGACGUUUUAUCAAAGGCCCCCUU | |
| | | UCUAUGUUCAUGUUAGUUUUGCUCCUUC | |
| | | UGUGUUUUUUUCUGAACCAUAUCCAUGU | |
| | | UGCUGACUUUUCCAAAUAAAGGUUUUCA | |
| | | CUCCUCUC | |
| 3′ UTR-013 | Calr; | AGAGGCCUGCCUCCAGGGCUGGACUGAG | 44 |
| | calreticulin | GCCUGAGCGCUCCUGCCGCAGAGCUGGCC | |
| | | GCGCCAAAUAAUGUCUCUGUGAGACUCG | |
| | | AGAACUUUCAUUUUUUUCCAGGCUGGUU | |
| | | CGGAUUUGGGGUGGAUUUUGGUUUUGUU | |
| | | CCCCUCCUCCACUCUCCCCCACCCCCUCCC | |
| | | CGCCCUUUUUUUUUUUUUUUUUUAAACU | |
| | | GGUAUUUUAUCUUUGAUUCUCCUUCAGC | |
| | | CCUCACCCCUGGUUCUCAUCUUUCUUGAU | |
| | | CAACAUCUUUUCUUGCCUCUGUCCCCUUC | |
| | | UCUCAUCUCUUAGCUCCCCUCCAACCUGG | |
| | | GGGGCAGUGGUGUGGAGAAGCCACAGGC | |
| | | CUGAGAUUUCAUCUGCUCUCCUUCCUGG | |
| | | AGCCCAGAGGAGGGCAGCAGAAGGGGGU | |
| | | GGUGUCUCCAACCCCCCAGCACUGAGGAA | |
| | | GAACGGGGCUCUUCUCAUUUCACCCCUCC | |
| | | CUUUCUCCCCUGCCCCCAGGACUGGGCCA | |
| | | CUUCUGGGUGGGGCAGUGGGUCCCAGAU | |
| | | UGGCUCACACUGAGAAUGUAAGAACUAC | |
| | | AAACAAAAUUUCUAUUAAAUUAAAUUUU | |
| | | GUGUCUCC | |
| 3′ UTR-014 | Colla1; | CUCCCUCCAUCCCAACCUGGCUCCCUCCC | 45 |
| | collagen, type | ACCCAACCAACUUUCCCCCCAACCCGGAA | |
| | I, alpha 1 | ACAGACAAGCAACCCAAACUGAACCCCCU | |
| | | CAAAAGCCAAAAAAUGGGAGACAAUUUC | |
| | | ACAUGGACUUUGGAAAAUAUUUUUUUCC | |
| | | UUUGCAUUCAUCUCUCAAACUUAGUUUU | |
| | | UAUCUUUGACCAACCGAACAUGACCAAA | |
| | | AACCAAAAGUGCAUUCAACCUUACCAAA | |
| | | AAAAAAAAAAAAAAAAGAAUAAAUAAAU | |
| | | AACUUUUUAAAAAAGGAAGCUUGGUCCA | |
| | | CUUGCUUGAAGACCCAUGCGGGGGUAAG | |
| | | UCCCUUUCUGCCCGUUGGGCUUAUGAAA | |
| | | CCCCAAUGCUGCCCUUUCUGCUCCUUUCU | |
| | | CCACACCCCCCUUGGGGCCUCCCCUCCAC | |
| | | UCCUUCCCAAAUCUGUCUCCCCAGAAGAC | |
| | | ACAGGAAACAAUGUAUUGUCUGCCCAGC | |
| | | AAUCAAAGGCAAUGCUCAAACACCCAAG | |
| | | UGGCCCCCACCCUCAGCCCGCUCCUGCCC | |
| | | GCCCAGCACCCCCAGGCCCUGGGGGACCU | |
| | | GGGGUUCUCAGACUGCCAAAGAAGCCUU | |
| | | GCCAUCUGGCGCUCCCAUGGCUCUUGCAA | |
| | | CAUCUCCCCUUCGUUUUUGAGGGGGUCA | |
| | | UGCCGGGGGAGCCACCAGCCCCUCACUGG | |
| | | GUUCGGAGGAGAGUCAGGAAGGGCCACG | |
| | | ACAAAGCAGAAACAUCGGAUUUGGGGAA | |
| | | CGCGUGUCAAUCCCUUGUGCCGCAGGGCU | |
| | | GGGCGGGAGAGACUGUUCUGUUCCUUGU | |
| | | GUAACUGUGUUGCUGAAAGACUACCUCG | |
| | | UUCUUGUCUUGAUGUGUCACCGGGGCAA | |
| | | CUGCCUGGGGGCGGGGAUGGGGGCAGGG | |
| | | UGGAAGCGGCUCCCCAUUUUAUACCAAA | |
| | | GGUGCUACAUCUAUGUGAUGGGUGGGGU | |
| | | GGGGAGGGAAUCACUGGUGCUAUAGAAA | |
| | | UUGAGAUGCCCCCCCAGGCCAGCAAAUGU | |
| | | UCCUUUUUGUUCAAAGUCUAUUUUUAUU | |
| | | CCUUGAUAUUUUUCUUUUUUUUUUUUUU | |
| | | UUUUUGUGGAUGGGGACUUGUGAAUUUU | |
| | | UCUAAAGGUGCUAUUUAACAUGGGAGGA | |
| | | GAGCGUGUGCGGCUCCAGCCCAGCCCGCU | |
| | | GCUCACUUUCCACCCUCUCUCCACCUGCC | |
| | | UCUGGCUUCUCAGGCCUCUGCUCUCCGAC | |
| | | CUCUCUCCUCUGAAACCCUCCUCCACAGC | |
| | | UGCAGCCCAUCCUCCCGGCUCCCUCCUAG | |
| | | UCUGUCCUGCGUCCUCUGUCCCCGGGUUU | |
| | | CAGAGACAACUUCCCAAAGCACAAAGCA | |
| | | GUUUUUCCCCCUAGGGGUGGGAGGAAGC | |
| | | AAAAGACUCUGUACCUAUUUUGUAUGUG | |
| | | UAUAAUAAUUUGAGAUGUUUUUAAUUAU | |
| | | UUUGAUUGCUGGAAUAAAGCAUGUGGAA | |
| | | AUGACCCAAACAUAAUCCGCAGUGGCCUC | |
| | | CUAAUUUCCUUCUUUGGAGUUGGGGGAG | |
| | | GGGUAGACAUGGGGAAGGGGCUUUGGGG | |
| | | UGAUGGGCUUGCCUUCCAUUCCUGCCCUU | |
| | | UCCCUCCCCACUAUUCUCUUCUAGAUCCC | |
| | | UCCAUAACCCCACUCCCCUUUCUCUCACC | |
| | | CUUCUUAUACCGCAAACCUUUCUACUUCC | |
| | | UCUUUCAUUUUCUAUUCUUGCAAUUUCC | |
| | | UUGCACCUUUUCCAAAUCCUCUUCUCCCC | |
| | | UGCAAUACCAUACAGGCAAUCCACGUGC | |
| | | ACAACACACACACACACUCUUCACAUCUG | |
| | | GGGUUGUCCAAACCUCAUACCCACUCCCC | |
| | | UUCAAGCCCAUCCACUCUCCACCCCCUGG | |
| | | AUGCCCUGCACUUGGUGGCGGUGGGAUG | |
| | | CUCAUGGAUACUGGGAGGGUGAGGGGAG | |
| | | UGGAACCCGUGAGGAGGACCUGGGGGCC | |
| | | UCUCCUUGAACUGACAUGAAGGGUCAUC | |
| | | UGGCCUCUGCUCCCUUCUCACCCACGCUG | |
| | | ACCUCCUGCCGAAGGAGCAACGCAACAGG | |
| | | AGAGGGGUCUGCUGAGCCUGGCGAGGGU | |
| | | CUGGGAGGGACCAGGAGGAAGGCGUGCU | |
| | | CCCUGCUCGCUGUCCUGGCCCUGGGGGAG | |
| | | UGAGGGAGACAGACACCUGGGAGAGCUG | |
| | | UGGGGAAGGCACUCGCACCGUGCUCUUG | |
| | | GGAAGGAAGGAGACCUGGCCCUGCUCAC | |
| | | CACGGACUGGGUGCCUCGACCUCCUGAAU | |
| | | CCCCAGAACACAACCCCCCUGGGCUGGGG | |
| | | UGGUCUGGGGAACCAUCGUGCCCCCGCCU | |
| | | CCCGCCUACUCCUUUUUAAGCUU | |
| 3′ UTR-015 | Plod1; | UUGGCCAGGCCUGACCCUCUUGGACCUUU | 46 |
| | procollagen- | CUUCUUUGCCGACAACCACUGCCCAGCAG | |
| | lysine, 2- | CCUCUGGGACCUCGGGGUCCCAGGGAACC | |
| | oxoglutarate | CAGUCCAGCCUCCUGGCUGUUGACUUCCC | |
| | 5- | AUUGCUCUUGGAGCCACCAAUCAAAGAG | |
| | dioxygenase 1 | AUUCAAAGAGAUUCCUGCAGGCCAGAGG | |
| | | CGGAACACACCUUUAUGGCUGGGGCUCU | |
| | | CCGUGGUGUUCUGGACCCAGCCCCUGGAG | |
| | | ACACCAUUCACUUUUACUGCUUUGUAGU | |
| | | GACUCGUGCUCUCCAACCUGUCUUCCUGA | |
| | | AAAACCAAGGCCCCCUUCCCCCACCUCUU | |
| | | CCAUGGGGUGAGACUUGAGCAGAACAGG | |
| | | GGCUUCCCCAAGUUGCCCAGAAAGACUG | |
| | | UCUGGGUGAGAAGCCAUGGCCAGAGCUU | |
| | | CUCCCAGGCACAGGUGUUGCACCAGGGAC | |
| | | UUCUGCUUCAAGUUUUGGGGUAAAGACA | |
| | | CCUGGAUCAGACUCCAAGGGCUGCCCUGA | |
| | | GUCUGGGACUUCUGCCUCCAUGGCUGGU | |
| | | CAUGAGAGCAAACCGUAGUCCCCUGGAG | |
| | | ACAGCGACUCCAGAGAACCUCUUGGGAG | |
| | | ACAGAAGAGGCAUCUGUGCACAGCUCGA | |
| | | UCUUCUACUUGCCUGUGGGGAGGGGAGU | |
| | | GACAGGUCCACACACCACACUGGGUCACC | |
| | | CUGUCCUGGAUGCCUCUGAAGAGAGGGA | |
| | | CAGACCGUCAGAAACUGGAGAGUUUCUA | |
| | | UUAAAGGUCAUUUAAACCA | |
| 3′ UTR-016 | Nucb1; | UCCUCCGGGACCCCAGCCCUCAGGAUUCC | 47 |
| | nucleobindin | UGAUGCUCCAAGGCGACUGAUGGGCGCU | |
| | 1 | GGAUGAAGUGGCACAGUCAGCUUCCCUG | |
| | | GGGGCUGGUGUCAUGUUGGGCUCCUGGG | |
| | | GCGGGGGCACGGCCUGGCAUUUCACGCA | |
| | | UUGCUGCCACCCCAGGUCCACCUGUCUCC | |
| | | ACUUUCACAGCCUCCAAGUCUGUGGCUCU | |
| | | UCCCUUCUGUCCUCCGAGGGGCUUGCCUU | |
| | | CUCUCGUGUCCAGUGAGGUGCUCAGUGA | |
| | | UCGGCUUAACUUAGAGAAGCCCGCCCCCU | |
| | | CCCCUUCUCCGUCUGUCCCAAGAGGGUCU | |
| | | GCUCUGAGCCUGCGUUCCUAGGUGGCUC | |
| | | GGCCUCAGCUGCCUGGGUUGUGGCCGCCC | |
| | | UAGCAUCCUGUAUGCCCACAGCUACUGG | |
| | | AAUCCCCGCUGCUGCUCCGGGCCAAGCUU | |
| | | CUGGUUGAUUAAUGAGGGCAUGGGGUGG | |
| | | UCCCUCAAGACCUUCCCCUACCUUUUGUG | |
| | | GAACCAGUGAUGCCUCAAAGACAGUGUC | |
| | | CCCUCCACAGCUGGGUGCCAGGGGCAGGG | |
| | | GAUCCUCAGUAUAGCCGGUGAACCCUGA | |
| | | UACCAGGAGCCUGGGCCUCCCUGAACCCC | |
| | | UGGCUUCCAGCCAUCUCAUCGCCAGCCUC | |
| | | CUCCUGGACCUCUUGGCCCCCAGCCCCUU | |
| | | CCCCACACAGCCCCAGAAGGGUCCCAGAG | |
| | | CUGACCCCACUCCAGGACCUAGGCCCAGC | |
| | | CCCUCAGCCUCAUCUGGAGCCCCUGAAGA | |
| | | CCAGUCCCACCCACCUUUCUGGCCUCAUC | |
| | | UGACACUGCUCCGCAUCCUGCUGUGUGUC | |
| | | CUGUUCCAUGUUCCGGUUCCAUCCAAAU | |
| | | ACACUUUCUGGAACAAA | |
| 3′ UTR-017 | α-globin-1 | GCUGGAGCCUCGGUGGCCAUGCUUCUUG | 48 |
| | | CCCCUUGGGCCUCCCCCCAGCCCCUCCUC | |
| | | CCCUUCCUGCACCCGUACCCCCGUGGUCU | |
| | | UUGAAUAAAGUCUGAGUGGGCGGC | |
| 3′ UTR-018 | Downstream | UAAUAGGCUGGAGCCUCGGUGGCCAUGC | 49 |
| | UTR | UUCUUGCCCCUUGGGCCUCCCCCCAGCCC | |
| | | CUCCUCCCCUUCCUGCACCCGUACCCCCG | |
| | | UGGUCUUUGAAUAAAGUCUGAGUGGGCG | |
| | | GC | |
| 3′ UTR-019 | Downstream | UGAUAAUAGGCUGGAGCCUCGGUGGCCA | 50 |
| | UTR | UGCUUCUUGCCCCUUGGGCCUCCCCCCAG | |
| | | CCCCUCCUCCCCUUCCUGCACCCGUACCC | |
| | | CCUGGUCUUUGAAUAAAGUCUGAGUGGG | |
| | | CGGC | |
| 3′ UTR-020 | Downstream | UGAUAAUAGGCUGGAGCCUCGGUGGCCA | 51 |
| | UTR | UGCUUCUUGCCCCUUGGGCCUCCCCCCAG | |
| | | CCCCUCCUCCCCUUCCUGCACCCGUACCC | |
| | | CCGUGGUCUUUGAAUAAAGUCUGAGUGG | |
| | | GCGGC | |

In certain embodiments, the 3′ UTR sequence useful for the disclosure comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of SEQ ID NOs: 1-2, 5-7, and 32-51 and any combination thereof. In some embodiments, the 3′ UTR sequence further comprises a miRNA binding site, e.g., miR-122-3p binding site, a miR-122-5p binding site, a miR-142-3p binding site. In other embodiments, a 3′UTR sequence useful for the disclosure comprises 3′ UTR-018 (SEQ ID NO: 53). In other embodiments, a 3′ UTR sequence useful for the disclosure comprises 3′ UTR comprised of nucleotide sequence set forth in SEQ ID NO: 1. In other embodiments, a 3′ UTR sequence useful for the disclosure comprises 3′ UTR comprised of nucleotide sequence set forth in SEQ ID NO: 51.

In certain embodiments, the 3′ UTR sequence comprises one or more miRNA binding sites, e.g., miR-122-3p binding site, a miR-122-5p binding site, a miR-142-3p binding site, or any other heterologous nucleotide sequences therein, without disrupting the function of the 3′ UTR. Some examples of 3′ UTR sequences comprising a miRNA binding site are listed in Table 7.

In some embodiments, a 3′UTR sequence comprises one or more miRNA binding sites, e.g., miR-122-3p binding site, a miR-122-5p binding site, a miR-142-3p binding site, or any other heterologous nucleotide sequences therein, wherein the 3′UTR is altered to be endonuclease resistant using a method of the disclosure. In some embodiments, a 3′UTR sequence comprising one or more miRNA binding sites is altered to increase or improve endonuclease resistance and/or decrease or reduce endonuclease susceptibility by altering one or more endonuclease sensitive sequence motifs comprising the nucleotide sequence WGA, wherein W=adenine (A) or uracil (U). In some embodiments, a 3′UTR comprises at least one miRNA binding site, wherein the miRNA binding site comprises one or more endonuclease sensitive sequence motifs that are altered to increase or improve endonuclease resistance and/or decrease or reduce endonuclease susceptibility. In some embodiments, altering a miRNA binding site in the 3′UTR that comprises one or more endonuclease sensitive sequence motifs does not disrupt the function of the miRNA binding site. In some embodiments, a 5′UTR sequence comprises one or more miRNA binding sites, e.g., miR-122-3p binding site, a miR-122-5p binding site, a miR-142-3p binding site, or any other heterologous nucleotide sequences therein, wherein the 5′UTR is altered to increase or improve endonuclease resistance and/or decrease or reduce endonuclease susceptibility. In some embodiments, a 5′UTR sequence comprising one or more miRNA binding sites is altered to increase or improve endonuclease resistance and/or decrease or reduce endonuclease susceptibility by altering one or more endonuclease sensitive sequence motifs comprising the nucleotide sequence WGA, wherein W=adenine (A) or uracil (U) using a method of the disclosure. In some embodiments, a 5′UTR comprises at least one miRNA binding site, wherein the miRNA binding site comprises one or more endonuclease sensitive sequence motifs that are altered to increase or improve endonuclease resistance and/or decrease or reduce endonuclease susceptibility. In some embodiments, altering a miRNA binding site in the 5′UTR that comprises one or more endonuclease sensitive sequence motifs does not disrupt the function of the miRNA binding site.

TABLE 7
Exemplary 3′ UTR with miRNA Binding Sites and alterations to remove endonuclease
sensitive sites

| 3′ UTR | | | SEQ |
|---|---|---|---|
| Identifier/miRNA | Name/ | | ID |
| binding site | Description | Sequence | NO. |
| 3′ UTR-018 + miR- | Downstream | UAAUAGGCUGGAGCCUCGGUGGCCAUGC | 52 |
| 122-5p binding site | UTR | UUCUUGCCCCUUGGGCCUCCCCCCAGCCC | |
| | | CUCCUCCCCUUCCUGCACCCGUACCCCCC | |
| | | AAACACCAUUGUCACACUCCAGUGGUCU | |
| | | UUGAAUAAAGUCUGAGUGGGCGGC | |
| 3′ UTR-018 + miR- | Downstream | UAAUAGGCUGGAGCCUCGGUGGCCAUGC | 53 |
| 122-3p binding site | UTR | UUCUUGCCCCUUGGGCCUCCCCCCAGCCC | |
| | | CUCCUCCCCUUCCUGCACCCGUACCCCCU | |
| | | AUUUAGUGUGAUAAUGGCGUUGUGGUC | |
| | | UUUGAAUAAAGUCUGAGUGGGCGGC | |
| 3′ UTR-019 + miR- | Downstream | UGAUAAUAGGCUGGAGCCUCGGUGGCCA | 54 |
| 122-5p binding site | UTR | UGCUUCUUGCCCCUUGGGCCUCCCCCCAG | |
| | | CCCCUCCUCCCCUUCCUGCACCCGUACCC | |
| | | CCCAAACACCAUUGUCACACUCCAGUGG | |
| | | UCUUUGAAUAAAGUCUGAGUGGGCGGC | |
| 3′ UTR + miR-142- | Downstream | GCUGGAGCCUCGGUGGCCAUGCUUCUUG | 55 |
| 3p binding site | UTR | CCCCUUGGGCCUCCCCCCAGCCCCUCCUC | |
| | | CCCUUCCUGCACCCGUACCCCCUCCAUAA | |
| | | AGUAGGAAACACUACAGUGGUCUUUGA | |
| | | AUAAAGUCUGAGUGGGCGGC | |
| Altered 3′ UTR-019 + | Downstream | UAAUAGGCUGGAGCCUCGGUGGCCAUGC | 56 |
| miR-122-5p | UTR | UUCUUGCCCCUUGGGCCUCCCCCCAGCCC | |
| binding site | | CUCCUCCCCUUCCUGCACCCGUACCCCCC | |
| | | AAACACCAUUGUCACACUCCAGUGGUCU | |
| | | UUCAAUAAAGUCUCAGUGGGCGGC | |
| Altered 3′ UTR + | Downstream | GCUGGAGCCUCGGUGGCCAUGCUUCUUG | 57 |
| miR-142-3p binding | UTR | CCCCUUGGGCCUCCCCCCAGCCCCUCCUC | |
| site endonuclease | | CCCUUCCUGCACCCGUACCCCCUCCAUAA | |
| resistant | | AGUAGGAAACACUACAGUGGUCUUUCAA | |
| | | UAAAGUCUCAGUGGGCGGC | |

*miRNA binding site is bolded.

In certain embodiments, the 3′ UTR sequence useful for the disclosure comprises a nucleotide sequence at least about 60%, at least about 70%, at least about 80%, at least about t90%, at least about 95%, at least about 96%, at least about 97%, at least about 98%, at least about 99%, or about 100% identical to the sequence set forth as SEQ ID NO: 1 or SEQ ID NO: 50.

### Regions Having a 5′ Cap

The polynucleotide comprising an mIRNA encoding a polypeptide of the present disclosure can further comprise a 5′ cap. The 5′ cap useful for polypeptide encoding mRNA can bind the mIRNA Cap Binding Protein (CBP), thereby increasing mRNA stability. The cap can further assist the removal of 5′ proximal introns removal during mIRNA splicing.

In some embodiments, the polynucleotide comprising an mIRNA encoding a polypeptide of the present disclosure comprises a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as α-methyl-phosphonate and seleno-phosphate nucleotides. Capping structures useful for preventing decapping and exonucleolytic degradation are further described by Kalek, et al (2005) Synthesis and biochemical properties of novel mRNA 5′cap analogs resistant to enzymatic hydrolysis, Nucleosides, Nucleotides and Nucleic Acids, 24:5-7, 615-621.

In certain embodiments, the 5′ cap comprises 2′-O-methylation of the ribose sugars of 5′-terminal and/or 5′-anteterminal nucleotides on the 2′-hydroxyl group of the sugar ring. In other embodiments, the caps for the polypeptide-encoding mRNA include cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs can be chemically (i.e. non-enzymatically) or enzymatically synthesized and/or linked to the polynucleotides of the disclosure.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m7G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m7Gm-ppp-G).

In some embodiments, the cap is a dinucleotide cap analog. As a non-limiting example, the dinucleotide cap analog can be modified at different phosphate positions with a boranophosphate group or a phosphoroselenoate group such as the dinucleotide cap analogs described in U.S. Pat. No. 8,519,110.

In another embodiment, the cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m3′-OG(5′)ppp(5′)G cap analog. See, e.g., the various cap analogs and the methods of synthesizing cap analogs described in Kore et al. (2013) Bioorganic & Medicinal Chemistry 21:4570-4574. In another embodiment, a cap analog of the present disclosure is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

An mRNA of the present disclosure can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects.

Non-limiting examples of more authentic 5′ cap structures of the present disclosure are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)NlmpNp (cap 1), and 7mG(5′)-ppp(5′)NlmpN2mp (cap 2).

According to the present disclosure, 5′ terminal caps can include endogenous caps or cap analogs. According to the present disclosure, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### 5′ Capping and 5′ Trinucleotide Cap

It is desirable to manufacture therapeutic RNAs enzymatically using in vitro transcription (IVT). In general, a DNA-dependent RNA polymerase transcribes a DNA template containing an appropriate promoter into an RNA transcript. The poly(A) tail can be generated co-transcriptionally by incorporating a poly(T) tract in the template DNA or separately by using a poly(A) polymerase. Eukaryotic mRNAs start with a 5′ cap (e.g., a 5′ m7GpppX cap). Typically, the 5′ cap begins with an inverted G with N⁷Me (required for eIF4E binding). A preferred cap, Cap1 contains 2′OMe at the +1 position) followed by any nucleoside at +2 position. This cap can be installed post-transcriptionally, e.g., enzymatically (after transcription) or co-transcriptionally (during transcription).

Post-transcriptional capping can be carried out using the vaccinia capping enzyme and allows for complete capping of the RNA, generating a cap 0 structure on RNA carrying a 5′ terminal triphosphate or diphosphate group, the cap 0 structure being required for efficient translation of the mRNA in vivo. The cap 0 structure can then be further modified into cap 1 using a cap-specific 2′O methyltransferase. Vaccinia capping enzyme and 2′O methyltransferase have been used to generate cap 0 and cap 1 structures on in vitro transcripts, for example, for use in transfecting eukaryotic cells or in mRNA therapeutic applications to drive protein synthesis. While post-transcriptional capping by vaccinia capping enzymes can yield either Cap 0 or Cap 1 structures, it is an expensive process when utilized for large-scale mRNA production, for example, vaccinia is costly and in limited supply and there can be difficulties in purifying an IVT mRNA (e.g., removing S-adenosylmethionine (SAM) and 2′O-methyltransferase). Moreover, capping can be incomplete due to inaccessibility of structured 5′ ends.

Co-transcriptional capping using a cap analog has certain advantages over vaccinia capping, for example, the process requires a simpler workflow (e.g., no need for a purification step between transcription and capping). Traditional co-transcriptional capping methods utilize the dinucleotide ARCA (anti-reverse cap analog) and yield Cap 0 structures. ARCA capping has drawbacks, however, for example, the resulting Cap 0 structures can be immunogenic and the process often results in low yields and/or poorly capped material. Another potential drawback of this approach is a theoretical capping efficiency of <100%, due to competition from the GTP for the starting nucleotide. For example, co-transcriptional capping using ARCA typically requires a 10:1 ratio of ARCA:GTP to achieve >90% capping (needed to outcompete GTP for initiation).

In some embodiments, mRNAs of the disclosure are comprised of trinucleotide mRNA cap analogs, prepared using co-transcriptional capping methods (e.g., featuring T7 RNA polymerase) for the in vitro synthesis of mRNA. Use of a trinucleotide cap analog may provide a solution to several of the above-described problems associated with vaccinia or ARCA capping. In addition, the methods of co-transcriptional capping described provide flexibility in modifying the penultimate nucleobase which may alter binding behavior, or affect the affinity of these caps towards decapping enzymes, or both, thus potentially improving stability of the respective mRNA. An exemplary trinucleotide for use in the herein-described co-transcriptional capping methods is the m7GpppAG (GAG) trinucleotide. Use of this trinucleotide results in the nucleotide at the +1 position being A instead of G. Both +1G and +1A are caps that can be found in naturally-occurring mRNAs.

T7 RNA polymerase prefers to initiate with 5′ GTP. Accordingly, most conventional mRNA transcripts start with 5′- [symbol] (based on transcription from a T7 promoter sequence such as 5′TAATACGACTCACTATA [symbol] NNNNNNNNN . . . 3′ (SEQ ID NO: 76; TATA being referred to as the “TATA box”). T7 RNA polymerase typically transcribes DNA downstream of a T7 promoter (5′ TAATACGACTCACTATAG 3′, referencing the coding strand (SEQ ID NO: 77)). T7 polymerase starts transcription at the underlined G in the promoter sequence. The polymerase then transcribes using the opposite strand as a template from 5′->3′. The first base in the transcript will be a G.

The herein-described processes capitalize on the fact that the T7 enzyme has limited initiation activity with the single nucleotide ATP, driving T7 to initiate with the trinucleotide rather than ATP. The process thus generates an mRNA product with >90% functional cap post-transcription. The process is an efficient “one-pot” mRNA production method that includes, for example, the GAG trinucleotide (GpppAG; ^{m}GpppAmG) in equimolar concentration with the NTPs, GTP, ATP, CTP and UTP. The process features an “A-start” DNA template that initiates transcription with 5′ adenosine (A). As defined herein, “A-start” and “G-start” DNA templates are double-stranded DNA having requisite nucleosides in the template strand, such that the coding strand (and corresponding mRNA) begin with A or G, respectively. For example, a G-start DNA template features a template strand having the nucleobases CC complementary to GG immediately downstream of the TATA box in the T7 promoter (referencing the coding strand), and an A-start DNA template features a template strand having the nucleobases TC complementary to the AG immediately downstream of the TATA box in the T7 promoter (referencing the coding strand).

An exemplary T7 promoter sequence featured in an A-start DNA template of the present disclosure is depicted here:

(SEQ ID NO: 78)
5′TAATACGACTCACTATA [symbol] NNNNNNNNNN . . . 3′
(SEQ ID NO: 79)
5′ATTATGCTGAGTGATAT [symbol] NNNNNNNNNN . . . 3′

The trinucleotide-based capping methods described herein provide flexibility in dictating the penultimate nucleobase. The trinucleotide capping methods of the present disclosure provide efficient production of capped mRNA, for example, 95-98% capped mRNA with a natural cap 1 structure.

### Trinucleotide Caps

Provided herein are co-transcriptional capping methods for ribonucleic acid (RNA) synthesis. That is, RNA is produced in a “one-pot” reaction, without the need for a separate capping reaction. Thus, the methods, in some embodiments, comprise reacting a DNA template with a T7 RNA polymerase variant, nucleoside triphosphates, and a cap analog under in vitro transcription reaction conditions to produce RNA transcript.

A cap analog may be, for example, a dinucleotide cap, a trinucleotide cap, or a tetranucleotide cap. In some embodiments, a cap analog is a dinucleotide cap. In some embodiments, a cap analog is a trinucleotide cap. In some embodiments, a cap analog is a tetranucleotide cap.

A trinucleotide cap, in some embodiments, comprises a compound of formula (I)

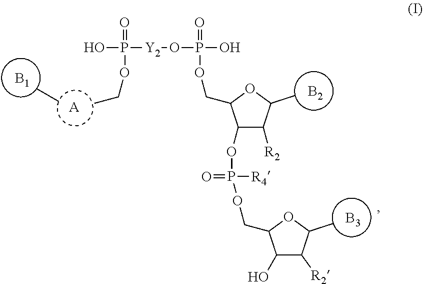

or a stereoisomer, tautomer or salt thereof, wherein

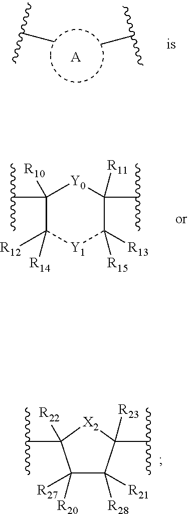

ring B₁ is a modified or unmodified Guanine;

ring B₂ and ring B₃ each independently is a nucleobase or a modified nucleobase;

X₂ is O, S(O)ₚ, NR₂₄, or CR₂₅R₂₆ in which p is 0, 1, or 2;

Y₀ is O or CR₆R₇;

Y1 is O, S(O)ₙ, CR₆R₇, or NR₈, in which n is 0, 1, or 2;

each [symbol] is a single bond or absent, wherein when each [symbol] is a single bond, Yi is O, S(O)ₙ, CR₆R₇, or NR₈; and when each [symbol] is absent, Y₁ is void;

Y₂ is (OP(O)R₄)m in which m is 0, 1, or 2, or —O—(CR₄₀R₄₁)u-Q₀-(CR₄₂R₄₃)v-, in which Q₀ is a bond, O, S(O)ᵣ, NR₄₄, or CR₄₅R₄₆, r is 0, 1, or 2, and each of u and v independently is 1, 2, 3 or 4;

each R₂ and R₂′ independently is halo, LNA, or OR₃;

each R₃ independently is H, C₁-C₆ alkyl, C₂-C₆ alkenyl, or C₂-C₆ alkynyl and R₃, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;

each R₄ and R₄′ independently is H, halo, C₁-C₆ alkyl, OH, SH, SeH, or BH₃⁻;

each of R₆, R₇, and R₈, independently, is -Q₁-T₁, in which Q₁ is a bond or C₁-C₃ alkyl linker optionally substituted with one or more of halo, cyano, OH and C₁-C₆ alkoxy, and T₁ is H, halo, OH, COOH, cyano, or Rₛ₁, in which Rₛ₁ is C₁-C₃ alkyl, C₂-C₆ alkenyl, C₂-C₆ alkynyl, C₁-C₆ alkoxyl, C(O)O—C₁-C₆ alkyl, C₃-C₈ cycloalkyl, C₆-C₁₀ aryl, NR₃₁R₃₂, (NR₃₁R₃₂R₃₃)⁺, 4 to 12-membered heterocycloalkyl, or 5- or 6-membered heteroaryl, and Rₛ₁ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl;

each of R₁₀, R₁₁, R₁₂, R₁₃ R₁₄, and R₁₅, independently, is -Q₂-T₂, in which Q₂ is a bond or C₁-C₃ alkyl linker optionally substituted with one or more of halo, cyano, OH and C₁-C₆ alkoxy, and T₂ is H, halo, OH, NH₂, cyano, NO₂, N₃, Rₛ₂, or ORₛ₂, in which Rₛ₂ is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, NR₃₁R₃₂, (NR₃₁R₃₂R₃₃)⁺, 4 to 12-membered heterocycloalkyl, or 5- or 6-membered heteroaryl, and Rₛ₂ is optionally substituted with one or more substituents selected from the group consisting of halo, OH, oxo, C₁-C₆ alkyl, COOH, C(O)O—C₁-C₆ alkyl, cyano, C₁-C₆ alkoxyl, NR₃₁R₃₂, (NR₃₁R₃₂R₃₃)⁺, C₃-C₈ cycloalkyl, C₆-C₁₀ aryl, 4 to 12-membered heterocycloalkyl, and 5- or 6-membered heteroaryl; or alternatively R₁₂ together with R₁₄ is oxo, or R₁₃ together with R₁₅ is oxo,

each of R₂₀, R₂₁, R₂₂, and R₂₃ independently is -Q₃-T₃, in which Q₃ is a bond or C₁-C₃ alkyl linker optionally substituted with one or more of halo, cyano, OH and C₁-C₆ alkoxy, and T₃ is H, halo, OH, NH₂, cyano, NO₂, N₃, R_{S3}, or OR_{S3}, in which R_{S3} is C₁-C₆ alkyl, C₂-C₆ alkenyl, C₂-C₆ alkynyl, C₃-C₈ cycloalkyl, C₆-C₁₀ aryl, NHC(O)—C₁-C₆ alkyl, mono-C₁-C₆ alkylamino, di-C₁-C₆ alkylamino, 4 to 12-membered heterocycloalkyl, or 5- or 6-membered heteroaryl, and Rₛ₃ is optionally substituted with one or more substituents selected from the group consisting of halo, OH, oxo, C₁-C₆ alkyl, COOH, C(O)O—C₁-C₆ alkyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, and 5- or 6-membered heteroaryl;

each of R₂₄, R₂₅, and R₂₆ independently is H or C₁-C₆ alkyl;

each of R₂₇ and R₂₈ independently is H or OR₂₉; or R₂₇ and R₂₈ together form O—R₃₀—O; each R₂₉ independently is H, C₁-C₆ alkyl, C₂-C₆ alkenyl, or C₂-C₆ alkynyl and R₂₉, when being C₁-C₆ alkyl, C₂-C₆ alkenyl, or C₂-C₆ alkynyl, is optionally substituted with one or more of halo, OH and C₁-C₆ alkoxyl that is optionally substituted with one or more OH or OC(O)—C₁-C₆ alkyl;

R₃₀ is C₁-C₆ alkylene optionally substituted with one or more of halo, OH and C₁-C₆ alkoxyl;

each of R₃₁, R₃₂, and R₃₃, independently is H, C₁-C₆ alkyl, C₃-C₈ cycloalkyl, C₆-C₁₀ aryl, 4 to 12-membered heterocycloalkyl, or 5- or 6-membered heteroaryl;

each of R₄₀, R₄₁, R₄₂, and R₄₃ independently is H, halo, OH, cyano, N₃, OP(O)R₄₇R₄₈, or C₁-C₆ alkyl optionally substituted with one or more OP(O)R₄₇R₄₈, or one R₄₁ and one R₄₃, together with the carbon atoms to which they are attached and Q₀, form C₄-C₁₀ cycloalkyl, 4- to 14-membered heterocycloalkyl, C₆-C₁₀ aryl, or 5- to 14-membered heteroaryl, and each of the cycloalkyl, heterocycloalkyl, phenyl, or 5- to 6-membered heteroaryl is optionally substituted with one or more of OH, halo, cyano, N₃, oxo, OP(O)R₄₇R₄₈, C₁-C₆ alkyl, C₁-C₆ haloalkyl, COOH, C(O)O—C₁-C₆ alkyl, C₁-C₆ alkoxyl, C₁-C₆ haloalkoxyl, amino, mono-C₁-C₆ alkylamino, and di-C₁-C₆ alkylamino;

R₄₄ is H, C₁-C₆ alkyl, or an amine protecting group;

each of R₄₅ and R₄₆ independently is H, OP(O)R₄₇R₄₈, or C₁-C₆ alkyl optionally substituted with one or more OP(O)R₄₇R₄₈, and

each of R₄₇ and R₄₈, independently is H, halo, C₁-C₆ alkyl, OH, SH, SeH, or BH₃⁻.

It should be understood that a cap analog, as provided herein, may include any of the cap analogs described in International Publication No. WO 2017/066797, published on 20 Apr. 2017, incorporated by reference herein in its entirety.

In some embodiments, the B₂ middle position is a non-ribose molecule, such as arabinose.

In some embodiments R₂ is ethyl-based.

Thus, in some embodiments, a trinucleotide cap comprises the following structure:

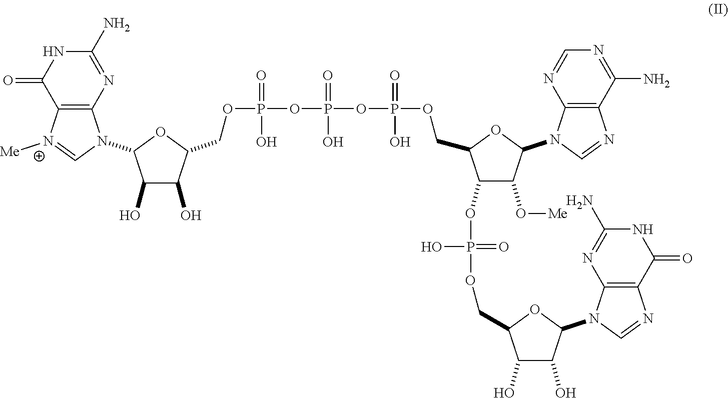

In other embodiments, a trinucleotide cap comprises the following structure:

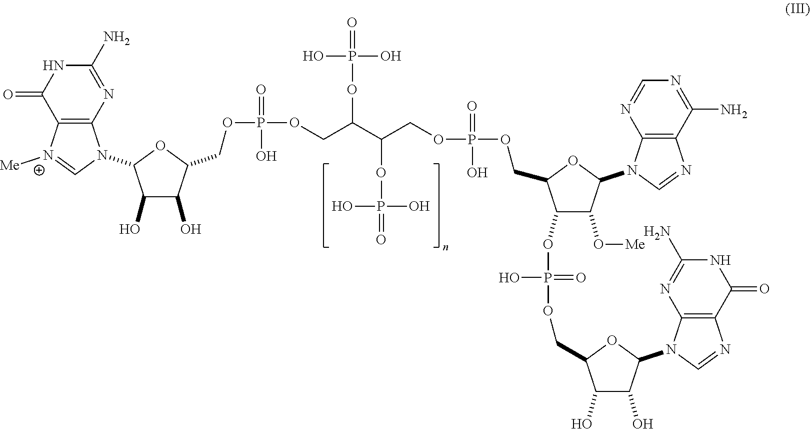

In yet other embodiments, a trinucleotide cap comprises the following structure:

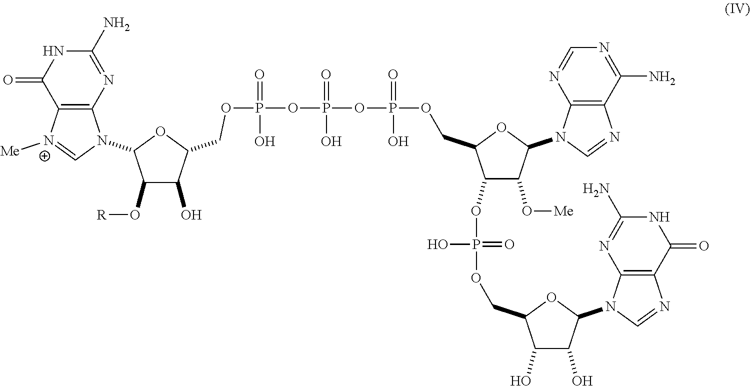

In still other embodiments, a trinucleotide cap comprises the following structure:

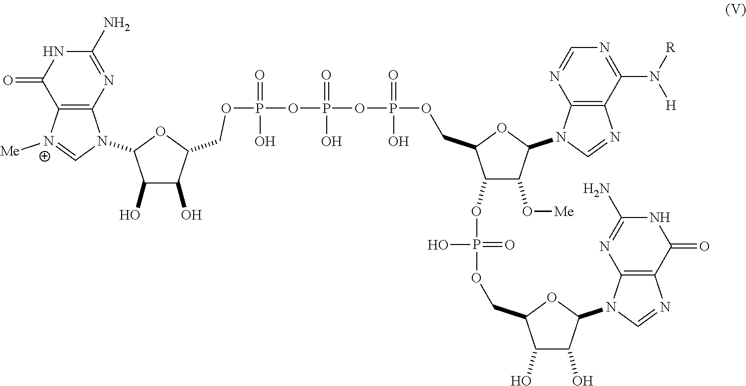

A trinucleotide cap, in some embodiments, comprises a sequence selected from the following sequences: GAA, GAC, GAG, GAU, GCA, GCC, GCG, GCU, GGA, GGC, GGG, GGU, GUA, GUC, GUG, and GUU.

In some embodiments, a trinucleotide cap comprises a sequence selected from the following sequences: m⁷GpppApA, m⁷GpppApC, m⁷GpppApG, m⁷GpppApU, m⁷GpppCpA, m⁷GpppCpC, m⁷GpppCpG, m⁷GpppCpU, m⁷GpppGpA, m⁷GpppGpC, m⁷GpppGpG, m⁷GpppGpU, m⁷GpppUpA, m⁷GpppUpC, m⁷GpppUpG, and m⁷GpppUpU.

A trinucleotide cap, in some embodiments, comprises a sequence selected from the following sequences: m⁷G_{3′OMe}pppApA, m⁷G_{3′OMe}pppApC, m⁷G_{3′OMe}pppApG, m⁷G_{3′OMe}pppApU, m⁷G_{3′OMe}pppCpA, m⁷G_{3′OMe}pppCpC, m⁷G_{3′OMe}pppCpG, m⁷G_{3′OMe}pppCpU, m⁷G_{3′OMe}pppGpA, m⁷G_{3′OMe}pppGpC, m⁷G_{3′OMe}pppGpG, m⁷G_{3′OMe}pppGpU, m⁷G_{3′OMe}pppUpA, m⁷G_{3′OMe}pppUpC, m⁷G_{3′OMe}pppUpG, and m⁷G_{3′OMe}pppUpU.

A trinucleotide cap, in other embodiments, comprises a sequence selected from the following sequences: m⁷G_{3′OMe}pppA_{2′OMe}pA, m⁷G_{3′OMe}pppA_{2′OMe}pC, m⁷G_{3′OMe}pppA_{2′OMe}pG, m⁷G_{3′OMe}pppA_{2′OMe}pU, m⁷G_{3′OMe}pppC_{2′OMe}pA, m⁷G_{3′OMe}pppC_{2′OMe}pC, m⁷G_{3′OMe}pppC_{2′OMe}pG, m⁷G_{3′OMe}pppC_{2′OMe}pU, m⁷G_{3′OMe}pppG_{2′OMe}pA, m⁷G_{3′OMe}pppG_{2′OMe}pC, m⁷G_{3′OMe}pppG_{2′OMe}pG, m⁷G_{3′OMe}pppG_{2′OMe}pU, m⁷G_{3′OMe}pppU_{2′OMe}pA, m⁷G_{3′OMe}pppU_{2′OMe}pC, m⁷G_{3′OMe}pppU_{2′OMe}pG, and m⁷G_{3′OMe}pppU_{2′OMe}pU.

A trinucleotide cap, in still other embodiments, comprises a sequence selected from the following sequences: m⁷GpppA_{2′OMe}pA, m⁷GpppA_{2′OMe}pC, m⁷GpppA_{2′OMe}pG, m⁷GpppA_{2′OMe}pU, m⁷GpppC_{2′OMe}pA, m⁷GpppC_{2′OMe}pC, m⁷GpppC_{2′OMe}pG, m⁷GpppC_{2′OMe}pU, m⁷GpppG_{2′OMe}pA, m⁷GpppG_{2′OMe}pC, m⁷GpppG_{2′OMe}pG, m⁷GpppG_{2′OMe}pU, m⁷GpppU_{2′OMe}pA, m⁷GpppU_{2′OMe}pC, m⁷GpppU_{2′OMe}pG, and m⁷GpppU_{2′OMe}pU.

A trinucleotide cap, in further embodiments, comprises a sequence selected from the following sequences: m⁷Gpppm⁶A_{2′OMe}pA, m⁷Gpppm⁶A_{2′OMe}pC, and m⁷Gpppm⁶A_{2′OMe}pG, m⁷Gpppm⁶A_{2′OMe}pU

A trinucleotide cap, in yet other embodiments, comprises a sequence selected from the following sequences: m⁷Gpppe⁶A_{2′OMe}pA, m⁷Gpppe⁶A_{2′OMe}pC, and m⁷Gpppe⁶A_{2′OMe}pG,

In some embodiments, a trinucleotide cap comprises GAG. In some embodiments, a trinucleotide cap comprises GCG. In some embodiments, a trinucleotide cap comprises GUG. In some embodiments, a trinucleotide cap comprises GGG.

### Transcription

Some aspects of the present disclosure provide co-transcriptional capping methods that comprise reacting a DNA template with an RNA polymerase (e.g., T7 RNA polymerase), nucleoside triphosphates, and a trinucleotide cap analog under in vitro transcription reaction conditions to produce RNA transcript. An RNA transcript, in some embodiments, is a messenger RNA (mRNA) that includes a nucleotide sequence encoding a polypeptide (e.g., protein or peptide) of interest (e.g., biologics, antibodies, antigens (vaccines), and therapeutic proteins) linked to a polyA tail. In some embodiments, the mRNA is modified mRNA (mmRNA), which includes at least one modified nucleotide. In some embodiments, a modified mRNA is comprised of one or more RNA elements.

IVT conditions typically require a purified linear DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application. Typical IVT reactions are performed by incubating a DNA template with an RNA polymerase and nucleoside triphosphates, including GTP, ATP, CTP, and UTP (or nucleotide analogs) in a transcription buffer. An RNA transcript having a 5′ terminal guanosine triphosphate is produced from this reaction.

A DNA template may encode a polypeptide of interest. A DNA template, in some embodiments, includes an RNA polymerase promoter (e.g., a T7 RNA polymerase promoter) located 5′ from and operably linked to a polynucleotide encoding a polypeptide of interest. A DNA template may also include a nucleotide sequence encoding a polyadenylation (polyA) tail located at the 3′ end of the gene of interest.

In some embodiments, the DNA template includes a 2′-deoxythymidine residue at template position +1. In some embodiments, the DNA template includes a 2′-deoxycytidine residue at template position +1. In some embodiments, the DNA template includes a 2′-deoxyadenosine residue at template position +1. In some embodiments, the DNA template includes a 2′-deoxyguanosine residue at template position +1.

In some embodiments, use of a DNA template that includes a 2′-deoxythymidine residue or 2′-deoxycytidine residue at template position +1 results in the production of RNA transcript, wherein greater than 80% (e.g., greater than 85%, greater than 90%, or greater than 95%) of the RNA transcript produced includes a functional cap. Thus, in some embodiments, a DNA template used, for example, in an IVT reaction, includes a 2′-deoxythymidine residue at template position +1. In other embodiments, a DNA template used, for example, in an IVT reaction, includes a 2′-deoxycytidine residue at template position +1.

The addition of nucleoside triphosphates (NTPs) to the 3′ end of a growing RNA strand is catalyzed by a RNA polymerase, such as T7 RNA polymerase. In some embodiments, the RNA polymerase is present in a reaction (e.g., an IVT reaction) at a concentration of 0.01 mg/ml to 1 mg/ml. For example, the RNA polymerase may be present in a reaction at a concentration of 0.01 mg/mL, 0.05 mg/ml, 0.1 mg/ml, 0.5 mg/ml or 1.0 mg/ml.

In some embodiments, a co-transcriptional capping method for RNA synthesis comprises reacting a DNA template with a RNA polymerase, nucleoside triphosphates, and a trinucleotide cap (e.g., comprising sequence GpppA_{2′Ome}pG), under in vitro transcription reaction conditions to produce RNA transcript, wherein the DNA template includes a 2′-deoxythymidine residue or a 2′-deoxycytidine residue at template position +1.

The combination of a RNA polymerase with a trinucleotide cap analog (e.g., GpppA_{2′Ome}pG), in an in vitro transcription reaction, for example, results in the production of RNA transcript, wherein greater than 80% of the RNA transcript produced includes a functional cap. In some embodiments, greater than 85% of the RNA transcript produced includes a functional cap. In some embodiments, greater than 90% of the RNA transcript produced includes a functional cap. In some embodiments, greater than 95% of the RNA transcript produced includes a functional cap. In some embodiments, greater than 96% of the RNA transcript produced includes a functional cap. In some embodiments, greater than 97% of the RNA transcript produced includes a functional cap. In some embodiments, greater than 98% of the RNA transcript produced includes a functional cap. In some embodiments, greater than 99% of the RNA transcript produced includes a functional cap.

In some embodiments, the disclosure provides an mRNA, wherein the 5′UTR is comprised of a 5′ trinucleotide cap and is altered to increase or improve endonuclease resistance and/or decrease or reduce endonuclease susceptibility as described herein. In some embodiments, the disclosure provides an mRNA, wherein the 5′UTR is comprised of a 5′ trinucleotide cap and a nucleotide sequence set forth by SEQ ID NO: 9 (v1.0 5′UTR-A), SEQ ID NO: 11 (v1.1 5′UTR-A), or SEQ ID NO: 58 (combo3_S065-A), wherein the 5′UTR is altered to increase or improve endonuclease resistance and/or decrease or reduce endonuclease susceptibility as described herein.

### Poly-A Tails

In some embodiments, a polynucleotide comprising an mRNA encoding a polypeptide of the present disclosure further comprises a poly A tail. In further embodiments, terminal groups on the poly-A tail can be incorporated for stabilization. In other embodiments, a poly-A tail comprises des-3′ hydroxyl tails. The useful poly-A tails can also include structural moieties or 2′-Omethyl modifications as taught by Li et al. (2005) Current Biology 15:1501-1507.

In one embodiment, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides of the present disclosure are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

### Start Codon Region

In some embodiments, an mRNA of the present disclosure further comprises regions that are analogous to or function like a start codon region.

In some embodiments, the translation of a polynucleotide initiates on a codon which is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG. See Touriol et al. (2003) Biology of the Cell 95:169-178 and Matsuda and Mauro (2010) PLoS ONE 5:11. As a non-limiting example, the translation of a polynucleotide begins on the alternative start codon ACG. As another non-limiting example, polynucleotide translation begins on the alternative start codon CUG. As yet another non-limiting example, the translation of a polynucleotide begins on the alternative start codon GUG.

Nucleotides flanking a codon that initiates translation such as, but not limited to, a start codon or an alternative start codon, are known to affect the translation efficiency, the length and/or the structure of the polynucleotide. See, e.g., Matsuda and Mauro (2010) PLoS ONE 5:11. Masking any of the nucleotides flanking a codon that initiates translation can be used to alter the position of translation initiation, translation efficiency, length and/or structure of a polynucleotide.

In some embodiments, a masking agent is used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs). See, e.g., Matsuda and Mauro (2010) PLoS ONE 5:11, describing masking agents LNA polynucleotides and EJCs.

In another embodiment, a masking agent is used to mask a start codon of a polynucleotide in order to increase the likelihood that translation will initiate on an alternative start codon. In some embodiments, a masking agent is used to mask a first start codon or alternative start codon in order to increase the chance that translation will initiate on a start codon or alternative start codon downstream to the masked start codon or alternative start codon.

In some embodiments, a start codon or alternative start codon is located within a perfect complement for a miR binding site. The perfect complement of a miR binding site can help control the translation, length and/or structure of the polynucleotide similar to a masking agent. As a non-limiting example, the start codon or alternative start codon is located in the middle of a perfect complement for a miR-122 binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide is removed from the polynucleotide sequence in order to have the translation of the polynucleotide begin on a codon which is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

### Stop Codon Region

In some embodiments, an mRNA of the present disclosure comprises one or more stop codons to terminate translation. In some embodiments, an mRNA of the disclosure comprises one stop codon in the 3′UTR. In some embodiments, an mRNA of the disclosure comprises two stop codons in the 3′UTR. In some embodiments, an mRNA of the disclosure comprises three stop codons in the 3′UTR. In some embodiments, an mRNA of the disclosure comprises four stop codons in the 3′UTR. In some embodiments, an mRNA of the disclosure comprises five stop codons in the 3′UTR.

In some embodiments, an mRNA of the disclosure comprises one or more stop codons in the 3′UTR wherein the one or more stop codons are selected from a group consisting of: UGA, UAA, and UAG. In some embodiments, the one or more stop codons comprise the same sequence selected form a group consisting of: UGA, UAA, and UAG. In some embodiments, the one or more stop codons comprise different sequences selected from a group consisting of: UGA, UAA, and UAG.

In some embodiments, an mRNA of the present disclosure comprises a stop codon UGA and two additional stop codons, wherein the first additional stop codon is UGA, UAA, or UAG and the second additional stop codon is UGA, UAA, or UAG. In some embodiments, an mRNA of the disclosure comprises a stop codon UGA and two additional stop codons, wherein the first additional stop codon is UAA and the second additional stop codon is UAG. In some embodiments, an mRNA of the disclosure comprising a stop codon UGA is altered by substitution or deletion of one or more nucleotides to increase or improve endonuclease resistance and/or decrease or reduce endonuclease susceptibility. In some embodiments, an mRNA of the disclosure comprising a stop codon UGA and one or more additional stop codons is altered by substitution or deletion to increase or improve endonuclease resistance and/or decrease or reduce endonuclease susceptibility. In some embodiments, the UGA stop codon is altered by substitution with a degenerate stop codon (e.g., UAA or UAG). In some embodiments, wherein the mRNA comprises a UGA stop codon and one or more additional stop codons, the UGA stop codon is altered by deletion. In some embodiments, altering a UGA stop codon of an mRNA of the disclosure increases or improves stability of the mRNA, increases or improves mRNA half-life, increases or improves mRNA potency, increases or improves endonuclease resistance, and/or decreases or reduces endonuclease susceptibility.

### Adjusted Uracil Content

In some embodiments of the disclosure, an mRNA may have adjusted uracil content. In some embodiments, the uracil content of the open reading frame (ORF) of the polynucleotide encoding a therapeutic polypeptide relative to the theoretical minimum uracil content of a nucleotide sequence encoding the therapeutic polypeptide (% U_{TM}), is between about 100% and about 150. In some embodiments, the uracil content of the ORF is between about 105% and about 145%, about 105% and about 140%, about 110% and about 140%, about 110% and about 145%, about 115% and about 135%, about 105% and about 135%, about 110% and about 135%, about 115% and about 145%, or about 115% and about 140% of the theoretical minimum uracil content in the corresponding wild-type ORF (% U_{TM}). In other embodiments, the uracil content of the ORF is between about 117% and about 134% or between 118% and 132% of the % U_{TM}. In some embodiments, the uracil content of the ORF encoding a polypeptide is about 115%, about 120%, about 125%, about 130%, about 135%, about 140%, about 145%, or about 150% of the % U_{TM}. In this context, the term “uracil” can refer to an alternative uracil and/or naturally occurring uracil.

In some embodiments, the uracil content of the ORF of the polynucleotide relative to the uracil content of the corresponding wild-type ORF (% U_{WT}) is less than 100%. In some embodiments, the % U_{WT} of the polynucleotide is less than about 95%, less than about 90%, less than about 85%, less than 80%, less than 79%, less than 78%, less than 77%, less than 76%, less than 75%, less than 74%, or less than 73%. In some embodiments, the % U_{WT} of the polynucleotide is between 65% and 73%.

In some embodiments, the uracil content in the ORF of the mRNA encoding a is less than about 50%, about 40%, about 30%, or about 20% of the total nucleobase content in the ORF. In some embodiments, the uracil content in the ORF is between about 15% and about 25% of the total nucleobase content in the ORF. In other embodiments, the uracil content in the ORF is between about 20% and about 30% of the total nucleobase content in the ORF. In one embodiment, the uracil content in the ORF of the mRNA encoding a polypeptide is less than about 20% of the total nucleobase content in the open reading frame. In this context, the term “uracil” can refer to an alternative uracil and/or naturally occurring uracil.

In further embodiments, the ORF of the mRNA encoding a polypeptide having adjusted uracil content has increased cytosine (C), guanine (G), or guanine/cytosine (G/C) content (absolute or relative). In some embodiments, the overall increase in C, G, or G/C content (absolute or relative) of the ORF is at least about 2%, at least about 3%, at least about 4%, at least about 5%, at least about 6%, at least about 7%, at least about 10%, at least about 15%, at least about 20%, at least about 40%, at least about 50%, at least about 60%, at least about 70%, at least about 80%, at least about 90%, at least about 95%, or at least about 100% relative to the G/C content (absolute or relative) of the wild-type ORF. In some embodiments, the G, the C, or the G/C content in the ORF is less than about 100%, less than about 90%, less than about 85%, or less than about 80% of the theoretical maximum G, C, or G/C content of the nucleotide sequence encoding the PBDG polypeptide (% G_{TMX}; % C_{TMX}, or % G/C_{TMX}). In other embodiments, the G, the C, or the G/C content in the ORF is between about 70% and about 80%, between about 71% and about 79%, between about 71% and about 78%, or between about 71% and about 77% of the % G_{TMX}, % C_{TMX}, or % G/C_{TMX}. In some embodiments, the guanine content of the ORF of the polynucleotide with respect to the theoretical maximum guanine content of a nucleotide sequence encoding the polypeptide (% G_{TMX}) is at least 69%, at least 70%, at least 75%, at least about 80%, at least about 85%, at least about 90%, at least about 95%, or about 100%. In some embodiments, the % G_{TMX} of the polynucleotide is between about 70% and about 80%, between about 71% and about 79%, between about 71% and about 78%, or between about 71% and about 77%. In some embodiments, the cytosine content of the ORF of the polynucleotide relative to the theoretical maximum cytosine content of a nucleotide sequence encoding the polypeptide (% C_{TMX}) is at least 59%, at least 60%, at least about 65%, at least about 70%, at least about 75%, at least about 80%, at least about 85%, at least about 90%, at least about 95%, or about 100%. In some embodiments, the % C_{TMX} of the ORF of the polynucleotide is between about 60% and about 80%, between about 62% and about 80%, between about 63% and about 79%, or between about 68% and about 76%. In some embodiments, the guanine and cytosine content (G/C) of the ORF of the polynucleotide relative to the theoretical maximum G/C content in a nucleotide sequence encoding the polypeptide (% G/C_{TMX}) is at least about 81%, at least about 85%, at least about 90%, at least about 95%, or about 100%. In some embodiments, the % G/C_{TMX} in the ORF of the polynucleotide is between about 80% and about 100%, between about 85% and about 99%, between about 90% and about 97%, or between about 91% and about 96%. In some embodiments, the G/C content in the ORF of the polynucleotide relative to the G/C content in the corresponding wild-type ORF (% G/C_{WT}) is at least 102%, at least 103%, at least 104%, at least 105%, at least 106%, at least 107%, at least 110%, at least 115%, or at least 120%. In some embodiments, the average G/C content in the 3rd codon position in the ORF of the polynucleotide is at least 20%, at least 21%, at least 22%, at least 23%, at least 24%, at least 25%, at least 26%, at least 27%, at least 28%, at least 29%, or at least 30% higher than the average G/C content in the 3rd codon position in the corresponding wild-type ORF. In some embodiments, the increases in G and/or C content (absolute or relative) described herein can be conducted by replacing synonymous codons with low G, C, or G/C content with synonymous codons having higher G, C, or G/C content. In other embodiments, the increase in G and/or C content (absolute or relative) is conducted by replacing a codon ending with U with a synonymous codon ending with G or C.

In further embodiments, the ORF of the mRNA encoding a polypeptide includes less uracil pairs (UU) and/or uracil triplets (UUU) and/or uracil quadruplets (UUUU) than the corresponding wild-type nucleotide sequence encoding the polypeptide. In some embodiments, the ORF of the mRNA encoding a polypeptide of the disclosure includes no uracil pairs and/or uracil triplets and/or uracil quadruplets. In some embodiments, uracil pairs and/or uracil triplets and/or uracil quadruplets are reduced below a certain threshold, e.g., no more than 1, 2, 3, 4, 5, 6, 7, 8, 9, 10, 11, 12, 13, 14, 15, 16, 17, 18, 19, or 20 occurrences in the ORF of the mRNA encoding the polypeptide. In a particular embodiment, the ORF of the mRNA encoding the polypeptide of the disclosure contains less than 20, 19, 18, 17, 16, 15, 14, 13, 12, 11, 10, 9, 8, 7, 6, 5, 4, 3, 2, or 1 non-phenylalanine uracil pairs and/or triplets. In another embodiment, the ORF of the mRNA encoding the polypeptide contains no non-phenylalanine uracil pairs and/or triplets.

In further embodiments, the ORF of the mRNA encoding a polypeptide of the disclosure includes less uracil-rich clusters than the corresponding wild-type nucleotide sequence encoding the polypeptide. In some embodiments, the ORF of the mRNA encoding the polypeptide of the disclosure contains uracil-rich clusters that are shorter in length than corresponding uracil-rich clusters in the corresponding wild-type nucleotide sequence encoding the polypeptide.

In further embodiments, alternative lower frequency codons are employed. In some embodiment, the ORF of the polynucleotide further comprises at least one low-frequency codon. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100% of the codons in the polypeptide-encoding ORF of the mRNA are substituted with alternative codons, each alternative codon having a codon frequency lower than the codon frequency of the substituted codon in the synonymous codon set. The ORF may also have adjusted uracil content, as described above. In some embodiments, at least one codon in the ORF of the mRNA encoding the polypeptide is substituted with an alternative codon having a codon frequency lower than the codon frequency of the substituted codon in the synonymous codon set.

In some embodiments, the polynucleotide is an mRNA that comprises an ORF that encodes a polypeptide, wherein the uracil content of the ORF is between about 115% and about 135% of the theoretical minimum uracil content in the corresponding wild-type ORF, and wherein the uracil content in the ORF encoding the polypeptide is less than about 30% of the total nucleobase content in the ORF. In some embodiments, the ORF that encodes the polypeptide is further modified to increase G/C content of the ORF (absolute or relative) by at least about 40%, as compared to the corresponding wild-type ORF. In yet other embodiments, the ORF encoding the polypeptide contains less than 20 non-phenylalanine uracil pairs and/or triplets. In some embodiments, at least one codon in the ORF of the mRNA encoding the polypeptide is further substituted with an alternative codon having a codon frequency lower than the codon frequency of the substituted codon in the synonymous codon set.

In some embodiments, the expression of the polypeptide encoded by an mRNA comprising an ORF, wherein the uracil content of the ORF has been adjusted (e.g., the uracil content is between about 115% and about 135% of the theoretical minimum uracil content in the corresponding wild-type ORF) is increased by at least about 10-fold when compared to expression of the polypeptide from the corresponding wild-type mRNA. In some embodiments, the innate immune response induced by the mRNA including an open ORF wherein the uracil content has been adjusted (e.g., the uracil content of the ORF is between about 115% and about 135% of the theoretical minimum uracil content in the corresponding wild-type ORF) is reduced by at least about 10-fold when compared to expression of the polypeptide from the corresponding wild-type mRNA. In some embodiments, the mRNA with adjusted uracil content does not substantially induce an innate immune response of a mammalian cell into which the mRNA is introduced.

In some embodiments, the disclosure provides a method of increasing or improving stability of an mRNA, comprising:

(i) providing an mRNA comprising an ORF wherein the uracil content of the ORF has been adjusted (e.g., the uracil content is between about 115% and 135% of the theoretical minimum uracil content of the corresponding wild-type ORF);

(ii) identifying at least one endonuclease sensitive sequence motif comprising the nucleotide sequence WGA, wherein W=adenine (A) or uracil (U); and

(iii) altering the at least one endonuclease sensitive sequence motif, thereby generating an endonuclease-resistant mRNA.

In some embodiments, the disclosure provides a method of increasing or improving stability of an mRNA, wherein the mRNA comprises at least one endonuclease sensitive sequence motif comprising the nucleotide sequence WGA, wherein W=adenine (A) or uracil (U), the method comprising (i) adjusting the uracil content of the ORF (e.g., adjusting the uracil content to between about 115% and 135% of the theoretical minimum uracil content of the corresponding wild-type ORF) and (ii) altering one or more remaining endonuclease sensitive sequence motifs, thereby increasing or improving stability of the mRNA.

In some embodiments, the disclosure provides a method of increasing or improving stability of an mRNA, wherein the mRNA comprises at least one endonuclease sensitive sequence motif comprising the nucleotide sequence WGA, wherein W=adenine (A) or uracil (U), the method comprising (i) altering at least one endonuclease sensitive sequence motifs; and (ii) adjusting the uracil content of the ORF (e.g., adjusting the uracil content to between about 115% and 135% of the theoretical minimum uracil content of the corresponding wild-type ORF), thereby increasing or improving stability of the mRNA.

In some embodiments, the stability of an mRNA comprising an ORF is increased, wherein the uracil content of the ORF has been adjusted (e.g., the uracil content is between about 115% and about 135% of the theoretical minimum uracil content in the corresponding wild-type ORF) and wherein the mRNA comprises at least one endonuclease sensitive sequence motif that is altered by substitution or deletion to thereby increase stability by at least about 5%, 10%, 20%, 30%, 40%, 50%, 60%, 70%, 80%, 90%, 100%, 105%, 110%, 115%, 120%, 125%, 130%, 135%, 140%, 150%, 160%, 170%, 175%, 180%, 190%, 200%, 250%, 300%, 350%, 400%, 450%, 500%, 600%, 700%, 800%, 900%, 1,000% or more relative to an unaltered wild-type mRNA counterpart. In some embodiments, the stability of the mRNA is increased by at least 1-fold, 2-fold, 3-fold, 4-fold, 5-fold, 6-fold, 7-fold, 8-fold, 9-fold, 10-fold or more relative to an unaltered mRNA counterpart.

In some embodiments, the half-life of an mRNA comprising an ORF is increased, wherein the uracil content of the ORF has been adjusted (e.g., the uracil content is between about 115% and about 135% of the theoretical minimum uracil content in the corresponding wild-type ORF) and wherein the mRNA comprises at least one endonuclease sensitive sequence motif that is altered by substitution or deletion to thereby increase stability by at least about 5%, 10%, 20%, 30%, 40%, 50%, 60%, 70%, 80%, 90%, 100%, 105%, 110%, 115%, 120%, 125%, 130%, 135%, 140%, 150%, 160%, 170%, 175%, 180%, 190%, 200%, 250%, 300%, 350%, 400%, 450%, 500%, 600%, 700%, 800%, 900%, 1,000% or more relative to an unaltered mRNA counterpart. In some embodiments, the half-life of the mRNA is increased by at least 1-fold, 2-fold, 3-fold, 4-fold, 5-fold, 6-fold, 7-fold, 8-fold, 9-fold, 10-fold or more relative to an unaltered mRNA counterpart.

In some embodiments, the uracil content of the mRNA is adjusted as described herein, and a modified nucleoside is partially or completely substituted for the uracil remaining in the mRNA following adjustment. As a non-limiting example, the natural nucleotide uridine may be substituted with a modified nucleoside as described herein. In some embodiments, the modified nucleoside comprises pseudouridine (ψ). In some embodiments, the modified nucleoside comprises 1-methyl-pseudouridine (m1ψ). In some embodiments, the modified nucleoside comprises 1-methyl-pseudouridine (m1ψ) and 5-methyl-cytidine (m5C). In some embodiments, the modified nucleoside comprises 2-thiouridine (s²U). In some embodiments, the modified nucleoside comprises 2-thiouridine and 5-methyl-cytidine (m5C). In some embodiments, the modified nucleoside comprises 5-methoxy-uridine (mo5U). In some embodiments, the modified nucleoside comprises 5-methoxy-uridine (mo5U) and 5-methyl-cytidine (m5C). In some embodiments, the modified nucleoside comprises 2′-O-methyl uridine. In some embodiments, the modified nucleoside comprises 2′-O-methyl uridine and 5-methyl-cytidine (m5C). In some embodiments, the modified nucleoside comprises N6-methyl-adenosine (m6A). In some embodiments, the modified nucleoside comprises N6-methyl-adenosine (m6A) and 5-methyl-cytidine (m5C).

### Chemical Modification of RNA

Numerous approaches for the chemical modification of mRNA to improve translation efficiency and reduce immunogenicity are known, including modifications at the 5′ cap, 5′ and 3′-UTRs, the open reading frame, and the poly(A) tail (Sahin et al., (2014) Nat Rev Drug Discovery 13:759-780). For example, pseudouridine (ψ) modified mRNA was shown to increased expression of encoded erythropoietin (Kariko et al., (2012) Mol Ther 20:948-953). A combination of 2-thiouridine (s²U) and 5-methylcytidine (5meC) in modified mRNAs was shown to extend the expression of encoded protein (Kormann et al., (2011) Nat Biotechnol 29:154-157). A recent study demonstrated the induction of vascular regeneration using modified (5meC and W) mRNA encoding human vascular endothelial growth factor (Zangi et al., (2013) Nat Biotechnol 31:898-907). These studies demonstrate the utility of incorporating chemically modified nucleotides to achieve mRNA structural and functional optimization.

Accordingly, in some embodiments, an mRNA described herein comprises a modification, wherein the modification is the incorporation of one or more chemically modified nucleotides. In some embodiments, one or more chemically modified nucleotides is incorporated into the initiation codon of the mRNA and functions to increases binding affinity between the initiation codon and the anticodon of the initiator Met-tRNAiMet. In some embodiments, the one or more chemically modified nucleotides is 2-thiouridine. In some embodiments, the one or more chemically modified nucleotides is 2′-O-methyl-2-thiouridine. In some embodiments, the one or more chemically modified nucleotides is 2-selenouridine. In some embodiments, the one or more chemically modified nucleotides is 2′-O-methyl ribose. In some embodiments, the one or more chemically modified nucleotides is selected from a locked nucleic acid, inosine, 2-methylguanosine, or 6-methyl-adenosine. In some embodiments, deoxyribonucleotides are incorporated into mRNA. An mRNA of the disclosure may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

Modified mRNAs

In some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (ψ), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine(τm⁵s²U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (Wm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine. In some aspects, the modified uridine is N1-methyl-pseudouridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N6-isopentenyl-adenosine (i⁶A), 2-methylthio-N6-isopentenyl-adenosine (ms²i⁶A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m62Am), 1,2′-O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (o₂yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²₂G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²₂Gm), 1-methyl-2′-O-methyl-guanosine (m⁷Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr(p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (m¹ψ), 5-methoxy-uridine (mo⁵U), 5-methyl-cytidine (m⁵C), pseudouridine (ψ), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises N6-methyl-adenosine (m⁶A). In some embodiments, the mRNA comprises N6-methyl-adenosine (m⁶A) and 5-methyl-cytidine (m⁵C).

In certain embodiments, an mRNA of the disclosure is uniformly modified (i.e., fully modified, modified through-out the entire sequence) for a particular modification. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

Examples of modified nucleosides and modified nucleoside combinations are provided below in Table 8 and Table 9. These combinations of modified nucleotides can be used to form the mmRNAs of the disclosure. In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

In some embodiments, a therapeutic, stabilized mRNA is provided by alteration of one or more endonuclease sensitive sequence motifs from an unaltered mRNA comprising a 5′UTR, an ORF encoding a polypeptide, and a 3′UTR. In some embodiments, a therapeutic, stabilized mRNA is produced by altering one or more endonuclease sensitive sequence motifs (e.g., WGA, wherein W=adenine (A) or uracil (U)) in the 5′UTR, the ORF, and/or the 3′UTR of an unaltered mRNA. In some embodiments, a method of altering an endonuclease sensitive sequence motif is by substitution with a modified nucleoside. Modified nucleosides useful for stabilization of RNA are known in the art, and including those described by Ahmad Dar, et al (2016) Scientific Reports 6:20031, Chiu et al (2003) RNA 9:1034-1048, and Gaglione, et al (2010) Mini review in med chem 10:578-595.

TABLE 8
Combinations of Nucleoside Modifications

| Modified | |
|---|---|
| Nucleotide | Modified Nucleotide Combination |
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are |
| | N1-methyl-pseudouridine and about |
| | 50% of uridines are pseudouridine |
| | pseudoisocytidine/about 25% of uridines are |
| | N1-methyl-pseudouridine and about |
| | 25% of uridines are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are |
| | 2-thio-uridine |
| | about 50% of uridines are 5-methyl-cytidine/ |
| | about 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/ |
| | about 50% of uridines are 2-thio-uridine |

TABLE 9
Modified Nucleosides and Combinations Thereof

1-(2,2,2-Trifluoroethyl)pseudo-UTP
1-Ethyl-pseudo-UTP
1-Methyl-pseudo-U-alpha-thio-TP
1-methyl-pseudouridine TP, ATP, GTP, CTP
1-methyl-pseudo-UTP/5-methyl-CTP/ATP/GTP
1-methyl-pseudo-UTP/CTP/ATP/GTP
1-Propyl-pseudo-UTP
25% 5-Aminoallyl-CTP + 75% CTP/25% 5-Methoxy-UTP +
75% UTP
25% 5-Aminoallyl-CTP + 75% CTP/75% 5-Methoxy-UTP +
25% UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP +
75% UTP
25% 5-Carboxy-CTP + 75% CTP/75% 5-Methoxy-UTP +
25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/ 25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75% CTP/25% 5-Methoxy-UTP +
75% UTP
25% 5-Hydroxymethyl-CTP + 75% CTP/75% 5-Methoxy-UTP +
25% UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP +
75% UTP
25% 5-Methoxy-CTP + 75% CTP/75% 5-Methoxy-UTP +
25% UTP
25% 5-Methyl-CTP + 75% CTP/25% 5-Methoxy-UTP +
75% 1-Methyl-pseudo-UTP
25% 5-Methyl-CTP + 75% CTP/25% 5-Methoxy-UTP + 75% UTP
25% 5-Methyl-CTP + 75% CTP/50% 5-Methoxy-UTP +
50% 1-Methyl-pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP +
25% 1-Methyl-pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP +
75% UTP
25% 5-Trifluoromethyl-CTP + 75% CTP/75% 5-Methoxy-UTP +
25% UTP
25% 5-Trifluoromethyl-CTP + 75% CTP/1-Methyl-pseudo-UTP
25% N4-Ac-CTP + 75% CTP/25% 5-Methoxy-UTP + 75% UTP
25% N4-Ac-CTP + 75% CTP/75% 5-Methoxy-UTP + 25% UTP
25% N4-Bz-CTP + 75% CTP/25% 5-Methoxy-UTP + 75% UTP
25% N4-Bz-CTP + 75% CTP/75% 5-Methoxy-UTP + 25% UTP
25% N4-Methyl-CTP + 75% CTP/25% 5-Methoxy-UTP +
75% UTP
25% N4-Methyl-CTP + 75% CTP/75% 5-Methoxy-UTP +
25% UTP
25% Pseudo-iso-CTP + 75% CTP/25% 5-Methoxy-UTP +
75% UTP
25% Pseudo-iso-CTP + 75% CTP/75% 5-Methoxy-UTP +
25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-metoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP +
75% 1-Methyl-pseudo-UTP
50% 5-Methyl-CTP + 50% CTP/25% 5-Methoxy-UTP + 75% UTP
50% 5-Methyl-CTP + 50% CTP/50% 5-Methoxy-UTP +
50% 1-Methyl-pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP +
25% 1-Methyl-pseudo-UTP
50% 5-Methyl-CTP + 50% CTP/75% 5-Methoxy-UTP + 25% UTP
50% 5-Trifluoromethyl-CTP + 50% CTP/1-Methyl-pseudo-UTP
50% 5-Bromo-CTP/50% CTP/Pseudo-UTP
50% 5-methoxy-UTP/25% 5-methyl-CTP/ATP/GTP
50% 5-methoxy-UTP/50% 5-methyl-CTP/ATP/GTP
50% 5-methoxy-UTP/75% 5-methyl-CTP/ATP/GTP
50% 5-methoxy-UTP/CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxy-methyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me-CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/N6-Isopentenyl-ATP
5-methoxy-UTP/25% 5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap 0)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6-Me-ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoro- methyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP +
75% UTP
75% 5-Aminoallyl-CTP + 25% CTP/75% 5-Methoxy-UTP +
25% UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP +
75% UTP
75% 5-Carboxy-CTP + 25% CTP/75% 5-Methoxy-UTP +
25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP +
75% UTP
75% 5-Hydroxymethyl-CTP + 25% CTP/75% 5-Methoxy-UTP +
25% UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP +
75% UTP
75% 5-Methoxy-CTP + 25% CTP/75% 5-Methoxy-UTP +
25% UTP
75% 5-methoxy-UTP/5-methyl-CTP/ATP/GTP
75% 5-Methyl-CTP + 25% CTP/25% 5-Methoxy-UTP +
75% 1-Methyl-pseudo-UTP
75% 5-Methyl-CTP + 25% CTP/25% 5-Methoxy-UTP + 75% UTP
75% 5-Methyl-CTP + 25% CTP/50% 5-Methoxy-UTP +
50% 1-Methyl-pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP +
25% 1-Methyl-pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP +
75% UTP
75% 5-Trifluoromethyl-CTP + 25% CTP/75% 5-Methoxy-UTP +
25% UTP
75% 5-Trifluoromethyl-CTP + 25% CTP/1-Methyl-pseudo-UTP
75% N4-Ac-CTP + 25% CTP/25% 5-Methoxy-UTP + 75% UTP
75% N4-Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP +
75% UTP
75% N4-Methyl-CTP + 25% CTP/75% 5-Methoxy-UTP +
25% UTP
75% Pseudo-iso-CTP + 25% CTP/25% 5-Methoxy-UTP +
75% UTP
75% Pseudo-iso-CTP + 25% CTP/75% 5-Methoxy-UTP +
25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP
CTP/5-Methoxy-UTP
CTP/5-Methoxy-UTP (cap 0)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP(No cap)
N1-Me-GTP
N4-Ac-CTP
N4Ac-CTP/1-Methyl-pseudo-UTP
N4Ac-CTP/5-Methoxy-UTP
N4-acetyl-cytidine TP, ATP, GTP, UTP
N4-Bz-CTP/5-Methoxy-UTP
N4-methyl CTP
N4-Methyl-CTP/5-Methoxy-UTP
Pseudo-iso-CTP/5-Methoxy-UTP
PseudoU-alpha-thio-TP
pseudouridine TP, ATP, GTP, CTP
pseudo-UTP/5-methyl-CTP/ATP/GTP
UTP-5-oxyacetic acid Me ester
Xanthosine

According to the disclosure, polynucleotides of the disclosure may be synthesized to comprise the combinations or single modifications of Table 8 or Table 9.

Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs and vectors that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1(3), 165-187 (1990).

MicroRNA (miRNA) Binding Sites

Nucleic acid molecules (e.g., RNA, e.g., mRNA) of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, nucleic acid molecules (e.g., RNA, e.g., mRNA) including such regulatory elements are referred to as including “sensor sequences.” Non-limiting examples of sensor sequences are described in U.S. Publication 2014/0200261, the contents of which are incorporated herein by reference in their entirety.

In some embodiments, a nucleic acid molecule (e.g., RNA, e.g., mRNA) of the disclosure comprises an open reading frame (ORF) encoding a polypeptide of interest and further comprises one or more miRNA binding site(s). Inclusion or incorporation of miRNA binding site(s) provides for regulation of nucleic acid molecules (e.g., RNA, e.g., mRNA) of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a nucleic acid molecule (e.g., RNA, e.g., mRNA) and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a nucleic acid molecule (e.g., RNA, e.g., mRNA) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a nucleic acid molecule, e.g., within a DNA or within an RNA transcript, including in the 5′UTR and/or 3′UTR, that has sufficient complementarity to all or a region of a miRNA to interact with, associate with or bind to the miRNA. In some embodiments, a nucleic acid molecule (e.g., RNA, e.g., mRNA) of the disclosure comprising an ORF encoding a polypeptide of interest and further comprises one or more miRNA binding site(s). In exemplary embodiments, a 5′UTR and/or 3′UTR of the nucleic acid molecule (e.g., RNA, e.g., mRNA) comprises the one or more miRNA binding site(s).

A miRNA binding site having sufficient complementarity to a miRNA refers to a degree of complementarity sufficient to facilitate miRNA-mediated regulation of a nucleic acid molecule (e.g., RNA, e.g., mRNA), e.g., miRNA-mediated translational repression or degradation of the nucleic acid molecule (e.g., RNA, e.g., mRNA). In exemplary aspects of the disclosure, a miRNA binding site having sufficient complementarity to the miRNA refers to a degree of complementarity sufficient to facilitate miRNA-mediated degradation of the nucleic acid molecule (e.g., RNA, e.g., mRNA), e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the nucleic acid molecule (e.g., RNA, e.g., mRNA) comprising the miRNA binding site. In other embodiments, the miRNA binding site has imperfect complementarity so that a RISC complex comprising the miRNA induces instability in the nucleic acid molecule (e.g., RNA, e.g., mRNA) comprising the miRNA binding site. In another embodiment, the miRNA binding site has imperfect complementarity so that a RISC complex comprising the miRNA represses transcription of the nucleic acid molecule (e.g., RNA, e.g., mRNA) comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a nucleic acid molecule (e.g., RNA, e.g., mRNA) of the disclosure, the nucleic acid molecule (e.g., RNA, e.g., mRNA) can be targeted for degradation or reduced translation, provided the miRNA in question is available. This can reduce off-target effects upon delivery of the nucleic acid molecule (e.g., RNA, e.g., mRNA). For example, if a nucleic acid molecule (e.g., RNA, e.g., mRNA) of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the nucleic acid molecule (e.g., RNA, e.g., mRNA).

For example, one of skill in the art would understand that one or more miR can be included in a nucleic acid molecule (e.g., an RNA, e.g., mRNA) to minimize expression in cell types other than lymphoid cells. In one embodiment, miR122 can be used. In another embodiment, miR126 can be used. In still another embodiment, multiple copies of these miRs or combinations may be used.

Conversely, miRNA binding sites can be removed from nucleic acid molecule (e.g., RNA, e.g., mRNA) sequences in which they naturally occur in order to increase protein expression in specific tissues. For example, a binding site for a specific miRNA can be removed from a nucleic acid molecule (e.g., RNA, e.g., mRNA) to improve protein expression in tissues or cells containing the miRNA.

In one embodiment, a nucleic acid molecule (e.g., RNA, e.g., mRNA) of the disclosure can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a nucleic acid molecule (e.g., RNA, e.g., mRNA) of the disclosure can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety).

miRNAs and miRNA binding sites can correspond to any known sequence, including non-limiting examples described in U.S. Publication Nos. 2014/0200261, 2005/0261218, and 2005/0059005, each of which are incorporated herein by reference in their entirety. Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and monocytes), monocytes, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a nucleic acid molecule (e.g., RNA, e.g., mRNA) can be shut-off by adding miR-142 binding sites to the 3′-UTR of the polynucleotide, enabling more stable gene transfer in tissues and cells. miR-142 efficiently degrades exogenous nucleic acid molecules (e.g., RNA, e.g., mRNA) in antigen presenting cells and suppresses cytotoxic elimination of transduced cells (e.g., Annoni A et al., blood, 2009, 114, 5152-5161; Brown B D, et al., Nat med. 2006, 12(5), 585-591; Brown B D, et al., blood, 2007, 110(13): 4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a nucleic acid molecule of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the nucleic acid molecule (e.g., RNA, e.g., mRNA). The nucleic acid molecule (e.g., RNA, e.g., mRNA) is then stably expressed in target tissues or cells without triggering cytotoxic elimination.

In one embodiment, binding sites for miRNAs that are known to be expressed in immune cells, in particular, antigen presenting cells, can be engineered into a nucleic acid molecule (e.g., RNA, e.g., mRNA) of the disclosure to suppress the expression of the nucleic acid molecule (e.g., RNA, e.g., mRNA) in antigen presenting cells through miRNA mediated RNA degradation, subduing the antigen-mediated immune response. Expression of the nucleic acid molecule (e.g., RNA, e.g., mRNA) is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a nucleic acid molecule of the disclosure.

To further drive the selective degradation and suppression in APCs and macrophage, a nucleic acid molecule (e.g., RNA, e.g., mRNA) of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p, miRNA binding sites from any liver specific miRNA can be introduced to or removed from a nucleic acid molecule (e.g., RNA, e.g., mRNA) of the disclosure to regulate expression of the nucleic acid molecule (e.g., RNA, e.g., mRNA) in the liver. Liver specific miRNA binding sites can be engineered alone or further in combination with immune cell (e.g., APC) miRNA binding sites in a nucleic acid molecule (e.g., RNA, e.g., mRNA) of the disclosure. In one embodiment, miRNA binding sites that promote degradation of mRNAs by hepatocytes are present in an mRNA molecule agent.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p, miRNA binding sites from any lung specific miRNA can be introduced to or removed from a nucleic acid molecule (e.g., RNA, e.g., mRNA) of the disclosure to regulate expression of the nucleic acid molecule (e.g., RNA, e.g., mRNA) in the lung. Lung specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p, miRNA binding sites from any heart specific microRNA can be introduced to or removed from a nucleic acid molecule (e.g., RNA, e.g., mRNA) of the disclosure to regulate expression of the nucleic acid molecule (e.g., RNA, e.g., mRNA) in the heart. Heart specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-²¹⁹-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p, miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657, miRNA binding sites from any CNS specific miRNA can be introduced to or removed from a nucleic acid molecule (e.g., RNA, e.g., mRNA) of the disclosure to regulate expression of the nucleic acid molecule (e.g., RNA, e.g., mRNA) in the nervous system. Nervous system specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. miRNA binding sites from any pancreas specific miRNA can be introduced to or removed from a nucleic acid molecule (e.g., RNA, e.g., mRNA) of the disclosure to regulate expression of the nucleic acid molecule (e.g., RNA, e.g., mRNA) in the pancreas. Pancreas specific miRNA binding sites can be engineered alone or further in combination with immune cell (e.g. APC) miRNA binding sites in a nucleic acid molecule (e.g., RNA, e.g., mRNA) of the disclosure.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. miRNA binding sites from any kidney specific miRNA can be introduced to or removed from a nucleic acid molecule (e.g., RNA, e.g., mRNA) of the disclosure to regulate expression of the nucleic acid molecule (e.g., RNA, e.g., mRNA) in the kidney. Kidney specific miRNA binding sites can be engineered alone or further in combination with immune cell (e.g., APC) miRNA binding sites in a nucleic acid molecule (e.g., RNA, e.g., mRNA) of the disclosure.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. miRNA binding sites from any muscle specific miRNA can be introduced to or removed from a nucleic acid molecule (e.g., RNA, e.g., mRNA) of the disclosure to regulate expression of the nucleic acid molecule (e.g., RNA, e.g., mRNA) in the muscle. Muscle specific miRNA binding sites can be engineered alone or further in combination with immune cell (e.g., APC) miRNA binding sites in a nucleic acid molecule (e.g., RNA, e.g., mRNA) of the disclosure.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a nucleic acid molecule (e.g., RNA, e.g., mRNA) of the disclosure to regulate expression of the nucleic acid molecule (e.g., RNA, e.g., mRNA) in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13(5), 757-764; Vidigal J A and Ventura A, Semin Cancer Biol. 2012, 22(5-6), 428-436; Goff L A et al., PLoS One, 2009, 4:e7192; Morin R D et al., Genome Res, 2008, 18, 610-621; Yoo J K et al., Stem Cells Dev. 2012, 21(11), 2049-2057, each of which is herein incorporated by reference in its entirety). miRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-548o-3p, miR-548o-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941,miR-96-3p, miR-96-5p, miR-99b-3p and miR-99b-5p. Many predicted novel miRNAs are discovered by deep sequencing in human embryonic stem cells (e.g., Morin R D et al., Genome Res, 2008, 18, 610-621; Goff L A et al., PLoS One, 2009, 4:e7192; Bar M et al., Stem cells, 2008, 26, 2496-2505, the content of each of which is incorporated herein by reference in its entirety).

In some embodiments, the binding sites of embryonic stem cell specific miRNAs can be included in or removed from the 3′UTR of a nucleic acid molecule (e.g., RNA, e.g., mRNA) of the disclosure to modulate the development and/or differentiation of embryonic stem cells, to inhibit the senescence of stem cells in a degenerative condition (e.g. degenerative diseases), or to stimulate the senescence and apoptosis of stem cells in a disease condition (e.g. cancer stem cells).

Many miRNA expression studies are conducted to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563), the content of each of which is incorporated herein by reference in its entirety.

As a non-limiting example, miRNA binding sites for miRNAs that are over-expressed in certain cancer and/or tumor cells can be removed from the 3′UTR of a nucleic acid molecule (e.g., RNA, e.g., mRNA) of the disclosure, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

miRNA can also regulate complex biological processes such as angiogenesis (e.g., miR-132) (Anand and Cheresh Curr Opin Hematol 2011 18:171-176). In the nucleic acid molecules (e.g., RNA, e.g., mRNA) of the disclosure, miRNA binding sites that are involved in such processes can be removed or introduced, in order to tailor the expression of the nucleic acid molecules (e.g., RNA, e.g., mRNA) to biologically relevant cell types or relevant biological processes. In this context, the nucleic acid molecules (e.g., RNA, e.g., mRNA) of the disclosure are defined as auxotrophic polynucleotides.

In some embodiments, the therapeutic window and/or differential expression (e.g., tissue-specific expression) of a polypeptide of the disclosure may be altered by incorporation of a miRNA binding site into a nucleic acid molecule (e.g., RNA, e.g., mRNA) encoding the polypeptide. In one example, a nucleic acid molecule (e.g., RNA, e.g., mRNA) may include one or more miRNA binding sites that are bound by miRNAs that have higher expression in one tissue type as compared to another. In another example, a nucleic acid molecule (e.g., RNA, e.g., mRNA) may include one or more miRNA binding sites that are bound by miRNAs that have lower expression in a cancer cell as compared to a non-cancerous cell of the same tissue of origin. When present in a cancer cell that expresses low levels of such an miRNA, the polypeptide encoded by the nucleic acid molecule (e.g., RNA, e.g., mRNA) typically will show increased expression.

Liver cancer cells (e.g., hepatocellular carcinoma cells) typically express low levels of miR-122 as compared to normal liver cells. Therefore, a nucleic acid molecule (e.g., RNA, e.g., mRNA) encoding a polypeptide that includes at least one miR-122 binding site (e.g., in the 3′-UTR of the mRNA) will typically express comparatively low levels of the polypeptide in normal liver cells and comparatively high levels of the polypeptide in liver cancer cells. If the polypeptide is able to induce immunogenic cell death, this can cause preferential immunogenic cell killing of liver cancer cells (e.g., hepatocellular carcinoma cells) as compared to normal liver cells.

In some embodiments, the nucleic acid molecule (e.g., RNA, e.g., mRNA) includes at least one miR-122 binding site, at least two miR-122 binding sites, at least three miR-122 binding sites, at least four miR-122 binding sites, or at least five miR-122 binding sites. In one aspect, the miRNA binding site binds miR-122 or is complementary to miR-122. In another aspect, the miRNA binding site binds to miR-122-3p or miR-122-5p. In a particular aspect, the miRNA binding site comprises a nucleotide sequence at least 80%, at least 85%, at least 90%, at least 95%, or 100% identical to SEQ ID NO: 69, wherein the miRNA binding site binds to miR-122. In another particular aspect, the miRNA binding site comprises a nucleotide sequence at least 80%, at least 85%, at least 90%, at least 95%, or 100% identical to SEQ ID NO: 68, wherein the miRNA binding site binds to miR-122. These sequences are shown below in Table 10.

In some embodiments, a nucleic acid molecule (e.g., RNA, e.g., mRNA) of the disclosure comprises a miRNA binding site, wherein the miRNA binding site comprises one or more nucleotide sequences selected from Table 10, including one or more copies of any one or more of the miRNA binding site sequences. In some embodiments, a nucleic acid molecule (e.g., RNA, e.g., mRNA) of the disclosure further comprises at least one, two, three, four, five, six, seven, eight, nine, ten, or more of the same or different miRNA binding sites selected from Table 10, including any combination thereof. In some embodiments, the miRNA binding site binds to miR-142 or is complementary to miR-142. In some embodiments, the miR-142 comprises SEQ ID NO: 61. In some embodiments, the miRNA binding site binds to miR-142-3p or miR-142-5p. In some embodiments, the miR-142-3p binding site comprises SEQ ID NO: 62. In some embodiments, the miR-142-5p binding site comprises SEQ ID NO: 63. In some embodiments, the miRNA binding site comprises a nucleotide sequence at least 80%, at least 85%, at least 90%, at least 95%, or 100% identical to SEQ ID NO: 62 or SEQ ID NO: 63.

In some embodiments, the miRNA binding site binds to miR-122 or is complementary to miR-122. In some embodiments, the miR-122 comprises SEQ ID NO: 65. In some embodiments, the miRNA binding site binds to miR-122-3p or miR-122-5p. In some embodiments, the miR-122-3p binding site comprises SEQ ID NO: 66. In some embodiments, the miR-122-5p binding site comprises SEQ ID NO: 67. In some embodiments, the miRNA binding site comprises a nucleotide sequence at least 80%, at least 85%, at least 90%, at least 95%, or 100% identical to SEQ ID NO: 62 or SEQ ID NO: 66. In some embodiments, the miRNA binding site comprises a nucleotide sequence at least 80%, at least 85%, at least 90%, at least 95%, or 100% identical to SEQ ID NO: 62 or SEQ ID NO: 67.

TABLE 10
Representative microRNAs and microRNA
binding sites

| SEQ | | |
|---|---|---|
| ID | | |
| NO. | Description | Sequence |
| 61 | miR-142 | GACAGUGCAGUCACCCAUAAAGUAGAAAGCACUAC |
| | | UAACAGCACUGGAGGGUGUAGUGUUUCCUACUUU |
| | | AUGGAUGAGUGUACUGUG |
| 59 | miR-142-3p | UGUAGUGUUUCCUACUUUAUGGA |
| 62 | miR-142-3p | UCCAUAAAGUAGGAAACACUACA |
| | binding | |
| | site | |
| 60 | miR-142-5p | CAUAAAGUAGAAAGCACUACU |
| 63 | miR-142-5p | AGUAGUGCUUUCUACUUUAUG |
| | binding | |
| | site | |
| 65 | miR-122 | CCUUAGCAGAGCUGUGGAGUGUGACAAUGGUGUU |
| | | UGUGUCUAAACUAUCAAACGCCAUUAUCACACUAA |
| | | AUAGCUACUGCUAGGC |
| 66 | miR-122-3p | AACGCCAUUAUCACACUAAAUA |
| 68 | miR-122-3p | UAUUUAGUGUGAUAAUGGCGUU |
| | binding | |
| | site | |
| 67 | miR-122-5p | UGGAGUGUGACAAUGGUGUUUG |
| 69 | miR-122-5p | CAAACACCAUUGUCACACUCCA |
| | binding | |
| | site | |

In some embodiments, a miRNA binding site is inserted in the nucleic acid molecule (e.g., RNA, e.g., mRNA) of the disclosure in any position of the nucleic acid molecule (e.g., RNA, e.g., mRNA) (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the nucleic acid molecule (e.g., RNA, e.g., mRNA) can be anywhere in the nucleic acid molecule (e.g., RNA, e.g., mRNA) as long as the insertion of the miRNA binding site in the nucleic acid molecule (e.g., RNA, e.g., mRNA) does not interfere with the translation of a functional polypeptide in the absence of the corresponding miRNA; and in the presence of the miRNA, the insertion of the miRNA binding site in the nucleic acid molecule (e.g., RNA, e.g., mRNA) and the binding of the miRNA binding site to the corresponding miRNA are capable of degrading the polynucleotide or preventing the translation of the nucleic acid molecule (e.g., RNA, e.g., mRNA).

In some embodiments, a miRNA binding site is inserted in at least about 30 nucleotides downstream from the stop codon of an ORF in a nucleic acid molecule (e.g., RNA, e.g., mRNA) of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a nucleic acid molecule (e.g., RNA, e.g., mRNA) of the disclosure.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The nucleic acid molecules (e.g., RNA, e.g., mRNA) of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a nucleic acid molecule (e.g., RNA, e.g., mRNA) of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a nucleic acid molecule (e.g., RNA, e.g., mRNA) of the disclosure. In one embodiment, miRNA binding sites incorporated into a nucleic acid molecule (e.g., RNA, e.g., mRNA) of the disclosure can be the same or can be different miRNA sites. A combination of different miRNA binding sites incorporated into a nucleic acid molecule (e.g., RNA, e.g., mRNA) of the disclosure can include combinations in which more than one copy of any of the different miRNA sites are incorporated. In another embodiment, miRNA binding sites incorporated into a nucleic acid molecule (e.g., RNA, e.g., mRNA) of the disclosure can target the same or different tissues in the body. As a non-limiting example, through the introduction of tissue-, cell-type-, or disease-specific miRNA binding sites in the 3′-UTR of a nucleic acid molecule (e.g., RNA, e.g., mRNA) of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a nucleic acid molecule (e.g., RNA, e.g., mRNA) of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In one embodiment, a nucleic acid molecule (e.g., RNA, e.g., mRNA) of the disclosure can be engineered to include more than one miRNA site expressed in different tissues or different cell types of a subject. As a non-limiting example, a nucleic acid molecule (e.g., RNA, e.g., mRNA) of the disclosure can be engineered to include miR-192 and miR-122 to regulate expression of the nucleic acid molecule (e.g., RNA, e.g., mRNA) in the liver and kidneys of a subject. In another embodiment, a nucleic acid molecule (e.g., RNA, e.g., mRNA) of the disclosure can be engineered to include more than one miRNA site for the same tissue.

In some embodiments, the therapeutic window and or differential expression associated with the polypeptide encoded by a nucleic acid molecule (e.g., RNA, e.g., mRNA) of the disclosure can be altered with a miRNA binding site. For example, a nucleic acid molecule (e.g., RNA, e.g., mRNA) encoding a polypeptide that provides a death signal can be designed to be more highly expressed in cancer cells by virtue of the miRNA signature of those cells. Where a cancer cell expresses a lower level of a particular miRNA, the nucleic acid molecule (e.g., RNA, e.g., mRNA) encoding the binding site for that miRNA (or miRNAs) would be more highly expressed. Hence, the polypeptide that provides a death signal triggers or induces cell death in the cancer cell. Neighboring noncancer cells, harboring a higher expression of the same miRNA would be less affected by the encoded death signal as the polynucleotide would be expressed at a lower level due to the effects of the miRNA binding to the binding site or “sensor” encoded in the 3′UTR. Conversely, cell survival or cytoprotective signals can be delivered to tissues containing cancer and non-cancerous cells where a miRNA has a higher expression in the cancer cells—the result being a lower survival signal to the cancer cell and a larger survival signal to the normal cell. Multiple nucleic acid molecule (e.g., RNA, e.g., mRNA) can be designed and administered having different signals based on the use of miRNA binding sites as described herein.

In some embodiments, the expression of a nucleic acid molecule (e.g., RNA, e.g., mRNA) of the disclosure can be controlled by incorporating at least one sensor sequence in the polynucleotide and formulating the nucleic acid molecule (e.g., RNA, e.g., mRNA) for administration. As a non-limiting example, a nucleic acid molecule (e.g., RNA, e.g., mRNA) of the disclosure can be targeted to a tissue or cell by incorporating a miRNA binding site and formulating the nucleic acid molecule (e.g., RNA, e.g., mRNA) in a lipid nanoparticle comprising a cationic lipid, including any of the lipids described herein.

A nucleic acid molecule (e.g., RNA, e.g., mRNA) of the disclosure can be engineered for more targeted expression in specific tissues, cell types, or biological conditions based on the expression patterns of miRNAs in the different tissues, cell types, or biological conditions. Through introduction of tissue-specific miRNA binding sites, a nucleic acid molecule (e.g., RNA, e.g., mRNA) of the disclosure can be designed for optimal protein expression in a tissue or cell, or in the context of a biological condition.

In some embodiments, a nucleic acid molecule (e.g., RNA, e.g., mRNA) of the disclosure can be designed to incorporate miRNA binding sites that either have 100% identity to known miRNA seed sequences or have less than 100% identity to miRNA seed sequences. In some embodiments, a nucleic acid molecule (e.g., RNA, e.g., mRNA) of the disclosure can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the nucleic acid molecule (e.g., RNA, e.g., mRNA). In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment, a translation enhancer element (TEE) can be incorporated on the 5′end of the stem of a stem loop and a miRNA seed can be incorporated into the stem of the stem loop. In another embodiment, a TEE can be incorporated on the 5′ end of the stem of a stem loop, a miRNA seed can be incorporated into the stem of the stem loop and a miRNA binding site can be incorporated into the 3′ end of the stem or the sequence after the stem loop. The miRNA seed and the miRNA binding site can be for the same and/or different miRNA sequences.

In one embodiment, the incorporation of a miRNA sequence and/or a TEE sequence changes the shape of the stem loop region which can increase and/or decrease translation. (see e.g, Kedde et al., “A Pumilio-induced RNA structure switch in p27-3′UTR controls miR-221 and miR-22 accessibility.” Nature Cell Biology. 2010, incorporated herein by reference in its entirety).

In one embodiment, the 5′-UTR of a nucleic acid molecule (e.g., RNA, e.g., mRNA) of the disclosure can comprise at least one miRNA sequence. The miRNA sequence can be, but is not limited to, a 19 or 22 nucleotide sequence and/or a miRNA sequence without the seed.

In one embodiment the miRNA sequence in the 5′UTR can be used to stabilize a nucleic acid molecule (e.g., RNA, e.g., mRNA) of the disclosure described herein.

In another embodiment, a miRNA sequence in the 5′UTR of a nucleic acid molecule (e.g., RNA, e.g., mRNA) of the disclosure can be used to decrease the accessibility of the site of translation initiation such as, but not limited to a start codon. See, e.g., Matsuda et al., PLoS One. 2010 11(5):e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A nucleic acid molecule (e.g., RNA, e.g., mRNA) of the disclosure can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site. As another non-limiting example, the site of translation initiation can be located within a miR-122 sequence such as the seed sequence or the mir-122 binding site.

In some embodiments, a nucleic acid molecule (e.g., RNA, e.g., mRNA) of the disclosure can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a nucleic acid molecule (e.g., RNA, e.g., mRNA) of the disclosure can be specific to the hematopoietic system. As another non-limiting example, a miRNA incorporated into a nucleic acid molecule (e.g., RNA, e.g., mRNA) of the disclosure to dampen antigen presentation is miR-142-3p.

In some embodiments, a nucleic acid molecule (e.g., RNA, e.g., mRNA) of the disclosure can include at least one miRNA in order to dampen expression of the encoded polypeptide in a tissue or cell of interest. As a non-limiting example, a nucleic acid molecule (e.g., RNA, e.g., mRNA) of the disclosure can include at least one miR-122 binding site in order to dampen expression of an encoded polypeptide of interest in the liver. As another non-limiting example a nucleic acid molecule (e.g., RNA, e.g., mRNA) of the disclosure can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a nucleic acid molecule (e.g., RNA, e.g., mRNA) of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a nucleic acid molecule (e.g., RNA, e.g., mRNA) of the disclosure more unstable in antigen presenting cells. Non-limiting examples of these miRNAs include mir-142-5p, mir-142-3p, mir-146a-5p, and mir-146-3p.

In one embodiment, a nucleic acid molecule (e.g., RNA, e.g., mRNA) of the disclosure comprises at least one miRNA sequence in a region of the nucleic acid molecule (e.g., RNA, e.g., mRNA) that can interact with a RNA binding protein.

In some embodiments, the nucleic acid molecule (e.g., RNA, e.g., mRNA) of the disclosure comprising (i) a sequence-optimized nucleotide sequence (e.g., an ORF) and (ii) a miRNA binding site (e.g., a miRNA binding site that binds to miR-142).

In some embodiments, a nucleic acid molecule (e.g., RNA, e.g., mRNA) of the disclosure comprising a 5′UTR, and ORF encoding a polypeptide, and a 3′UTR comprises one or more miR binding sites and is altered by substitution or deletion to increase or improve endonuclease resistance and/or decrease or reduce endonuclease susceptibility as described herein. In some embodiments, an endonuclease-resistant nucleic acid molecule (e.g., RNA, e.g., mRNA) of the disclosure comprising a 5′UTR, and ORF encoding a polypeptide, and a 3′UTR comprises one or more miR binding sites that binds to miR-142. In some embodiments, an endonuclease-resistant nucleic acid molecule (e.g., RNA, e.g., mRNA) of the disclosure comprising a 5′UTR, and ORF encoding a polypeptide, and a 3′UTR comprises one or more miR binding sites that binds to miR-122.

In some embodiments, the nucleic acid molecule (e.g., RNA, e.g., mRNA) of the disclosure comprises a uracil-modified sequence encoding a polypeptide disclosed herein and a miRNA binding site disclosed herein, e.g., a miRNA binding site that binds to miR-142. In some embodiments, the uracil-modified sequence encoding a polypeptide comprises at least one chemically modified nucleobase, e.g., 5-methoxyuracil. In some embodiments, at least 95% of a type of nucleobase (e.g., uracil) in a uracil-modified sequence encoding a polypeptide of the disclosure are modified nucleobases. In some embodiments, at least 95% of uracil in a uracil-modified sequence encoding a polypeptide is 5-methoxyuridine. In some embodiments, the nucleic acid molecule (e.g., RNA, e.g., mRNA) comprising a nucleotide sequence encoding a polypeptide disclosed herein and a miRNA binding site is formulated with a delivery agent.

### 3′-Stabilizing Region

In some embodiments, the mRNAs of the disclosure comprise a 3′-stabilizing region including one or more nucleosides (e.g., 1 to 500 nucleosides such as 1 to 200, 1 to 400, 1 to 10, 5 to 15, 10 to 20, 15 to 25, 20 to 30, 25 to 35, 30 to 40, 35 to 45, 40 to 50, 45 to 65, 50 to 70, 65 to 85, 70 to 90, 85 to 105, 90 to 110, 105 to 135, 120 to 150, 130 to 170, 150 to 200 or 1, 2, 3, 4, 5, 6, 7, 8, 9, 10, 20, 30, 40, 50, 60, 70, 80, 90, 100, 110, 120, 130, 140, 150, 160, 170, 180, 190, or 200 nucleosides). In some embodiments, the 3′-stabilizing region contains one or more alternative nucleosides having an alternative nucleobase, sugar, or backbone (e.g., a 2′-deoxynucleoside, a 3′-deoxynucleoside, a 2′,3′-dideoxynucleoside, a 2′-O-methylnucleoside, a 3′-O-methylnucleoside, a 3′-O-ethyl-nucleoside, 3′-arabinoside, an L-nucleoside, alpha-thio-2′-O-methyl-adenosine, 2′-fluoro-adenosine, arabino-adenosine, hexitol-adenosine, LNA-adenosine, PNA-adenosine, inverted thymidine, or 3′-azido-2′,3′-dideoxyadenosine). In some embodiments, the 3′-stabilizing region includes a plurality of alternative nucleosides. In some embodiments, the 3′-stabilizing region includes at least one non-nucleoside (e.g., an abasic ribose) at the 5′-terminus, the 3′-terminus, or at an internal position of the 3′-stabilizing region.

In some embodiments, the 3′-stabilizing region consists of one nucleoside (e.g., a 2′-deoxynucleoside, a 3′-deoxynucleoside, a 2′,3′-dideoxynucleoside, a 2′-O-methylnucleoside, a 3′-O-methylnucleoside, a 3′-O-ethyl-nucleoside, 3′-arabinoside, an L-nucleoside, alpha-thio-2′-O-methyl-adenosine, 2′-fluoro-adenosine, arabino-adenosine, hexitol-adenosine, LNA-adenosine, PNA-adenosine, inverted thymidine, or 3′-azido-2′,3′-dideoxyadenosine).

In some embodiments, one or more nucleosides in the 3′-stabilizing region include the structure:

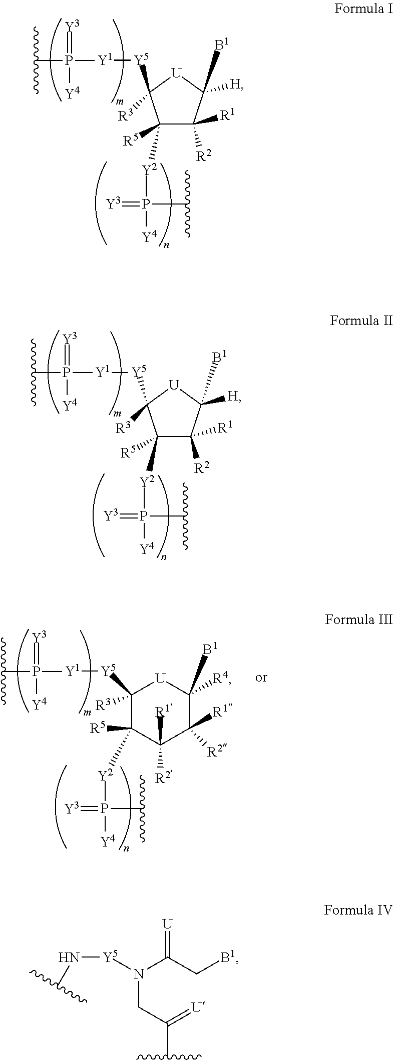

wherein B¹ is a nucleobase;

each U and U′ is, independently, O, S, N(R^{U})ₙᵤ, or C(R^{U})ₙᵤ, wherein nu is 1 or 2 (e.g., 1 for N(R^{U})ₙᵤ and 2 for C(R^{U})ₙᵤ) and each R^{U} is, independently, H, halo, or optionally substituted C₁-C₆ alkyl;

each of R¹, R^{1′}, R^{1″}, R², R^{2′}, R^{2″}, R³, R⁴, and R⁵ is, independently, H, halo, hydroxy, thiol, optionally substituted C₁-C₆ alkyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted amino, azido, optionally substituted C₆-C₁₀ aryl; or R³ and/or R⁵ can join together with one of R¹, R^{1′}, R^{1″}, R², R^{2′}, or R^{2″} to form together with the carbons to which they are attached an optionally substituted C₃-C₁₀ carbocycle or an optionally substituted C₃-C₉heterocyclyl; each of m and n is independently, 0, 1, 2, 3, 4, or 5;

each of Y¹, Y², and Y³, is, independently, O, S, Se, —NR^{N1}—, optionally substituted C₁-C₆ alkylene, or optionally substituted C₁-C₆heteroalkylene, wherein R^{N1} is H, optionally substituted C₁-C₆ alkyl, optionally substituted C₂-C₆ alkenyl, optionally substituted C₂-C₆ alkynyl, or optionally substituted C₆-C₁₀ aryl; and

each Y⁴ is, independently, H, hydroxy, protected hydroxy, halo, thiol, boranyl, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, or optionally substituted amino; and

Y⁵ is O, S, Se, optionally substituted C₁-C₆ alkylene, or optionally substituted C₁-C₆ heteroalkylene;

or is a salt thereof.

In some embodiments, the 3′-stabilizing region includes a plurality of adenosines. In some embodiments, all of the nucleosides of the 3′-stabilizing region are adenosines. In some embodiments, the 3′-stabilizing region includes at least one (e.g., at least two, at least three, at least four, at least five, at least six, at least seven, at least eight, at least nine, or at least ten) alternative nucleosides (e.g., an L-nucleoside such as L-adenosine, 2′-O-methyl-adenosine, alpha-thio-2′-O-methyl-adenosine, 2′-fluoro-adenosine, arabino-adenosine, hexitol-adenosine, LNA-adenosine, PNA-adenosine, or inverted thymidine). In some embodiments, the alternative nucleoside is an L-adenosine, a 2′-O-methyl-adenosine, or an inverted thymidine. In some embodiments, the 3′-stabilizing region includes a plurality of alternative nucleosides. In some embodiments, all of the nucleotides in the 3′-stabilizing region are alternative nucleosides. In some embodiments, the 3′-stabilizing region includes at least two different alternative nucleosides. In some embodiments, at least one alternative nucleoside is 2′-O-methyl-adenosine. In some embodiments, at least one alternative nucleoside is inverted thymidine. In some embodiments, at least one alternative nucleoside is 2′-O-methyl-adenosine, and at least one alternative nucleoside is inverted thymidine.

In some embodiments, the stabilizing region includes the structure:

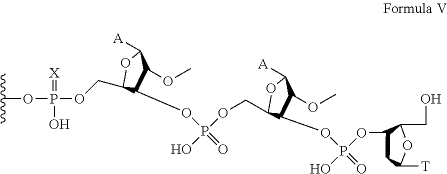

or a salt thereof;

wherein each X is, independently O or S; and

A represents adenine and T represents thymine.

In some embodiments, each X is O. In some embodiments, each X is S.

In some embodiments, all of the plurality of alternative nucleosides are the same (e.g., all of the alternative nucleosides are L-adenosine). In some embodiments, the 3′-stabilizing region includes ten nucleosides. In some embodiments, the 3′-stabilizing region includes eleven nucleosides. In some embodiments, the 3′-stabilizing region comprises at least five L-adenosines (e.g., at least ten L-adenosines, or at least twenty L-adenosines). In some embodiments, the 3′-stabilizing region consists of five L-adenosines. In some embodiments, the 3′-stabilizing region consists of ten L-adenosines. In some embodiments, the 3′-stabilizing region consists of twenty L-adenosines.

Further examples of 3′-stabilized regions are known in the art, e.g., as described in International Patent Publication Nos. WO2013/103659, WO2017/049275, and WO2017/049286, the 3′-stabilized regions of which are herein incorporated by references.

In some embodiments, the 5′-terminus of the 3′-stabilizing region is conjugated to the 3′-terminus of the 3′-UTR. In some embodiments, the 5′-terminus of the 3′-stabilizing region is conjugated to the 3′-terminus of the poly-A region. In some embodiments, the 5′-terminus of the 3′-stabilizing region is conjugated to the 3′-terminus of the poly-C region. In some embodiments of any of the foregoing polynucleotides, the 3′-stabilizing region includes the 3′-terminus of the polynucleotide.

In some embodiments, the 3′-stabilizing tail is conjugated to the remainder of the polynucleotide, e.g., at the 3′-terminus of the 3′-UTR or poly-A region via a phosphate linkage. In some embodiments, the phosphate linkage is a natural phosphate linkage. In some embodiments, the conjugation of the 3′-stabilizing tail and the remainder of the polynucleotide is produced via enzymatic or splint ligation.

In some embodiments, the 3′-stabilizing tail is conjugated to the remainder of the polynucleotide, e.g., at the 3′-terminus of the 3′-UTR or poly-A region via a chemical linkage. In some embodiments, the chemical linkage includes the structure of Formula VI:

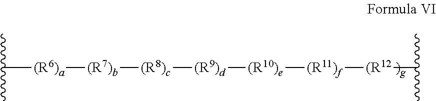

wherein a, b, c, e, f, and g are each, independently, 0 or 1;

d is 0, 1, 2, or 3;

each of R⁶, R⁸, R¹⁰, and R¹², is, independently, optionally substituted C₁-C₆ alkylene, optionally substituted C₁-C₆ heteroalkylene, optionally substituted C₂-C₆ alkenylene, optionally substituted C₂-C₆ alkynylene, or optionally substituted C₆-C₁₀ arylene, O, S, Se, and NR³;

R⁷ and R¹¹ are each, independently, carbonyl, thiocarbonyl, sulfonyl, or phosphoryl, wherein, if R⁷ is phosphoryl, —(R⁹)_{d}— is a bond, and e, f, and g are 0, then at least one of R⁶ or R⁸ is not O; and if R¹¹ is phosphoryl, —(R⁹)_{d}— is a bond, and a, b, and c are 0, then at least one of R¹⁰ or R¹² is not O;

each R⁹ is optionally substituted C₁-C₁₀ alkylene, optionally substituted C₂-C₁₀ alkenylene, optionally substituted C₂-C₁₀ alkynylene, optionally substituted C₂-C₁₀ heterocyclylene, optionally substituted C₆-C₁₂ arylene, optionally substituted C₂-C₁₀₀ polyethylene glycolene, or optionally substituted C₁-C₁₀ heteroalkylene, or a bond linking (R⁶)ₐ—(R⁷)_{b}—(R⁸)_{c} to (R¹⁰)ₑ—(R¹¹)_{f}—(R¹²)_{g}, wherein if —(R⁹)_{d}— is a bond, then at least one of a, b, c, e, f, or g is 1; and

R¹³ is hydrogen, optionally substituted C₁-C₄ alkyl, optionally substituted C₂-C₄ alkenyl, optionally substituted C₂-C₄ alkynyl, optionally substituted C₂-C₆ heterocyclyl, optionally substituted C₆-C₁₂ aryl, or optionally substituted C₁-C₇ heteroalkyl.

In some embodiments, the chemical linkage comprises the structure of Formula VII:

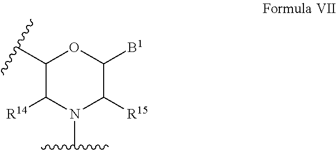

wherein B¹ is a nucleobase, hydrogen, halo, hydroxy, thiol, optionally substituted C₁-C₆ alkyl, optionally substituted C₂-C₆ alkenyl, optionally substituted C₂-C₆ alkynyl, optionally substituted C₁-C₆ heteroalkyl, optionally substituted C₂-C₆ heteroalkenyl, optionally substituted C₂-C₆ heteroalkynyl, optionally substituted amino, azido, optionally substituted C₃-C₁₀ cycloalkyl, optionally substituted C₆-C₁₀ aryl, optionally substituted C₂-C₉heterocycle; and

R¹⁴ and R¹⁵ are each, independently, hydrogen or hydroxy.

In some embodiments, the chemical linkage includes the structure:

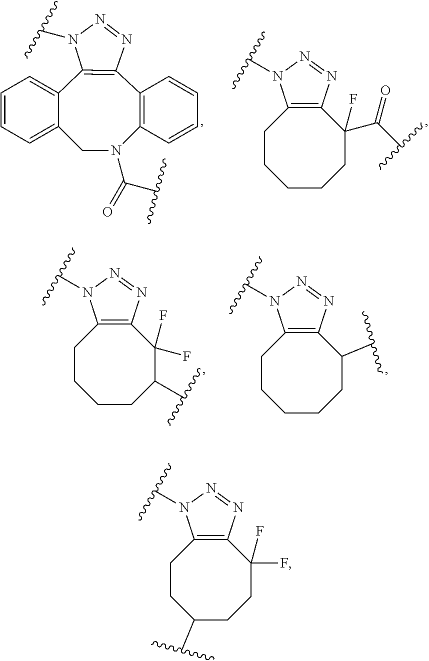 [SMILES: *C(=O)N1Cc2ccccc2-c2c(nnn2*)-c2ccccc21 ; *C(=O)C1(F)CCCCCc2c1nnn2* ; *C1CCCCc2c(nnn2*)C1(F)F ; *C1CCCCCc2c1nnn2* ; *C1CCc2c(nnn2*)C(F)(F)CC1]

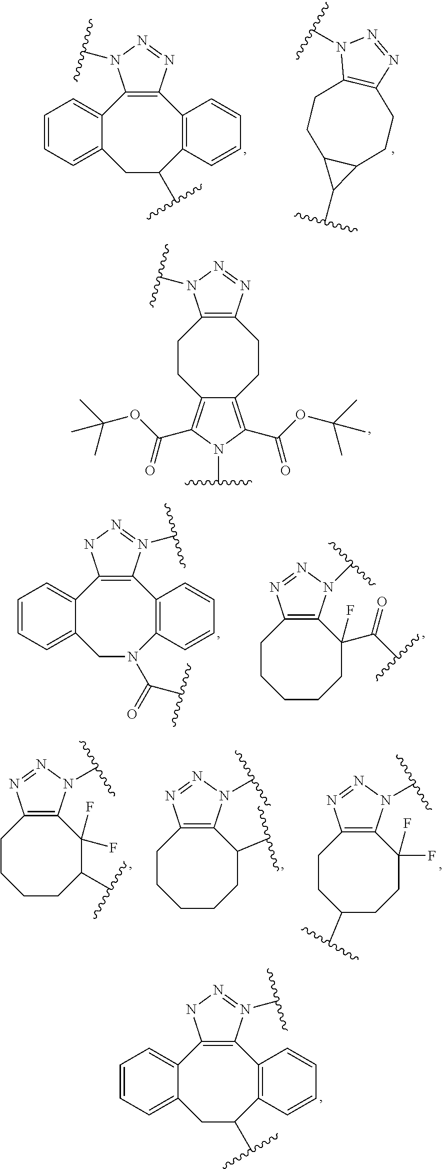

or an amide bond.

Further examples of chemical linkages to conjugate 3′-stabilized regions to the remainder of the polynucleotide are known in the art, e.g., as described in International Patent Publication Nos. WO2017/049275 and WO2017/049286, the chemical linkers of which are herein incorporated by reference.

In some embodiments, the disclosure provides a stabilized, therapeutic mRNA with increased or improved endonuclease resistance and/or decrease or reduce endonuclease susceptibility as described herein, wherein the mRNA comprises a 5′UTR, an ORF encoding a polypeptide, and a 3′UTR wherein the 3′UTR comprises a poly-A region at the 3′-terminus. In some embodiments, a stabilized, therapeutic mRNA comprises a 3′UTR wherein a 3′-stabilizing region is conjugated to the remainder of the polynucleotide, e.g., at the 3′-terminus of the 3′-UTR or poly-A region via a chemical linkage. In some embodiments, a stabilized, therapeutic mRNA of the disclosure comprises a poly-A region at the 3′-terminus of the 3′UTR that is conjugated to a 3′-stabilizing tail via a chemical linkage. In some embodiments, a stabilized, therapeutic mRNA of the disclosure comprising a poly-A region at the 3′-terminus is conjugated to a 3′-stabilizing tail comprised of L-adenosines. In some embodiments, a stabilized, therapeutic mRNA of the disclosure comprises a poly-A region at the 3′-terminus comprising at least five L-adenosines (e.g., at least ten L-adenosines, or at least twenty L-adenosines). In some embodiments, a stabilized, therapeutic mRNA of the disclosure comprises a poly-A region at the 3′-terminus comprising at least five L-adenosines. In some embodiments, a stabilized, therapeutic mRNA of the disclosure comprises a poly-A region at the 3′-terminus comprising at least ten L-adenosines. In some embodiments, a stabilized, therapeutic mRNA of the disclosure comprises a poly-A region at the 3′-terminus comprising at least twenty L-adenosines.

In some embodiments, a stabilized, therapeutic mRNA of the disclosure comprising a poly-A region at the 3′-terminus that is conjugated to a 3′-stabilizing region has increased half-life when compared to an unaltered mRNA not conjugated to a 3′-stabilizing region. In some embodiments, a stabilized, therapeutic mRNA of the disclosure comprising a poly-A region at the 3′-terminus that is conjugated to a 3′-stabilizing region has a half-life that is increased by at least 1-fold, at least 2-fold, at least 3-fold, at least 4-fold, at least 5-fold, at least 6-fold, at least 7-fold, at least 8-fold, at least 9-fold, or at least 10-fold when compared to an unaltered mRNA not conjugated to a 3′-stabilizing region.

### Delivery Agents

### Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In certain embodiments, the present application provides pharmaceutical compositions comprising:

(a) an mRNA comprising a nucleotide sequence encoding a polypeptide; and

(b) a delivery agent.

### Lipid Nanoparticle Formulations

In some embodiments, nucleic acids of the invention (e.g. mRNA) are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Nucleic acids of the present disclosure (e.g. mRNA) are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol. In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid.

### Ionizable Lipids

In some aspects, the ionizable lipids of the present disclosure may be one or more of compounds of Formula (I):

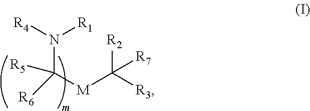

or their N-oxides, or salts or isomers thereof, wherein:

R₁ is selected from the group consisting of C₅₋₃₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of hydrogen, a C₃₋₆ carbocycle, —(CH₂)ₙQ, —(CH₂)ₙCHQR,

—CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —N(R)S(O)₂R₈, —O(CH₂)˜OR, —N(R)C(═NR₉)N(R)₂, —N(R)C(═CHR₉)N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉)N(R)₂, —N(OR)C(═CHR₉)N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected

from —C(O)O—, —OC(O)—, —OC(O)-M″-C(O)O—, —C(O)N(R′)—,

—N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group, in which M″ is a bond, C₁₋₁₃ alkyl or C₂₋₁₃ alkenyl;

R₇ is selected from the group consisting of C₁₋₃ alkyl, C₂₋₃ alkenyl, and H;

R₈ is selected from the group consisting of C₃₋₆ carbocycle and heterocycle;

R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₅ alkyl and C₃₋₁₅ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13; and wherein when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

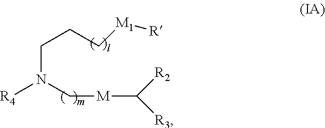

or its N-oxide, or a salt or isomer thereof, wherein 1 is selected from 1, 2, 3, 4, and 5; m is selected from 5, 6, 7, 8, and 9; M₁ is a bond or M′; R₄ is hydrogen, unsubstituted C₁₋₃ alkyl, or —(CH₂)ₙQ, in which Q is

OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group, and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (IB):

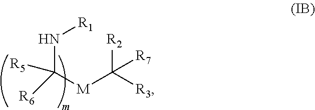

or its N-oxide, or a salt or isomer thereof in which all variables are as defined herein. For example, m is selected from 5, 6, 7, 8, and 9; R₄ is hydrogen, unsubstituted C₁₋₃ alkyl, or —(CH₂)ₙQ, in which Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, m is 5, 7, or 9. For example, Q is OH, —NHC(S)N(R)₂, or —NHC(O)N(R)₂. For example, Q is —N(R)C(O)R, or —N(R)S(O)₂R.

In certain embodiments, a subset of compounds of Formula (I) includes those of Formula (II):

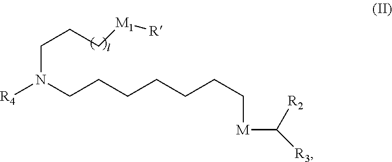

or its N-oxide, or a salt or isomer thereof, wherein 1 is selected from 1, 2, 3, 4, and 5; M₁ is a bond or M′; R₄ is hydrogen, unsubstituted C₁₋₃ alkyl, or —(CH₂)ₙQ, in which n is 2, 3, or 4, and Q is
OH, —NHC(S)N(R)₂, —NHC(O)N(R)₂, —N(R)C(O)R, —N(R)S(O)₂R, —N(R)R₈, —NHC(═NR₉)N(R)₂, —NHC(═CHR₉)N(R)₂, —OC(O)N(R)₂, —N(R)C(O)OR, heteroaryl or heterocycloalkyl; M and M′ are independently selected
from —C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl.

In one embodiment, the compounds of Formula (I) are of Formula (IIa),

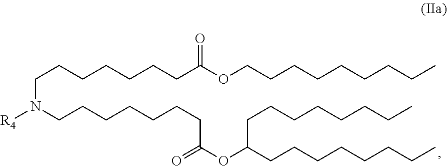

or their N-oxides, or salts or isomers thereof, wherein R₄ is as described herein.

In another embodiment, the compounds of Formula (I) are of Formula (IIb),

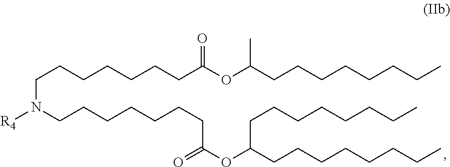

or their N-oxides, or salts or isomers thereof, wherein R₄ is as described herein.

In another embodiment, the compounds of Formula (I) are of Formula (IIc) or (IIe):

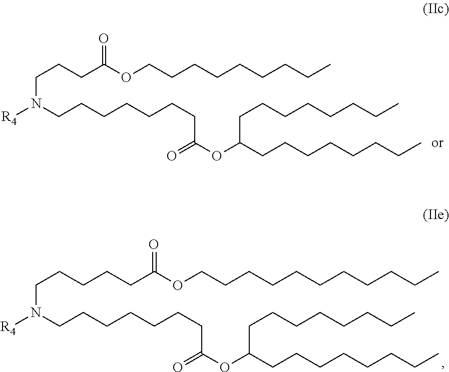

or their N-oxides, or salts or isomers thereof, wherein R₄ is as described herein.

In another embodiment, the compounds of Formula (I) are of Formula (IIf):

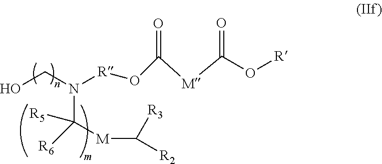

or their N-oxides, or salts or isomers thereof,

wherein M is —C(O)O— or —OC(O)—, M″ is C₁₋₆ alkyl or C₂₋₆ alkenyl, R₂ and R₃ are independently selected from the group consisting of C₅₋₁₄ alkyl and C₅₋₁₄ alkenyl, and n is selected from 2, 3, and 4.

In a further embodiment, the compounds of Formula (I) are of Formula (IId),

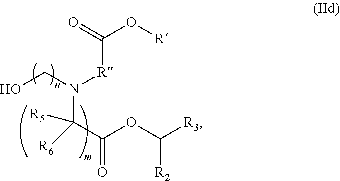

or their N-oxides, or salts or isomers thereof, wherein n is 2, 3, or 4; and m, R′, R″, and R₂ through R₆ are as described herein. For example, each of R₂ and R₃ may be independently selected from the group consisting of C₅₋₁₄ alkyl and C₅₋₁₄ alkenyl.

In a further embodiment, the compounds of Formula (I) are of Formula (IIg),

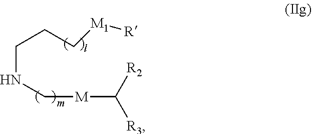

or their N-oxides, or salts or isomers thereof, wherein 1 is selected from 1, 2, 3, 4, and 5; m is selected from 5, 6, 7, 8, and 9; M₁ is a bond or M′; M and M′ are independently selected from

—C(O)O—, —OC(O)—, —OC(O)-M″-C(O)O—, —C(O)N(R′)—, —P(O)(OR′)O—, —S—S—, an aryl group, and a heteroaryl group; and R₂ and R₃ are independently selected from the group consisting of H, C₁₋₁₄ alkyl, and C₂₋₁₄ alkenyl. For example, M″ is C₁₋₆ alkyl (e.g., C₁₋₄ alkyl) or C₂₋₆ alkenyl (e.g. C₂₋₄ alkenyl). For example, R₂ and R₃ are independently selected from the group consisting of C₅₋₁₄ alkyl and C₅₋₁₄ alkenyl.

In some embodiments, the ionizable lipids are one or more of the compounds described in U.S. Application Nos. 62/220,091, 62/252,316, 62/253,433, 62/266,460, 62/333,557, 62/382,740, 62/393,940, 62/471,937, 62/471,949, 62/475,140, and 62/475,166, and PCT Application No. PCT/US2016/052352.

In some embodiments, the ionizable lipids are selected from Compounds 1-280 described in U.S. Application No. 62/475,166.

In some embodiments, the ionizable lipid is

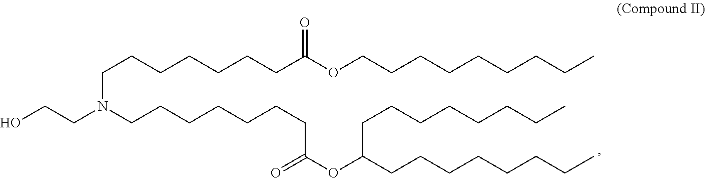 [SMILES: CCCCCCCCCOC(=O)CCCCCCCN(CCO)CCCCCCCC(=O)OC(CCCCCCCC)CCCCCCCC]

or a salt thereof.

In some embodiments, the ionizable lipid is

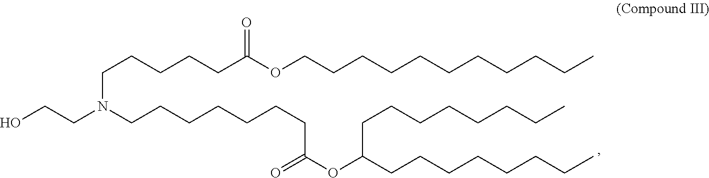 [SMILES: CCCCCCCCCCCOC(=O)CCCCCN(CCO)CCCCCCCC(=O)OC(CCCCCCCC)CCCCCCCC]

or a salt thereof.

In some embodiments, the ionizable lipid is

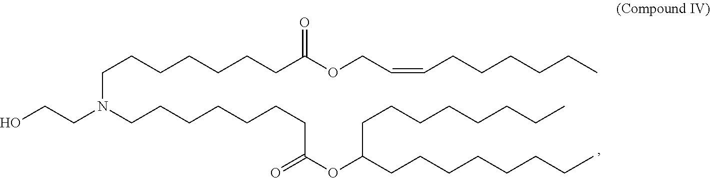 [SMILES: CCCCCC/C=C\COC(=O)CCCCCCCN(CCO)CCCCCCCC(=O)OC(CCCCCCCC)CCCCCCCC]

or a salt thereof.

In some embodiments, the ionizable lipid is

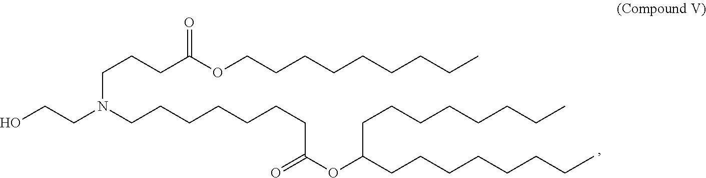 [SMILES: CCCCCCCCCOC(=O)CCCN(CCO)CCCCCCCC(=O)OC(CCCCCCCC)CCCCCCCC]

or a salt thereof.

The central amine moiety of a lipid according to Formula (I), (IA), (IB), (II), (IIa), (IIb), (IIc), (IId), (IIe), (IIf), or (IIg) may be protonated at a physiological pH. Thus, a lipid may have a positive or partial positive charge at physiological pH. Such lipids may be referred to as cationic or ionizable (amino) lipids. Lipids may also be zwitterionic, i.e., neutral molecules having both a positive and a negative charge.

In some aspects, the ionizable lipids of the present disclosure may be one or more of compounds of formula (III),

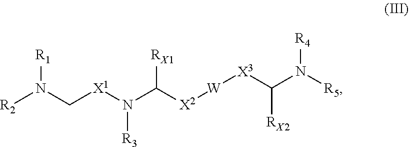

or salts or isomers thereof, wherein

W is

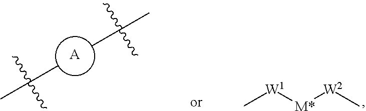

ring A is

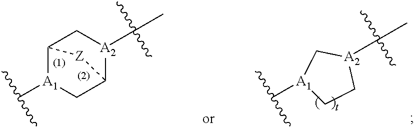

t is 1 or 2;

A₁ and A₂ are each independently selected from CH or N;

Z is CH₂ or absent wherein when Z is CH₂, the dashed lines (1) and (2) each represent a single bond; and when Z is absent, the dashed lines (1) and (2) are both absent;

R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;

R_{X1} and R_{X2} are each independently H or C₁₋₃ alkyl;

each M is independently selected from the group consisting

of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, —C(O)S—, —SC(O)—, an aryl group, and a heteroaryl group;

M* is C₁-C₆ alkyl,

W¹ and W² are each independently selected from the group consisting of —O— and —N(R₆)—;

each R₆ is independently selected from the group consisting of H and C₁₋₅ alkyl;

X¹, X², and X³ are independently selected from the group consisting of a bond, —CH₂—, —(CH₂)₂—, —CHR—, —CHY—, —C(O)—, —C(O)O—, —OC(O)—, —(CH₂)ₙ—C(O)—, —C(O)—(CH₂)ₙ—, —(CH₂)ₙ—C(O)O—, —OC(O)—(CH₂)ₙ—, —(CH₂)˜—OC(O)—, —C(O)O—(CH₂)ₙ—, —CH(OH)—, —C(S)—, and —CH(SH)—;

each Y is independently a C₃₋₆ carbocycle;

each R* is independently selected from the group consisting of C₁₋₁₂ alkyl and C₂₋₁₂ alkenyl;

each R is independently selected from the group consisting of C₁₋₃ alkyl and a C₃₋₆ carbocycle;

each R′ is independently selected from the group consisting of C₁₋₁₂ alkyl, C₂₋₁₂ alkenyl, and H;

each R″ is independently selected from the group consisting of C₃₋₁₂ alkyl, C₃₋₁₂ alkenyl and —R*MR′; and

n is an integer from 1-6;

when ring A is

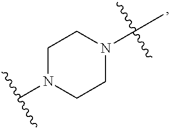 [SMILES: *N1CCN(*)CC1]

then

i) at least one of X¹, X², and X³ is not —CH₂—; and/or

ii) at least one of R₁, R₂, R₃, R₄, and R₅ is —R″MR′.

In some embodiments, the compound is of any of formulae (IIIa1)-(IIIa8):

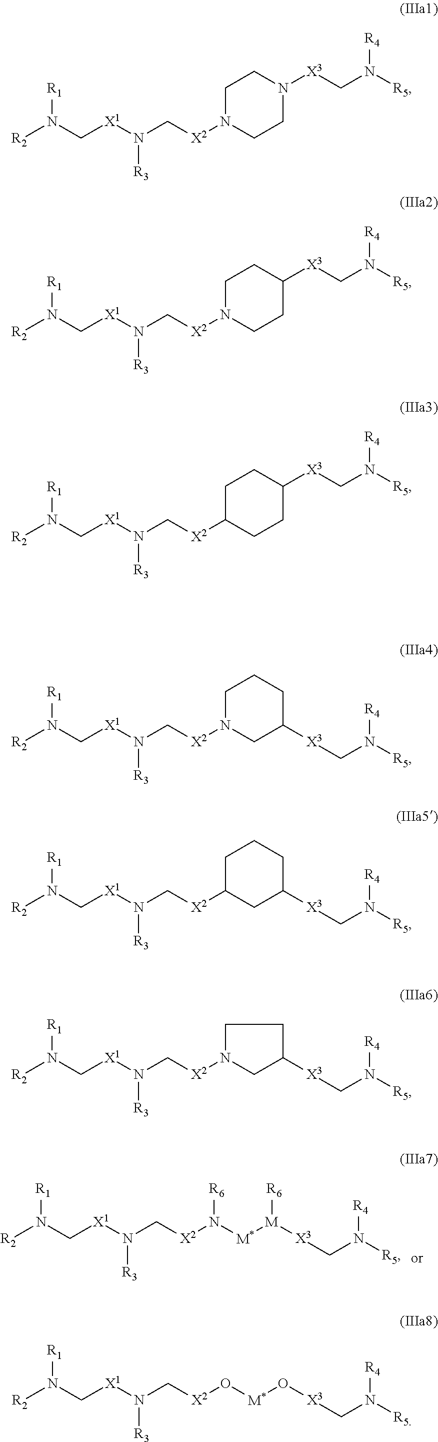

In some embodiments, the ionizable lipids are one or more of the compounds described in U.S. Application Nos. 62/271,146, 62/338,474, 62/413,345, and 62/519,826, and PCT Application No. PCT/US2016/068300.

In some embodiments, the ionizable lipids are selected from Compounds 1-156 described in U.S. Application No. 62/519,826.

In some embodiments, the ionizable lipids are selected from Compounds 1-16, 42-66, 68-76, and 78-156 described in U.S. Application No. 62/519,826.

In some embodiments, the ionizable lipid is

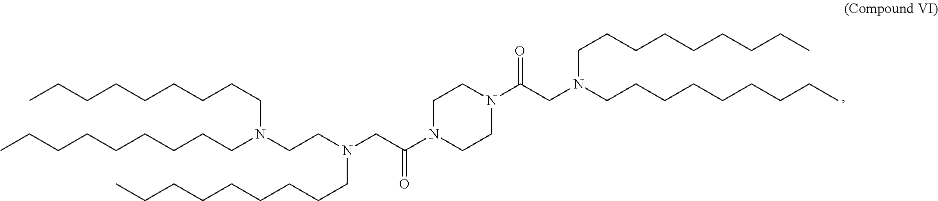 [SMILES: CCCCCCCCCN(CCCCCCCCC)CCN(CCCCCCCCC)CC(=O)N1CCN(C(=O)CN(CCCCCCCCC)CCCCCCCCC)CC1]

or a salt thereof.

In some embodiments, the ionizable lipid is (Compound VII), or a salt thereof.

The central amine moiety of a lipid according to Formula (III), (IIIa1), (IIIa2), (IIIa3), (IIIa4), (IIIa5), (IIIa6), (IIIa7), or (IIIa8) may be protonated at a physiological pH. Thus, a lipid may have a positive or partial positive charge at physiological pH. Such lipids may be referred to as cationic or ionizable (amino)lipids. Lipids may also be zwitterionic, i.e., neutral molecules having both a positive and a negative charge.

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the invention comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine,1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

In certain embodiments, a phospholipid useful or potentially useful in the present invention is an analog or variant of DSPC. In certain embodiments, a phospholipid useful or potentially useful in the present invention is a compound of Formula (IV):

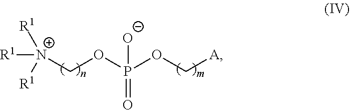

or a salt thereof, wherein:

each R¹ is independently optionally substituted alkyl; or optionally two R¹ are joined together with the intervening atoms to form optionally substituted monocyclic carbocyclyl or optionally substituted monocyclic heterocyclyl; or optionally three R¹ are joined together with the intervening atoms to form optionally substituted bicyclic carbocyclyl or optionally substitute bicyclic heterocyclyl;

n is 1, 2, 3, 4, 5, 6, 7, 8, 9, or 10;

m is 0, 1, 2, 3, 4, 5, 6, 7, 8, 9, or 10;

A is of the formula:

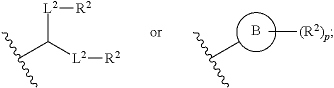

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or —NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, —C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2;

provided that the compound is not of the formula:

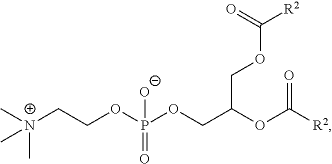

wherein each instance of R² is independently unsubstituted alkyl, unsubstituted alkenyl, or unsubstituted alkynyl.

In some embodiments, the phospholipids may be one or more of the phospholipids described in U.S. Application No. 62/520,530.

### (i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IV), at least one of R¹ is not methyl. In certain embodiments, at least one of R¹ is not hydrogen or methyl. In certain embodiments, the compound of Formula (IV) is of one of the following formulae:

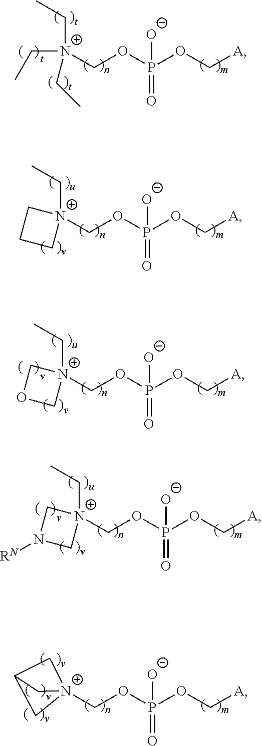

or a salt thereof, wherein:

each t is independently 1, 2, 3, 4, 5, 6, 7, 8, 9, or 10;

each u is independently 0, 1, 2, 3, 4, 5, 6, 7, 8, 9, or 10; and

each v is independently 1, 2, or 3.

In certain embodiments, a compound of Formula (IV) is of Formula (IV-a):

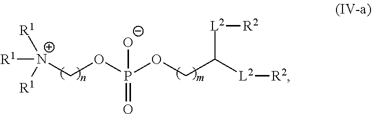

or a salt thereof.

In certain embodiments, a phospholipid useful or potentially useful in the present invention comprises a cyclic moiety in place of the glyceride moiety. In certain embodiments, a phospholipid useful in the present invention is DSPC, or analog thereof, with a cyclic moiety in place of the glyceride moiety. In certain embodiments, the compound of Formula (IV) is of Formula (IV-b):

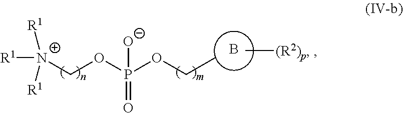

or a salt thereof.

### (ii) Phospholipid Tail Modifications

In certain embodiments, a phospholipid useful or potentially useful in the present invention comprises a modified tail. In certain embodiments, a phospholipid useful or potentially useful in the present invention is DSPC, or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IV) is of Formula (IV-a), or a salt thereof, wherein at least one instance of R² is each instance of R² is optionally substituted C₁₋₃₀ alkyl, wherein one or more methylene units of R² are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), —NR^{N}C(═NR^{N})N(R^{N}), C(S), C(S)N(R^{N}), NR^{N}C(S), NR^{N}C(S)N(R^{N}), S(O), OS(O), S(O)O, OS(O)O, —OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or N(R^{N})S(O)₂O.

In certain embodiments, the compound of Formula (IV) is of Formula (IV-c):

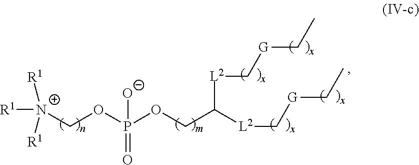

or a salt thereof, wherein:

each x is independently an integer between 0-30, inclusive; and

each instance is G is independently selected from the group consisting of optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), —C(O)O, OC(O), OC(O)O, OC(O)N(R^{N}), NR^{N}C(O)O, C(O)S, SC(O), C(═NR^{N}), C(═NR^{N})N(R^{N}), —NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), —S(O)O, OS(O)O, OS(O)₂, S(O)₂O, OS(O)₂O, N(R^{N})S(O), S(O)N(R^{N}), N(R^{N})S(O)N(R^{N}), —OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O. Each possibility represents a separate embodiment of the present invention.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IV), wherein n is 1, 3, 4, 5, 6, 7, 8, 9, or 10. For example, in certain embodiments, a compound of Formula (IV) is of one of the following formulae:

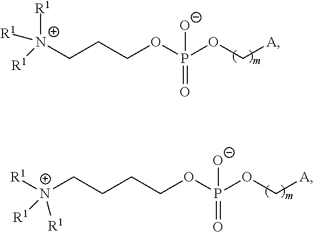

or a salt thereof.

### Alternative Lipids

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the invention is oleic acid.

In certain embodiments, the alternative lipid is one of the following:

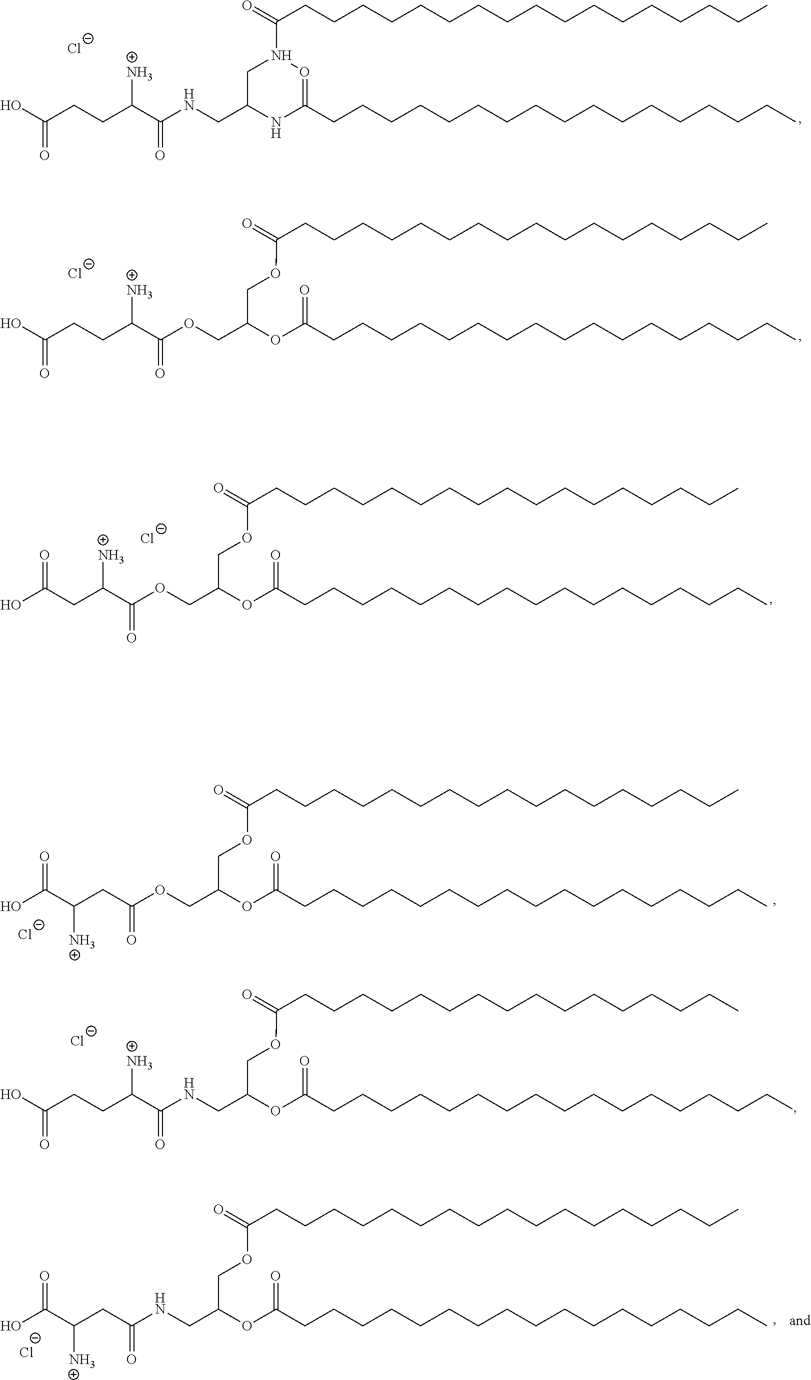

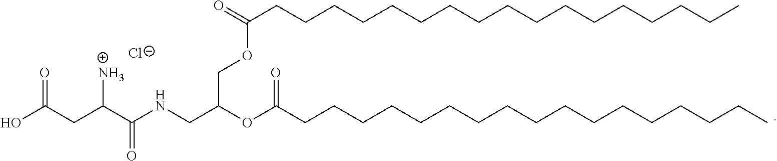 [SMILES: CCCCCCCCCCCCCCCCCC(=O)OCC(CNC(=O)C([NH3+])CC(=O)O)OC(=O)CCCCCCCCCCCCCCCCC ; [Cl-]]

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. Application No. 62/520,530.

### Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

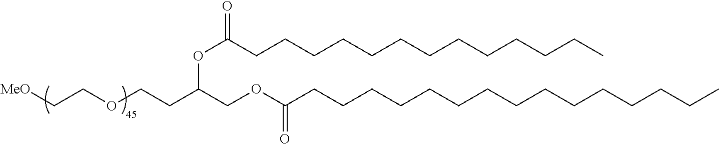

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (V). Provided herein are compounds of Formula (V):

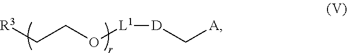

or salts thereof, wherein:

R³ is —OR^{O};

R^{O} is hydrogen, optionally substituted alkyl, or an oxygen protecting group;

r is an integer between 1 and 100, inclusive;

L¹ is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, OC(O), OC(O)O, OC(O)N(R^{N}), —NR^{N}C(O)O, or NR^{N}C(O)N(R^{N});

D is a moiety obtained by click chemistry or a moiety cleavable under physiological conditions;

m is 0, 1, 2, 3, 4, 5, 6, 7, 8, 9, or 10;

A is of the formula:

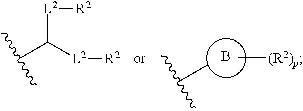

each instance of L² is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, OC(O)N(R^{N}), NR^{N}C(O)O, or —NR^{N}C(O)N(R^{N});

each instance of R² is independently optionally substituted C₁₋₃₀ alkyl, optionally substituted C₁₋₃₀ alkenyl, or optionally substituted C₁₋₃₀ alkynyl; optionally wherein one or more methylene units of R² are independently replaced with optionally substituted carbocyclylene, optionally substituted heterocyclylene, optionally substituted arylene, optionally substituted heteroarylene, N(R^{N}), O, S, —C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O)S, SC(O), C(═NR^{N}), C(═NR^{N})N(R^{N}), NR^{N}C(═NR^{N}), NR^{N}C(═NR^{N})N(R^{N}), C(S), C(S)N(R^{N}), —NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), —N(R^{N})S(O)₂N(R^{N}), OS(O)₂N(R^{N}), or N(R^{N})S(O)₂O;

each instance of R^{N} is independently hydrogen, optionally substituted alkyl, or a nitrogen protecting group;

Ring B is optionally substituted carbocyclyl, optionally substituted heterocyclyl, optionally substituted aryl, or optionally substituted heteroaryl; and

p is 1 or 2.

In certain embodiments, the compound of Formula (V) is a PEG-OH lipid (i.e., R³ is —OR^{O}, and R^{O} is hydrogen). In certain embodiments, the compound of Formula (V) is of Formula (V-OH):

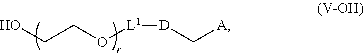

or a salt thereof.

In certain embodiments, a PEG lipid useful in the present invention is a PEGylated fatty acid. In certain embodiments, a PEG lipid useful in the present invention is a compound of Formula (VI). Provided herein are compounds of Formula (VI):

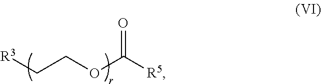

or a salts thereof, wherein:

R³ is —OR^{O};

R^{O} is hydrogen, optionally substituted alkyl or an oxygen protecting group;

r is an integer between 1 and 100, inclusive;

R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O)S, SC(O), C(═NR^{N}), —C(═NR^{N})N(R^{N}), NR^{N}C(═NR^{N}), NR^{N}C(═NR^{N})N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N})S(O), S(O)N(R^{N}), N(R^{N})S(O)N(R^{N}), OS(O)N(R^{N}), N(R^{N})S(O)O, S(O)₂, N(R^{N})S(O)₂, S(O)₂N(R^{N}), N(R^{N})S(O)₂N(R^{N}), OS(O)₂N(R^{N}), or —N(R^{N})S(O)₂O; and

each instance of R^{N} is independently hydrogen, optionally substituted alkyl, or a nitrogen protecting group.

In certain embodiments, the compound of Formula (VI) is of Formula (VI-OH):

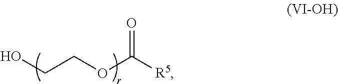

or a salt thereof. In some embodiments, r is 45.

In yet other embodiments the compound of Formula (VI) is:

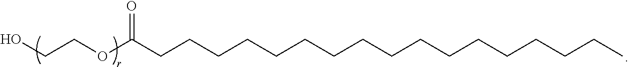

or a salt thereof.

In one embodiment, the compound of Formula (VI) is

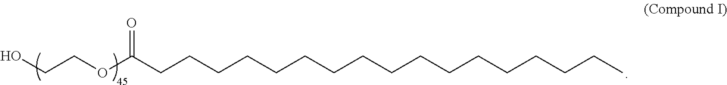

In some aspects, the lipid composition of the pharmaceutical compositions disclosed herein does not comprise a PEG-lipid.

In some embodiments, the PEG-lipids may be one or more of the PEG lipids described in U.S. Application No. 62/520,530.

In some embodiments, a PEG lipid of the invention comprises a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some embodiments, the PEG-modified lipid is PEG-DMG, PEG-c-DOMG (also referred to as PEG-DOMG), PEG-DSG and/or PEG-DPG.

In some embodiments, a LNP of the invention comprises an ionizable cationic lipid of any of Formula I, II or III, a phospholipid comprising DSPC, a structural lipid, and a PEG lipid comprising PEG-DMG.

In some embodiments, a LNP of the invention comprises an ionizable cationic lipid of any of Formula I, II or III, a phospholipid comprising DSPC, a structural lipid, and a PEG lipid comprising a compound having Formula VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid comprising a compound having Formula IV, a structural lipid, and the PEG lipid comprising a compound having Formula V or VI.

In some embodiments, a LNP of the invention comprises an ionizable cationic lipid of Formula I, II or III, a phospholipid having Formula IV, a structural lipid, and a PEG lipid comprising a compound having Formula VI.

In some embodiments, a LNP of the invention comprises an ionizable cationic lipid of

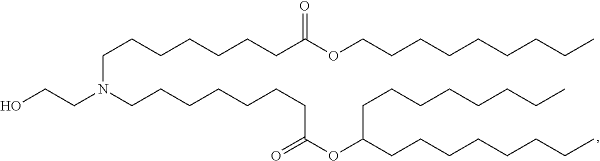 [SMILES: CCCCCCCCCOC(=O)CCCCCCCN(CCO)CCCCCCCC(=O)OC(CCCCCCCC)CCCCCCCC]

and a PEG lipid comprising Formula VI.

In some embodiments, a LNP of the invention comprises an ionizable cationic lipid of

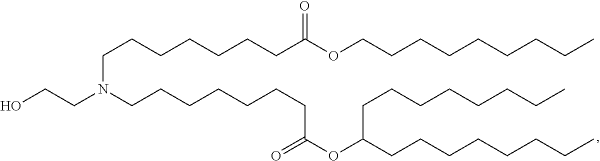 [SMILES: CCCCCCCCCOC(=O)CCCCCCCN(CCO)CCCCCCCC(=O)OC(CCCCCCCC)CCCCCCCC]

and an alternative lipid comprising oleic acid.

In some embodiments, a LNP of the invention comprises an ionizable cationic lipid of

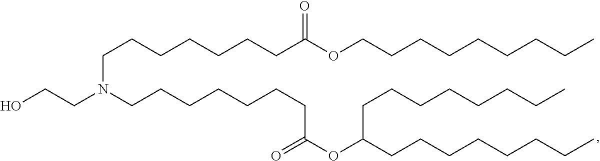 [SMILES: CCCCCCCCCOC(=O)CCCCCCCN(CCO)CCCCCCCC(=O)OC(CCCCCCCC)CCCCCCCC]

an alternative lipid comprising oleic acid, a structural lipid comprising cholesterol, and a PEG lipid comprising a compound having Formula VI.

In some embodiments, a LNP of the invention comprises an ionizable cationic lipid of

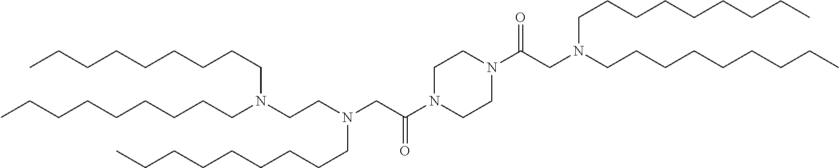 [SMILES: CCCCCCCCCN(CCCCCCCCC)CCN(CCCCCCCCC)CC(=O)N1CCN(C(=O)CN(CCCCCCCCC)CCCCCCCCC)CC1]

a phospholipid comprising DOPE, a structural lipid comprising cholesterol, and a PEG lipid comprising a compound having Formula VI.

In some embodiments, a LNP of the invention comprises an ionizable cationic lipid of

a phospholipid comprising DOPE, a structural lipid comprising cholesterol, and a PEG lipid comprising a compound having Formula VII.

In some embodiments, a LNP of the invention comprises an N:P ratio of from about 2:1 to about 30:1.

In some embodiments, a LNP of the invention comprises an N:P ratio of about 6:1.

In some embodiments, a LNP of the invention comprises an N:P ratio of about 3:1.

In some embodiments, a LNP of the invention comprises a wt/wt ratio of the ionizable cationic lipid component to the RNA of from about 10:1 to about 100:1.

In some embodiments, a LNP of the invention comprises a wt/wt ratio of the ionizable cationic lipid component to the RNA of about 20:1.

In some embodiments, a LNP of the invention comprises a wt/wt ratio of the ionizable cationic lipid component to the RNA of about 10:1.

In some embodiments, a LNP of the invention has a mean diameter from about 50 nm to about 150 nm.

In some embodiments, a LNP of the invention has a mean diameter from about 70 nm to about 120 nm.

As used herein, the term “alkyl”, “alkyl group”, or “alkylene” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms), which is optionally substituted. The notation “C1-14 alkyl” means an optionally substituted linear or branched, saturated hydrocarbon including 1 14 carbon atoms. Unless otherwise specified, an alkyl group described herein refers to both unsubstituted and substituted alkyl groups.

As used herein, the term “alkenyl”, “alkenyl group”, or “alkenylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond, which is optionally substituted. The notation “C2-14 alkenyl” means an optionally substituted linear or branched hydrocarbon including 2 14 carbon atoms and at least one carbon-carbon double bond. An alkenyl group may include one, two, three, four, or more carbon-carbon double bonds. For example, C18 alkenyl may include one or more double bonds. A C18 alkenyl group including two double bonds may be a linoleyl group. Unless otherwise specified, an alkenyl group described herein refers to both unsubstituted and substituted alkenyl groups.

As used herein, the term “alkynyl”, “alkynyl group”, or “alkynylene” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one carbon-carbon triple bond, which is optionally substituted. The notation “C2-14 alkynyl” means an optionally substituted linear or branched hydrocarbon including 2 14 carbon atoms and at least one carbon-carbon triple bond. An alkynyl group may include one, two, three, four, or more carbon-carbon triple bonds. For example, C18 alkynyl may include one or more carbon-carbon triple bonds. Unless otherwise specified, an alkynyl group described herein refers to both unsubstituted and substituted alkynyl groups.

As used herein, the term “carbocycle” or “carbocyclic group” means an optionally substituted mono- or multi-cyclic system including one or more rings of carbon atoms. Rings may be three, four, five, six, seven, eight, nine, ten, eleven, twelve, thirteen, fourteen, fifteen, sixteen, seventeen, eighteen, nineteen, or twenty membered rings. The notation “C3-6 carbocycle” means a carbocycle including a single ring having 3-6 carbon atoms. Carbocycles may include one or more carbon-carbon double or triple bonds and may be non-aromatic or aromatic (e.g., cycloalkyl or aryl groups). Examples of carbocycles include cyclopropyl, cyclopentyl, cyclohexyl, phenyl, naphthyl, and 1,2 dihydronaphthyl groups. The term “cycloalkyl” as used herein means a non-aromatic carbocycle and may or may not include any double or triple bond. Unless otherwise specified, carbocycles described herein refers to both unsubstituted and substituted carbocycle groups, i.e., optionally substituted carbocycles.

As used herein, the term “heterocycle” or “heterocyclic group” means an optionally substituted mono- or multi-cyclic system including one or more rings, where at least one ring includes at least one heteroatom. Heteroatoms may be, for example, nitrogen, oxygen, or sulfur atoms. Rings may be three, four, five, six, seven, eight, nine, ten, eleven, twelve, thirteen, or fourteen membered rings. Heterocycles may include one or more double or triple bonds and may be non-aromatic or aromatic (e.g., heterocycloalkyl or heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. The term “heterocycloalkyl” as used herein means a non-aromatic heterocycle and may or may not include any double or triple bond. Unless otherwise specified, heterocycles described herein refers to both unsubstituted and substituted heterocycle groups, i.e., optionally substituted heterocycles.

As used herein, the term “heteroalkyl”, “heteroalkenyl”, or “heteroalkynyl”, refers respectively to an alkyl, alkenyl, alkynyl group, as defined herein, which further comprises one or more (e.g., 1, 2, 3, or 4) heteroatoms (e.g., oxygen, sulfur, nitrogen, boron, silicon, phosphorus) wherein the one or more heteroatoms is inserted between adjacent carbon atoms within the parent carbon chain and/or one or more heteroatoms is inserted between a carbon atom and the parent molecule, i.e., between the point of attachment. Unless otherwise specified, heteroalkyls, heteroalkenyls, or heteroalkynyls described herein refers to both unsubstituted and substituted heteroalkyls, heteroalkenyls, or heteroalkynyls, i.e., optionally substituted heteroalkyls, heteroalkenyls, or heteroalkynyls.

As used herein, a “biodegradable group” is a group that may facilitate faster metabolism of a lipid in a mammalian entity. A biodegradable group may be selected from the group consisting of, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)2-, an aryl group, and a heteroaryl group. As used herein, an “aryl group” is an optionally substituted carbocyclic group including one or more aromatic rings. Examples of aryl groups include phenyl and naphthyl groups. As used herein, a “heteroaryl group” is an optionally substituted heterocyclic group including one or more aromatic rings. Examples of heteroaryl groups include pyrrolyl, furyl, thiophenyl, imidazolyl, oxazolyl, and thiazolyl. Both aryl and heteroaryl groups may be optionally substituted. For example, M and M′ can be selected from the non-limiting group consisting of optionally substituted phenyl, oxazole, and thiazole. In the formulas herein, M and M′ can be independently selected from the list of biodegradable groups above. Unless otherwise specified, aryl or heteroaryl groups described herein refers to both unsubstituted and substituted groups, i.e., optionally substituted aryl or heteroaryl groups.

Alkyl, alkenyl, and cyclyl (e.g., carbocyclyl and heterocyclyl) groups may be optionally substituted unless otherwise specified. Optional substituents may be selected from the group consisting of, but are not limited to, a halogen atom (e.g., a chloride, bromide, fluoride, or iodide group), a carboxylic acid (e.g., C(O)OH), an alcohol (e.g., a hydroxyl, OH), an ester (e.g., C(O)OR OC(O)R), an aldehyde (e.g., C(O)H), a carbonyl (e.g., C(O)R, alternatively represented by C═O), an acyl halide (e.g., C(O)X, in which X is a halide selected from bromide, fluoride, chloride, and iodide), a carbonate (e.g., OC(O)OR), an alkoxy (e.g., OR), an acetal (e.g., C(OR)2R″″, in which each OR are alkoxy groups that can be the same or different and R″″ is an alkyl or alkenyl group), a phosphate (e.g., P(O)43-), a thiol (e.g., SH), a sulfoxide (e.g., S(O)R), a sulfinic acid (e.g., S(O)OH), a sulfonic acid (e.g., S(O)2OH), a thial (e.g., C(S)H), a sulfate (e.g., S(O)42-), a sulfonyl (e.g., S(O)₂), an amide (e.g., C(O)NR₂, or N(R)C(O)R), an azido (e.g., N3), a nitro (e.g., NO2), a cyano (e.g., CN), an isocyano (e.g., NC), an acyloxy (e.g., OC(O)R), an amino (e.g., NR2, NRH, or NH2), a carbamoyl (e.g., OC(O)NR2, OC(O)NRH, or OC(O)NH2), a sulfonamide (e.g., S(O)2NR2, S(O)2NRH, S(O)2NH2, N(R)S(O)2R, N(H)S(O)2R, N(R)S(O)2H, or N(H)S(O)2H), an alkyl group, an alkenyl group, and a cyclyl (e.g., carbocyclyl or heterocyclyl) group. In any of the preceding, R is an alkyl or alkenyl group, as defined herein. In some embodiments, the substituent groups themselves may be further substituted with, for example, one, two, three, four, five, or six substituents as defined herein. For example, a C1 6 alkyl group may be further substituted with one, two, three, four, five, or six substituents as described herein.

Compounds of the disclosure that contain nitrogens can be converted to N-oxides by treatment with an oxidizing agent (e.g., 3-chloroperoxybenzoic acid (mCPBA) and/or hydrogen peroxides) to afford other compounds of the disclosure. Thus, all shown and claimed nitrogen-containing compounds are considered, when allowed by valency and structure, to include both the compound as shown and its N-oxide derivative (which can be designated as N□O or N+-O—). Furthermore, in other instances, the nitrogens in the compounds of the disclosure can be converted to N-hydroxy or N-alkoxy compounds. For example, N-hydroxy compounds can be prepared by oxidation of the parent amine by an oxidizing agent such as m CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C1-C 6 alkyl, C1-C6 alkenyl, C1-C6 alkynyl, 3-14-membered carbocycle or 3-14-membered heterocycle) derivatives.

### Other Lipid Composition Components

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates.

The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In some embodiments, the pharmaceutical composition disclosed herein can contain more than one polypeptides. For example, a pharmaceutical composition disclosed herein can contain two or more polynucleotides (e.g., RNA, e.g., mRNA).

In one embodiment, the lipid nanoparticles described herein can comprise polynucleotides (e.g., mRNA) in a lipid:polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1,from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

### Nanoparticle Compositions

In some embodiments, the pharmaceutical compositions disclosed herein are formulated as lipid nanoparticles (LNP). Accordingly, the present disclosure also provides nanoparticle compositions comprising (i) a lipid composition comprising a delivery agent such as compound as described herein, and (ii) at least one mRNA encoding a polypeptide. In such nanoparticle composition, the lipid composition disclosed herein can encapsulate the at least one mRNA encoding a polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a sterol and a structural lipid. In some embodiments, the LNP has a molar ratio of about 20-60% ionizable lipid: about 5-25% structural lipid: about 25-55% sterol; and about 0.5-15% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

In addition to these, an ionizable lipid may also be a lipid including a cyclic amine group.

In one embodiment, the ionizable lipid may be selected from, but not limited to, a ionizable lipid described in International Publication Nos. WO2013086354 and WO2013116126; the contents of each of which are herein incorporated by reference in their entirety.

In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a polypeptide is formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition.

In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

### Other Delivery Agents

### Liposomes, Lipoplexes, and Lipid Nanoparticles

In some embodiments, the compositions or formulations of the present disclosure comprise a delivery agent, e.g., a liposome, a lioplexes, a lipid nanoparticle, or any combination thereof. The polynucleotides described herein (e.g., a polynucleotide comprising a nucleotide sequence encoding a polypeptide) can be formulated using one or more liposomes, lipoplexes, or lipid nanoparticles. Liposomes, lipoplexes, or lipid nanoparticles can be used to improve the efficacy of the mRNAs directed protein production as these formulations can increase cell transfection by the mRNA; and/or increase the translation of encoded protein. The liposomes, lipoplexes, or lipid nanoparticles can also be used to increase the stability of the mRNAs.

Liposomes are artificially-prepared vesicles that can primarily be composed of a lipid bilayer and can be used as a delivery vehicle for the administration of pharmaceutical formulations. Liposomes can be of different sizes. A multilamellar vesicle (MLV) can be hundreds of nanometers in diameter, and can contain a series of concentric bilayers separated by narrow aqueous compartments. A small unicellular vesicle (SUV) can be smaller than 50 nm in diameter, and a large unilamellar vesicle (LUV) can be between 50 and 500 nm in diameter. Liposome design can include, but is not limited to, opsonins or ligands to improve the attachment of liposomes to unhealthy tissue or to activate events such as, but not limited to, endocytosis. Liposomes can contain a low or a high pH value in order to improve the delivery of the pharmaceutical formulations.

The formation of liposomes can depend on the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimal size, polydispersity and the shelf-life of the vesicles for the intended application, and the batch-to-batch reproducibility and scale up production of safe and efficient liposomal products, etc.

As a non-limiting example, liposomes such as synthetic membrane vesicles can be prepared by the methods, apparatus and devices described in U.S. Pub. Nos. US20130177638, US20130177637, US20130177636, US20130177635, US20130177634, US20130177633, US20130183375, US20130183373, and US20130183372. In some embodiments, the mRNAs described herein can be encapsulated by the liposome and/or it can be contained in an aqueous core that can then be encapsulated by the liposome as described in, e.g., Intl. Pub. Nos. WO2012031046, WO2012031043, WO2012030901, WO2012006378, and WO2013086526; and U.S. Pub. Nos. US20130189351, US20130195969 and US20130202684. Each of the references in herein incorporated by reference in its entirety.

In some embodiments, the mRNAs described herein can be formulated in a cationic oil-in-water emulsion where the emulsion particle comprises an oil core and a cationic lipid that can interact with the mRNA anchoring the molecule to the emulsion particle. In some embodiments, the mRNAs described herein can be formulated in a water-in-oil emulsion comprising a continuous hydrophobic phase in which the hydrophilic phase is dispersed. Exemplary emulsions can be made by the methods described in Intl. Pub. Nos. WO2012006380 and WO201087791, each of which is herein incorporated by reference in its entirety.

In some embodiments, the mRNAs described herein can be formulated in a lipid-polycation complex. The formation of the lipid-polycation complex can be accomplished by methods as described in, e.g., U.S. Pub. No. US20120178702. As a non-limiting example, the polycation can include a cationic peptide or a polypeptide such as, but not limited to, polylysine, polyornithine and/or polyarginine and the cationic peptides described in Intl. Pub. No. WO2012013326 or U.S. Pub. No. US20130142818. Each of the references is herein incorporated by reference in its entirety.

In some embodiments, the mRNAs described herein can be formulated in a lipid nanoparticle (LNP) such as those described in Intl. Pub. Nos. WO2013123523, WO2012170930, WO2011127255 and WO2008103276; and U.S. Pub. No. US20130171646, each of which is herein incorporated by reference in its entirety.

Lipid nanoparticle formulations typically comprise one or more lipids. In some embodiments, the lipid is an ionizable lipid (e.g., an ionizable amino lipid), sometimes referred to in the art as an “ionizable cationic lipid”. In some embodiments, lipid nanoparticle formulations further comprise other components, including a phospholipid, a structural lipid, and a molecule capable of reducing particle aggregation, for example a PEG or PEG-modified lipid.

Exemplary ionizable lipids include, but not limited to, any one of Compounds 1-342 disclosed herein, DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. Other exemplary ionizable lipids include, (13Z,16Z)—N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcyclopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecylcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z, 16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)—N,N-dimethylnonacosa-11,20,2-trien-10-amine, and any combination thereof.

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, the phospholipids are DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof. In some embodiments, the phospholipids are MPPC, MSPC, PMPC, PSPC, SMPC, SPPC, DHAPE, DOPG, and any combination thereof. In some embodiments, the amount of phospholipids (e.g., DSPC) in the lipid composition ranges from about 1 mol % to about 20 mol %.

The structural lipids include sterols and lipids containing sterol moieties. In some embodiments, the structural lipids include cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the amount of the structural lipids (e.g., cholesterol) in the lipid composition ranges from about 20 mol % to about 60 mol %.

The PEG-modified lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG DMPE, PEG-DPPC, or a PEG-DSPE lipid. In some embodiments, the PEG-lipid are 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA). In some embodiments, the PEG moiety has a size of about 1000, 2000, 5000, 10,000, 15,000 or 20,000 daltons. In some embodiments, the amount of PEG-lipid in the lipid composition ranges from about 0 mol % to about 5 mol %.

In some embodiments, the LNP formulations described herein can additionally comprise a permeability enhancer molecule. Non-limiting permeability enhancer molecules are described in U.S. Pub. No. US20050222064, herein incorporated by reference in its entirety.

The LNP formulations can further contain a phosphate conjugate. The phosphate conjugate can increase in vivo circulation times and/or increase the targeted delivery of the nanoparticle. Phosphate conjugates can be made by the methods described in, e.g., Intl. Pub. No. WO2013033438 or U.S. Pub. No. US20130196948. The LNP formulation can also contain a polymer conjugate (e.g., a water soluble conjugate) as described in, e.g., U.S. Pub. Nos. US20130059360, US20130196948, and US20130072709. Each of the references is herein incorporated by reference in its entirety.

The LNP formulations can comprise a conjugate to enhance the delivery of nanoparticles of the present invention in a subject. Further, the conjugate can inhibit phagocytic clearance of the nanoparticles in a subject. In some embodiments, the conjugate can be a “self” peptide designed from the human membrane protein CD47 (e.g., the “self” particles described by Rodriguez et al, Science 2013 339, 971-975, herein incorporated by reference in its entirety). As shown by Rodriguez et al. the self peptides delayed macrophage-mediated clearance of nanoparticles which enhanced delivery of the nanoparticles.

The LNP formulations can comprise a carbohydrate carrier. As a non-limiting example, the carbohydrate carrier can include, but is not limited to, an anhydride-modified phytoglycogen or glycogen-type material, phytoglycogen octenyl succinate, phytoglycogen beta-dextrin, anhydride-modified phytoglycogen beta-dextrin (e.g., Intl. Pub. No. WO2012109121, herein incorporated by reference in its entirety).

The LNP formulations can be coated with a surfactant or polymer to improve the delivery of the particle. In some embodiments, the LNP can be coated with a hydrophilic coating such as, but not limited to, PEG coatings and/or coatings that have a neutral surface charge as described in U.S. Pub. No. US20130183244, herein incorporated by reference in its entirety.

The LNP formulations can be engineered to alter the surface properties of particles so that the lipid nanoparticles can penetrate the mucosal barrier as described in U.S. Pat. No. 8,241,670 or Intl. Pub. No. WO2013110028, each of which is herein incorporated by reference in its entirety.

The LNP engineered to penetrate mucus can comprise a polymeric material (i.e., a polymeric core) and/or a polymer-vitamin conjugate and/or a tri-block co-polymer. The polymeric material can include, but is not limited to, polyamines, polyethers, polyamides, polyesters, polycarbamates, polyureas, polycarbonates, poly(styrenes), polyimides, polysulfones, polyurethanes, polyacetylenes, polyethylenes, polyethyeneimines, polyisocyanates, polyacrylates, polymethacrylates, polyacrylonitriles, and polyarylates.

LNP engineered to penetrate mucus can also include surface altering agents such as, but not limited to, mRNAs, anionic proteins (e.g., bovine serum albumin), surfactants (e.g., cationic surfactants such as for example dimethyldioctadecyl-ammonium bromide), sugars or sugar derivatives (e.g., cyclodextrin), nucleic acids, polymers (e.g., heparin, polyethylene glycol and poloxamer), mucolytic agents (e.g., N-acetylcysteine, mugwort, bromelain, papain, clerodendrum, acetylcysteine, bromhexine, carbocisteine, eprazinone, mesna, ambroxol, sobrerol, domiodol, letosteine, stepronin, tiopronin, gelsolin, thymosin β 4 dornase alfa, neltenexine, erdosteine) and various DNases including rhDNase.

In some embodiments, the mucus penetrating LNP can be a hypotonic formulation comprising a mucosal penetration enhancing coating. The formulation can be hypotonic for the epithelium to which it is being delivered. Non-limiting examples of hypotonic formulations can be found in, e.g., Intl. Pub. No. WO2013110028, herein incorporated by reference in its entirety.

In some embodiments, the mRNA described herein is formulated as a lipoplex, such as, without limitation, the ATUPLEX™ system, the DACC system, the DBTC system and other siRNA-lipoplex technology from Silence Therapeutics (London, United Kingdom), STEMFECT™ from STEMGENT® (Cambridge, Mass.), and polyethylenimine (PEI) or protamine-based targeted and non-targeted delivery of nucleic acids (Aleku et al. Cancer Res. 2008 68:9788-9798; Strumberg et al. Int J Clin Pharmacol Ther 2012 50:76-78; Santel et al., Gene Ther 2006 13:1222-1234; Santel et al., Gene Ther 2006 13:1360-1370; Gutbier et al., Pulm Pharmacol. Ther. 2010 23:334-344; Kaufmann et al. Microvasc Res 2010 80:286-293Weide et al. J Immunother. 2009 32:498-507; Weide et al. J Immunother. 2008 31:180-188; Pascolo Expert Opin. Biol. Ther. 4:1285-1294; Fotin-Mleczek et al., 2011 J. Immunother. 34:1-15; Song et al., Nature Biotechnol. 2005, 23:709-717; Peer et al., Proc Natl Acad Sci USA. 2007 6; 104:4095-4100; deFougerolles Hum Gene Ther. 2008 19:125-132; all of which are incorporated herein by reference in its entirety).

In some embodiments, the mRNAs described herein are formulated as a solid lipid nanoparticle (SLN), which can be spherical with an average diameter between 10 to 1000 nm. SLN possess a solid lipid core matrix that can solubilize lipophilic molecules and can be stabilized with surfactants and/or emulsifiers. Exemplary SLN can be those as described in Intl. Pub. No. WO2013105101, herein incorporated by reference in its entirety.

In some embodiments, the mRNAs described herein can be formulated for controlled release and/or targeted delivery. As used herein, “controlled release” refers to a pharmaceutical composition or compound release profile that conforms to a particular pattern of release to effect a therapeutic outcome. In one embodiment, the mRNAs can be encapsulated into a delivery agent described herein and/or known in the art for controlled release and/or targeted delivery. As used herein, the term “encapsulate” means to enclose, surround or encase. As it relates to the formulation of the compounds of the invention, encapsulation can be substantial, complete or partial. The term “substantially encapsulated” means that at least greater than 50, 60, 70, 80, 85, 90, 95, 96, 97, 98, 99, or greater than 99% of the pharmaceutical composition or compound of the invention can be enclosed, surrounded or encased within the delivery agent. “Partially encapsulation” means that less than 10, 10, 20, 30, 40 50 or less of the pharmaceutical composition or compound of the invention can be enclosed, surrounded or encased within the delivery agent.

Advantageously, encapsulation can be determined by measuring the escape or the activity of the pharmaceutical composition or compound of the invention using fluorescence and/or electron micrograph. For example, at least 1, 5, 10, 20, 30, 40, 50, 60, 70, 80, 85, 90, 95, 96, 97, 98, 99, 99.9, or greater than 99% of the pharmaceutical composition or compound of the invention are encapsulated in the delivery agent.

In some embodiments, the mRNAs described herein can be encapsulated in a therapeutic nanoparticle, referred to herein as “therapeutic nanoparticle mRNAs.” Therapeutic nanoparticles can be formulated by methods described in, e.g., Intl. Pub. Nos. WO2010005740, WO2010030763, WO2010005721, WO2010005723, and WO2012054923; and U.S. Pub. Nos. US20110262491, US20100104645, US20100087337, US20100068285, US20110274759, US20100068286, US20120288541, US20120140790, US20130123351 and US20130230567; and U.S. Pat. Nos. 8,206,747, 8,293,276, 8,318,208 and 8,318,211, each of which is herein incorporated by reference in its entirety.

In some embodiments, the therapeutic nanoparticle mRNA can be formulated for sustained release. As used herein, “sustained release” refers to a pharmaceutical composition or compound that conforms to a release rate over a specific period of time. The period of time can include, but is not limited to, hours, days, weeks, months and years. As a non-limiting example, the sustained release nanoparticle of the mRNAs described herein can be formulated as disclosed in Intl. Pub. No. WO2010075072 and U.S. Pub. Nos. US20100216804, US20110217377, US20120201859 and US20130150295, each of which is herein incorporated by reference in their entirety.

In some embodiments, the therapeutic nanoparticle mRNA can be formulated to be target specific, such as those described in Intl. Pub. Nos. WO2008121949, WO2010005726, WO2010005725, WO2011084521 and WO2011084518; and U.S. Pub. Nos. US20100069426, US20120004293 and US20100104655, each of which is herein incorporated by reference in its entirety.

The LNPs can be prepared using microfluidic mixers or micromixers. Exemplary microfluidic mixers can include, but are not limited to, a slit interdigital micromixer including, but not limited to those manufactured by Microinnova (Allerheiligen bei Wildon, Austria) and/or a staggered herringbone micromixer (SHM) (see Zhigaltsev et. al.., “Bottom-up design and synthesis of limit size lipid nanoparticle systems with aqueous and triglyceride cores using millisecond microfluidic mixing,” Langmuir 28:3633-40 (2012); Belliveau et al., “Microfluidic synthesis of highly potent limit-size lipid nanoparticles for in vivo delivery of siRNA,” Molecular Therapy-Nucleic Acids. 1:e37 (2012); Chen et al., “Rapid discovery of potent siRNA-containing lipid nanoparticles enabled by controlled microfluidic formulation,” J. Am. Chem. Soc. 134(16):6948-51 (2012); each of which is herein incorporated by reference in its entirety). Exemplary micromixers include Slit Interdigital Microstructured Mixer (SIMM-V2) or a Standard Slit Interdigital Micro Mixer (SSIMM) or Caterpillar (CPMM) or Impinging-jet (IJMM,) from the Institut fur Mikrotechnik Mainz GmbH, Mainz Germany. In some embodiments, methods of making LNP using SHM further comprise mixing at least two input streams wherein mixing occurs by microstructure-induced chaotic advection (MICA). According to this method, fluid streams flow through channels present in a herringbone pattern causing rotational flow and folding the fluids around each other. This method can also comprise a surface for fluid mixing wherein the surface changes orientations during fluid cycling. Methods of generating LNPs using SHM include those disclosed in U.S. Pub. Nos. US20040262223 and US20120276209, each of which is incorporated herein by reference in their entirety.

In some embodiments, the mRNAs described herein can be formulated in lipid nanoparticles using microfluidic technology (see Whitesides, George M., “The Origins and the Future of Microfluidics,” Nature 442: 368-373 (2006); and Abraham et al., “Chaotic Mixer for Microchannels,” Science 295: 647-651 (2002); each of which is herein incorporated by reference in its entirety). In some embodiments, the mRNAs can be formulated in lipid nanoparticles using a micromixer chip such as, but not limited to, those from Harvard Apparatus (Holliston, Mass.) or Dolomite Microfluidics (Royston, UK). A micromixer chip can be used for rapid mixing of two or more fluid streams with a split and recombine mechanism.

In some embodiments, the mRNAs described herein can be formulated in lipid nanoparticles having a diameter from about 1 nm to about 100 nm such as, but not limited to, about 1 nm to about 20 nm, from about 1 nm to about 30 nm, from about 1 nm to about 40 nm, from about 1 nm to about 50 nm, from about 1 nm to about 60 nm, from about 1 nm to about 70 nm, from about 1 nm to about 80 nm, from about 1 nm to about 90 nm, from about 5 nm to about from 100 nm, from about 5 nm to about 10 nm, about 5 nm to about 20 nm, from about 5 nm to about 30 nm, from about 5 nm to about 40 nm, from about 5 nm to about 50 nm, from about 5 nm to about 60 nm, from about 5 nm to about 70 nm, from about 5 nm to about 80 nm, from about 5 nm to about 90 nm, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In some embodiments, the lipid nanoparticles can have a diameter from about 10 to 500 nm. In one embodiment, the lipid nanoparticle can have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the mRNAs can be delivered using smaller LNPs. Such particles can comprise a diameter from below 0.1 μm up to 100 nm such as, but not limited to, less than 0.1 μm, less than 1.0 μm, less than 5 μm, less than 10 μm, less than 15 um, less than 20 um, less than 25 um, less than 30 um, less than 35 um, less than 40 um, less than 50 um, less than 55 um, less than 60 um, less than 65 um, less than 70 um, less than 75 um, less than 80 um, less than 85 um, less than 90 um, less than 95 um, less than 100 um, less than 125 um, less than 150 um, less than 175 um, less than 200 um, less than 225 um, less than 250 um, less than 275 um, less than 300 um, less than 325 um, less than 350 um, less than 375 um, less than 400 um, less than 425 um, less than 450 um, less than 475 um, less than 500 um, less than 525 um, less than 550 um, less than 575 um, less than 600 um, less than 625 um, less than 650 um, less than 675 um, less than 700 um, less than 725 um, less than 750 um, less than 775 um, less than 800 um, less than 825 um, less than 850 um, less than 875 um, less than 900 um, less than 925 um, less than 950 um, or less than 975 um.

The nanoparticles and microparticles described herein can be geometrically engineered to modulate macrophage and/or the immune response. The geometrically engineered particles can have varied shapes, sizes and/or surface charges to incorporate the mRNAs described herein for targeted delivery such as, but not limited to, pulmonary delivery (see, e.g., Intl. Pub. No. WO20130821 11, herein incorporated by reference in its entirety). Other physical features the geometrically engineering particles can include, but are not limited to, fenestrations, angled arms, asymmetry and surface roughness, charge that can alter the interactions with cells and tissues.

In some embodiment, the nanoparticles described herein are stealth nanoparticles or target-specific stealth nanoparticles such as, but not limited to, those described in U.S. Pub. No. US20130172406, herein incorporated by reference in its entirety. The stealth or target-specific stealth nanoparticles can comprise a polymeric matrix, which can comprise two or more polymers such as, but not limited to, polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, polyamines, polyesters, polyanhydrides, polyethers, polyurethanes, polymethacrylates, polyacrylates, polycyanoacrylates, or combinations thereof.

### Lipidoids

In some embodiments, the compositions or formulations of the present disclosure comprise a delivery agent, e.g., a lipidoid. The mRNAs described herein (e.g., an mRNA comprising a nucleotide sequence encoding a polypeptide) can be formulated with lipidoids. Complexes, micelles, liposomes or particles can be prepared containing these lipidoids and therefore to achieve an effective delivery of the mRNA, as judged by the production of an encoded protein, following the injection of a lipidoid formulation via localized and/or systemic routes of administration. Lipidoid complexes of mRNAs can be administered by various means including, but not limited to, intravenous, intramuscular, or subcutaneous routes.

The synthesis of lipidoids is described in literature (see Mahon et al., Bioconjug. Chem. 2010 21:1448-1454; Schroeder et al., J Intern Med. 2010 267:9-21; Akinc et al., Nat Biotechnol. 2008 26:561-569; Love et al., Proc Natl Acad Sci USA. 2010 107:1864-1869; Siegwart et al., Proc Natl Acad Sci USA. 2011 108:12996-3001; all of which are incorporated herein in their entireties).

Formulations with the different lipidoids, including, but not limited to penta[3-(1-laurylaminopropionyl)]-triethylenetetramine hydrochloride (TETA-5LAP; also known as 98N12-5, see Murugaiah et al., Analytical Biochemistry, 401:61 (2010)), C12-200 (including derivatives and variants), and MD1, can be tested for in vivo activity. The lipidoid “98N12-5” is disclosed by Akinc et al., Mol Ther. 2009 17:872-879. The lipidoid “C12-200” is disclosed by Love et al., Proc Natl Acad Sci USA. 2010 107:1864-1869 and Liu and Huang, Molecular Therapy. 2010 669-670. Each of the references is herein incorporated by reference in its entirety.

In one embodiment, the mRNAs described herein can be formulated in an aminoalcohol lipidoid. Aminoalcohol lipidoids can be prepared by the methods described in U.S. Pat. No. 8,450,298 (herein incorporated by reference in its entirety).

The lipidoid formulations can include particles comprising either 3 or 4 or more components in addition to mRNAs. Lipidoids and mRNA formulations comprising lipidoids are described in Intl. Pub. No. WO 2015051214 (herein incorporated by reference in its entirety.

### Polypeptides of Interest

In some aspects, the present disclosure provides mRNAs (e.g., endonuclease-resistant mRNAs) comprising an open reading frame (ORF) encoding polypeptides of interest (e.g., therapeutic polypeptides). In some embodiments, the polypeptide of interest is a therapeutic polypeptide. In some embodiments, the disclosure provides method of generating an endonuclease-resistant mRNA comprising an ORF that encodes a polypeptide of interest (e.g., a therapeutic polypeptide), typically a protein or peptide having therapeutic properties for use in a subject. The polypeptides of interest can be essentially any protein or polypeptide that can be encoded by an mRNA.

In some embodiments, an mRNA of the disclosure encodes a polypeptide of interest that is a full-length protein. In some embodiments, an mRNA of the disclosure encodes a polypeptide of interest that is a functional fragment of a full-length protein (e.g., a fragment of the full-length protein that includes one or more functional domains such that the functional activity of the full-length protein is retained). In some embodiments, an mRNA of the disclosure encodes a polypeptide of interest that is not naturally occurring. In some embodiments, an mRNA of the disclosure encodes a polypeptide of interest that is a modified protein comprised of one or more heterologous domains (e.g., a protein that is a fusion protein comprised of one or more domains that do not naturally occur in the protein such that the function of the protein is altered).

Exemplary types of proteins (e.g., infectious disease antigens, tumor cell antigens, soluble effector molecules, antibodies, enzymes, recruitment factors, transcription factors, membrane bound receptors or ligands) that are encoded by an mRNA of the disclosure are described in detail in the following subsections.

### Naturally Occurring Targets

In some embodiments, an mRNA of the disclosure encodes a polypeptide of interest that is a naturally occurring target. In some embodiments, an mRNA encodes a polypeptide of interest that when expressed, modulates a naturally occurring target (e.g., up- or down-regulates the activity of a naturally occurring target). In some embodiments, a naturally occurring target is a soluble protein that is secreted by a cell. In some embodiments, a naturally occurring target is a protein that is retained within a cell (e.g., an intracellular protein). In some embodiments, a naturally occurring target is a membrane-bound or transmembrane protein. Non-limiting examples of naturally occurring targets include soluble proteins (e.g., chemokines, cytokines, growth factors, antibodies, enzymes), intracellular proteins (e.g., intracellular signaling proteins, transcription factors, enzymes, structural proteins) and membrane-bound or transmembrane proteins (e.g., receptors, adhesion molecules, enzymes).

In some embodiments, an mRNA encodes a polypeptide of interest that when expressed is a full-length naturally occurring target (i.e., a full-length protein). In some embodiments, an mRNA encodes a polypeptide of interest that when expressed is a fragment or portion of a naturally occurring target (i.e., a fragment or portion of a full-length protein). For example, in one embodiment, the protein or fragment thereof can be an immunogenic polypeptide that can be used as a vaccine.

In some embodiments, an mRNA encodes a polypeptide that when expressed, modulates a naturally occurring target (e.g., by encoding the target itself or by functioning to modulate the activity of the target). In some embodiments, a polypeptide of interest acts in an autocrine fashion, i.e., the polypeptide exerts an effect directly on the cell into which the mRNA is delivered. In some embodiments, an encoded polypeptide of interest acts in a paracrine fashion, i.e., the encoded polypeptide exerts an indirect effect on a cell that is not the cell into which the mRNA is delivered (e.g., delivery of the mRNA into one type of cell results in secretion of a molecule that exerts an effects on another type of cell, such as a bystander cell). In some embodiments, an encoded polypeptide of interest acts in both an autocrine fashion and a paracrine fashion.

### Naturally Occurring Soluble Targets

In some embodiments, an mRNA encodes a polypeptide of interest that modulates the activity of a naturally occurring soluble target, for example by encoding the soluble target itself or by modulating the expression (e.g., transcription or translation) of the soluble target. Non-limiting examples of naturally occurring soluble targets include cytokines, chemokines, growth factors, enzymes, and antibodies.

In some embodiments, an mRNA encoding a polypeptide of interest stimulates (e.g., upregulates, enhances) the activation or activity of a cell type, for example in situations where stimulation of an immune response is desirable, such as in cancer therapy or treatment of an infectious disease (e.g., a viral, bacterial, fungal, protozoal or parasitic infection). In another embodiment, an mRNA encoding a polypeptide of interest inhibits (e.g., downregulates, reduces) the activation or activity of a cell, for example in situations where inhibition of an immune response is desirable, such as in autoimmune diseases, allergies and transplantation.

In some embodiments, an mRNA of the disclosure encodes a soluble target that is a cytokine or chemokine with desirable uses for stimulating or inhibiting immune responses, e.g., that is useful in treating cancer as described further below.

In some embodiments, an mRNA of the disclosure encodes a soluble target that is a cytokine that stimulates the activation or activity of a cell such as an immune cell.

In some embodiments, an mRNA of the disclosure encodes a chemokine or a chemokine receptor which is useful for stimulating the activation or activity of an immune cell. Chemokines have been demonstrated to control the trafficking of inflammatory cells (including granulocytes and monocytes/monocytes), as well as regulating the movement of a wide variety of immune cells (including lymphocytes, natural killer cells and dendritic cells). Thus, chemokines are involved both in regulating inflammatory responses and immune responses. Moreover, chemokines have been shown to have effects on the proliferative and invasive properties of cancer cells (for a review of chemokines, see e.g., Mukaida, N. et al. (2014) Mediators of Inflammation, Article ID 170381, pg. 1-15).

In some embodiments, an mRNA of the disclosure encodes a recruitment factor which is useful to stimulate the homing, activation or activity of a cell. In one embodiment, the cell is an immune cell and the “recruitment factor” refers to a protein that promotes recruitment of an immune cell to a desired location (e.g., to a tumor site or an inflammatory site). For example, certain chemokines, chemokine receptors and cytokines have been shown to be involved in the recruitment of lymphocytes (see e.g., Oelkrug, C. and Ramage, J. M. (2014) Clin. Exp. Immunol. 178:1-8).

In some embodiments, an mRNA of the disclosure encodes an inhibitory cytokine or an antagonist of a stimulatory cytokine which is useful for inhibiting immune responses.

In some embodiments, an mRNA of the disclosure encodes a soluble target that is an antibody. As used herein, the term “antibody” refers to a whole antibody comprising two light chain polypeptides and two heavy chain polypeptides, or an antigen-binding fragment thereof. In some embodiments, a soluble target is a monoclonal antibody (e.g., full length monoclonal antibody) that displays a single binding specificity and affinity for a particular epitope. In some embodiments, a soluble target is an antigen binding fragment of a monoclonal antibody that retains the ability to bind a target antigen. Such fragments include, e.g., a single chain antibody, a single chain Fv fragment (scFv), an Fd fragment, an Fab fragment, an Fab′ fragment, or an F(ab′)₂ fragment.

In some embodiments, an mRNA of the disclosure encodes an antibody that recognizes a tumor antigen, against which a protective or a therapeutic immune response is desired, e.g., antigens expressed by a tumor cell. In some embodiments, a suitable antigen includes tumor associated antigens for the prevention or treatment of cancers.

In some embodiments, an mRNA of the disclosure encodes an antibody that recognizes an infectious disease antigen, against which protective or therapeutic immune responses are desired, e.g., an antigen present on a pathogen or infectious agent. In some embodiments, a suitable antigen includes an infectious disease associated antigen for the prevention or treatment of an infectious disease. Methods for identification of antigens on infectious disease agents that comprise protective epitopes (e.g., epitopes that when recognized by an antibody enable neutralization or blocking of infection caused by an infectious disease agent) are described in the art as detailed by Sharon, J. et al. (2013) Immunology 142:1-23. In some embodiments, an infectious disease antigen is present on a virus or on a bacterial cell.

In some embodiments, an mRNA of the disclosure encodes a soluble target that is a growth factor with desirable uses for modulating tissue healing and repair. A growth factor is a protein that stimulates the survival, growth, proliferation, migration or differentiation of cells, often for the purposes of promoting growth of lost tissue or enhancing the body's innate healing and repair mechanisms. In some embodiments, a growth factor is used to manipulate cells that include, but are not limited to, stromal cells (e.g., fibroblasts), immune cells, vascular cells (e.g., epithelial cells, platelets, pericytes), neural cells (e.g., astrocytes, neural stem cells, microglial cells), or bone cells (e.g., osteocyte, osteoblast, osteoclast, osteogenic cells).

In some embodiments, an mRNA of the disclosure encodes a soluble target that is an enzyme with desirable uses for modulating metabolism or growth in a subject. In some embodiments, an enzyme is administered to replace an endogenous enzyme that is absent or dysfunctional as described in Brady, R. et al, (2004) Lancet Neurol. 3:752. In some embodiments, an enzyme is used to treat a metabolic storage disease. A metabolic storage disease results from the systemic accumulation of metabolites due to the absence or dysfunction of an endogenous enzyme. Such metabolites include lipids, glycoproteins, and mucopolysaccharides. In some embodiments, an enzyme is used to reduce or eliminate the accumulation of monosaccharides, polysaccharides, glycoproteins, glycopeptides, glycolipids or lipids due to a metabolic storage disease.

### Naturally Occurring Intracellular Targets

In some embodiments, an mRNA of the disclosure encodes a polypeptide of interest that modulates the activity of a naturally occurring intracellular target, for example by encoding the intracellular target itself or by modulating the expression (e.g., transcription or translation) of the intracellular target in a cell. Non-limiting examples of naturally-occurring intracellular targets include transcription factors and cell signaling cascade molecules, including enzymes, that modulate cell growth, differentiation and communication. Additional examples include intracellular targets that regulate cell metabolism.

Suitable transcription factors and intracellular signaling cascade molecules for particular uses in stimulating or inhibiting cellular activity or responses are described in the art. In some embodiments, an mRNA of the disclosure encodes a transcription factor useful for stimulating the activation or activity of an immune cell. As used herein, a “transcription factor” refers to a DNA-binding protein that regulates the transcription of a gene. In some embodiments, an mRNA of the disclosure encodes a transcription factor that increases or polarizes an immune response.

In some embodiments, an mRNA of the disclosure encodes an intracellular adaptor protein (e.g., in a signal transduction pathway) useful for stimulating the activation or activity of a cell.

In some embodiments, an mRNA of the disclosure encodes an intracellular signaling protein useful for stimulating the activation or activity of a cell. In some embodiments, an mRNA of the disclosure encodes a tolerogenic transcription factor useful for inhibiting the activation or activity of an immune cell.

In some embodiments, an mRNA of the disclosure encodes an intracellular target that is a protein that is used to treat a metabolic disease or disorder.

In some embodiments, an mRNA of the disclosure encodes a polypeptide of interest that is a fully-functional mitochondrial protein (e.g., wild-type). In some embodiments, an mRNA of the disclosure encodes a mitochondrial protein encoded by mitochondrial DNA (e.g., a mitochondrial-encoded mitochondrial protein). In some embodiments, an mRNA of the disclosure encodes a mitochondrial protein encoded by nuclear DNA (e.g., a nuclear-encoded mitochondrial protein). In some embodiments, an mRNA of the disclosure is used to treat a mitochondrial disease resulting from a mutation in a mitochondrial protein. In some embodiments, translation of an mRNA encoding a mitochondrial protein provides sufficient quantity and/or activity of the protein to ameliorate a mitochondrial disease. In some embodiments, an mRNA encodes a polypeptide of interest that is a mitochondrial protein described in the MitoCarta2.0 mitochondrial protein inventory.

### Naturally Occurring Membrane Bound/Transmembrane Targets

In some embodiments, an mRNA of the disclosure encodes a polypeptide of interest that modulates the activity of a naturally-occurring membrane-bound/transmembrane target, for example by encoding the membrane-bound/transmembrane target itself or by modulating the expression (e.g., transcription or translation) of the membrane-bound/transmembrane target. Non-limiting examples of naturally-occurring membrane-bound/transmembrane targets include Cell surface receptors, growth factor receptors, costimulatory molecules, immune checkpoint molecules, homing receptors and HLA molecules.

In one embodiment, the membrane-bound/transmembrane targets are useful in stimulating or inhibiting immune responses are described herein. In some embodiments, an mRNA of the disclosure encodes a costimulatory factor that upregulates an immune response or is an antagonist of a costimulatory factor that downregulates an immune response. In some embodiments, an mRNA of the disclosure encodes an immune checkpoint protein that down-regulates immune cells (e.g., T cells). In some embodiments, an mRNA of the disclosure encodes a membrane-bound/transmembrane protein target that serves as a homing signal.

In some embodiments, an mRNA of the disclosure encodes a membrane-bound/transmembrane protein target that is an immune receptor, e.g., on a lymphocyte or monocyte.

### Modified Targets

In some embodiments, an mRNA of the disclosure encodes a polypeptide of interest that is a modified polypeptide. In some embodiments, an mRNA of the disclosure encodes a polypeptide of interest that modulates a modified target (e.g., up- or down-regulates the activity of a non-naturally-occurring target). Typically, an mRNA of the disclosure encodes a modified target. Alternatively, if a cell expresses a modified target, an mRNA-encoded polypeptide functions to modulate the activity of the modified target in the cell. In some embodiments, a non-naturally occurring target is a full-length target, such as a full-length modified protein. In some embodiments, a non-naturally occurring target is a fragment or portion of a non-naturally-occurring target, such as a fragment or portion of a modified protein. In some embodiments, an mRNA-encoded polypeptide when expressed acts in an autocrine fashion to modulate a modified target, i.e., exerts an effect directly on the cell into which the mRNA is delivered. Additionally or alternatively, an mRNA-encoded polypeptide when expressed acts in a paracrine fashion to modulates a modified target, i.e., exerts an effect indirectly on a cell other than the cell into which the mRNA is delivered (e.g., delivery of the mRNA into one type of cell results in secretion of a molecule that exerts effects on another type of cell, such as bystander cells). Non-limiting examples of modified proteins include modified soluble proteins (e.g., secreted proteins), modified intracellular proteins (e.g., intracellular signaling proteins, transcription factors) and modified membrane-bound or transmembrane proteins (e.g., receptors).

### Modified Soluble Targets

In some embodiments, an mRNA of the disclosure encodes a polypeptide of interest that modulates a modified soluble target (e.g., up- or down-regulates the activity of a non-naturally-occurring soluble target). In some embodiments, an mRNA of the disclosure encodes a polypeptide of interest that is a modified soluble target. In some embodiments, a modified soluble target is a soluble protein that has been modified to alter (e.g., increase or decrease) the half-life (e.g., serum half-life) of the protein. Modified soluble proteins with altered half-life include modified cytokines and chemokines. In some embodiments, a modified soluble target is a soluble protein that has been modified to incorporate a tether such that the soluble protein becomes tethered to a cell surface. Modified soluble proteins incorporating a tether include tethered cytokines and chemokines.

### Modified Intracellular Targets

In some embodiments, an mRNA of the disclosure encodes a polypeptide of interest that modulates a modified intracellular target (e.g., up- or down-regulates the activity of a non-naturally-occurring intracellular target). In some embodiments, an mRNA of the disclosure encodes polypeptide of interest that is a modified intracellular target. In some embodiments, a modified intracellular target is a constitutively active mutant of an intracellular protein, such as a constitutively active transcription factor or intracellular signaling molecule. In some embodiments, a modified intracellular target is a dominant negative mutant of an intracellular protein, such as a dominant negative mutant of a transcription factor or intracellular signaling molecule. In some embodiments, a modified intracellular target is an altered (e.g., mutated) enzyme, such as a mutant enzyme with increased or decreased activity within an intracellular signaling cascade.

Modified Membrane bound/Transmembrane Targets

In some embodiments, an mRNA of the disclosure encodes a polypeptide of interest that modulates a modified membrane-bound/transmembrane target (e.g., up- or down-regulates the activity of a non-naturally-occurring membrane-bound/transmembrane target). In some embodiments, an mRNA of the disclosure encodes a polypeptide of interest that is a modified membrane-bound/transmembrane target. In some embodiments, a modified membrane-bound/transmembrane target is a constitutively active mutant of a membrane-bound/transmembrane protein, such as a constitutively active cell surface receptor (i.e., activates intracellular signaling through the receptor without the need for ligand binding). In some embodiments, a modified membrane-bound/transmembrane target is a dominant negative mutant of a membrane-bound/transmembrane protein, such as a dominant negative mutant of a cell surface receptor. In some embodiments, a modified membrane-bound/transmembrane target is a molecule that inverts signaling of a cellular synapse (e.g., agonizes or antagonizes signaling of a receptor). In some embodiments, a modified membrane-bound/transmembrane target is a chimeric membrane-bound/transmembrane protein, such as a chimeric cell surface receptor.

As used herein, the term “chimeric antigen receptor (CAR)” refers to an artificial transmembrane protein receptor comprising an extracellular domain capable of binding to a predetermined CAR ligand or antigen, an intracellular segment comprising one or more cytoplasmic domains derived from signal transducing proteins different from the polypeptide from which the extracellular domain is derived, and a transmembrane domain.

### Expression and Activity of Polypeptides of Interest

In some embodiments, the disclosure provides methods of generating an endonuclease-resistant mRNA comprising an ORF encoding a polypeptide of interest, wherein the endonuclease resistant of the mRNA results in an increase or enhancement of expression and/or activity of the polypeptide of interest. In some embodiments, expression and/or activity of a polypeptide of interest is enhanced or increased in cells administered the mRNA (e.g. an endonuclease-resistant mRNA) encoding a polypeptide of interest described herein. In some embodiments, the increase in expression and/or activity of a polypeptide of interest encoded by an endonuclease-resistant mRNA is relative to the expression and/or activity of the polypeptide of interest encoded by an endonuclease-susceptible mRNA.

In some embodiments, the expression and/or activity of a polypeptide of interest is increased or enhanced by at least 1-fold, 2-fold, 3-fold, 4-fold, 5-fold, 6-fold, 7-fold, 8-fold, 9-fold, or 10-fold as determined by a method described herein. In some embodiments, the expression and/or activity of a polypeptide of interest is increased or enhanced by at least 10%, at least 20%, at least 30%, at least 40%, at least 50%, at least 60%, at least 70% at least 80%, at least 90% or at least 100% as determined by a method described herein.

Methods for determining expression and/or activity of a polypeptide of interest described herein are known to those of skill in the art and described herein. Such methods include, but are not limited to, quantitative immunofluorescence (QIF), flow cytometry, reverse transcription polymerase chain reaction (RT-PCR), competitive RT-PCR, real-time RT-PCR, RNase protection assay (RPA), northern blotting, nucleic acid microarray using DNA, western blotting, enzyme-linked immunosorbent assay (ELISA), radioimmunoassay (RIA), tissue immunostaining, immunoprecipitation assay, complement fixation assay, fluorescence-activated cell sorting (FACS), mass spectrometry, magnetic bead-antibody immunoprecipitation, or protein chip.

### Pharmaceutical Compositions

The present disclosure includes pharmaceutical compositions comprising an mRNA (e.g., a therapeutic, stabilized mRNA) or a nanoparticle (e.g., a lipid nanoparticle) described herein, in combination with one or more pharmaceutically acceptable excipient, carrier or diluent. In particular embodiments, the mRNA is present in a nanoparticle, e.g., a lipid nanoparticle. In particular embodiments, the mRNA or nanoparticle is present in a pharmaceutical composition.

Pharmaceutical compositions may optionally include one or more additional active substances, for example, therapeutically and/or prophylactically active substances. Pharmaceutical compositions of the present disclosure may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference in its entirety). In particular embodiments, a pharmaceutical composition comprises an mRNA and a lipid nanoparticle, or complexes thereof.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100%, e.g., between 0.5% and 70%, between 1% and 30%, between 5% and 80%, or at least 80% (w/w) active ingredient.

The mRNAs of the disclosure can be formulated using one or more excipients to: (1) increase stability; (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the disclosure can include one or more excipients, each in an amount that together increases the stability of the mRNA, increases cell transfection by the mRNA, increases the expression of a polypeptide encoded by the mRNA, and/or alters the release profile of an mRNA-encoded polypeptide. Further, the mRNAs of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, the formulations described herein may include at least one pharmaceutically acceptable salt. Examples of pharmaceutically acceptable salts that may be included in a formulation of the disclosure include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In some embodiments, the formulations described herein may contain at least one type of mRNA. As a non-limiting example, the formulations may contain 1, 2, 3, 4, 5 or more than 5 mRNAs described herein. In some embodiments, the formulations described herein may contain at least one mRNA encoding a polypeptide and at least one nucleic acid sequence such as, but not limited to, an siRNA, an shRNA, a snoRNA, and an miRNA.

Liquid dosage forms for e.g., parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and/or suspending agents. In certain embodiments for parenteral administration, compositions are mixed with solubilizing agents such as CREMAPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, pharmaceutical compositions including at least one mRNA described herein are administered to mammals (e.g., humans). Although the descriptions of pharmaceutical compositions provided herein are principally directed to pharmaceutical compositions which are suitable for administration to humans, it will be understood by the skilled artisan that such compositions are generally suitable for administration to any other animal, e.g., to a non-human mammal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys. In particular embodiments, a subject is provided with two or more mRNAs described herein. In particular embodiments, the first and second mRNAs are provided to the subject at the same time or at different times, e.g., sequentially. In particular embodiments, the first and second mRNAs are provided to the subject in the same pharmaceutical composition or formulation, e.g., to facilitate uptake of both mRNAs by the same cells.

The present disclosure also includes kits comprising a container comprising a mRNA (e.g., a stabilized, therapeutic mRNA) encoding a polypeptide that enhances an immune response. In another embodiment, the kit comprises a container comprising a mRNA encoding a polypeptide that enhances an immune response, as well as one or more additional mRNAs encoding one or more antigens or interest. In other embodiments, the kit comprises a first container comprising the mRNA encoding a polypeptide that enhances an immune response and a second container comprising one or more mRNAs encoding one or more antigens of interest. In particular embodiments, the mRNAs for enhancing an immune response and the mRNA(s) encoding an antigen(s) are present in the same or different nanoparticles and/or pharmaceutical compositions. In particular embodiments, the mRNAs are lyophilized, dried, or freeze-dried.

### Kits

In some embodiments, the disclosure provides a kit comprising an mRNA (e.g., a therapeutic, stabilized mRNA), or composition (e.g. lipid nanoparticle) comprising an mRNA (e.g., a therapeutic, stabilized mRNA), as described herein. In some embodiments, a kit comprises a container comprising a pharmaceutical composition comprising a lipid nanoparticle comprising an mRNA described herein; and a pharmaceutically acceptable carrier, and a package insert comprising instructions for administration of the mRNA.

In some embodiments, a kit comprises a container comprising a pharmaceutical composition comprising a lipid nanoparticle comprising an mRNA encoding a polypeptide described herein; and a pharmaceutically acceptable carrier, and a package insert comprising instructions for administration of the mRNA and instruction for use in combination with a second composition comprising a second therapeutic agent.

In some embodiments, a kit comprises a container comprising a lipid nanoparticle encapsulating the mRNA described herein, and an optional pharmaceutically acceptable carrier, or a pharmaceutical composition, and a package insert comprising instructions for administration of the lipid nanoparticle or pharmaceutical composition. In some embodiments, a kit comprises a container comprising a lipid nanoparticle encapsulating the mRNA described herein, and an optional pharmaceutically acceptable carrier, or a pharmaceutical composition, and a package insert comprising instructions for administration of the lipid nanoparticle or pharmaceutical composition for treating or delaying progression of an disease or disorder in an individual. In some aspects, the package insert further comprises instructions for administration of the lipid nanoparticle or pharmaceutical composition in combination with a composition comprising a second therapeutic agent and an optional pharmaceutically acceptable carrier for treating or delaying progression of a disease or disorder in a patient.

In some embodiments, a kit comprises a medicament comprising a lipid nanoparticle encapsulating an mRNA described herein, and an optional pharmaceutically acceptable carrier, or a pharmaceutical composition, and a package insert comprising instructions for administration of the medicament alone or in combination with a composition comprising a second therapeutic agent and an optional pharmaceutically acceptable carrier.

### Methods and Use

The disclosure provides methods using the mRNAs (e.g., therapeutic, stabilized mRNAs), compositions, lipid nanoparticles, or pharmaceutical compositions disclosed herein. In some aspects, the mRNAs (e.g, therapeutic, stabilized mRNAs) described herein are used to increase the amount and/or quality of a polypeptide (e.g., a therapeutic polypeptide) encoded by and translated from the mRNA. In some embodiments, the mRNAs described herein are useful for increasing the potency of an mRNA a polypeptide.

In some embodiments, the disclosure provides a method of increasing an amount of a polypeptide translated from an open reading frame comprising an mRNA, the method comprising contacting a cell with an mRNA, a composition, a lipid nanoparticle, or a pharmaceutical composition according to the disclosure.

In some embodiments, the disclosure provides a method of increasing potency of a polypeptide translated from an mRNA, the method comprising contacting a cell with an mRNA, a composition, a lipid nanoparticle, or a pharmaceutical composition according to the disclosure.

In one embodiment, the method comprises administering to the subject a composition of the disclosure (or lipid nanoparticle thereof, or pharmaceutical composition thereof) comprising at least one mRNA construct encoding a polypeptide (e.g., a therapeutic polypeptide)

Compositions of the disclosure are administered to the subject at an effective amount or effective dose. In general, an effective amount of the composition will allow for efficient production of the encoded polypeptide in the cell. Metrics for efficiency may include polypeptide translation (indicated by polypeptide expression), level of mRNA degradation, and immune response indicators.

### Therapeutic Methods

The methods of the disclosure for preparing a stabilized, therapeutic mRNA further comprise treating or delaying progression of a disease or disorder in a clinical, prophylactic or therapeutic application that would benefit from expression of a therapeutic polypeptide encoded by an mRNA. For example, a disease or disorder that would benefit from increased expression of an infectious disease antigen, a tumor cell antigen, a soluble effector molecule, an antibody, an enzyme, a recruitment factor, a transcription factor, a membrane bound receptor, a membrane bound ligand or any fragment or variant thereof. A method of treating a patient with a disease or disorder that would benefit from increased expression of a therapeutic polypeptide comprises providing to the subject an effective amount of a stabilized, therapeutic mRNA (e.g., a therapeutic, stabilized mRNA) that comprises an ORF encoding the therapeutic polypeptide.

Accordingly, in one aspect, the disclosure pertains to a method of increased expression of a therapeutic polypeptide in a subject in need thereof, the method comprising administering to the subject a composition of the disclosure comprising a stabilized, therapeutic mRNA comprising an ORF encoding the therapeutic polypeptide. In some embodiments, the subject is provided with or administered a nanoparticle (e.g., a lipid nanoparticle) comprising the mRNA. In some embodiments, the subject is provided with or administered a pharmaceutical composition of the disclosure comprising the mRNA. In some embodiments, the pharmaceutical composition comprises an mRNA encoding a polypeptide of interest described herein, or it comprises a nanoparticle comprising the mRNA. In some embodiments, the mRNA, nanoparticle, or pharmaceutical composition is administered to the patient parenterally. In particular embodiments, the subject is a mammal, e.g., a human. In various embodiments, the subject is provided with an effective amount of the mRNA.

A pharmaceutical composition including one or more stabilized, therapeutic mRNAs of the disclosure may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the disclosure may be administered that delivers a stabilized, therapeutic mRNA of the disclosure at a dosage level from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In some embodiments, a dose of about 0.005 mg/kg to about 5 mg/kg of mRNA or nanoparticle of the disclosure may be administrated.

In some embodiments, the therapeutic composition comprises a dosage of a stabilized, therapeutic mRNA of the disclosure that is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 100-300 μg, 120-250 μg, 150-250 μg, 180-280 μg, 200-300 μg, 30-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the therapeutic composition is administered to the subject by intradermal or intramuscular injection. In some embodiments, the therapeutic composition is administered to the subject on day zero. In some embodiments, a second dose of the therapeutic composition is administered to the subject on day seven, or day fourteen or day twenty one.

In some embodiments, a dosage of 25 micrograms of a stabilized, therapeutic mRNA of the disclosure is included in the therapeutic composition administered to the subject. In some embodiments, a dosage of 10 micrograms of a stabilized, therapeutic mRNA of the disclosure is included in the therapeutic composition administered to the subject. In some embodiments, a dosage of 30 micrograms of a stabilized, therapeutic mRNA of the disclosure is included in the therapeutic composition administered to the subject. In some embodiments, a dosage of 100 micrograms of a stabilized, therapeutic mRNA of the disclosure is included in the therapeutic composition administered to the subject. In some embodiments, a dosage of 50 micrograms of a stabilized, therapeutic mRNA of the disclosure is included in the therapeutic composition administered to the subject. In some embodiments, a dosage of 75 micrograms of a stabilized, therapeutic mRNA of the disclosure is included in the therapeutic composition administered to the subject. In some embodiments, a dosage of 150 micrograms of a stabilized, therapeutic mRNA of the disclosure is included in the therapeutic composition administered to the subject. In some embodiments, a dosage of 400 micrograms of a stabilized, therapeutic mRNA of the disclosure is included in the therapeutic composition administered to the subject. In some embodiments, a dosage of 300 micrograms of a stabilized, therapeutic mRNA of the disclosure is included in the therapeutic composition administered to the subject. In some embodiments, a dosage of 200 micrograms of a stabilized, therapeutic mRNA of the disclosure is included in the therapeutic composition administered to the subject. In some embodiments, the therapeutic composition is chemically modified and in other embodiments the therapeutic composition is not chemically modified.

In some embodiments, the effective amount is a total dose of 1-100 μg of a stabilized, therapeutic mRNA (e.g., endonuclease resistant mRNA). In some embodiments, the effective amount is a total dose of 100 μg of a stabilized, therapeutic mRNA of the disclosure. In some embodiments, the effective amount is a dose of 25 μg of a stabilized, therapeutic mRNA of the disclosure administered to the subject a total of one or two times. In some embodiments, the effective amount is a dose of 100 μg of a stabilized, therapeutic mRNA of the disclosure administered to the subject a total of two times. In some embodiments, the effective amount is a dose of 1 μg-10 μg, 1 μg-20 μg, 1 μg-30 μg, 5 μg-10 μg, 5 μg-20 μg, 5 μg-30 μg, 5 μg-40 μg, 5 μg-50 μg, 10 μg-15 μg, 10 μg-20 μg, 10 μg-25 μg, 10 μg-30 μg, 10 μg-40 μg, 10 μg-50 μg, 10 μg-60 μg, 15 μg-20 μg, 15 μg-25 μg, 15 μg-30 μg, 15 μg-40 μg, 15 μg-50 μg, 20 μg-25 μg, 20 μg-30 μg, 20 μg-40 μg 20 μg-50 μg, 20 μg-60 μg, 20 μg-70 μg, 20 μg-75 μg, 30 μg-35 μg, 30 μg-40 μg, 30 μg-45 μg 30 μg-50 μg, 30 μg-60 μg, 30 μg-70 μg, 30 μg-75 μg of a stabilized, therapeutic mRNA of the disclosure which may be administered to the subject a total of one or two times or more.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). For example, in certain embodiments, a composition of the disclosure is administered at least two times wherein the second dose is administered at least one day, or at least 3 days, or least 7 days, or at least 10 days, or at least 14 days, or at least 21 days, or at least 28 days, or at least 35 days, or at least 42 days or at least 48 days after the first dose is administered. In certain embodiments, a first and second dose are administered on days 0 and 2, respectively, or on days 0 and 7 respectively, or on days 0 and 14, respectively, or on days 0 and 21, respectively, or on days 0 and 48, respectively. Additional doses (i.e., third doses, fourth doses, etc.) can be administered on the same or a different schedule on which the first two doses were administered. For example, in some embodiments, the first and second dosages are administered 7 days apart and then one or more additional doses are administered weekly thereafter. In another embodiment, the first and second dosages are administered 7 days apart and then one or more additional doses are administered every two weeks thereafter.

In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, a pharmaceutical composition of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

In some embodiments, the present disclosure provides methods of administration of LNP encapsulated mRNAs encoding a polypeptide of interest for the treatment of a disease or disorder, such as those described herein. In some embodiments, the methods described herein comprise administering to the subject an LNP encapsulating an endonuclease-resistant mRNA encoding a polypeptide of interest (e.g., a therapeutic polypeptide.

Compositions of the disclosure are administered to the subject in an effective amount. In general, an effective amount of the composition will allow for efficient production of the encoded polypeptide in cells of the subject. Metrics for efficiency may include polypeptide translation (indicated by polypeptide expression), level of mRNA degradation, and immune response indicators.

In some embodiments, a subject is administered at least one mRNA composition described herein. In related embodiments, the subject is provided with or administered a nanoparticle (e.g., a lipid nanoparticle) comprising the mRNA(s). In further related embodiments, the subject is provided with or administered a pharmaceutical composition of the disclosure to the subject. In particular embodiments, the pharmaceutical composition comprises an mRNA(s) as described herein, or it comprises a nanoparticle comprising the mRNA(s). In particular embodiments, the mRNA(s) is present in a nanoparticle, e.g., a lipid nanoparticle. In particular embodiments, the mRNA(s) or nanoparticle is present in a pharmaceutical composition.

Suitable doses for human patients can be evaluated in, e.g., a Phase I dose escalation study. Data obtained from the cell culture assays and animal studies can be used in formulating a range of dosage for use in humans. The dosage of such mRNA described herein lies generally within a range of local concentrations of the mRNA that include the ED50 with little or no toxicity. The dosage may vary within this range depending upon the dosage form employed and the route of administration utilized. For the mRNA and compositions described herein, the therapeutically effective dose can be estimated initially from cell culture assays. A dose can be formulated in animal models to achieve a therapeutically effective concentration within the local site that includes the IC50 (i.e., the concentration of the mRNA which achieves a half-maximal inhibition of symptoms) as determined in cell culture. Such information can be used to more accurately determine useful doses in humans.

In some embodiments, the mRNA or composition is administered as a single dose or as two or more doses (which may or may not contain the same amount of the desired molecule) over time, or as a continuous infusion via an implantation device or catheter. Further refinement of the appropriate dosage is routinely made by those of ordinary skill in the art. In certain embodiments, appropriate dosages can be ascertained through use of appropriate dose-response data. In some embodiments, the specified time period is determined by a clinician.

In some embodiments, the dosing regimen is determined by the pharmacodynamics effects of the therapeutic polypeptide. In some embodiments, the frequency of dosing will take into account the pharmacokinetic parameters of the mRNA in the formulation used. In certain embodiments, a clinician will administer the composition until a dosage is reached that achieves or maintains the desired effect. In some embodiments, achievement of a desired effect occurs immediately after administration of a dose. In some embodiments, achievement occurs at any point in time following administration. In some embodiments, achievement occurs at any point in time during a dosing interval. In some embodiments, achievement of a desired effect is determined by analyzing a biological sample (e.g., biopsy) immediately after administration of a dose, at any point in time following administration of a dose, at any point in time during a doing interval, or combinations thereof.

In some embodiments, maintenance of a desired effect (e.g., death-inducing protein expression) is determined by analyzing a biological sample (e.g., biopsy) at least once during a dosing interval. In some embodiments, maintenance of a desired effect (e.g., death-inducing protein expression) is determined by analyzing a biological sample (e.g., biopsy) at regular intervals during a dosing interval. In some embodiments, maintenance of a desired effect (e.g., death-inducing protein expression) is determined by analyzing a biological sample (e.g., biopsy) before a subsequent dose is administered.

In some embodiments, dosing occurs until a positive therapeutic outcome is achieved. In some embodiments, dosing of a composition comprising mRNAs encoding polypeptides of interest will occur indefinitely, or until a positive therapeutic outcome is achieved. In some embodiments, the dosing interval remains consistent. In some embodiments, the dosing interval changes as needed based on a clinician's assessment. In some embodiments, dosing occurs indefinitely to maintain remission of a cancer.

### Definitions

Altering: As used herein, “altered” or “altering” refers to a change in the chemical composition, structure, or functionality of an mRNA. In some embodiments, an mRNA of the disclosure is altered to achieve increased potency or increased stability relative to an unaltered mRNA. For example, in some embodiments, an mRNA is altered to increase potency and/or stability by reducing the sensitivity of the mRNA to endonuclease-mediated cleavage. In some embodiments, altering an mRNA to reduce sensitivity to endonuclease-mediated cleavage comprises substitution, insertion, or deletion of the mRNA sequence that is sensitive to endonuclease cleavage. In some embodiments, altering an mRNA to remove one or more endonuclease cleavage site(s) comprises substitution of the endonuclease sensitive sequence with one or more degenerate codons that are less susceptible to endonuclease activity. In another embodiment, altering an mRNA to remove one or more endonuclease cleavage site(s) comprises substitution of the endonuclease sensitive sequence with at least one or more chemically modified nucleotides that are less susceptible to endonuclease activity. In some embodiments, an mRNA is altered to have increased half-life following contacting the mRNA with a cell.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Cap structure or 5′ cap structure: As used herein, the terms “cap structure”, “5′ cap structure” and “5′ cap” refer to a non-extendible dinucleotide that facilitates translation or localization, and/or prevents degradation of an RNA transcript when incorporated at the 5′ end of an RNA transcript, wherein the cap structure can be a natural cap, a derivative of a natural cap, or any chemical group that protects the 5′end of an RNA from degradation and/or is essential for translation initiation. In nature, the modified base 7-methylguanosine is joined in the opposite orientation, 5′ to 5′ rather than 5′ to 3′, to the rest of the molecule via three phosphate groups (i.e., P1-guanosine-5′-yl P3-7-methylguanosine-5′-yl triphosphate (m⁷G5′ppp5′G)). In some embodiments, the mRNA provided herein comprises a “cap analog”, which refers to a structural derivative of an RNA cap that may differ by as little as a single element.

Codon: As used herein, the term “codon” refers to a sequence of three nucleotides that together form a unit of genetic code in a DNA or RNA molecule. A codon is operationally defined by the initial nucleotide from which translation starts and sets the frame for a run of successive nucleotide triplets, which is known as an “open reading frame” (ORF). For example, the string GGGAAACCC, if read from the first position, contains the codons GGG, AAA, and CCC; if read from the second position, it contains the codons GGA and AAC; and if read from the third position, GAA and ACC. Thus, every nucleic sequence read in its 5′→3′ direction comprises three reading frames, each producing a possibly distinct amino acid sequence (in the given example, Gly-Lys-Pro, Gly-Asn, or Glu-Thr, respectively). DNA is double-stranded defining six possible reading frames, three in the forward orientation on one strand and three reverse on the opposite strand. Open reading frames encoding polypeptides are typically defined by a start codon, usually the first AUG codon in the sequence.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., an isolated mRNA, nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

Degenerate Codon: As used herein, the term “degenerate codon” or “alternative codon” refers to a codon that contains a different nucleotide sequence. The degeneracy of codons is known to one skilled in the art and refers to the redundancy of the genetic code, exhibited as the multiplicity of three base pair codon combinations that specify an amino acid. The standard genetic code, which shows the redundancy of codons for each amino acid is shown in Table 11.

TABLE 11
The Genetic Code

| | |
|---|---|
| Ala | GCU, GCC, GCA, GCG |
| Arg | CGU, CGC, CGA, CGG, AGA, AGG |
| Asn | AAU, AAC |
| Asp | GAU, GAC |
| Cys | UGU, UGC |
| Gln | CAA, CAG |
| Glu | GAA, GAG |
| Gly | GGU, GGC, GGA, GGG |
| His | CAU, CAC |
| Ile | AUU, AUC, AUA |
| Start | AUG |
| Glu | GAA, GAG |
| Gly | GGU, GGC, GGA, GGG |
| His | CAU, CAC |
| Leu | UUA, UUG, CUU, CUC, CUA, CUG |
| Lys | AAA, AAG |
| Met | AUG |
| Phe | UUU, UUC |
| Pro | CCU, CCC, CCA, CCG |
| Ser | UCU, UCC, UCA, UCG, AGU, AGC |
| Thr | ACU, ACC, ACA, ACG |
| Trp | UGG |
| Tyr | UAU, UAC |
| Val | GUU, GUC, GUA, GUG |
| Stop | UAG, UGA, UAA |
| Thr | ACU, ACC, ACA, ACG |
| Trp | UGG |
| Tyr | UAU, UAC |

Endonuclease: As used herein, “endonuclease” refers to a cellular enzyme that cleaves the phosphodiester bond with a polynucleotide chain. An endonuclease differs from an exonuclease that cleaves terminal phosphodiester bonds of polynucleotides. In some embodiments, an endonuclease refers to an enzyme that cleaves a phosphodiester bond of DNA, of RNA or of both DNA and RNA. In some embodiments, an endonuclease refers to an enzyme that cleaves the phosphodiester bond within RNA. In some embodiments, an endonuclease cleaves a phosphodiester bond within an RNA to generate a 5′ RNA product and a 3′ RNA product, wherein the 5′ RNA product comprises a 3′ hydroxyl terminus and the 3′ RNA product comprises a 5′ phosphate group. In some embodiments, an endonuclease cleaves a phosphodiester bond within an RNA to generate a 5′ RNA product and a 3′ RNA product, wherein the 5′ RNA product comprises a 2′3′ cyclic phosphate and the 3′ RNA product comprises a 5′ hydroxyl group. In some embodiments, an endonuclease cleaves a phosphodiester bond non-specifically, wherein cleavage occurs at any site within an RNA regardless of the surrounding RNA sequence or structure. In some embodiments, an endonuclease cleaves a phosphodiester bond specifically, wherein cleavage occurs at specific sites within an RNA that is dependent upon the surrounding RNA sequence or structure.

Endonuclease activity: As used herein, “endonuclease activity” refers to the efficiency of enzymatic cleavage of a polynucleotide by one or more endonucleases. The efficiency of enzymatic cleavage of a polynucleotide is determined by measuring the rate of cleavage of a polynucleotide as a function of concentration of the polynucleotide according to methods known in the art. In some embodiments, the efficiency of enzymatic cleavage is measured after contacting the polynucleotide with a recombinant endonuclease, after contacting the polynucleotide with a mixture of recombinant endonucleases, after contacting the polynucleotide with cellular lysate, or after contacting the polynucleotide with a cell. In some embodiments, endonuclease activity refers to the efficiency of enzymatic cleavage of a polynucleotide at a single site within the polynucleotide. In some embodiments, endonuclease activity refers to the efficiency of enzymatic cleavage of a polynucleotide at more than one sites within the polynucleotide.

In some embodiments, the efficiency of enzymatic cleavage of a polynucleotide by one or more endonucleases is measured for an altered polynucleotide compared to an equivalent unaltered polynucleotide counterpart. Increased resistance to endonuclease activity for an altered polynucleotide is defined as decreased efficiency of enzymatic cleavage compared to an unaltered polynucleotide counterpart.

Increases Potency: As used herein, the term “increases potency” “increase potency of an mRNA” refers to the need to administer less of the mRNA to achieve the same functional effect as a less potent mRNA, as a result of, e.g., an increase in functional protein translated from an mRNA (e.g., an endonuclease-resistant mRNA). In some embodiments, an increase in potency occurs owing to an increase in the endonuclease resistance of an mRNA, resulting in an increase in total protein output translated from the mRNA. In some embodiments, an increase in potency occurs due to an increase in the half-life of an mRNA, resulting in an increase in total protein output translated from the mRNA. In some embodiments, an increase in half-life of an mRNA occurs due to (i) reduced sensitivity to an endonuclease cleavage event, (ii) reduced sensitivity to an exonuclease cleavage event, or (iii) reduced stop codon read through. In some embodiments, an increase in half-life increases the number of polypeptide molecules translated per mRNA.

Increases Stability: As used herein, the term “increases stability” or “increases stability of an mRNA” refers to an increase in the ability of the mRNA to resist, reduce or inhibit degradation, and/or increase or improve mRNA half-life. mRNA degradation can occur through physical (e.g., shear or UV radiation), chemical (e.g., hydrolysis), or enzymatic (e.g. nuclease activity) means. Degradation of an mRNA occurs both prior to contacting the mRNA with a cell or after contacting the mRNA with a cell. Upon contacting an mRNA with a cell, cellular machinery induces mRNA degradation, e.g., by enzymatic cleavage of the mRNA. In some aspects, the disclosure relates to altering an mRNA to reduce susceptibility of the mRNA to enzyme-mediated degradation (e.g., exonuclease or endonuclease-mediated degradation) either prior to or following contacting the mRNA with a cell. Reducing the rate of enzymatic degradation of an mRNA in a cell results in increased mRNA half-life or stability. In some embodiments, increased stability of an altered mRNA is measured relative to an unaltered mRNA counterpart (e.g., the starting mRNA prior to altering endonuclease sensitive motifs). In some embodiments, the unaltered mRNA counterpart is endonuclease sensitive, and altering the mRNA yields a stabilized mRNA, wherein the endonuclease resistant mRNA has an increased half-life relative to the endonuclease sensitive unaltered mRNA.

Insertion: As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule.

Insertion Site: As used herein, an “insertion site” is a position or region of a scaffold polypeptide that is amenable to insertion of an amino acid sequence of a heterologous polypeptide. It is to be understood that an insertion site also may refer to the position or region of the polynucleotide that encodes the polypeptide (e.g., a codon of a polynucleotide that codes for a given amino acid in the scaffold polypeptide). In some embodiments, insertion of an amino acid sequence of a heterologous polypeptide into a scaffold polypeptide has little to no effect on the stability (e.g., conformational stability), expression level, or overall secondary structure of the scaffold polypeptide.

microRNA (miRNA) binding site: As used herein, a “microRNA (miRNA) binding site” refers to a miRNA target site or a miRNA recognition site, or any nucleotide sequence to which a miRNA binds or associates. In some embodiments, a miRNA binding site represents a nucleotide location or region of an mRNA to which at least the “seed” region of a miRNA binds. It should be understood that “binding” may follow traditional Watson-Crick hybridization rules or may reflect any stable association of the miRNA with the target sequence at or adjacent to the microRNA site.

miRNA seed: As used herein, a “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, an miRNA seed sequence is to be understood as having complementarity (e.g., partial, substantial, or complete complementarity) with the seed sequence of the miRNA that binds to the miRNA binding site.

Modified mRNA: As used herein, the term “modified mRNA”, includes a non-naturally occurring mRNA that encodes a therapeutically relevant protein and that can be translated. As used herein, the term “modified” includes mRNA molecules which comprise at least one alteration which renders the mRNA molecule more resistant to nucleases than a naturally occurring mRNA molecule encoding the same protein. Exemplary modifications to a nucleic acid sequence of an mRNA molecule which increase the stability of an mRNA molecule include, for example, the depletion of a base (e.g., by deletion or by the substitution of one nucleotide for another). Modifications also include the modification of a base, e.g., the chemical modification of a base. The term “chemical modifications” as used herein, includes modifications which introduce chemistries which differ from those seen in naturally occurring mRNA. For example, chemical modifications include covalent modifications such as the introduction of modified nucleotides, e.g., nucleotide analogs, or the inclusion of pendant groups which are not naturally found in mRNA molecules.

In addition to modifications which include alterations in individual nucleotides of a codon of an mRNA molecule, the term “modification” also includes alteration of more than one nucleotide, e.g., a sequence of nucleotides. In addition, the term modification includes the addition of bases to a sequence (e.g., the inclusion of a poly A tail), alteration of the 3′ or 5′ ends of the mRNA molecule, complexing an mRNA molecule with an agent (e.g., a protein or a complementary nucleic acid molecule) as well as the inclusion of elements which change the structure of an mRNA molecule (e.g., which form secondary structures).

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Therapeutic protein: The term “therapeutically relevant protein” or “therapeutic protein” includes a protein that can be used in the treatment of a subject where the expression of a protein would be of benefit, e.g., in ameliorating the symptoms of a disease or disorder. For example, a therapeutically relevant protein can replace or augment protein expression in a cell which does not normally express a protein or which misexpresses a protein, e.g., a therapeutically relevant protein can compensate for a mutation by supplying a desirable protein. In addition, a “therapeutically relevant protein” can produce a beneficial outcome in a subject, e.g., can be used to produce a protein to which vaccinates a subject against an infectious disease.

Nuclease: As used herein, the term “nuclease” refers to an enzyme with the capability to degrade or otherwise digest polynucleotides or nucleic acid molecules (e.g., DNA or RNA). Representative examples of nucleases include ribonucleases (RNase) which digests RNA, and deoxyribonuclease (DNase) which digests DNA. Unless otherwise specified, the term “nuclease” generally encompasses nuclease enzymes that are capable of degrading single-stranded polynucleotides (e.g., mRNA) and/or double stranded polynucleotides (e.g., DNA). Nucleases are a class of enzymes that are responsible for the cleavage or hydrolysis of the phosphodiester bonds that covalently link the nucleotides that comprise DNA or RNA molecules. Nucleases that cleave or hydrolyze the phosphodiester bonds of DNA are referred to herein as “deoxyribonucleases”. Nucleases that cleave the phosphodiester bonds of RNA are referred to herein as “ribonucleases”.

Stability: As used herein to characterize a polynucleotide (e.g., an mRNA)), the term “stable” or “stability” refers to a reduced susceptibility to degradation or destruction (e.g., a reduced susceptibility to nuclease cleavage). For example, the term “stable” may be used to refer to a reduction in the rate of nuclease degradation (e.g., by endonuclease-mediated cleavage) of an mRNA. In certain embodiments, the half-life (t½) of an mRNA represents an objective measurement of its stability. Similarly, in certain embodiments, the amount, expression level, or enzymatic activity of an expression product that is produced following the expression (e.g., translation) of a stable or nuclease-resistant mRNA represents an objective measurement of its stability.

Variant: As used herein, the term “variant” or “variant mRNA” refers to an mRNA that is altered to increase or improve stability, increase or improve mRNA half-life, increase or improve mRNA potency, increase or improve mRNA resistance to endonuclease activity and/or decrease or reduce mRNA susceptibility to endonuclease activity. In some embodiments, a variant mRNA comprises a sequence that is altered by substitution, insertion, or deletion or by chemical modification to alter an endonuclease sensitive sequence motif relative to a reference mRNA that is unaltered. In some embodiments, a variant mRNA comprises a sequence that is altered in the 5′UTR, the open reading frame, the 3′UTR or any combination thereof. In some embodiments, a variant mRNA comprises altering one or more sites that are sensitive to endonuclease cleavage. In some embodiments, altering one or more sites that are sensitive to endonuclease cleavage generates a variant mRNA that is a stabilized mRNA. In some embodiments, a variant mRNA that is a stabilized mRNA has increased half-life when compared to an unaltered mRNA. In some embodiments, a variant mRNA that is endonuclease resistant has increased potency relative to an unaltered mRNA.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### Example 1: Identification of Nucleotide Sequence Motifs Upstream of Nuclease Cleavage Sites in mRNA

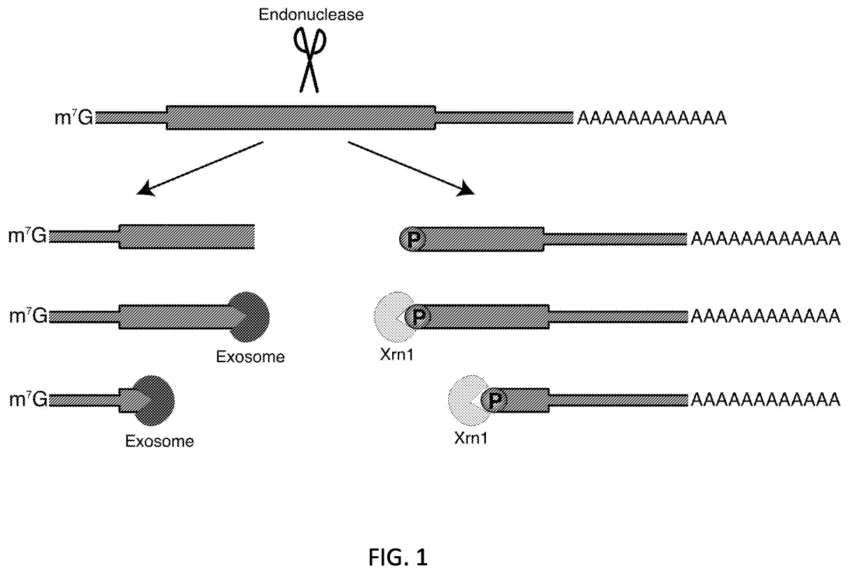

Turnover of mRNA in cells can occur by numerous degradation pathways mediated, in part, by both endonucleases and exonucleases. As shown in FIG. 1, the endonucleolytic cleavage of an mRNA can result in the formation of at least two cleavage products, the 3′ fragment contains a 5′ end containing a 5′ monophosphate group (5P). Recognition of the 5P of an endonucleolytic mRNA fragment (5P mRNA) by a 5′-3′ RNA exonuclease (e.g., Xrn1) can result in the processive enzymatic hydrolysis of the 5P mRNA endonucleolytic fragment in the 5′ to 3′ direction. The 5′ endonucleolytic mRNA product can be hydrolyzed in the 3′ to 5′ direction by the 3′-5′ exonuclease activity of exosome-associated nucleases (Parker (2012) Genetics 191(3):671-702).

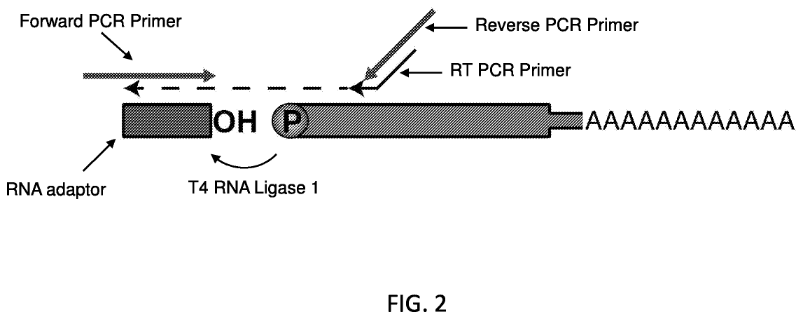

To evaluate mRNA degradation, 5PSeq was used to characterize cellular mRNA degradation intermediates. 5PSeq is a high-throughput RNA sequencing method that profiles mRNA fragments featuring a 5′ monophosphate (see e.g., Pelechano et al., (2016) Nat Protoc 11(2):359-376). Briefly, total RNA was extracted and purified from mouse livers or from AML-12 cells (ATCC CRL-2254) grown in culture. Ribosomal RNA was depleted using an rRNA depletion kit. The 5′ monophosphate of 5P mRNAs present within the pool of total RNA were targeted by direct ligation of an oligonucleotide of known sequence and containing a stretch of random ribonucleotides at the 3′ end. The sequence of 10, 11, or 12 random ribonucleotides at the 3′ end of the oligonucleotide allowed for the identification of PCR duplicates originating from the same molecular ligation event. The resulting oligonucleotide-ligated 5P mRNAs were subject to reverse transcription with an RT primer comprised of a fixed sequence at the 5′ end and a random region at the 3′ end of the primer. This was followed by PCR and high-throughput sequence analysis of cDNA molecules containing the sequence of the oligonucleotide (FIG. 2).

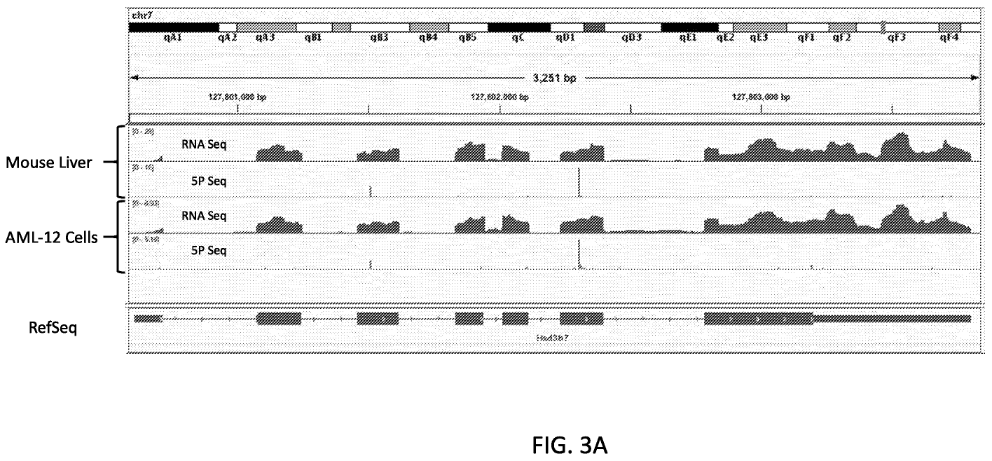

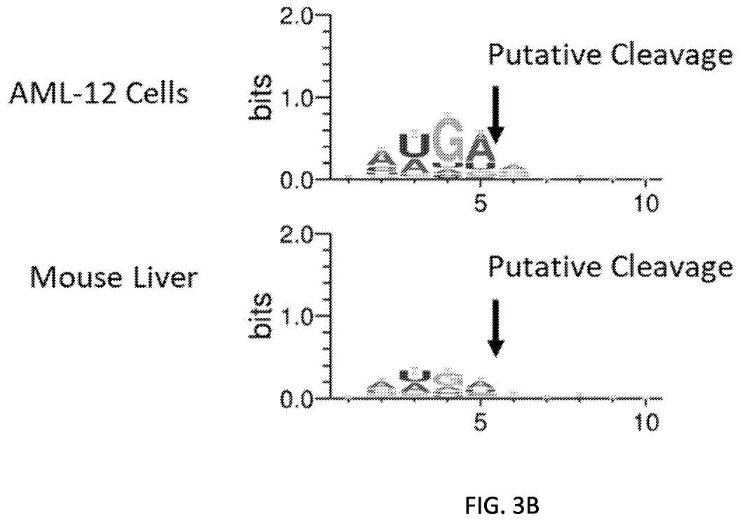

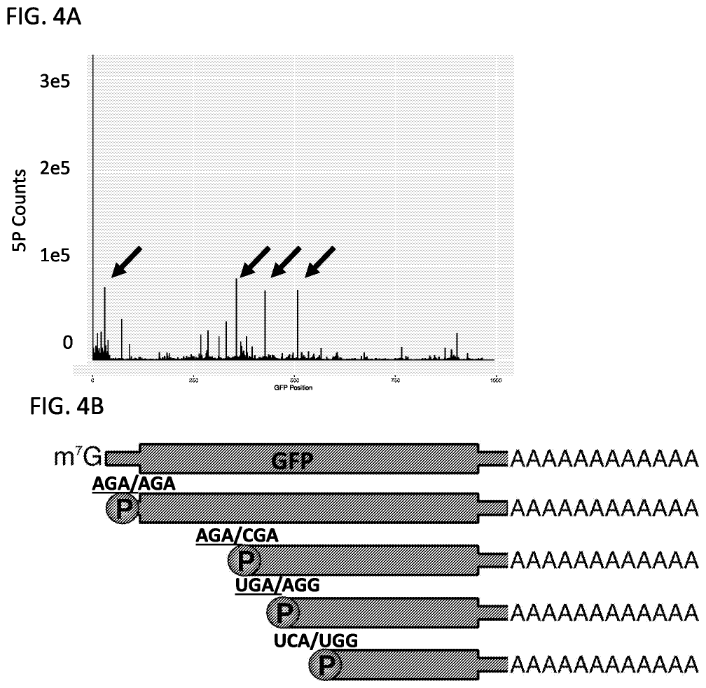

5PSeq analysis of cellular mRNAs isolated from AML-12 cells or mouse liver identified specific 5P mRNAs, suggesting the presence of distinct nuclease cleavage sites rather than random degradation. FIG. 3A shows the frequency distribution of 5P sites within the exonic regions of an mRNA encoding 3 beta-hydroxysteroid dehydrogenase type 7 (Hsd3b7) from both mouse liver and AML-12 cells, as indicated. Aggregating the 5PSeq signal across all mRNAs in transcriptomes from mouse liver and AML-12 cells revealed the presence of distinct nucleotide sequence motifs (UGA or AGA) immediately upstream of the nuclease cleavage sites (FIG. 3B). Enrichment for 5P signal downstream of this motif was also observed in an mRNA encoding eGFP-degron (FIG. 4A). Transfection of eGFP-degron mRNA into AML-12 cells and subsequent analysis by 5PSeq confirmed the presence of eGFP-degron mRNA degradation intermediates with 5′ phosphorylated ends. Of the four most abudant 5P intermediates in eGFPdegron, three were located immediately downstream of identified endonuclease sensitive motifs (FIG. 4B).

These results indicate the presence of specific nuclease cleavage sites in mRNAs that are preceded by the nucleotide sequence motifs UGA or AGA. Further, these results show that both endogenous cellular mRNAs and transfected mRNAs containing these motifs are susceptible to mRNA cleavage at a location that is immediately downstream of these motifs. These results suggest that the stability of an mRNA in cells is affected, in part, by the presence of these motifs within the nucleotide sequence of the mRNA.

### Example 2: Endonuclease-Mediated Cleavage of mRNA Occurs Downstream of Nucleotide Sequence Motifs

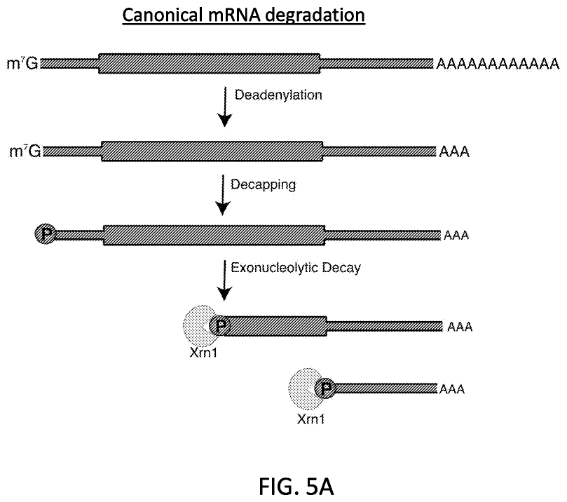

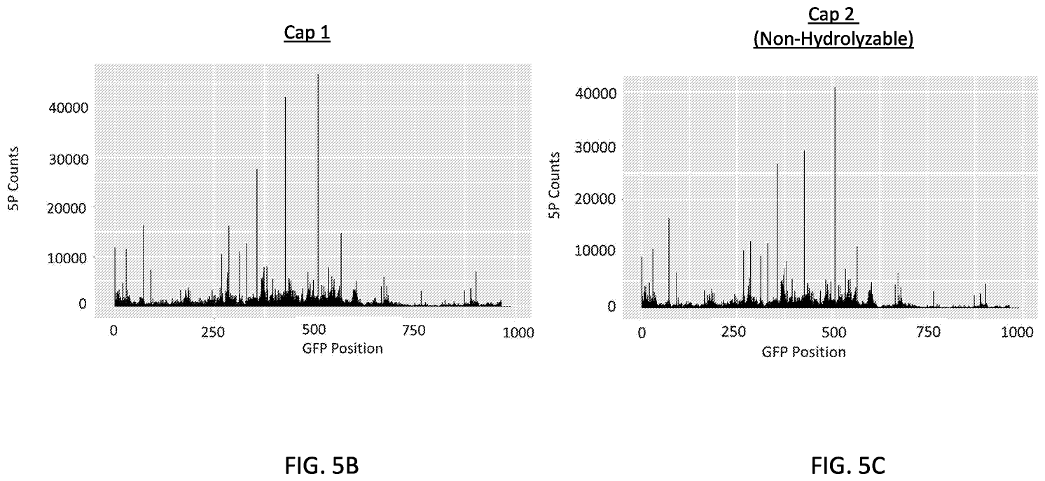

The 5′ cap is a modified 5′ nucleotide that promotes the stability of an mRNA by preventing 5′ exonuclease activity. The process of mRNA decapping occurs during mRNA degradation and results in the hydrolysis of the 5′ cap exposing a 5′ monophosphate (5P). In eukaryotes, this 5′ monophosphate is a substrate for the 5′ exonuclease Xrn1 and decapped mRNAs are further hydrolyzed in a 5′ to 3′ manner by Xrn1 exonuclease activity (FIG. 5A). 5′ caps that are resistant to hydrolysis and function to block decapping and subsequent mRNA degradation are known, including those described by Kalek, et al (2005) Synthesis and biochemical properties of novel mRNA 5′cap analogs resistant to enzymatic hydrolysis, Nucleosides, Nucleotides and Nucleic Acids, 24:5-7, 615-621.

To determine if the 5P mRNAs described in Example 1 were generated by endonuclease activity or by decapping and exonuclease activity, 5P mRNAs from transfected mRNA containing either a hydrolyzable 5′ cap or a non-hydrolyzable 5′ cap resistant to decapping were evaluated by 5PSeq. mRNAs encoding eGFP-degron and containing a standard 5′ cap (Cap 1) or a non-hydrolyzable, synthetic 5′ cap (Cap 2) were transfected into AML-12 cells and 5P mRNA was analyzed as described in Example 1.

As shown in FIGS. 5B-5C, the frequency distribution of 5P mRNAs generated from mRNA containing a standard 5′ cap (Cap 1; FIG. 5B) or a non-hydrolyzable, synthetic 5′ cap (Cap 2; FIG. 5C) was similar. These results indicate that 5P mRNAs from transfected mRNAs are generated from endonucleolytic cleavage and not from decapping and exonuclease activity. These results suggest that the canonical exonucleolytic mRNA degradation pathway initiated at the 5′ and 3′ ends of full-length mRNAs is not the only determinant of mRNA stability in cells, however, the endonuclease responsible for this sequence-specific mRNA cleavage remains unknown.

### Example 3: Endonuclease-Mediated Cleavage of mRNA Occurs Downstream of Nucleotide Sequence Motifs is Translation-Independent

To determine if the endonuclease-mediated cleavage of mRNA described in Examples 1 and 2 was dependent on translation, 5P mRNAs from eGFP-degron-encoding mRNAs containing either a standard 5′ cap (Cap 1) or a synthetic 5′ cap (PglycS NoMe) that blocks translation were transfected into AML-12 cells with lipofectamine 2000. Synthetic caps that block translation are further described by International Patent Application No. PCT/US2016/057384 and PCT/US2016/057405. Additionally, the mRNA were prepared with a 100 nucleotide poly(A) tail conjugated to a 3′-stabilizing region comprising 10 L-adenosine nucleotides. Further description of a 3′-stabilizing region and methods of conjugating the 3′-stabilizing region to an mRNA poly(A) tail are described by International Patent Application No. WO2017049275, filed Sep. 19, 2016, the entire contents of which are expressly incorporated herein by reference.

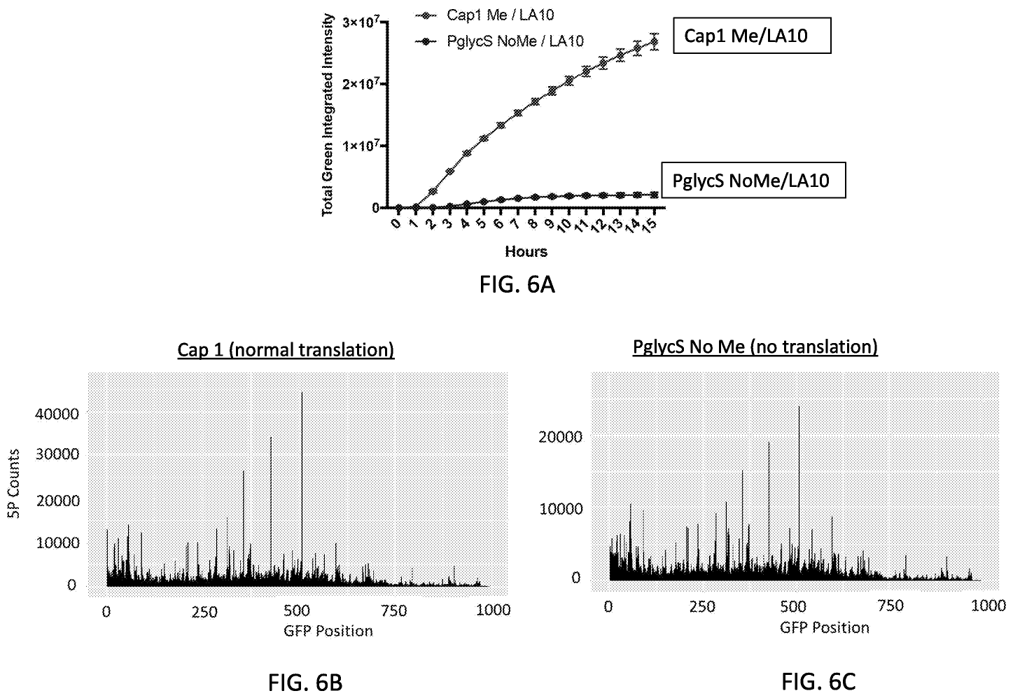

5P mRNA was analyzed by 5PSeq as described in Example 1. To monitor translation, the fluorescent signal from eGFP-degron in cells transfected with the mRNAs was recorded over time. As shown in FIG. 6A, the eGFP fluorescent signal from cells transfected with an eGFP-encoding mRNA containing Cap 1 increased over time relative to cells transfected with an eGFP-encoding mRNA containing the PglycS NoME cap. These results demonstrate that the eGFP-encoding mRNA containing Cap 1 is translated after transfection, while the PglycS NoMe cap effectively blocks translation of the eGFP-degron-encoding mRNA.

As shown in FIGS. 6B-6C, the frequency distribution of 5P mRNAs generated from mRNA containing a standard 5′ cap (Cap 1; FIG. 6B) or the translation-blocking synthetic 5′ cap (PglycS; FIG. 6C) was similar. These results indicate that the 5P mRNAs generated by endonuclease-mediated cleavage of transfected mRNA is not dependent on mRNA translation.

### Example 4: Removal of mRNA Endonuclease Sensitive Sequence Motifs from the Extends Half-Life of mRNA

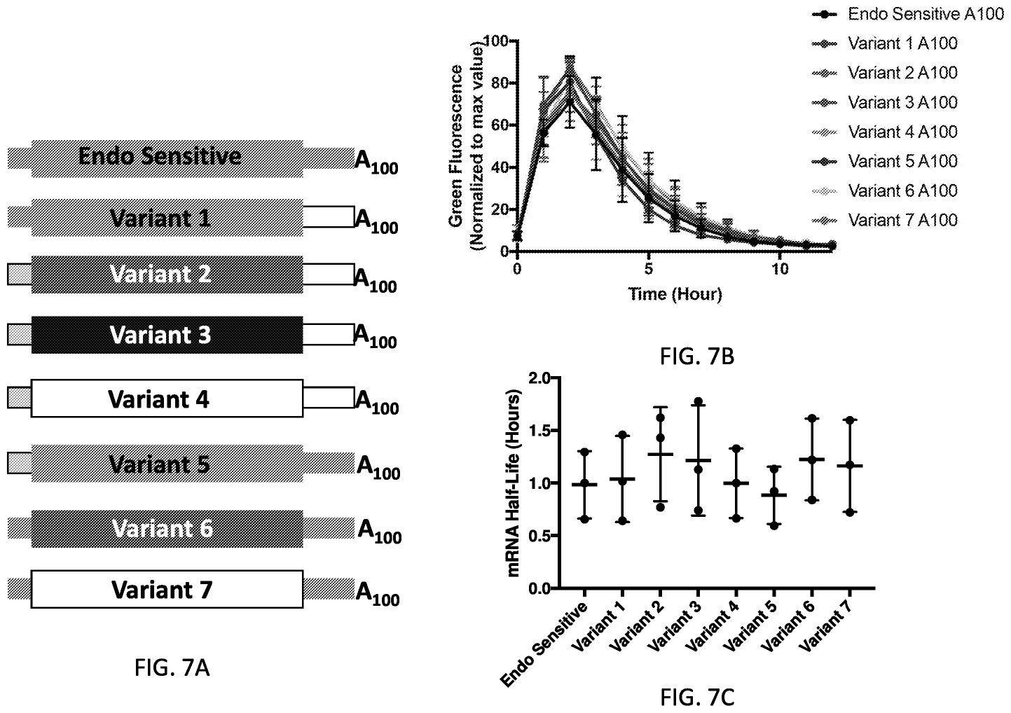

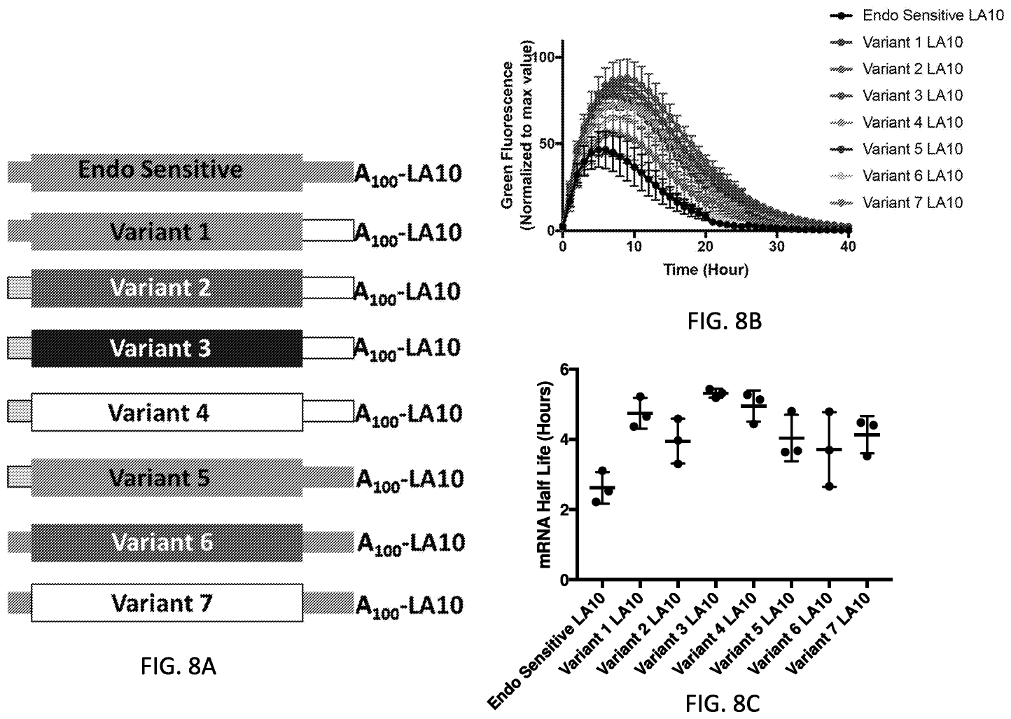

To determine if removal of endonuclease sensitive sequence motifs from an mRNA increases mRNA functional half-life, wild-type eGFP-degron mRNA reporter constructs (Endonuclease sensitive A100 and Endonuclease sensitive LA10) containing two 3′ ends (A100 poly(A) tail or A100 poly(A) tail with LA10 modification) were generated which contain the endonuclease sensitive sequence motifs AGA and UGA throughout the 5′ UTR, the ORF, and the 3′ UTR of the constructs. Variants 1-7 as shown in FIG. 7A and FIG. 8A comprised these mRNA constructs that were altered by substitution of multiple endonuclease sensitive sequence motifs (AGA and UGA sequences) in the 5′UTR, ORF, and/or 3′UTR. The sequences of the 5′UTR and 3′UTR that were altered for removal of endonuclease sensitive motifs are shown in Table 12. The sequences of the ORF encoding GFP (SEQ ID NO: 64) that were altered for removal endonuclease sensitive motifs are shown by SEQ ID NO: 70 (Variant 2, 6), SEQ ID NO: 71 (Variant 3), SEQ ID NO: 72 (Variant 4, 7). The reporter mRNA comprised a triple stop codon with a nucleotide sequence shown in SEQ ID NO: 73 to prevent ribosomal read-through of the ORF. In variant mRNA comprising substitution of endonuclease sensitive sites in the 3′UTR, the first UGA of the triple stop codon was deleted from the sequence.

The mRNA were prepared by in vitro transcription and were fully modified with N1-methyl pseudouridine in place of uracil. The mRNA were either prepared with a 100 nucleotide poly(A) tail alone or conjugated with a 3′-stabilizing region comprising 10 L-adenosine nucleotides (LA10).

Endonuclease sensitive and variant mRNA constructs were transfected into AML12 cells and mRNA functional half-life was monitored by measuring the fluorescent signal from eGFP in cells transfected with the mRNAs over time.

TABLE 12
Nucleotide Sequences of Exemplary Tested 5′ UTR and 3′ UTRs

| UTR | Sequence | SEQ ID NO: |
|---|---|---|
| Endonuclease sensitive | GGGAAAUAAGAGAGAAAAGAAGAGUAAGAAGA | 3 |
| 5′UTR | AAUAUAAGAGCCACC | |
| Variant 2, 3, 4, 5 | GGGAAAUAAGAGACAAAACAACAGUAACAACA | 80 |
| 5′UTR | AAUAUAACAGCCACC | |
| Endonuclease sensitive | UGAUAAUAGGCUGGAGCCUCGGUGGCCUAGCUU | 1 |
| 3′ UTR | CUUGCCCCUUGGGCCUCCCCCCAGCCCCUCCUCC | |
| (A100 and LA10) | CCUUCCUGCACCCGUACCCCCGUGGUCUUUGAA | |
| | UAAAGUCUGAGUGGGCGGC | |
| Variant 1, 2, 3, 4 3′UTR | UAAUAGGCUGGAGCCUCGGUGGCCUAGCUUCUU | 2 |
| (A100 and LA10) | GCCCCUUGGGCCUCCCCCCAGCCCCUCCUCCCCU | |
| | UCCUGCACCCGUACCCCCGUGGUCUUUCAAUAA | |
| | AGUCUCAGUGGGCGGC | |

(endonuclease sensitive sequence motifs before substitution shown underlined, after substitution shown in bold, stop codon of upstream ORF shown in italics. The endonuclease sensitive 3′UTR has a triple stop codon, and the first UGA stop codon was deleted in endonuclease resistant variants)

Variant A100 mRNA construct variants derived from the endonuclease sensitive A100 mRNA construct were generated with substitutions in endonuclease sensitive sequence motifs found in the endonuclease sensitive A100 3′ UTR alone (Variant 1 A100), the 5′ UTR alone (Variant 5 A100), the eGFP-degron ORF alone (Variant 6 A100 and Variant 7 A100), or in the 5′ UTR, the ORF, and the A100 3′ UTR (Variant 2 A100, Variant 3 A100, and Variant 4 A100), as shown in FIG. 7A. The A100 mRNA constructs (endonuclease sensitive and variants) were electroporated into AML12 cells and the fluorescent signal from eGFP-degron polypeptide translated from the mRNAs was monitored over time, as shown in FIG. 7B. The functional half-life of the endonuclease sensitive and Variant A100 mRNA constructs was determined indirectly by monitoring the fluorescent curve from the eGFP-degron polypeptide, and is shown in FIG. 7C.

LA10 mRNA construct variants derived from the endonuclease sensitive LA10 mRNA construct were generated with substitutions in endonuclease sensitive sequence motifs found in the endonuclease sensitive 3′ UTR alone (Variant 1 LA10), the 5′ UTR alone (Variant 5 LA10), the eGFP-degron ORF alone (Variant 6 LA10 and Variant 7 LA10), or in the 5′ UTR, the ORF, and the A100 3′ UTR (Variant 2 LA10, Variant 3 LA10, and Variant 4 LA10), as shown in FIG. 8A. The LA10 mRNA constructs (endonuclease sensitive and variants) were electroporated into AML12 cells and the fluorescent signal from eGFP-degron polypeptide translated from the mRNAs was monitored over time, as shown in FIG. 8B. The functional half-life of the endonuclease sensitive and variant LA10 mRNA constructs was determined indirectly by monitoring the fluorescent curve from the eGFP-degron polypeptide, and is shown FIG. 8C.

While the functional half-lives of all A100 mRNA construct variants were similar to the endonuclease sensitive A100 mRNA construct (FIG. 7C), all LA10 mRNA construct variants exhibited an increase in functional half-life compared to the endonuclease sensitive LA10 mRNA construct and several Variant LA10 mRNA construct variants exhibited an ˜2-fold increase in functional half-life relative to the endonuclease sensitive LA10 mRNA (FIG. 8C). These results demonstrate that the removal of endonuclease sensitive sequence motifs from an mRNA by substitution extends the functional half-life of the mRNA in the context of an LA10 3′ tail, with removal of endonuclease sensitive sequence motifs from the 3′ UTR (Variants 1,2,3,4 LA10) having the greatest effect on functional half-life (FIG. 8C).

SEQUENCE LISTING

| | | | SEQ |
|---|---|---|---|
| Name | Description | Sequence | ID NO: |
| Downstream | v1.1 3′ UTR | UGAUAAUAGGCUGGAGCCUCGGUGGCCUAG | 1 |
| UTR | (+UGA1, | CUUCUUGCCCCUUGGGCCUCCCCCCAGCCCC | |
| | +UGA2,) | UCCUCCCCUUCCUGCACCCGUACCCCCGUGG | |
| | +UGA3) | UCUUUGAAUAAAGUCUGAGUGGGCGGC | |
| Downstream | v1.1 3′ UTR | UAAUAGGCUGGAGCCUCGGUGGCCUAGCUU | 2 |
| UTR | (−UGA1 | CUUGCCCCUUGGGCCUCCCCCCAGCCCCUCC | |
| | −UGA2 | UCCCCUUCCUGCACCCGUACCCCCGUGGUCU | |
| | −UGA3) | UUCAAUAAAGUCUCAGUGGGCGGC | |
| Upstream | V1.0 5′ | GGGAAAUAAGAGAGAAAAGAAGAGUAAGA | 3 |
| UTR | UTR | AGAAAUAUAAGAGCCACC | |
| Upstream | V1.1 5′UTR | GGGAAAUAACACACAAAACAACAGUAACA | 4 |
| UTR | (no AGA) | ACAAAUAUAACAGCCACC | |
| Downstream | v1.1 3′ UTR | UAAUAGGCUGGAGCCUCGGUGGCCUAGCUU | 5 |
| UTR | (−UGA1 | CUUGCCCCUUGGGCCUCCCCCCAGCCCCUCC | |
| | +UGA2 | UCCCCUUCCUGCACCCGUACCCCCGUGGUCU | |
| | +UGA3) | UUGAAUAAAGUCUGAGUGGGCGGC | |
| Downstream | v1.1 3′ UTR | UAAUAGGCUGGAGCCUCGGUGGCCUAGCUU | 6 |
| UTR | (−UGA1 | CUUGCCCCUUGGGCCUCCCCCCAGCCCCUCC | |
| | +UGA2) | UCCCCUUCCUGCACCCGUACCCCCGUGGUCU | |
| | | UUCAAUAAAGUCUGAGUGGGCGGC | |
| Downstream | v1.1 3′ UTR | UAAUAGGCUGGAGCCUCGGUGGCCUAGCUU | 7 |
| UTR | (−UGA1 | CUUGCCCCUUGGGCCUCCCCCCAGCCCCUCC | |
| | +UGA2) | UCCCCUUCCUGCACCCGUACCCCCGUGGUCU | |
| | −UGA3 | UUGAAUAAAGUCUCAGUGGGCGGC | |
| Upstream | V1.1 5′UTR | GGGAAAUAAGAGAGAAAAGAAGAGUAAGA | 8 |
| UTR | | AGAAAUAUAAGACCCCGGCGCCGCCACC | |
| Upstream | V1.0 5′ | AGGAAAUAAGAGAGAAAAGAAGAGUAAGA | 9 |
| UTR | UTR-A | AGAAAUAUAAGAGCCACC | |
| Upstream | V1.0 | UAAGAGAGAAAAGAAGAGUAAGAAGAAAU | 10 |
| UTR | 5′UTR-core | AUAAGAGCCACC | |
| Upstream | V1.1 | AGGAAAUAAGAGAGAAAAGAAGAGUAAGA | 11 |
| UTR | 5′UTR-A | AGAAAUAUAAGACCCCGGCGCCGCCACC | |
| Upstream | V1.1 | UAAGAGAGAAAAGAAGAGUAAGAAGAAAU | 12 |
| UTR | 5′UTR-core | AUAAGACCCCGGCGCCGCCACC | |
| Upstream | 5UTR-001 | GGGAGAUCAGAGAGAAAAGAAGAGUAAGA | 13 |
| UTR | | AGAAAUAUAAGAGCCACC | |
| Upstream | 5′UTR-002 | GGAAUAAAAGUCUCAACACAACAUAUACAA | 14 |
| UTR | | AACAAACGAAUCUCAAGCAAUCAAGCAUUC | |
| | | UACUUCUAUUGCAGCAAUUUAAAUCAUUUC | |
| | | UUUUAAAGCAAAAGCAAUUUUCUGAAAAUU | |
| | | UUCACCAUUUACGAACGAUAGCAAC | |
| Upstream | 5′UTR-003 | GGGAGACAAGCUUGGCAUUCCGGUACUGUU | 15 |
| UTR | | GGUAAAGCCACC | |
| Upstream | 5′UTR-004 | GGGAAUUAACAGAGAAAAGAAGAGUAAGA | 16 |
| UTR | | AGAAAUAUAAGAGCCACC | |
| Upstream | 5′UTR-005 | GGGAAAUUAGACAGAAAAGAAGAGUAAGA | 17 |
| UTR | | AGAAAUAUAAGAGCCACC | |
| Upstream | 5′UTR-006 | GGGAAAUAAGAGAGUAAAGAACAGUAAGA | 18 |
| UTR | | AGAAAUAUAAGAGCCACC | |
| Upstream | 5′UTR-007 | GGGAAAAAAGAGAGAAAAGAAGACUAAGA | 19 |
| UTR | | AGAAAUAUAAGAGCCACC | |
| Upstream | 5′UTR-008 | GGGAAAUAAGAGAGAAAAGAAGAGUAAGA | 20 |
| UTR | | AGAUAUAUAAGAGCCACC | |
| Upstream | 5′UTR-009 | GGGAAAUAAGAGACAAAACAAGAGUAAGA | 21 |
| UTR | | AGAAAUAUAAGAGCCACC | |
| Upstream | 5′UTR-010 | GGGAAAUUAGAGAGUAAAGAACAGUAAGU | 22 |
| UTR | | AGAAUUAAAAGAGCCACC | |
| Upstream | 5′UTR-011 | GGGAAAUAAGAGAGAAUAGAAGAGUAAGA | 23 |
| UTR | | AGAAAUAUAAGAGCCACC | |
| Upstream | 5′UTR-012 | GGGAAAUAAGAGAGAAAAGAAGAGUAAGA | 24 |
| UTR | | AGAAAAUUAAGAGCCACC | |
| Upstream | 5′UTR-013 | GGGAAAUAAGAGAGAAAAGAAGAGUAAGA | 25 |
| UTR | | AGAAAUUUAAGAGCCACC | |
| Upstream | 5′UTR-014 | UCAAGCUUUUGGACCCUCGUACAGAAGCUA | 26 |
| UTR | | AUACGACUCACUAUAGGGAAAUAAGAGAGA | |
| | | AAAGAAGAGUAAGAAGAAAUAUAAGAGCC | |
| | | ACC | |
| Upstream | 5′UTR-015 | GGACAGAUCGCCUGGAGACGCCAUCCACGC | 27 |
| UTR | | UGUUUUGACCUCCAUAGAAGACACCGGGAC | |
| | | CGAUCCAGCCUCCGCGGCCGGGAACGGUGC | |
| | | AUUGGAACGCGGAUUCCCCGUGCCAAGAGU | |
| | | GACUCACCGUCCUUGACACG | |
| Upstream | 5′UTR-016 | GGCGCUGCCUACGGAGGUGGCAGCCAUCUC | 28 |
| UTR | | CUUCUCGGCAUC | |
| Upstream | S065 core | CCUCAUAUCCAGGCUCAAGAAUAGAGCUCA | 29 |
| UTR | | GUGUUUUGUUGUUUAAUCAUUCCGACGUGU | |
| | | UUUGCGAUAUUCGCGCAAAGCAGCCAGUCG | |
| | | CGCGCUUGCUUUUAAGUAGAGUUGUUUUUC | |
| | | CACCCGUUUGCCAGGCAUCUUUAAUUUAAC | |
| | | AUAUUUUUAUUUUUCAGGCUAACCUA | |
| Upstream | S065 | GGGAGACCUCAUAUCCAGGCUCAAGAAUAG | 30 |
| UTR | | AGCUCAGUGUUUUGUUGUUUAAUCAUUCCG | |
| | | ACGUGUUUUGCGAUAUUCGCGCAAAGCAGC | |
| | | CAGUCGCGCGCUUGCUUUUAAGUAGAGUUG | |
| | | UUUUUCCACCCGUUUGCCAGGCAUCUUUAA | |
| | | UUUAACAUAUUUUUAUUUUUCAGGCUAACC | |
| | | UAAAGCAGAGAA | |
| Upstream | combo3_S065 | GGGAGACCUCAUAUCCAGGCUCAAGAAUAG | 31 |
| UTR | (S065 | AGCUCAGUGUUUUGUUGUUUAAUCAUUCCG | |
| | ExtKozak) | ACGUGUUUUGCGAUAUUCGCGCAAAGCAGC | |
| | | CAGUCGCGCGCUUGCUUUUAAGUAGAGUUG | |
| | | UUUUUCCACCCGUUUGCCAGGCAUCUUUAA | |
| | | UUUAACAUAUUUUUAUUUUUCAGGCUAACC | |
| | | UACGCCGCCACC | |
| Downstream | Creatine | GCGCCUGCCCACCUGCCACCGACUGCUGGA | 32 |
| UTR | Kinase | ACCCAGCCAGUGGGAGGGCCUGGCCCACCA | |
| | | GAGUCCUGCUCCCUCACUCCUCGCCCCGCCC | |
| | | CCUGUCCCAGAGUCCCACCUGGGGGCUCUC | |
| | | UCCACCCUUCUCAGAGUUCCAGUUUCAACC | |
| | | AGAGUUCCAACCAAUGGGCUCCAUCCUCUG | |
| | | GAUUCUGGCCAAUGAAAUAUCUCCCUGGCA | |
| | | GGGUCCUCUUCUUUUCCCAGAGCUCCACCC | |
| | | CAACCAGGAGCUCUAGUUAAUGGAGAGCUC | |
| | | CCAGCACACUCGGAGCUUGUGCUUUGUCUC | |
| | | CACGCAAAGCGAUAAAUAAAAGCAUUGGUG | |
| | | GCCUUUGGUCUUUGAAUAAAGCCUGAGUAG | |
| | | GAAGUCUAGA | |
| Downstream | Myoglobin | GCCCCUGCCGCUCCCACCCCCACCCAUCUGG | 33 |
| UTR | | GCCCCGGGUUCAAGAGAGAGCGGGGUCUGA | |
| | | UCUCGUGUAGCCAUAUAGAGUUUGCUUCUG | |
| | | AGUGUCUGCUUUGUUUAGUAGAGGUGGGCA | |
| | | GGAGGAGCUGAGGGGCUGGGGCUGGGGUGU | |
| | | UGAAGUUGGCUUUGCAUGCCCAGCGAUGCG | |
| | | CCUCCCUGUGGGAUGUCAUCACCCUGGGAA | |
| | | CCGGGAGUGGCCCUUGGCUCACUGUGUUCU | |
| | | GCAUGGUUUGGAUCUGAAUUAAUUGUCCUU | |
| | | UCUUCUAAAUCCCAACCGAACUUCUUCCAA | |
| | | CCUCCAAACUGGCUGUAACCCCAAAUCCAA | |
| | | GCCAUUAACUACACCUGACAGUAGCAAUUG | |
| | | UCUGAUUAAUCACUGGCCCCUUGAAGACAG | |
| | | CAGAAUGUCCCUUUGCAAUGAGGAGGAGAU | |
| | | CUGGGCUGGGCGGGCCAGCUGGGGAAGCAU | |
| | | UUGACUAUCUGGAACUUGUGUGUGCCUCCU | |
| | | CAGGUAUGGCAGUGACUCACCUGGUUUUAA | |
| | | UAAAACAACCUGCAACAUCUCAUGGUCUUU | |
| | | GAAUAAAGCCUGAGUAGGAAGUCUAGA | |
| Downstream | α-actin | ACACACUCCACCUCCAGCACGCGACUUCUC | 34 |
| UTR | | AGGACGACGAAUCUUCUCAAUGGGGGGGCG | |
| | | GCUGAGCUCCAGCCACCCCGCAGUCACUUU | |
| | | CUUUGUAACAACUUCCGUUGCUGCCAUCGU | |
| | | AAACUGACACAGUGUUUAUAACGUGUACAU | |
| | | ACAUUAACUUAUUACCUCAUUUUGUUAUUU | |
| | | UUCGAAACAAAGCCCUGUGGAAGAAAAUGG | |
| | | AAAACUUGAAGAAGCAUUAAAGUCAUUCUG | |
| | | UUAAGCUGCGUAAAUGGUCUUUGAAUAAAG | |
| | | CCUGAGUAGGAAGUCUAGA | |
| Downstream | Albumin | CAUCACAUUUAAAAGCAUCUCAGCCUACCA | 35 |
| UTR | | UGAGAAUAAGAGAAAGAAAAUGAAGAUCA | |
| | | AAAGCUUAUUCAUCUGUUUUUCUUUUUCGU | |
| | | UGGUGUAAAGCCAACACCCUGUCUAAAAAA | |
| | | CAUAAAUUUCUUUAAUCAUUUUGCCUCUUU | |
| | | UCUCUGUGCUUCAAUUAAUAAAAAAUGGAA | |
| | | AGAAUCUAAUAGAGUGGUACAGCACUGUUA | |
| | | UUUUUCAAAGAUGUGUUGCUAUCCUGAAAA | |
| | | UUCUGUAGGUUCUGUGGAAGUUCCAGUGUU | |
| | | CUCUCUUAUUCCACUUCGGUAGAGGAUUUC | |
| | | UAGUUUCUUGUGGGCUAAUUAAAUAAAUCA | |
| | | UUAAUACUCUUCUAAUGGUCUUUGAAUAAA | |
| | | GCCUGAGUAGGAAGUCUAGA | |
| Downstream | α-globin | GCUGCCUUCUGCGGGGCUUGCCUUCUGGCC | 36 |
| UTR | | AUGCCCUUCUUCUCUCCCUUGCACCUGUAC | |
| | | CUCUUGGUCUUUGAAUAAAGCCUGAGUAGG | |
| | | AAGGCGGCCGCUCGAGCAUGCAUCUAGA | |
| Downstream | G-CSF | GCCAAGCCCUCCCCAUCCCAUGUAUUUAUC | 37 |
| UTR | | UCUAUUUAAUAUUUAUGUCUAUUUAAGCCU | |
| | | CAUAUUUAAAGACAGGGAAGAGCAGAACGG | |
| | | AGCCCCAGGCCUCUGUGUCCUUCCCUGCAU | |
| | | UUCUGAGUUUCAUUCUCCUGCCUGUAGCAG | |
| | | UGAGAAAAAGCUCCUGUCCUCCCAUCCCCU | |
| | | GGACUGGGAGGUAGAUAGGUAAAUACCAAG | |
| | | UAUUUAUUACUAUGACUGCUCCCCAGCCCU | |
| | | GGCUCUGCAAUGGGCACUGGGAUGAGCCGC | |
| | | UGUGAGCCCCUGGUCCUGAGGGUCCCCACC | |
| | | UGGGACCCUUGAGAGUAUCAGGUCUCCCAC | |
| | | GUGGGAGACAAGAAAUCCCUGUUUAAUAUU | |
| | | UAAACAGCAGUGUUCCCCAUCUGGGUCCUU | |
| | | GCACCCCUCACUCUGGCCUCAGCCGACUGC | |
| | | ACAGCGGCCCCUGCAUCCCCUUGGCUGUGA | |
| | | GGCCCCUGGACAAGCAGAGGUGGCCAGAGC | |
| | | UGGGAGGCAUGGCCCUGGGGUCCCACGAAU | |
| | | UUGCUGGGGAAUCUCGUUUUUCUUCUUAAG | |
| | | ACUUUUGGGACAUGGUUUGACUCCCGAACA | |
| | | UCACCGACGCGUCUCCUGUUUUUCUGGGUG | |
| | | GCCUCGGGACACCUGCCCUGCCCCCACGAG | |
| | | GGUCAGGACUGUGACUCUUUUUAGGGCCAG | |
| | | GCAGGUGCCUGGACAUUUGCCUUGCUGGAC | |
| | | GGGGACUGGGGAUGUGGGAGGGAGCAGACA | |
| | | GGAGGAAUCAUGUCAGGCCUGUGUGUGAAA | |
| | | GGAAGCUCCACUGUCACCCUCCACCUCUUC | |
| | | ACCCCCCACUCACCAGUGUCCCCUCCACUGU | |
| | | CACAUUGUAACUGAACUUCAGGAUAAUAAA | |
| | | GUGUUUGCCUCCAUGGUCUUUGAAUAAAGC | |
| | | CUGAGUAGGAAGGCGGCCGCUCGAGCAUGC | |
| | | AUCUAGA | |
| Downstream | Col1a2; | ACUCAAUCUAAAUUAAAAAAGAAAGAAAUU | 38 |
| UTR | collagen, | UGAAAAAACUUUCUCUUUGCCAUUUCUUCU | |
| | type I, alpha | UCUUCUUUUUUAACUGAAAGCUGAAUCCUU | |
| | 2 | CCAUUUCUUCUGCACAUCUACUUGCUUAAA | |
| | | UUGUGGGCAAAAGAGAAAAAGAAGGAUUG | |
| | | AUCAGAGCAUUGUGCAAUACAGUUUCAUUA | |
| | | ACUCCUUCCCCCGCUCCCCCAAAAAUUUGA | |
| | | AUUUUUUUUUCAACACUCUUACACCUGUUA | |
| | | UGGAAAAUGUCAACCUUUGUAAGAAAACCA | |
| | | AAAUAAAAAUUGAAAAAUAAAAACCAUAA | |
| | | ACAUUUGCACCACUUGUGGCUUUUGAAUAU | |
| | | CUUCCACAGAGGGAAGUUUAAAACCCAAAC | |
| | | UUCCAAAGGUUUAAACUACCUCAAAACACU | |
| | | UUCCCAUGAGUGUGAUCCACAUUGUUAGGU | |
| | | GCUGACCUAGACAGAGAUGAACUGAGGUCC | |
| | | UUGUUUUGUUUUGUUCAUAAUACAAAGGU | |
| | | GCUAAUUAAUAGUAUUUCAGAUACUUGAAG | |
| | | AAUGUUGAUGGUGCUAGAAGAAUUUGAGA | |
| | | AGAAAUACUCCUGUAUUGAGUUGUAUCGUG | |
| | | UGGUGUAUUUUUUAAAAAAUUUGAUUUAG | |
| | | CAUUCAUAUUUUCCAUCUUAUUCCCAAUUA | |
| | | AAAGUAUGCAGAUUAUUUGCCCAAAUCUUC | |
| | | UUCAGAUUCAGCAUUUGUUCUUUGCCAGUC | |
| | | UCAUUUUCAUCUUCUUCCAUGGUUCCACAG | |
| | | AAGCUUUGUUUCUUGGGCAAGCAGAAAAAU | |
| | | UAAAUUGUACCUAUUUUGUAUAUGUGAGA | |
| | | UGUUUAAAUAAAUUGUGAAAAAAAUGAAA | |
| | | UAAAGCAUGUUUGGUUUUCCAAAAGAACAU | |
| | | AU | |
| Downstream | Col6a2; | CGCCGCCGCCCGGGCCCCGCAGUCGAGGGU | 39 |
| UTR | collagen, | CGUGAGCCCACCCCGUCCAUGGUGCUAAGC | |
| | type VI, | GGGCCCGGGUCCCACACGGCCAGCACCGCU | |
| | alpha 2 | GCUCACUCGGACGACGCCCUGGGCCUGCAC | |
| | | CUCUCCAGCUCCUCCCACGGGGUCCCCGUA | |
| | | GCCCCGGCCCCCGCCCAGCCCCAGGUCUCCC | |
| | | CAGGCCCUCCGCAGGCUGCCCGGCCUCCCUC | |
| | | CCCCUGCAGCCAUCCCAAGGCUCCUGACCU | |
| | | ACCUGGCCCCUGAGCUCUGGAGCAAGCCCU | |
| | | GACCCAAUAAAGGCUUUGAACCCAU | |
| Downstream | RPN1; | GGGGCUAGAGCCCUCUCCGCACAGCGUGGA | 40 |
| UTR | ribophorin I | GACGGGGCAAGGAGGGGGGUUAUUAGGAU | |
| | | UGGUGGUUUUGUUUUGCUUUGUUUAAAGCC | |
| | | GUGGGAAAAUGGCACAACUUUACCUCUGUG | |
| | | GGAGAUGCAACACUGAGAGCCAAGGGGUGG | |
| | | GAGUUGGGAUAAUUUUUAUAUAAAAGAAG | |
| | | UUUUUCCACUUUGAAUUGCUAAAAGUGGCA | |
| | | UUUUUCCUAUGUGCAGUCACUCCUCUCAUU | |
| | | UCUAAAAUAGGGACGUGGCCAGGCACGGUG | |
| | | GCUCAUGCCUGUAAUCCCAGCACUUUGGGA | |
| | | GGCCGAGGCAGGCGGCUCACGAGGUCAGGA | |
| | | GAUCGAGACUAUCCUGGCUAACACGGUAAA | |
| | | ACCCUGUCUCUACUAAAAGUACAAAAAAUU | |
| | | AGCUGGGCGUGGUGGUGGGCACCUGUAGUC | |
| | | CCAGCUACUCGGGAGGCUGAGGCAGGAGAA | |
| | | AGGCAUGAAUCCAAGAGGCAGAGCUUGCAG | |
| | | UGAGCUGAGAUCACGCCAUUGCACUCCAGC | |
| | | CUGGGCAACAGUGUUAAGACUCUGUCUCAA | |
| | | AUAUAAAUAAAUAAAUAAAUAAAUAAAUA | |
| | | AAUAAAUAAAAAUAAAGCGAGAUGUUGCCC | |
| | | UCAAA | |
| Downstream | LRP1; low | GGCCCUGCCCCGUCGGACUGCCCCCAGAAA | 41 |
| UTR | density | GCCUCCUGCCCCCUGCCAGUGAAGUCCUUC | |
| | lipoprotein | AGUGAGCCCCUCCCCAGCCAGCCCUUCCCUG | |
| | receptor- | GCCCCGCCGGAUGUAUAAAUGUAAAAAUGA | |
| | related | AGGAAUUACAUUUUAUAUGUGAGCGAGCAA | |
| | protein 1 | GCCGGCAAGCGAGCACAGUAUUAUUUCUCC | |
| | | AUCCCCUCCCUGCCUGCUCCUUGGCACCCCC | |
| | | AUGCUGCCUUCAGGGAGACAGGCAGGGAGG | |
| | | GCUUGGGGCUGCACCUCCUACCCUCCCACC | |
| | | AGAACGCACCCCACUGGGAGAGCUGGUGGU | |
| | | GCAGCCUUCCCCUCCCUGUAUAAGACACUU | |
| | | UGCCAAGGCUCUCCCCUCUCGCCCCAUCCCU | |
| | | GCUUGCCCGCUCCCACAGCUUCCUGAGGGC | |
| | | UAAUUCUGGGAAGGGAGAGUUCUUUGCUGC | |
| | | CCCUGUCUGGAAGACGUGGCUCUGGGUGAG | |
| | | GUAGGCGGGAAAGGAUGGAGUGUUUUAGU | |
| | | UCUUGGGGGAGGCCACCCCAAACCCCAGCC | |
| | | CCAACUCCAGGGGCACCUAUGAGAUGGCCA | |
| | | UGCUCAACCCCCCUCCCAGACAGGCCCUCCC | |
| | | UGUCUCCAGGGCCCCCACCGAGGUUCCCAG | |
| | | GGCUGGAGACUUCCUCUGGUAAACAUUCCU | |
| | | CCAGCCUCCCCUCCCCUGGGGACGCCAAGG | |
| | | AGGUGGGCCACACCCAGGAAGGGAAAGCGG | |
| | | GCAGCCCCGUUUUGGGGACGUGAACGUUUU | |
| | | AAUAAUUUUUGCUGAAUUCCUUUACAACUA | |
| | | AAUAACACAGAUAUUGUUAUAAAUAAAAU | |
| | | UGU | |
| Downstream | Nnt1; | AUAUUAAGGAUCAAGCUGUUAGCUAAUAAU | 42 |
| UTR | cardiotrophin-like | GCCACCUCUGCAGUUUUGGGAACAGGCAAA | |
| | cytokine | UAAAGUAUCAGUAUACAUGGUGAUGUACAU | |
| | factor 1 | CUGUAGCAAAGCUCUUGGAGAAAAUGAAGA | |
| | | CUGAAGAAAGCAAAGCAAAAACUGUAUAGA | |
| | | GAGAUUUUUCAAAAGCAGUAAUCCCUCAAU | |
| | | UUUAAAAAAGGAUUGAAAAUUCUAAAUGU | |
| | | CUUUCUGUGCAUAUUUUUUGUGUUAGGAAU | |
| | | CAAAAGUAUUUUAUAAAAGGAGAAAGAAC | |
| | | AGCCUCAUUUUAGAUGUAGUCCUGUUGGAU | |
| | | UUUUUAUGCCUCCUCAGUAACCAGAAAUGU | |
| | | UUUAAAAAACUAAGUGUUUAGGAUUUCAA | |
| | | GACAACAUUAUACAUGGCUCUGAAAUAUCU | |
| | | GACACAAUGUAAACAUUGCAGGCACCUGCA | |
| | | UUUUAUGUUUUUUUUUUCAACAAAUGUGAC | |
| | | UAAUUUGAAACUUUUAUGAACUUCUGAGCU | |
| | | GUCCCCUUGCAAUUCAACCGCAGUUUGAAU | |
| | | UAAUCAUAUCAAAUCAGUUUUAAUUUUUUA | |
| | | AAUUGUACUUCAGAGUCUAUAUUUCAAGGG | |
| | | CACAUUUUCUCACUACUAUUUUAAUACAUU | |
| | | AAAGGACUAAAUAAUCUUUCAGAGAUGCUG | |
| | | GAAACAAAUCAUUUGCUUUAUAUGUUUCAU | |
| | | UAGAAUACCAAUGAAACAUACAACUUGAAA | |
| | | AUUAGUAAUAGUAUUUUUGAAGAUCCCAUU | |
| | | UCUAAUUGGAGAUCUCUUUAAUUUCGAUCA | |
| | | ACUUAUAAUGUGUAGUACUAUAUUAAGUGC | |
| | | ACUUGAGUGGAAUUCAACAUUUGACUAAUA | |
| | | AAAUGAGUUCAUCAUGUUGGCAAGUGAUGU | |
| | | GGCAAUUAUCUCUGGUGACAAAAGAGUAAA | |
| | | AUCAAAUAUUUCUGCCUGUUACAAAUAUCA | |
| | | AGGAAGACCUGCUACUAUGAAAUAGAUGAC | |
| | | AUUAAUCUGUCUUCACUGUUUAUAAUACGG | |
| | | AUGGAUUUUUUUUCAAAUCAGUGUGUGUU | |
| | | UUGAGGUCUUAUGUAAUUGAUGACAUUUG | |
| | | AGAGAAAUGGUGGCUUUUUUUAGCUACCUC | |
| | | UUUGUUCAUUUAAGCACCAGUAAAGAUCAU | |
| | | GUCUUUUUAUAGAAGUGUAGAUUUUCUUU | |
| | | GUGACUUUGCUAUCGUGCCUAAAGCUCUAA | |
| | | AUAUAGGUGAAUGUGUGAUGAAUACUCAG | |
| | | AUUAUUUGUCUCUCUAUAUAAUUAGUUUGG | |
| | | UACUAAGUUUCUCAAAAAAUUAUUAACACA | |
| | | UGAAAGACAAUCUCUAAACCAGAAAAAGAA | |
| | | GUAGUACAAAUUUUGUUACUGUAAUGCUCG | |
| | | CGUUUAGUGAGUUUAAAACACACAGUAUCU | |
| | | UUUGGUUUUAUAAUCAGUUUCUAUUUUGCU | |
| | | GUGCCUGAGAUUAAGAUCUGUGUAUGUGUG | |
| | | UGUGUGUGUGUGUGCGUUUGUGUGUUAAA | |
| | | GCAGAAAAGACUUUUUUAAAAGUUUUAAG | |
| | | UGAUAAAUGCAAUUUGUUAAUUGAUCUUA | |
| | | GAUCACUAGUAAACUCAGGGCUGAAUUAUA | |
| | | CCAUGUAUAUUCUAUUAGAAGAAAGUAAAC | |
| | | ACCAUCUUUAUUCCUGCCCUUUUUCUUCUC | |
| | | UCAAAGUAGUUGUAGUUAUAUCUAGAAAG | |
| | | AAGCAAUUUUGAUUUCUUGAAAAGGUAGU | |
| | | UCCUGCACUCAGUUUAAACUAAAAAUAAUC | |
| | | AUACUUGGAUUUUAUUUAUUUUUGUCAUA | |
| | | GUAAAAAUUUUAAUUUAUAUAUAUUUUUA | |
| | | UUUAGUAUUAUCUUAUUCUUUGCUAUUUGC | |
| | | CAAUCCUUUGUCAUCAAUUGUGUUAAAUGA | |
| | | AUUGAAAAUUCAUGCCCUGUUCAUUUUAUU | |
| | | UUACUUUAUUGGUUAGGAUAUUUAAAGGA | |
| | | UUUUUGUAUAUAUAAUUUCUUAAAUUAAU | |
| | | AUUCCAAAAGGUUAGUGGACUUAGAUUAUA | |
| | | AAUUAUGGCAAAAAUCUAAAAACAACAAAA | |
| | | AUGAUUUUUAUACAUUCUAUUUCAUUAUUC | |
| | | CUCUUUUUCCAAUAAGUCAUACAAUUGGUA | |
| | | GAUAUGACUUAUUUUAUUUUUGUAUUAUU | |
| | | CACUAUAUCUUUAUGAUAUUUAAGUAUAAA | |
| | | UAAUUAAAAAAAUUUAUUGUACCUUAUAG | |
| | | UCUGUCACCAAAAAAAAAAAAUUAUCUGUA | |
| | | GGUAGUGAAAUGCUAAUGUUGAUUUGUCU | |
| | | UUAAGGGCUUGUUAACUAUCCUUUAUUUUC | |
| | | UCAUUUGUCUUAAAUUAGGAGUUUGUGUU | |
| | | UAAAUUACUCAUCUAAGCAAAAAAUGUAUA | |
| | | UAAAUCCCAUUACUGGGUAUAUACCCAAAG | |
| | | GAUUAUAAAUCAUGCUGCUAUAAAGACACA | |
| | | UGCACACGUAUGUUUAUUGCAGCACUAUUC | |
| | | ACAAUAGCAAAGACUUGGAACCAACCCAAA | |
| | | UGUCCAUCAAUGAUAGACUUGAUUAAGAAA | |
| | | AUGUGCACAUAUACACCAUGGAAUACUAUG | |
| | | CAGCCAUAAAAAAGGAUGAGUUCAUGUCCU | |
| | | UUGUAGGGACAUGGAUAAAGCUGGAAACCA | |
| | | UCAUUCUGAGCAAACUAUUGCAAGGACAGA | |
| | | AAACCAAACACUGCAUGUUCUCACUCAUAG | |
| | | GUGGGAAUUGAACAAUGAGAACACUUGGAC | |
| | | ACAAGGUGGGGAACACCACACACCAGGGCC | |
| | | UGUCAUGGGGUGGGGGGAGUGGGGAGGGA | |
| | | UAGCAUUAGGAGAUAUACCUAAUGUAAAUG | |
| | | AUGAGUUAAUGGGUGCAGCACACCAACAUG | |
| | | GCACAUGUAUACAUAUGUAGCAAACCUGCA | |
| | | CGUUGUGCACAUGUACCCUAGAACUUAAAG | |
| | | UAUAAUUAAAAAAAAAAAGAAAACAGAAG | |
| | | CUAUUUAUAAAGAAGUUAUUUGCUGAAAU | |
| | | AAAUGUGAUCUUUCCCAUUAAAAAAAUAAA | |
| | | GAAAUUUUGGGGUAAAAAAACACAAUAUA | |
| | | UUGUAUUCUUGAAAAAUUCUAAGAGAGUG | |
| | | GAUGUGAAGUGUUCUCACCACAAAAGUGAU | |
| | | AACUAAUUGAGGUAAUGCACAUAUUAAUUA | |
| | | GAAAGAUUUUGUCAUUCCACAAUGUAUAUA | |
| | | UACUUAAAAAUAUGUUAUACACAAUAAAUA | |
| | | CAUACAUUAAAAAAUAAGUAAAUGUA | |
| Downstream | Col6a1; | CCCACCCUGCACGCCGGCACCAAACCCUGUC | 43 |
| UTR | collagen, | CUCCCACCCCUCCCCACUCAUCACUAAACAG | |
| | type VI, | AGUAAAAUGUGAUGCGAAUUUUCCCGACCA | |
| | alpha 1 | ACCUGAUUCGCUAGAUUUUUUUUAAGGAAA | |
| | | AGCUUGGAAAGCCAGGACACAACGCUGCUG | |
| | | CCUGCUUUGUGCAGGGUCCUCCGGGGCUCA | |
| | | GCCCUGAGUUGGCAUCACCUGCGCAGGGCC | |
| | | CUCUGGGGCUCAGCCCUGAGCUAGUGUCAC | |
| | | CUGCACAGGGCCCUCUGAGGCUCAGCCCUG | |
| | | AGCUGGCGUCACCUGUGCAGGGCCCUCUGG | |
| | | GGCUCAGCCCUGAGCUGGCCUCACCUGGGU | |
| | | UCCCCACCCCGGGCUCUCCUGCCCUGCCCUC | |
| | | CUGCCCGCCCUCCCUCCUGCCUGCGCAGCUC | |
| | | CUUCCCUAGGCACCUCUGUGCUGCAUCCCA | |
| | | CCAGCCUGAGCAAGACGCCCUCUCGGGGCC | |
| | | UGUGCCGCACUAGCCUCCCUCUCCUCUGUC | |
| | | CCCAUAGCUGGUUUUUCCCACCAAUCCUCA | |
| | | CCUAACAGUUACUUUACAAUUAAACUCAAA | |
| | | GCAAGCUCUUCUCCUCAGCUUGGGGCAGCC | |
| | | AUUGGCCUCUGUCUCGUUUUGGGAAACCAA | |
| | | GGUCAGGAGGCCGUUGCAGACAUAAAUCUC | |
| | | GGCGACUCGGCCCCGUCUCCUGAGGGUCCU | |
| | | GCUGGUGACCGGCCUGGACCUUGGCCCUAC | |
| | | AGCCCUGGAGGCCGCUGCUGACCAGCACUG | |
| | | ACCCCGACCUCAGAGAGUACUCGCAGGGGC | |
| | | GCUGGCUGCACUCAAGACCCUCGAGAUUAA | |
| | | CGGUGCUAACCCCGUCUGCUCCUCCCUCCCG | |
| | | CAGAGACUGGGGCCUGGACUGGACAUGAGA | |
| | | GCCCCUUGGUGCCACAGAGGGCUGUGUCUU | |
| | | ACUAGAAACAACGCAAACCUCUCCUUCCUC | |
| | | AGAAUAGUGAUGUGUUCGACGUUUUAUCAA | |
| | | AGGCCCCCUUUCUAUGUUCAUGUUAGUUUU | |
| | | GCUCCUUCUGUGUUUUUUUCUGAACCAUAU | |
| | | CCAUGUUGCUGACUUUUCCAAAUAAAGGUU | |
| | | UUCACUCCUCUC | |
| Downstream | Calr; | AGAGGCCUGCCUCCAGGGCUGGACUGAGGC | 44 |
| UTR | calreticulin | CUGAGCGCUCCUGCCGCAGAGCUGGCCGCG | |
| | | CCAAAUAAUGUCUCUGUGAGACUCGAGAAC | |
| | | UUUCAUUUUUUUCCAGGCUGGUUCGGAUUU | |
| | | GGGGUGGAUUUUGGUUUUGUUCCCCUCCUC | |
| | | CACUCUCCCCCACCCCCUCCCCGCCCUUUUU | |
| | | UUUUUUUUUUUUUAAACUGGUAUUUUAUC | |
| | | UUUGAUUCUCCUUCAGCCCUCACCCCUGGU | |
| | | UCUCAUCUUUCUUGAUCAACAUCUUUUCUU | |
| | | GCCUCUGUCCCCUUCUCUCAUCUCUUAGCU | |
| | | CCCCUCCAACCUGGGGGGCAGUGGUGUGGA | |
| | | GAAGCCACAGGCCUGAGAUUUCAUCUGCUC | |
| | | UCCUUCCUGGAGCCCAGAGGAGGGCAGCAG | |
| | | AAGGGGGUGGUGUCUCCAACCCCCCAGCAC | |
| | | UGAGGAAGAACGGGGCUCUUCUCAUUUCAC | |
| | | CCCUCCCUUUCUCCCCUGCCCCCAGGACUGG | |
| | | GCCACUUCUGGGUGGGGCAGUGGGUCCCAG | |
| | | AUUGGCUCACACUGAGAAUGUAAGAACUAC | |
| | | AAACAAAAUUUCUAUUAAAUUAAAUUUUG | |
| | | UGUCUCC | |
| Downstream | Colla1; | CUCCCUCCAUCCCAACCUGGCUCCCUCCCAC | 45 |
| UTR | collagen, | CCAACCAACUUUCCCCCCAACCCGGAAACA | |
| | type I, alpha | GACAAGCAACCCAAACUGAACCCCCUCAAA | |
| | 1 | AGCCAAAAAAUGGGAGACAAUUUCACAUGG | |
| | | ACUUUGGAAAAUAUUUUUUUCCUUUGCAUU | |
| | | CAUCUCUCAAACUUAGUUUUUAUCUUUGAC | |
| | | CAACCGAACAUGACCAAAAACCAAAAGUGC | |
| | | AUUCAACCUUACCAAAAAAAAAAAAAAAAA | |
| | | AAGAAUAAAUAAAUAACUUUUUAAAAAAG | |
| | | GAAGCUUGGUCCACUUGCUUGAAGACCCAU | |
| | | GCGGGGGUAAGUCCCUUUCUGCCCGUUGGG | |
| | | CUUAUGAAACCCCAAUGCUGCCCUUUCUGC | |
| | | UCCUUUCUCCACACCCCCCUUGGGGCCUCCC | |
| | | CUCCACUCCUUCCCAAAUCUGUCUCCCCAG | |
| | | AAGACACAGGAAACAAUGUAUUGUCUGCCC | |
| | | AGCAAUCAAAGGCAAUGCUCAAACACCCAA | |
| | | GUGGCCCCCACCCUCAGCCCGCUCCUGCCCG | |
| | | CCCAGCACCCCCAGGCCCUGGGGGACCUGG | |
| | | GGUUCUCAGACUGCCAAAGAAGCCUUGCCA | |
| | | UCUGGCGCUCCCAUGGCUCUUGCAACAUCU | |
| | | CCCCUUCGUUUUUGAGGGGGUCAUGCCGGG | |
| | | GGAGCCACCAGCCCCUCACUGGGUUCGGAG | |
| | | GAGAGUCAGGAAGGGCCACGACAAAGCAGA | |
| | | AACAUCGGAUUUGGGGAACGCGUGUCAAUC | |
| | | CCUUGUGCCGCAGGGCUGGGCGGGAGAGAC | |
| | | UGUUCUGUUCCUUGUGUAACUGUGUUGCUG | |
| | | AAAGACUACCUCGUUCUUGUCUUGAUGUGU | |
| | | CACCGGGGCAACUGCCUGGGGGCGGGGAUG | |
| | | GGGGCAGGGUGGAAGCGGCUCCCCAUUUUA | |
| | | UACCAAAGGUGCUACAUCUAUGUGAUGGGU | |
| | | GGGGUGGGGAGGGAAUCACUGGUGCUAUAG | |
| | | AAAUUGAGAUGCCCCCCCAGGCCAGCAAAU | |
| | | GUUCCUUUUUGUUCAAAGUCUAUUUUUAUU | |
| | | CCUUGAUAUUUUUCUUUUUUUUUUUUUUUU | |
| | | UUUGUGGAUGGGGACUUGUGAAUUUUUCU | |
| | | AAAGGUGCUAUUUAACAUGGGAGGAGAGCG | |
| | | UGUGCGGCUCCAGCCCAGCCCGCUGCUCAC | |
| | | UUUCCACCCUCUCUCCACCUGCCUCUGGCU | |
| | | UCUCAGGCCUCUGCUCUCCGACCUCUCUCC | |
| | | UCUGAAACCCUCCUCCACAGCUGCAGCCCA | |
| | | UCCUCCCGGCUCCCUCCUAGUCUGUCCUGC | |
| | | GUCCUCUGUCCCCGGGUUUCAGAGACAACU | |
| | | UCCCAAAGCACAAAGCAGUUUUUCCCCCUA | |
| | | GGGGUGGGAGGAAGCAAAAGACUCUGUACC | |
| | | UAUUUUGUAUGUGUAUAAUAAUUUGAGAU | |
| | | GUUUUUAAUUAUUUUGAUUGCUGGAAUAA | |
| | | AGCAUGUGGAAAUGACCCAAACAUAAUCCG | |
| | | CAGUGGCCUCCUAAUUUCCUUCUUUGGAGU | |
| | | UGGGGGAGGGGUAGACAUGGGGAAGGGGC | |
| | | UUUGGGGUGAUGGGCUUGCCUUCCAUUCCU | |
| | | GCCCUUUCCCUCCCCACUAUUCUCUUCUAG | |
| | | AUCCCUCCAUAACCCCACUCCCCUUUCUCUC | |
| | | ACCCUUCUUAUACCGCAAACCUUUCUACUU | |
| | | CCUCUUUCAUUUUCUAUUCUUGCAAUUUCC | |
| | | UUGCACCUUUUCCAAAUCCUCUUCUCCCCU | |
| | | GCAAUACCAUACAGGCAAUCCACGUGCACA | |
| | | ACACACACACACACUCUUCACAUCUGGGGU | |
| | | UGUCCAAACCUCAUACCCACUCCCCUUCAA | |
| | | GCCCAUCCACUCUCCACCCCCUGGAUGCCCU | |
| | | GCACUUGGUGGCGGUGGGAUGCUCAUGGAU | |
| | | ACUGGGAGGGUGAGGGGAGUGGAACCCGUG | |
| | | AGGAGGACCUGGGGGCCUCUCCUUGAACUG | |
| | | ACAUGAAGGGUCAUCUGGCCUCUGCUCCCU | |
| | | UCUCACCCACGCUGACCUCCUGCCGAAGGA | |
| | | GCAACGCAACAGGAGAGGGGUCUGCUGAGC | |
| | | CUGGCGAGGGUCUGGGAGGGACCAGGAGGA | |
| | | AGGCGUGCUCCCUGCUCGCUGUCCUGGCCC | |
| | | UGGGGGAGUGAGGGAGACAGACACCUGGGA | |
| | | GAGCUGUGGGGAAGGCACUCGCACCGUGCU | |
| | | CUUGGGAAGGAAGGAGACCUGGCCCUGCUC | |
| | | ACCACGGACUGGGUGCCUCGACCUCCUGAA | |
| | | UCCCCAGAACACAACCCCCCUGGGCUGGGG | |
| | | UGGUCUGGGGAACCAUCGUGCCCCCGCCUC | |
| | | CCGCCUACUCCUUUUUAAGCUU | |
| Downstream | Plod1; | UUGGCCAGGCCUGACCCUCUUGGACCUUUC | 46 |
| UTR | procollagen- | UUCUUUGCCGACAACCACUGCCCAGCAGCC | |
| | lysine, 2- | UCUGGGACCUCGGGGUCCCAGGGAACCCAG | |
| | oxoglutarate | UCCAGCCUCCUGGCUGUUGACUUCCCAUUG | |
| | 5- | CUCUUGGAGCCACCAAUCAAAGAGAUUCAA | |
| | dioxygenase | AGAGAUUCCUGCAGGCCAGAGGCGGAACAC | |
| | 1 | ACCUUUAUGGCUGGGGCUCUCCGUGGUGUU | |
| | | CUGGACCCAGCCCCUGGAGACACCAUUCAC | |
| | | UUUUACUGCUUUGUAGUGACUCGUGCUCUC | |
| | | CAACCUGUCUUCCUGAAAAACCAAGGCCCC | |
| | | CUUCCCCCACCUCUUCCAUGGGGUGAGACU | |
| | | UGAGCAGAACAGGGGCUUCCCCAAGUUGCC | |
| | | CAGAAAGACUGUCUGGGUGAGAAGCCAUGG | |
| | | CCAGAGCUUCUCCCAGGCACAGGUGUUGCA | |
| | | CCAGGGACUUCUGCUUCAAGUUUUGGGGUA | |
| | | AAGACACCUGGAUCAGACUCCAAGGGCUGC | |
| | | CCUGAGUCUGGGACUUCUGCCUCCAUGGCU | |
| | | GGUCAUGAGAGCAAACCGUAGUCCCCUGGA | |
| | | GACAGCGACUCCAGAGAACCUCUUGGGAGA | |
| | | CAGAAGAGGCAUCUGUGCACAGCUCGAUCU | |
| | | UCUACUUGCCUGUGGGGAGGGGAGUGACAG | |
| | | GUCCACACACCACACUGGGUCACCCUGUCC | |
| | | UGGAUGCCUCUGAAGAGAGGGACAGACCGU | |
| | | CAGAAACUGGAGAGUUUCUAUUAAAGGUCA | |
| | | UUUAAACCA | |
| Downstream | Nucb1; | UCCUCCGGGACCCCAGCCCUCAGGAUUCCU | 47 |
| UTR | nucleobindin | GAUGCUCCAAGGCGACUGAUGGGCGCUGGA | |
| | 1 | UGAAGUGGCACAGUCAGCUUCCCUGGGGGC | |
| | | UGGUGUCAUGUUGGGCUCCUGGGGCGGGGG | |
| | | CACGGCCUGGCAUUUCACGCAUUGCUGCCA | |
| | | CCCCAGGUCCACCUGUCUCCACUUUCACAG | |
| | | CCUCCAAGUCUGUGGCUCUUCCCUUCUGUC | |
| | | CUCCGAGGGGCUUGCCUUCUCUCGUGUCCA | |
| | | GUGAGGUGCUCAGUGAUCGGCUUAACUUAG | |
| | | AGAAGCCCGCCCCCUCCCCUUCUCCGUCUGU | |
| | | CCCAAGAGGGUCUGCUCUGAGCCUGCGUUC | |
| | | CUAGGUGGCUCGGCCUCAGCUGCCUGGGUU | |
| | | GUGGCCGCCCUAGCAUCCUGUAUGCCCACA | |
| | | GCUACUGGAAUCCCCGCUGCUGCUCCGGGC | |
| | | CAAGCUUCUGGUUGAUUAAUGAGGGCAUGG | |
| | | GGUGGUCCCUCAAGACCUUCCCCUACCUUU | |
| | | UGUGGAACCAGUGAUGCCUCAAAGACAGUG | |
| | | UCCCCUCCACAGCUGGGUGCCAGGGGCAGG | |
| | | GGAUCCUCAGUAUAGCCGGUGAACCCUGAU | |
| | | ACCAGGAGCCUGGGCCUCCCUGAACCCCUG | |
| | | GCUUCCAGCCAUCUCAUCGCCAGCCUCCUCC | |
| | | UGGACCUCUUGGCCCCCAGCCCCUUCCCCAC | |
| | | ACAGCCCCAGAAGGGUCCCAGAGCUGACCC | |
| | | CACUCCAGGACCUAGGCCCAGCCCCUCAGCC | |
| | | UCAUCUGGAGCCCCUGAAGACCAGUCCCAC | |
| | | CCACCUUUCUGGCCUCAUCUGACACUGCUC | |
| | | CGCAUCCUGCUGUGUGUCCUGUUCCAUGUU | |
| | | CCGGUUCCAUCCAAAUACACUUUCUGGAAC | |
| | | AAA | |
| Downstream | α-globin-1 | GCUGGAGCCUCGGUGGCCAUGCUUCUUGCC | 48 |
| UTR | | CCUUGGGCCUCCCCCCAGCCCCUCCUCCCCU | |
| | | UCCUGCACCCGUACCCCCGUGGUCUUUGAA | |
| | | UAAAGUCUGAGUGGGCGGC | |
| Downstream | 3′UTR-018 | UAAUAGGCUGGAGCCUCGGUGGCCAUGCUU | 49 |
| UTR | | CUUGCCCCUUGGGCCUCCCCCCAGCCCCUCC | |
| | | UCCCCUUCCUGCACCCGUACCCCCGUGGUCU | |
| | | UUGAAUAAAGUCUGAGUGGGCGGC | |
| Downstream | 3′UTR-019 | UGAUAAUAGGCUGGAGCCUCGGUGGCCAUG | 50 |
| UTR | | CUUCUUGCCCCUUGGGCCUCCCCCCAGCCCC | |
| | | UCCUCCCCUUCCUGCACCCGUACCCCCUGGU | |
| | | CUUUGAAUAAAGUCUGAGUGGGCGGC | |
| Downstream | 3′UTR-020 | UGAUAAUAGGCUGGAGCCUCGGUGGCCAUG | 51 |
| UTR | | CUUCUUGCCCCUUGGGCCUCCCCCCAGCCCC | |
| | | UCCUCCCCUUCCUGCACCCGUACCCCCGUGG | |
| | | UCUUUGAAUAAAGUCUGAGUGGGCGGC | |
| Downstream | 3′ UTR-018 + | UAAUAGGCUGGAGCCUCGGUGGCCAUGCUU | 52 |
| UTR | miR-122- | CUUGCCCCUUGGGCCUCCCCCCAGCCCCUCC | |
| | 5p binding | UCCCCUUCCUGCACCCGUACCCCCCAAACAC | |
| | site | CAUUGUCACACUCCAGUGGUCUUUGAAUAA | |
| | | AGUCUGAGUGGGCGGC | |
| Downstream | 3′ UTR-018 + | UAAUAGGCUGGAGCCUCGGUGGCCAUGCUU | 53 |
| UTR | miR-122- | CUUGCCCCUUGGGCCUCCCCCCAGCCCCUCC | |
| | 3p binding | UCCCCUUCCUGCACCCGUACCCCCUAUUUA | |
| | site | GUGUGAUAAUGGCGUUGUGGUCUUUGAAU | |
| | | AAAGUCUGAGUGGGCGGC | |
| Downstream | 3′ UTR-019 + | UGAUAAUAGGCUGGAGCCUCGGUGGCCAUG | 54 |
| UTR | miR-122- | CUUCUUGCCCCUUGGGCCUCCCCCCAGCCCC | |
| | 5p binding | UCCUCCCCUUCCUGCACCCGUACCCCCCAAA | |
| | site | CACCAUUGUCACACUCCAGUGGUCUUUGAA | |
| | | UAAAGUCUGAGUGGGCGGC | |
| Downstream | 3′ UTR + | GCUGGAGCCUCGGUGGCCAUGCUUCUUGCC | 55 |
| UTR | miR-142-3p | CCUUGGGCCUCCCCCCAGCCCCUCCUCCCCU | |
| | binding site | UCCUGCACCCGUACCCCCUCCAUAAAGUAG | |
| | | GAAACACUACAGUGGUCUUUGAAUAAAGUC | |
| | | UGAGUGGGCGGC | |
| Downstream | Altered 3′ | UAAUAGGCUGGAGCCUCGGUGGCCAUGCUU | 56 |
| UTR | UTR-019 + | CUUGCCCCUUGGGCCUCCCCCCAGCCCCUCC | |
| | miR-122-5p | UCCCCUUCCUGCACCCGUACCCCCCAAACAC | |
| | binding site | CAUUGUCACACUCCAGUGGUCUUUCAAUAA | |
| | | AGUCUCAGUGGGCGGC | |
| Downstream | Altered 3′ | GCUGGAGCCUCGGUGGCCAUGCUUCUUGCC | 57 |
| UTR | UTR + miR- | CCUUGGGCCUCCCCCCAGCCCCUCCUCCCCU | |
| | 142-3p | UCCUGCACCCGUACCCCCUCCAUAAAGUAG | |
| | binding site | GAAACACUACAGUGGUCUUUCAAUAAAGUC | |
| | endonuclease | UCAGUGGGCGGC | |
| | resistant | | |
| Upstream | combo3_S065 - A | AGGAGACCUCAUAUCCAGGCUCAAGAAUAG | 58 |
| UTR | (S065 | AGCUCAGUGUUUUGUUGUUUAAUCAUUCCG | |
| | ExtKozak) | ACGUGUUUUGCGAUAUUCGCGCAAAGCAGC | |
| | | CAGUCGCGCGCUUGCUUUUAAGUAGAGUUG | |
| | | UUUUUCCACCCGUUUGCCAGGCAUCUUUAA | |
| | | UUUAACAUAUUUUUAUUUUUCAGGCUAACC | |
| | | UACGCCGCCACC | |
| miR | miR-142-3p | UGUAGUGUUUCCUACUUUAUGGA | 59 |
| miR | miR-142-5p | CAUAAAGUAGAAAGCACUACU | 60 |
| miR | miR-142 | GACAGUGCAGUCACCCAUAAAGUAGAAAGC | 61 |
| | | ACUACUAACAGCACUGGAGGGUGUAGUGUU | |
| | | UCCUACUUUAUGGAUGAGUGUACUGUG | |
| miR | miR-142-3p | UCCAUAAAGUAGGAAACACUACA | 62 |
| binding site | binding site | | |
| miR | miR-142-5p | AGUAGUGCUUUCUACUUUAUG | 63 |
| binding site | binding site | | |
| ORF | GFP (with | AUGGUGUCCAAGGGUGAGGAAUUGUUUACC | 64 |
| | endonuclease | GGGGUGGUGCCUAUUCUCGUCGAACUUGAC | |
| | sensitive | GGGGAUGUGAAUGGACACAAGUUUUCGGUA | |
| | sites) | UCCGGAGAAGGAGAGGGUGACGCCACAUAC | |
| | | GGAAAGCUUACACUCAAAUUCAUCUGUACG | |
| | | ACGGGGAAACUGCCCGUACCCUGGCCUACG | |
| | | CUCGUAACCACGCUGACUUAUGGAGUGCAG | |
| | | UGCUUUAGCAGAUACCCCGACCAUAUGAAG | |
| | | CAGCACGACUUCUUCAAGUCGGCGAUGCCC | |
| | | GAGGGGUACGUGCAAGAGAGGACCAUUUUC | |
| | | UUCAAAGACGAUGGCAAUUACAAAACACGC | |
| | | GCAGAAGUCAAGUUUGAGGGCGAUACUCUG | |
| | | GUCAAUCGGAUCGAAUUGAAGGGAAUCGAU | |
| | | UUCAAAGAAGAUGGAAACAUCCUUGGCCAU | |
| | | AAGCUCGAGUACAACUAUAACUCGCAUAAU | |
| | | GUCUAUAUCAUGGCUGACAAGCAGAAAAAC | |
| | | GGUAUCAAAGUCAACUUUAAGAUCCGACAC | |
| | | AAUAUUGAGGACGGUUCGGUGCAGCUUGCG | |
| | | GACCACUAUCAACAGAAUACGCCGAUUGGG | |
| | | GAUGGUCCGGUCCUUUUGCCGGAUAACCAU | |
| | | UAUCUCUCAACCCAGUCAGCCCUGAGCAAA | |
| | | GAUCCAAACGAGAAGAGGGACCACAUGGUC | |
| | | UUGCUCGAAUUCGUGACAGCGGCAGGGAUC | |
| | | ACUCUGGGAAUGGACGAGUUGUACAAGAGA | |
| | | UCUCGAGAUAUCAGCCAUGGCUUCCCGCCG | |
| | | GCGGUGGCGGCGCAGGAUGAUGGCACGCUG | |
| | | CCCAUGUCUUGUGCCCAGGAGAGCGGGAUG | |
| | | GACCGUCACCCUGCAGCCUGUGCUUCUGCU | |
| | | AGGAUCAAUGUG | |
| miR | miR-122 | CCUUAGCAGAGCUGUGGAGUGUGACAAUGG | 65 |
| | | UGUUUGUGUCUAAACUAUCAAACGCCAUUA | |
| | | UCACACUAAAUAGCUACUGCUAGGC | |
| miR | miR-122-3p | AACGCCAUUAUCACACUAAAUA | 66 |
| miR | miR-122-5p | UGGAGUGUGACAAUGGUGUUUG | 67 |
| miR | miR-122-3p | UAUUUAGUGUGAUAAUGGCGUU | 68 |
| binding site | binding site | | |
| miR | miR-122-5p | CAAACACCAUUGUCACACUCCA | 69 |
| binding site | binding site | | |
| ORF | GFP variant | AUGGUGUCCAAGGGCGAGGAAUUGUUUACC | 70 |
| | 1 | GGGGUGGUGCCUAUUCUCGUCGAACUCGAC | |
| | | GGGGAUGUCAAUGGACACAAGUUUUCGGUA | |
| | | UCCGGCGAAGGCGAGGGCGACGCCACAUAC | |
| | | GGAAAGCUUACACUCAAAUUCAUCUGUACG | |
| | | ACGGGGAAACUGCCCGUACCCUGGCCUACG | |
| | | CUCGUAACCACGCUCACUUAUGGAGUGCAG | |
| | | UGCUUUAGCAGGUACCCCGACCAUAUGAAG | |
| | | CAGCACGACUUCUUCAAGUCGGCGAUGCCC | |
| | | GAGGGGUACGUGCAGGAAAGGACCAUUUUC | |
| | | UUCAAGGACGAUGGCAAUUACAAAACACGC | |
| | | GCCGAAGUCAAGUUCGAGGGCGAUACUCUG | |
| | | GUCAAUCGGAUCGAAUUAAAGGGAAUCGAU | |
| | | UUCAAGGAGGAUGGAAACAUCCUUGGCCAU | |
| | | AAGCUCGAGUACAACUAUAACUCGCAUAAU | |
| | | GUCUAUAUUAUGGCCGACAAGCAGAAAAAC | |
| | | GGUAUCAAAGUCAACUUUAAAAUCCGACAC | |
| | | AAUAUCGAGGACGGUUCGGUGCAGCUUGCG | |
| | | GACCACUAUCAACAAAAUACGCCGAUUGGG | |
| | | GAUGGUCCGGUCCUUUUGCCGGAUAACCAU | |
| | | UAUCUCUCAACCCAGUCAGCCCUCAGCAAG | |
| | | GAUCCAAACGAAAAAAGGGACCACAUGGUC | |
| | | UUGCUCGAAUUCGUCACAGCGGCAGGGAUC | |
| | | ACUCUGGGAAUGGACGAGUUGUACAAAAGG | |
| | | UCUCGCGAUAUCAGCCAUGGCUUCCCGCCG | |
| | | GCGGUGGCGGCGCAGGACGAUGGCACGCUG | |
| | | CCCAUGUCUUGUGCCCAGGAAAGCGGGAUG | |
| | | GACCGUCACCCUGCAGCCUGUGCUUCUGCU | |
| | | AGGAUCAAUGUG | |
| ORF | GFP Variant | AUGGUGUCCAAGGGCGAGGAAUUGUUUACC | 71 |
| | 2 | GGGGUGGUGCCUAUUCUCGUCGAACUCGAC | |
| | | GGGGAUGUCAAUGGACACAAGUUUUCGGUA | |
| | | UCCGGCGAAGGCGAGGGCGACGCCACAUAC | |
| | | GGAAAGCUUACACUCAAAUUCAUCUGUACG | |
| | | ACGGGGAAACUGCCCGUACCCUGGCCUACG | |
| | | CUCGUAACCACGCUCACUUAUGGAGUGCAG | |
| | | UGCUUUAGCAGGUACCCCGACCAUAUGAAG | |
| | | CAGCACGACUUCUUCAAGUCGGCGAUGCCC | |
| | | GAGGGGUACGUGCAGGAAAGGACCAUUUUC | |
| | | UUCAAGGACGAUGGCAAUUACAAAACACGC | |
| | | GCCGAAGUCAAGUUCGAGGGCGAUACUCUG | |
| | | GUCAAUCGGAUCGAAUUAAAGGGAAUCGAU | |
| | | UUCAAGGAGGAUGGAAACAUCCUUGGCCAU | |
| | | AAGCUCGAGUACAACUAUAACUCGCAUAAU | |
| | | GUCUAUAUCAUGGCCGACAAGCAGAAAAAC | |
| | | GGUAUCAAAGUCAACUUUAAAAUCCGACAC | |
| | | AAUAUCGAGGACGGUUCGGUGCAGCUUGCG | |
| | | GACCACUAUCAACAAAAUACGCCGAUUGGG | |
| | | GAUGGUCCGGUCCUUUUGCCGGAUAACCAU | |
| | | UAUCUCUCAACCCAGUCAGCCCUCAGCAAG | |
| | | GAUCCAAACGAAAAAAGGGACCACAUGGUC | |
| | | UUGCUCGAAUUCGUCACAGCGGCAGGGAUC | |
| | | ACUCUGGGAAUGGACGAGUUGUACAAAAGG | |
| | | UCUCGCGAUAUCAGCCAUGGCUUCCCGCCG | |
| | | GCGGUGGCGGCGCAGGACGAUGGCACGCUG | |
| | | CCCAUGUCUUGUGCCCAGGAAAGCGGGAUG | |
| | | GACCGUCACCCUGCAGCCUGUGCUUCUGCU | |
| | | AGGAUCAAUGUG | |
| ORF | GFP Variant | AUGGUGUCCAAGGGUGAGGAAUUGUUUACC | 72 |
| | 3 | GGGGUGGUGCCUAUUCUCGUCGAACUUGAC | |
| | | GGGGAUGUGAAUGGACACAAGUUUUCGGUA | |
| | | UCCGGAGAAGGAGAGGGUGACGCCACAUAC | |
| | | GGAAAGCUUACACUCAAAUUCAUCUGUACG | |
| | | ACGGGGAAACUGCCCGUACCCUGGCCUACG | |
| | | CUCGUAACCACGCUGACUUAUGGAGUGCAG | |
| | | UGCUUUAGCAGAUACCCCGACCAUAUGAAG | |
| | | CAGCACGACUUCUUCAAGUCGGCGAUGCCC | |
| | | GAGGGGUACGUGCAAGAGAGGACCAUUUUC | |
| | | UUCAAGGACGAUGGCAAUUACAAAACACGC | |
| | | GCAGAAGUCAAGUUUGAGGGCGAUACUCUG | |
| | | GUCAAUCGGAUCGAAUUAAAGGGAAUCGAU | |
| | | UUCAAAGAAGAUGGAAACAUCCUUGGCCAU | |
| | | AAGCUCGAGUACAACUAUAACUCGCAUAAU | |
| | | GUCUAUAUUAUGGCUGACAAGCAGAAAAAC | |
| | | GGUAUCAAAGUCAACUUUAAGAUCCGACAC | |
| | | AAUAUUGAGGACGGUUCGGUGCAGCUUGCG | |
| | | GACCACUAUCAACAGAAUACGCCGAUUGGG | |
| | | GAUGGUCCGGUCCUUUUGCCGGAUAACCAU | |
| | | UAUCUCUCAACCCAGUCAGCCCUGAGCAAA | |
| | | GAUCCAAACGAGAAGAGGGACCACAUGGUC | |
| | | UUGCUCGAAUUCGUGACAGCGGCAGGGAUC | |
| | | ACUCUGGGAAUGGACGAGUUGUACAAGAGA | |
| | | UCUCGAGAUAUCAGCCAUGGCUUCCCGCCG | |
| | | GCGGUGGCGGCGCAGGAUGAUGGCACGCUG | |
| | | CCCAUGUCUUGUGCCCAGGAGAGCGGGAUG | |
| | | GACCGUCACCCUGCAGCCUGUGCUUCUGCU | |
| | | AGGAUCAAUGUG | |
| Stop | 3xstop | UGAUAAUAG | 73 |
| Codon | | | |
| Upstream | combo3_S065 | AGGAGACCUCAUAUCCAGGCUCAAGAAUAG | 74 |
| UTR | (S065 | AGCUCAGUGUUUUGUUGUUUAAUCAUUCCG | |
| | ExtKozak)- | UUUGCGAUAUUCGCGCAAAGCAGCCAGUCG | |
| | minus leader | CGCGCUUGCUUUUAAGUAGAGUUGUUUUUC | |
| | | CACCCGUUUGCCAGGCAUCUUUAAUUUAAC | |
| | | AUAUUUUUAUUUUUCAGGCUAACCUACGCC | |
| | | GCCACC | |
| Upstream | 5′UTR Core | UAAGAGAGAAAAGAAGAGUAAGAAGAAAU | 75 |
| UTR | | AUAAGA | |
| T7 | | TAATACGACTCACTATA [symbol] NNNNNNNNN | 76 |
| promoter | | | |
| T7 | | TAATACGACTCACTATAG | 77 |
| promoter | | | |
| T7 | | TAATACGACTCACTATA [symbol] NNNNNNNNNN | 78 |
| promoter | | | |
| T7 | | ATTATGCTGAGTGATAT [symbol] NNNNNNNNNN | 79 |
| promoter | | | |
| Upstream | Variant 2, 3, | GGGAAAUAAGAGACAAAACAACAGUAACAA | 80 |
| UTR | 4, 5 5′UTR | CAAAUAUAACAGCCACC | |

1. A method of increasing stability of an mRNA, comprising:
(i) providing an mRNA comprising at least one endonuclease sensitive sequence motif, wherein the endonuclease sensitive sequence motif comprises the nucleotide sequence WGA, wherein W=adenine (A) or uracil (U); and
(ii) altering the at least one endonuclease sensitive sequence motif, thereby increasing stability of the mRNA.

2. A method of increasing resistance and/or decreasing susceptibility of an mRNA to endonuclease activity, wherein the mRNA comprises at least one endonuclease sensitive sequence motif, wherein the endonuclease sensitive sequence motif comprises the nucleotide sequence WGA, wherein W=adenine (A) or uracil (U), the method comprising altering the at least one endonuclease sensitive sequence motif, thereby increasing resistance and/or decreasing susceptibility of the mRNA to endonuclease activity.

3. A method of increasing the half-life of an mRNA, wherein the mRNA comprises at least one endonuclease sensitive sequence motif, wherein the endonuclease sensitive sequence motif comprises the nucleotide sequence WGA, wherein W=adenine (A) or uracil (U), the method comprising altering the at least one endonuclease sensitive sequence motif, thereby increasing the half-life of the mRNA.

4. The method of any one of claims 1-3, wherein the mRNA comprises a 5′ untranslated region (5′ UTR), an open reading frame (ORF) encoding a polypeptide, and a 3′ UTR, and wherein the 5′ UTR, ORF and/or 3′ UTR comprise at least one endonuclease sensitive sequence motif.

5. The method of claim 4, wherein the 5′ UTR comprises the at least one endonuclease sensitive sequence motif.

6. The method of claim 4, wherein the ORF comprises the at least one endonuclease sensitive sequence motif.

7. The method of claim 4, wherein the 3′UTR comprises the at least one endonuclease sensitive sequence motif.

8. The method of claim 4, wherein the 5′UTR and ORF each comprise at least one endonuclease sensitive sequence motif, and wherein the at least one endonuclease sensitive sequence motif is the same or is different.

9. The method of claim 4, wherein the 5′UTR and 3′UTR each comprise at least one endonuclease sensitive sequence motif, and wherein the at least one endonuclease sensitive sequence motif is the same or is different.

10. The method of claim 4, wherein the ORF and the 3′UTR each comprise at least one endonuclease sensitive sequence motif, and wherein the at least one endonuclease sensitive sequence motif is the same or is different.

11. The method of claim 4, wherein the 5′UTR, ORF and 3′UTR each comprise at least one endonuclease sensitive sequence motif, and wherein the at least one endonuclease sensitive sequence motif is the same or is different.

12. The method of any one of claims 1-11, wherein the endonuclease sensitive sequence motif is about 3-4, about 4-6, about 6-10, about 3, 4, 5, 6, 7, 8, 9, or 10 nucleotides in length.

13. The method of claim 12, wherein the endonuclease sensitive sequence motif is 3 nucleotides in length.

14. The method of any one of claims 1-13, wherein altering the at least one endonuclease sensitive sequence motif comprises making a substitution or chemical modification of at least one nucleotide in the endonuclease sensitive sequence motif.

15. The method of any one of claims 1-13, wherein altering the at least one endonuclease sensitive sequence motif comprises inserting one or more nucleotides into the motif, deleting one or more nucleotides from the motif, substituting one or more nucleotides in the motif, or a combination thereof.

16. The method of any one of claims 14-15, wherein W=adenine (A), and wherein W is substituted with cytosine (C), guanine (G), or uracil (U).

17. The method of any one of claims 14-15, wherein W=uracil (U), and wherein W is substituted with cytosine (C), guanine (G), or adenine (A).

18. The method of any one of claims 14-15, wherein W=adenine (A), wherein W is preceded by an AG, and wherein AG is substituted with GC.

19. The method of claim 18, wherein W is substituted with cytosine (C), guanine (G), or uracil (U).

20. The method of any one of claims 14-15, wherein W=adenine (A), wherein W is substituted with guanine (G), wherein W is preceded by CG, and wherein the C is substituted with A.

21. The method of any one of claims 14-15, wherein W=adenine (A), wherein W is substituted with cytosine (C) or uracil (U), wherein W is preceded by uracil (U) and cytosine (C), and wherein UC is substituted with AG.

22. The method of any one of claims 14-15, wherein W=adenine (A), wherein W is substituted with cytosine (C), guanine (G), or uracil (U), wherein W is preceded by a UU, and wherein the first U comprising the UU is substituted with C.

23. The method of any one of claims 14-15, wherein W=uracil (U), wherein W is substituted with cytosine (C), guanine (G), or adenine (A), wherein W is preceded by an AG, and wherein the AG is substituted with UC.

24. The method of any one of claims 14-15, wherein W=uracil (U), wherein W is substituted with adenine (A) or guanine (G), wherein W is preceded by a CU, and wherein the C is substituted with U.

25. The method of any one of claims 14-15, wherein W=uracil (U), wherein W is substituted with cytosine (C), wherein W is preceded by a UC, and wherein the UC is substituted with AG.

26. The method of any one of claims 14-15, wherein W=adenine (A), and wherein the adenine (A) following the guanine (G) of the sequence motif is substituted with guanine (G).

27. The method of any one of claims 4, 7, 9, 10, and 11, wherein the sequence motif comprises the stop codon UGA, wherein the G is substituted with A, thereby forming the stop codon UAA.

28. The method of any one of claims 4, 7, 9, 10, and 11, wherein the sequence motif comprises the stop codon UGA, wherein the GA is substituted with an AG, thereby forming the stop codon UAG.

29. The method of any one of claims 4, 6, 8, 10 and 11, wherein the at least one endonuclease sensitive sequence motif comprises the nucleotide sequence AGA, wherein the endonuclease sensitive sequence motif is a codon comprising the ORF, wherein the codon encodes the amino acid arginine, and wherein altering the at least one sequence motif results in the formation of a degenerate codon encoding arginine, and wherein the codon and the degenerate codon are different.

30. The method of claim 29 wherein the degenerate codon is selected from the group consisting of: AGG, CGU, CGC, CGA, and CGG.

31. The method of any one of claims 4, 6, 8, 10 and 11 wherein the at least one endonuclease sensitive sequence motif comprises the nucleotide sequence AGA, wherein the 5′ A is the 3^{rd} position (wobble position) of a codon comprising the ORF, wherein the codon encodes an amino acid selected from the group consisting of: lysine, threonine, arginine, isoleucine, glutamine, proline, leucine, glutamic acid, alanine, glycine, valine, and serine; wherein altering the at least one endonuclease sensitive sequence motif results in the formation of a degenerate codon encoding the amino acid, wherein the codon and the degenerate codon are different.

32. The method of any one of claims 4, 6, 8, 10 and 11 wherein the at least one endonuclease sensitive sequence motif comprises the nucleotide sequence UGA, wherein the U is the 3^{rd} position (wobble position) of a codon comprising the ORF, wherein the codon encodes an amino acid selected from the group consisting of: asparagine, threonine, serine, isoleucine, histidine, proline arginine, leucine, aspartic acid, alanine, glycine, valine, tyrosine, cysteine, and phenylalanine, wherein altering the at least one endonuclease sensitive sequence motif results in the formation of a degenerate codon encoding the amino acid, and wherein the codon and the degenerate codon are different.

33. The method of any one of claims 4, 6, 8, 10, and 11, wherein the at least one endonuclease sensitive sequence motif comprises the nucleotide sequence AGA, wherein the G is the 3^{rd} position (wobble position) of a codon comprising the ORF, wherein the codon encodes an amino acid selected from the group consisting of: lysine, glutamine, and glutamic acid, wherein altering the at least one endonuclease sensitive sequence motif results in the formation of degenerate codon encoding the amino acid, and wherein the codon and the degenerate codon are different.

34. The method of any one of claims 4, 6, 8, 10, and 11, wherein the at least one endonuclease sensitive sequence motif comprises the nucleotide sequence UGA, wherein the G is the 3^{rd} position (wobble position) of a codon comprising the ORF, wherein the codon encodes an amino acid selected from the group consisting of: leucine and valine, wherein altering the at least one endonuclease sensitive sequence motif results in the formation of a degenerate codon encoding the amino acid, and wherein the codon and the degenerate codon are different.

35. The method of any one of claims 1-34, wherein altering the at least one endonuclease sensitive sequence motif increases stability of the mRNA.

36. The method of any one of claims 1-35, wherein altering the at least one endonuclease sensitive sequence motif increases the half-life of the mRNA.

37. The method of any one of claims 2 and 4-36, wherein increased resistance of the mRNA to endonuclease activity is determined relative to an unaltered mRNA, wherein the unaltered mRNA contains at least one additional endonuclease sensitive sequence motif relative to the altered mRNA.

38. An mRNA produced by any one of the preceding methods.

39. An mRNA comprising:
(i) a 5′ UTR;
(ii) an ORF encoding a polypeptide; and
(iii) a 3′ UTR, wherein the 3′ UTR comprises the nucleotide sequence of SEQ ID NO: 1, comprising from 5′ to 3′ a first endonuclease sensitive sequence motif (UGA1), a second endonuclease sensitive sequence motif (UGA2), and a third endonuclease sensitive sequence motif (UGA3), wherein at least one of UGA1, UGA2 and/or UGA3 is altered by deletion, substitution or insertion.

40. The mRNA of claim 39, wherein UGA1 is altered by deletion.

41. The mRNA of claim 39 or 40, wherein UGA2 is altered by a substitution.

42. The mRNA of any one of claims 39-41, wherein UGA3 is altered by a substitution.

43. The mRNA of any one of claims 39-40, wherein UGA2 is altered by substitution of G with cytosine (C) or adenine (A).

44. The mRNA of claim 43, wherein UGA2 is altered by substitution of G with cytosine (C).

45. The mRNA of any one of claims 39-40, wherein UGA3 is altered by substitution of G with cytosine (C), or adenine (A).

46. The mRNA of claim 45, wherein UGA3 is altered by substitution of G with cytosine (C).

47. The mRNA of any one of claims 39-40, wherein UGA2 and UGA3 are altered by substitution of G with cytosine (C), or adenine (A).

48. The mRNA of claim 47, wherein UGA2 and UGA3 are altered by substitution of G with cytosine (C).

49. An mRNA comprising:
(i) a 5′ UTR;
(ii) an ORF encoding a polypeptide; and
(iii) a 3′ UTR comprising the nucleotide sequence of SEQ ID NO: 2.

50. The mRNA of any one of claims 39-49, wherein the ORF encodes a polypeptide of interest.

51. The mRNA of claim 50, wherein the polypeptide of interest is a therapeutic polypeptide.

52. The mRNA of any of claims 39-51, wherein the 3′ UTR comprises a poly-A region.

53. The mRNA of claim 52, wherein the 3′ UTR further comprises a terminal 3′-stabilizing region comprising 1 to 500 nucleosides.

54. The mRNA of claim 53, wherein the 3′-stabilizing region comprises a plurality of alternative nucleosides.

55. The mRNA of claim 54, wherein the alternative nucleoside is L-adenosine

56. The mRNA of any one of claims 53-55, wherein the 3′-stabilizing region is conjugated to the 3′ UTR by a linker.

57. A lipid nanoparticle comprising the mRNA of any one of claims 38-56.

58. A pharmaceutical composition comprising the mRNA of any one of claims 38-56, or the lipid nanoparticle of claim 57, and a pharmaceutically acceptable carrier, diluent or excipient.

59. The lipid nanoparticle of claim 57, and an optional pharmaceutically acceptable carrier, diluent, or excipient, or the pharmaceutical composition of claim 58, for use in treating or delaying progression of a disease or disorder in a subject, wherein the treating or delaying progression of the disease or disorder comprises administration of the lipid nanoparticle, and an optional pharmaceutically acceptable carrier, diluent, or excipient, or the pharmaceutical composition.

60. Use of a lipid nanoparticle of claim 57, and an optional pharmaceutically acceptable carrier, or the pharmaceutical composition of claim 58, in the manufacture of a medicament for treating or delaying progression of a disease or disorder in a subject, wherein the medicament comprises the lipid nanoparticle, and an optional pharmaceutically acceptable carrier, or the pharmaceutical composition and wherein the treatment comprises administration of the medicament.

61. A kit comprising a container comprising the mRNA of any one of claims 38-56, and an optional pharmaceutically acceptable carrier, the lipid nanoparticle of claim 57, and an optional pharmaceutically acceptable carrier, or the pharmaceutical composition of claim 58, and a package insert comprising instructions for administration of the mRNA, the lipid nanoparticle, or the pharmaceutical composition for treating or delaying progression of a disease or disorder in a subject.

62. A method of treating or delaying progression of a disease or disorder in a subject in need thereof, the method comprising administering the mRNA according to any one of claims 38-56, the pharmaceutical composition according to claim 58, or the lipid nanoparticle according to claim 57, thereby treating or delaying progression of the disease or disorder in the subject.


SEQUENCE LISTING (81 sequences)

SEQ ID NO: 1 - RNA, 119 nt, Artificial Sequence
  Synthetic Downstream UTR; v1.1 3 UTR (+UGA1, +UGA2, +UGA3)
ugauaauaggcuggagccucgguggccuagcuucuugccccuugggccuccccccagccccuccuccccuuccugcacccguacccccguggucuuugaauaaagucugagugggcggc

SEQ ID NO: 2 - RNA, 116 nt, Artificial Sequence
  Synthetic Downstream UTR; v1.1 3 UTR (-UGA1 UGA2 -UGA3)
uaauaggcuggagccucgguggccuagcuucuugccccuugggccuccccccagccccuccuccccuuccugcacccguacccccguggucuuucaauaaagucucagugggcggc

SEQ ID NO: 3 - RNA, 47 nt, Artificial Sequence
  Synthetic Upstream UTR; V1.0 5 UTR
gggaaauaagagagaaaagaagaguaagaagaaauauaagagccacc

SEQ ID NO: 4 - RNA, 47 nt, Artificial Sequence
  Synthetic Upstream UTR; V1.1 5′UTR (no AGA)
gggaaauaacacacaaaacaacaguaacaacaaauauaacagccacc

SEQ ID NO: 5 - RNA, 116 nt, Artificial Sequence
  Synthetic Downstream UTR; v1.1 3 UTR (-UGA1 +UGA2 +UGA3)
uaauaggcuggagccucgguggccuagcuucuugccccuugggccuccccccagccccuccuccccuuccugcacccguacccccguggucuuugaauaaagucugagugggcggc

SEQ ID NO: 6 - RNA, 116 nt, Artificial Sequence
  Synthetic Downstream UTR; v1.1 3 UTR (-UGA1 + UGA2)
uaauaggcuggagccucgguggccuagcuucuugccccuugggccuccccccagccccuccuccccuuccugcacccguacccccguggucuuucaauaaagucugagugggcggc

SEQ ID NO: 7 - RNA, 116 nt, Artificial Sequence
  Synthetic Downstream UTR; v1.1 3 UTR (-UGA1 + UGA2) -UGA3
uaauaggcuggagccucgguggccuagcuucuugccccuugggccuccccccagccccuccuccccuuccugcacccguacccccguggucuuugaauaaagucucagugggcggc

SEQ ID NO: 8 - RNA, 57 nt, Artificial Sequence
  Synthetic Upstream UTR; V1.1 5UTR
gggaaauaagagagaaaagaagaguaagaagaaauauaagaccccggcgccgccacc

SEQ ID NO: 9 - RNA, 47 nt, Artificial Sequence
  Synthetic Upstream UTR; V1.0 5 UTR-A
aggaaauaagagagaaaagaagaguaagaagaaauauaagagccacc

SEQ ID NO: 10 - RNA, 41 nt, Artificial Sequence
  Synthetic Upstream UTR; V1.0 5UTR-core
uaagagagaaaagaagaguaagaagaaauauaagagccacc

SEQ ID NO: 11 - RNA, 57 nt, Artificial Sequence
  Synthetic Upstream UTR; V1.1 5UTR-A
aggaaauaagagagaaaagaagaguaagaagaaauauaagaccccggcgccgccacc

SEQ ID NO: 12 - RNA, 51 nt, Artificial Sequence
  Synthetic Upstream UTR; V1.1 5UTR-core
uaagagagaaaagaagaguaagaagaaauauaagaccccggcgccgccacc

SEQ ID NO: 13 - RNA, 47 nt, Artificial Sequence
  Synthetic Upstream UTR; 5UTR-001
gggagaucagagagaaaagaagaguaagaagaaauauaagagccacc

SEQ ID NO: 14 - RNA, 145 nt, Artificial Sequence
  Synthetic Upstream UTR; 5′UTR-002
ggaauaaaagucucaacacaacauauacaaaacaaacgaaucucaagcaaucaagcauucuacuucuauugcagcaauuuaaaucauuucuuuuaaagcaaaagcaauuuucugaaaauuuucaccauuuacgaacgauagcaac

SEQ ID NO: 15 - RNA, 42 nt, Artificial Sequence
  Synthetic Upstream UTR; 5′UTR-003
gggagacaagcuuggcauuccgguacuguugguaaagccacc

SEQ ID NO: 16 - RNA, 47 nt, Artificial Sequence
  Synthetic Upstream UTR; 5′UTR-004
gggaauuaacagagaaaagaagaguaagaagaaauauaagagccacc

SEQ ID NO: 17 - RNA, 47 nt, Artificial Sequence
  Synthetic Upstream UTR; 5′UTR-005
gggaaauuagacagaaaagaagaguaagaagaaauauaagagccacc

SEQ ID NO: 18 - RNA, 47 nt, Artificial Sequence
  Synthetic Upstream UTR; 5′UTR-006
gggaaauaagagaguaaagaacaguaagaagaaauauaagagccacc

SEQ ID NO: 19 - RNA, 47 nt, Artificial Sequence
  Synthetic Upstream UTR; 5′UTR-007
gggaaaaaagagagaaaagaagacuaagaagaaauauaagagccacc

SEQ ID NO: 20 - RNA, 47 nt, Artificial Sequence
  Synthetic Upstream UTR; 5′UTR-008
gggaaauaagagagaaaagaagaguaagaagauauauaagagccacc

SEQ ID NO: 21 - RNA, 47 nt, Artificial Sequence
  Synthetic Upstream UTR; 5′UTR-009
gggaaauaagagacaaaacaagaguaagaagaaauauaagagccacc

SEQ ID NO: 22 - RNA, 47 nt, Artificial Sequence
  Synthetic Upstream UTR; 5′UTR-010
gggaaauuagagaguaaagaacaguaaguagaauuaaaagagccacc

SEQ ID NO: 23 - RNA, 47 nt, Artificial Sequence
  Synthetic Upstream UTR; 5′UTR-011
gggaaauaagagagaauagaagaguaagaagaaauauaagagccacc

SEQ ID NO: 24 - RNA, 47 nt, Artificial Sequence
  Synthetic Upstream UTR; 5′UTR-012
gggaaauaagagagaaaagaagaguaagaagaaaauuaagagccacc

SEQ ID NO: 25 - RNA, 47 nt, Artificial Sequence
  Synthetic Upstream UTR; 5′UTR-013
gggaaauaagagagaaaagaagaguaagaagaaauuuaagagccacc

SEQ ID NO: 26 - RNA, 92 nt, Artificial Sequence
  Synthetic Upstream UTR; 5′UTR-014
ucaagcuuuuggacccucguacagaagcuaauacgacucacuauagggaaauaagagagaaaagaagaguaagaagaaauauaagagccacc

SEQ ID NO: 27 - RNA, 140 nt, Artificial Sequence
  Synthetic Upstream UTR; 5′UTR-015
ggacagaucgccuggagacgccauccacgcuguuuugaccuccauagaagacaccgggaccgauccagccuccgcggccgggaacggugcauuggaacgcggauuccccgugccaagagugacucaccguccuugacacg

SEQ ID NO: 28 - RNA, 42 nt, Artificial Sequence
  Synthetic Upstream UTR; 5′UTR-016
ggcgcugccuacggagguggcagccaucuccuucucggcauc

SEQ ID NO: 29 - RNA, 176 nt, Artificial Sequence
  Synthetic Upstream UTR; S065 core
ccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccua

SEQ ID NO: 30 - RNA, 192 nt, Artificial Sequence
  Synthetic Upstream UTR; S065
gggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuaaagcagagaa

SEQ ID NO: 31 - RNA, 192 nt, Artificial Sequence
  Synthetic Upstream UTR; combo3_S065 (S065 ExtKozak)
gggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 32 - RNA, 371 nt, Artificial Sequence
  Synthetic Downstream UTR; Creatine Kinase
gcgccugcccaccugccaccgacugcuggaacccagccagugggagggccuggcccaccagaguccugcucccucacuccucgccccgcccccugucccagagucccaccugggggcucucuccacccuucucagaguuccaguuucaaccagaguuccaaccaaugggcuccauccucuggauucuggccaaugaaauaucucccuggcaggguccucuucuuuucccagagcuccaccccaaccaggagcucuaguuaauggagagcucccagcacacucggagcuugugcuuugucuccacgcaaagcgauaaauaaaagcauugguggccuuuggucuuugaauaaagccugaguaggaagucuaga

SEQ ID NO: 33 - RNA, 568 nt, Artificial Sequence
  Synthetic Downstream UTR; Myoglobin
gccccugccgcucccacccccacccaucugggccccggguucaagagagagcggggucugaucucguguagccauauagaguuugcuucugagugucugcuuuguuuaguagaggugggcaggaggagcugaggggcuggggcugggguguugaaguuggcuuugcaugcccagcgaugcgccucccugugggaugucaucacccugggaaccgggaguggcccuuggcucacuguguucugcaugguuuggaucugaauuaauuguccuuucuucuaaaucccaaccgaacuucuuccaaccuccaaacuggcuguaaccccaaauccaagccauuaacuacaccugacaguagcaauugucugauuaaucacuggccccuugaagacagcagaaugucccuuugcaaugaggaggagaucugggcugggcgggccagcuggggaagcauuugacuaucuggaacuugugugugccuccucagguauggcagugacucaccugguuuuaauaaaacaaccugcaacaucucauggucuuugaauaaagccugaguaggaagucuaga

SEQ ID NO: 34 - RNA, 289 nt, Artificial Sequence
  Synthetic Downstream UTR; alpha-actin
acacacuccaccuccagcacgcgacuucucaggacgacgaaucuucucaaugggggggcggcugagcuccagccaccccgcagucacuuucuuuguaacaacuuccguugcugccaucguaaacugacacaguguuuauaacguguacauacauuaacuuauuaccucauuuuguuauuuuucgaaacaaagcccuguggaagaaaauggaaaacuugaagaagcauuaaagucauucuguuaagcugcguaaauggucuuugaauaaagccugaguaggaagucuaga

SEQ ID NO: 35 - RNA, 379 nt, Artificial Sequence
  Synthetic Downstream UTR; Albumin
caucacauuuaaaagcaucucagccuaccaugagaauaagagaaagaaaaugaagaucaaaagcuuauucaucuguuuuucuuuuucguugguguaaagccaacacccugucuaaaaaacauaaauuucuuuaaucauuuugccucuuuucucugugcuucaauuaauaaaaaauggaaagaaucuaauagagugguacagcacuguuauuuuucaaagauguguugcuauccugaaaauucuguagguucuguggaaguuccaguguucucucuuauuccacuucgguagaggauuucuaguuucuugugggcuaauuaaauaaaucauuaauacucuucuaauggucuuugaauaaagccugaguaggaagucuaga

SEQ ID NO: 36 - RNA, 118 nt, Artificial Sequence
  Synthetic Downstream UTR; alpha-globin
gcugccuucugcggggcuugccuucuggccaugcccuucuucucucccuugcaccuguaccucuuggucuuugaauaaagccugaguaggaaggcggccgcucgagcaugcaucuaga

SEQ ID NO: 37 - RNA, 908 nt, Artificial Sequence
  Synthetic Downstream UTR; G-CSF
gccaagcccuccccaucccauguauuuaucucuauuuaauauuuaugucuauuuaagccucauauuuaaagacagggaagagcagaacggagccccaggccucuguguccuucccugcauuucugaguuucauucuccugccuguagcagugagaaaaagcuccuguccucccauccccuggacugggagguagauagguaaauaccaaguauuuauuacuaugacugcuccccagcccuggcucugcaaugggcacugggaugagccgcugugagccccugguccugaggguccccaccugggacccuugagaguaucaggucucccacgugggagacaagaaaucccuguuuaauauuuaaacagcaguguuccccaucuggguccuugcaccccucacucuggccucagccgacugcacagcggccccugcauccccuuggcugugaggccccuggacaagcagagguggccagagcugggaggcauggcccuggggucccacgaauuugcuggggaaucucguuuuucuucuuaagacuuuugggacaugguuugacucccgaacaucaccgacgcgucuccuguuuuucuggguggccucgggacaccugcccugcccccacgagggucaggacugugacucuuuuuagggccaggcaggugccuggacauuugccuugcuggacggggacuggggaugugggagggagcagacaggaggaaucaugucaggccugugugugaaaggaagcuccacugucacccuccaccucuucaccccccacucaccaguguccccuccacugucacauuguaacugaacuucaggauaauaaaguguuugccuccauggucuuugaauaaagccugaguaggaaggcggccgcucgagcaugcaucuaga

SEQ ID NO: 38 - RNA, 835 nt, Artificial Sequence
  Synthetic Downstream UTR; Col1a2; collagen, type I, alpha 2
acucaaucuaaauuaaaaaagaaagaaauuugaaaaaacuuucucuuugccauuucuucuucuucuuuuuuaacugaaagcugaauccuuccauuucuucugcacaucuacuugcuuaaauugugggcaaaagagaaaaagaaggauugaucagagcauugugcaauacaguuucauuaacuccuucccccgcucccccaaaaauuugaauuuuuuuuucaacacucuuacaccuguuauggaaaaugucaaccuuuguaagaaaaccaaaauaaaaauugaaaaauaaaaaccauaaacauuugcaccacuuguggcuuuugaauaucuuccacagagggaaguuuaaaacccaaacuuccaaagguuuaaacuaccucaaaacacuuucccaugagugugauccacauuguuaggugcugaccuagacagagaugaacugagguccuuguuuuguuuuguucauaauacaaaggugcuaauuaauaguauuucagauacuugaagaauguugauggugcuagaagaauuugagaagaaauacuccuguauugaguuguaucgugugguguauuuuuuaaaaaauuugauuuagcauucauauuuuccaucuuauucccaauuaaaaguaugcagauuauuugcccaaaucuucuucagauucagcauuuguucuuugccagucucauuuucaucuucuuccaugguuccacagaagcuuuguuucuugggcaagcagaaaaauuaaauuguaccuauuuuguauaugugagauguuuaaauaaauugugaaaaaaaugaaauaaagcauguuugguuuuccaaaagaacauau

SEQ ID NO: 39 - RNA, 297 nt, Artificial Sequence
  Synthetic Downstream UTR; Col6a2; collagen, type VI, alpha 2
cgccgccgcccgggccccgcagucgagggucgugagcccaccccguccauggugcuaagcgggcccgggucccacacggccagcaccgcugcucacucggacgacgcccugggccugcaccucuccagcuccucccacgggguccccguagccccggcccccgcccagccccaggucuccccaggcccuccgcaggcugcccggccucccucccccugcagccaucccaaggcuccugaccuaccuggccccugagcucuggagcaagcccugacccaauaaaggcuuugaacccau

SEQ ID NO: 40 - RNA, 602 nt, Artificial Sequence
  Synthetic Downstream UTR; RPN1; ribophorin I
ggggcuagagcccucuccgcacagcguggagacggggcaaggagggggguuauuaggauuggugguuuuguuuugcuuuguuuaaagccgugggaaaauggcacaacuuuaccucugugggagaugcaacacugagagccaaggggugggaguugggauaauuuuuauauaaaagaaguuuuuccacuuugaauugcuaaaaguggcauuuuuccuaugugcagucacuccucucauuucuaaaauagggacguggccaggcacgguggcucaugccuguaaucccagcacuuugggaggccgaggcaggcggcucacgaggucaggagaucgagacuauccuggcuaacacgguaaaacccugucucuacuaaaaguacaaaaaauuagcugggcgugguggugggcaccuguagucccagcuacucgggaggcugaggcaggagaaaggcaugaauccaagaggcagagcuugcagugagcugagaucacgccauugcacuccagccugggcaacaguguuaagacucugucucaaauauaaauaaauaaauaaauaaauaaauaaauaaauaaaaauaaagcgagauguugcccucaaa

SEQ ID NO: 41 - RNA, 785 nt, Artificial Sequence
  Synthetic Downstream UTR; LRP1; low density lipoprotein receptor-related protein 1
ggcccugccccgucggacugcccccagaaagccuccugcccccugccagugaaguccuucagugagccccuccccagccagcccuucccuggccccgccggauguauaaauguaaaaaugaaggaauuacauuuuauaugugagcgagcaagccggcaagcgagcacaguauuauuucuccauccccucccugccugcuccuuggcacccccaugcugccuucagggagacaggcagggagggcuuggggcugcaccuccuacccucccaccagaacgcaccccacugggagagcugguggugcagccuuccccucccuguauaagacacuuugccaaggcucuccccucucgccccaucccugcuugcccgcucccacagcuuccugagggcuaauucugggaagggagaguucuuugcugccccugucuggaagacguggcucugggugagguaggcgggaaaggauggaguguuuuaguucuugggggaggccaccccaaaccccagccccaacuccaggggcaccuaugagauggccaugcucaaccccccucccagacaggcccucccugucuccagggcccccaccgagguucccagggcuggagacuuccucugguaaacauuccuccagccuccccuccccuggggacgccaaggaggugggccacacccaggaagggaaagcgggcagccccguuuuggggacgugaacguuuuaauaauuuuugcugaauuccuuuacaacuaaauaacacagauauuguuauaaauaaaauugu

SEQ ID NO: 42 - RNA, 3001 nt, Artificial Sequence
  Synthetic Downstream UTR; Nnt1; cardiotrophin- like cytokine factor 1
auauuaaggaucaagcuguuagcuaauaaugccaccucugcaguuuugggaacaggcaaauaaaguaucaguauacauggugauguacaucuguagcaaagcucuuggagaaaaugaagacugaagaaagcaaagcaaaaacuguauagagagauuuuucaaaagcaguaaucccucaauuuuaaaaaaggauugaaaauucuaaaugucuuucugugcauauuuuuuguguuaggaaucaaaaguauuuuauaaaaggagaaagaacagccucauuuuagauguaguccuguuggauuuuuuaugccuccucaguaaccagaaauguuuuaaaaaacuaaguguuuaggauuucaagacaacauuauacauggcucugaaauaucugacacaauguaaacauugcaggcaccugcauuuuauguuuuuuuuuucaacaaaugugacuaauuugaaacuuuuaugaacuucugagcuguccccuugcaauucaaccgcaguuugaauuaaucauaucaaaucaguuuuaauuuuuuaaauuguacuucagagucuauauuucaagggcacauuuucucacuacuauuuuaauacauuaaaggacuaaauaaucuuucagagaugcuggaaacaaaucauuugcuuuauauguuucauuagaauaccaaugaaacauacaacuugaaaauuaguaauaguauuuuugaagaucccauuucuaauuggagaucucuuuaauuucgaucaacuuauaauguguaguacuauauuaagugcacuugaguggaauucaacauuugacuaauaaaaugaguucaucauguuggcaagugauguggcaauuaucucuggugacaaaagaguaaaaucaaauauuucugccuguuacaaauaucaaggaagaccugcuacuaugaaauagaugacauuaaucugucuucacuguuuauaauacggauggauuuuuuuucaaaucaguguguguuuugaggucuuauguaauugaugacauuugagagaaaugguggcuuuuuuuagcuaccucuuuguucauuuaagcaccaguaaagaucaugucuuuuuauagaaguguagauuuucuuugugacuuugcuaucgugccuaaagcucuaaauauaggugaaugugugaugaauacucagauuauuugucucucuauauaauuaguuugguacuaaguuucucaaaaaauuauuaacacaugaaagacaaucucuaaaccagaaaaagaaguaguacaaauuuuguuacuguaaugcucgcguuuagugaguuuaaaacacacaguaucuuuugguuuuauaaucaguuucuauuuugcugugccugagauuaagaucuguguaugugugugugugugugugugcguuuguguguuaaagcagaaaagacuuuuuuaaaaguuuuaagugauaaaugcaauuuguuaauugaucuuagaucacuaguaaacucagggcugaauuauaccauguauauucuauuagaagaaaguaaacaccaucuuuauuccugcccuuuuucuucucucaaaguaguuguaguuauaucuagaaagaagcaauuuugauuucuugaaaagguaguuccugcacucaguuuaaacuaaaaauaaucauacuuggauuuuauuuauuuuugucauaguaaaaauuuuaauuuauauauauuuuuauuuaguauuaucuuauucuuugcuauuugccaauccuuugucaucaauuguguuaaaugaauugaaaauucaugcccuguucauuuuauuuuacuuuauugguuaggauauuuaaaggauuuuuguauauauaauuucuuaaauuaauauuccaaaagguuaguggacuuagauuauaaauuauggcaaaaaucuaaaaacaacaaaaaugauuuuuauacauucuauuucauuauuccucuuuuuccaauaagucauacaauugguagauaugacuuauuuuauuuuuguauuauucacuauaucuuuaugauauuuaaguauaaauaauuaaaaaaauuuauuguaccuuauagucugucaccaaaaaaaaaaaauuaucuguagguagugaaaugcuaauguugauuugucuuuaagggcuuguuaacuauccuuuauuuucucauuugucuuaaauuaggaguuuguguuuaaauuacucaucuaagcaaaaaauguauauaaaucccauuacuggguauauacccaaaggauuauaaaucaugcugcuauaaagacacaugcacacguauguuuauugcagcacuauucacaauagcaaagacuuggaaccaacccaaauguccaucaaugauagacuugauuaagaaaaugugcacauauacaccauggaauacuaugcagccauaaaaaaggaugaguucauguccuuuguagggacauggauaaagcuggaaaccaucauucugagcaaacuauugcaaggacagaaaaccaaacacugcauguucucacucauaggugggaauugaacaaugagaacacuuggacacaagguggggaacaccacacaccagggccugucaugggguggggggaguggggagggauagcauuaggagauauaccuaauguaaaugaugaguuaaugggugcagcacaccaacauggcacauguauacauauguagcaaaccugcacguugugcacauguacccuagaacuuaaaguauaauuaaaaaaaaaaagaaaacagaagcuauuuauaaagaaguuauuugcugaaauaaaugugaucuuucccauuaaaaaaauaaagaaauuuugggguaaaaaaacacaauauauuguauucuugaaaaauucuaagagaguggaugugaaguguucucaccacaaaagugauaacuaauugagguaaugcacauauuaauuagaaagauuuugucauuccacaauguauauauacuuaaaaauauguuauacacaauaaauacauacauuaaaaaauaaguaaaugua

SEQ ID NO: 43 - RNA, 1037 nt, Artificial Sequence
  Synthetic Downstream UTR; Col6a1; collagen, type VI, alpha 1
cccacccugcacgccggcaccaaacccuguccucccaccccuccccacucaucacuaaacagaguaaaaugugaugcgaauuuucccgaccaaccugauucgcuagauuuuuuuuaaggaaaagcuuggaaagccaggacacaacgcugcugccugcuuugugcaggguccuccggggcucagcccugaguuggcaucaccugcgcagggcccucuggggcucagcccugagcuagugucaccugcacagggcccucugaggcucagcccugagcuggcgucaccugugcagggcccucuggggcucagcccugagcuggccucaccuggguuccccaccccgggcucuccugcccugcccuccugcccgcccucccuccugccugcgcagcuccuucccuaggcaccucugugcugcaucccaccagccugagcaagacgcccucucggggccugugccgcacuagccucccucuccucuguccccauagcugguuuuucccaccaauccucaccuaacaguuacuuuacaauuaaacucaaagcaagcucuucuccucagcuuggggcagccauuggccucugucucguuuugggaaaccaaggucaggaggccguugcagacauaaaucucggcgacucggccccgucuccugaggguccugcuggugaccggccuggaccuuggcccuacagcccuggaggccgcugcugaccagcacugaccccgaccucagagaguacucgcaggggcgcuggcugcacucaagacccucgagauuaacggugcuaaccccgucugcuccucccucccgcagagacuggggccuggacuggacaugagagccccuuggugccacagagggcugugucuuacuagaaacaacgcaaaccucuccuuccucagaauagugauguguucgacguuuuaucaaaggcccccuuucuauguucauguuaguuuugcuccuucuguguuuuuuucugaaccauauccauguugcugacuuuuccaaauaaagguuuucacuccucuc

SEQ ID NO: 44 - RNA, 577 nt, Artificial Sequence
  Synthetic Downstream UTR; Calr; calreticulin
agaggccugccuccagggcuggacugaggccugagcgcuccugccgcagagcuggccgcgccaaauaaugucucugugagacucgagaacuuucauuuuuuuccaggcugguucggauuugggguggauuuugguuuuguuccccuccuccacucucccccacccccuccccgcccuuuuuuuuuuuuuuuuuuaaacugguauuuuaucuuugauucuccuucagcccucaccccugguucucaucuuucuugaucaacaucuuuucuugccucuguccccuucucucaucucuuagcuccccuccaaccuggggggcagugguguggagaagccacaggccugagauuucaucugcucuccuuccuggagcccagaggagggcagcagaaggggguggugucuccaaccccccagcacugaggaagaacggggcucuucucauuucaccccucccuuucuccccugcccccaggacugggccacuucuggguggggcagugggucccagauuggcucacacugagaauguaagaacuacaaacaaaauuucuauuaaauuaaauuuugugucucc

SEQ ID NO: 45 - RNA, 2212 nt, Artificial Sequence
  Synthetic Downstream UTR; Colla1; collagen, type I, alpha 1
cucccuccaucccaaccuggcucccucccacccaaccaacuuuccccccaacccggaaacagacaagcaacccaaacugaacccccucaaaagccaaaaaaugggagacaauuucacauggacuuuggaaaauauuuuuuuccuuugcauucaucucucaaacuuaguuuuuaucuuugaccaaccgaacaugaccaaaaaccaaaagugcauucaaccuuaccaaaaaaaaaaaaaaaaaaagaauaaauaaauaacuuuuuaaaaaaggaagcuugguccacuugcuugaagacccaugcggggguaagucccuuucugcccguugggcuuaugaaaccccaaugcugcccuuucugcuccuuucuccacaccccccuuggggccuccccuccacuccuucccaaaucugucuccccagaagacacaggaaacaauguauugucugcccagcaaucaaaggcaaugcucaaacacccaaguggcccccacccucagcccgcuccugcccgcccagcacccccaggcccugggggaccugggguucucagacugccaaagaagccuugccaucuggcgcucccauggcucuugcaacaucuccccuucguuuuugagggggucaugccgggggagccaccagccccucacuggguucggaggagagucaggaagggccacgacaaagcagaaacaucggauuuggggaacgcgugucaaucccuugugccgcagggcugggcgggagagacuguucuguuccuuguguaacuguguugcugaaagacuaccucguucuugucuugaugugucaccggggcaacugccugggggcggggaugggggcaggguggaagcggcuccccauuuuauaccaaaggugcuacaucuaugugaugggugggguggggagggaaucacuggugcuauagaaauugagaugcccccccaggccagcaaauguuccuuuuuguucaaagucuauuuuuauuccuugauauuuuucuuuuuuuuuuuuuuuuuuuguggauggggacuugugaauuuuucuaaaggugcuauuuaacaugggaggagagcgugugcggcuccagcccagcccgcugcucacuuuccacccucucuccaccugccucuggcuucucaggccucugcucuccgaccucucuccucugaaacccuccuccacagcugcagcccauccucccggcucccuccuagucuguccugcguccucuguccccggguuucagagacaacuucccaaagcacaaagcaguuuuucccccuaggggugggaggaagcaaaagacucuguaccuauuuuguauguguauaauaauuugagauguuuuuaauuauuuugauugcuggaauaaagcauguggaaaugacccaaacauaauccgcaguggccuccuaauuuccuucuuuggaguugggggagggguagacauggggaaggggcuuuggggugaugggcuugccuuccauuccugcccuuucccuccccacuauucucuucuagaucccuccauaaccccacuccccuuucucucacccuucuuauaccgcaaaccuuucuacuuccucuuucauuuucuauucuugcaauuuccuugcaccuuuuccaaauccucuucuccccugcaauaccauacaggcaauccacgugcacaacacacacacacacucuucacaucugggguuguccaaaccucauacccacuccccuucaagcccauccacucuccacccccuggaugcccugcacuugguggcggugggaugcucauggauacugggagggugaggggaguggaacccgugaggaggaccugggggccucuccuugaacugacaugaagggucaucuggccucugcucccuucucacccacgcugaccuccugccgaaggagcaacgcaacaggagaggggucugcugagccuggcgagggucugggagggaccaggaggaaggcgugcucccugcucgcuguccuggcccugggggagugagggagacagacaccugggagagcuguggggaaggcacucgcaccgugcucuugggaaggaaggagaccuggcccugcucaccacggacugggugccucgaccuccugaauccccagaacacaaccccccugggcugggguggucuggggaaccaucgugcccccgccucccgccuacuccuuuuuaagcuu

SEQ ID NO: 46 - RNA, 729 nt, Artificial Sequence
  Synthetic Downstream UTR; Plod1; procollagen- lysine, 2-oxoglutarate 5-dioxygenase 1
uuggccaggccugacccucuuggaccuuucuucuuugccgacaaccacugcccagcagccucugggaccucggggucccagggaacccaguccagccuccuggcuguugacuucccauugcucuuggagccaccaaucaaagagauucaaagagauuccugcaggccagaggcggaacacaccuuuauggcuggggcucuccgugguguucuggacccagccccuggagacaccauucacuuuuacugcuuuguagugacucgugcucuccaaccugucuuccugaaaaaccaaggcccccuucccccaccucuuccauggggugagacuugagcagaacaggggcuuccccaaguugcccagaaagacugucugggugagaagccauggccagagcuucucccaggcacagguguugcaccagggacuucugcuucaaguuuugggguaaagacaccuggaucagacuccaagggcugcccugagucugggacuucugccuccauggcuggucaugagagcaaaccguaguccccuggagacagcgacuccagagaaccucuugggagacagaagaggcaucugugcacagcucgaucuucuacuugccuguggggaggggagugacagguccacacaccacacugggucacccuguccuggaugccucugaagagagggacagaccgucagaaacuggagaguuucuauuaaaggucauuuaaacca

SEQ ID NO: 47 - RNA, 847 nt, Artificial Sequence
  Synthetic Downstream UTR; Nucb1; nucleobindin 1
uccuccgggaccccagcccucaggauuccugaugcuccaaggcgacugaugggcgcuggaugaaguggcacagucagcuucccugggggcuggugucauguugggcuccuggggcgggggcacggccuggcauuucacgcauugcugccaccccagguccaccugucuccacuuucacagccuccaagucuguggcucuucccuucuguccuccgaggggcuugccuucucucguguccagugaggugcucagugaucggcuuaacuuagagaagcccgcccccuccccuucuccgucugucccaagagggucugcucugagccugcguuccuagguggcucggccucagcugccuggguuguggccgcccuagcauccuguaugcccacagcuacuggaauccccgcugcugcuccgggccaagcuucugguugauuaaugagggcaugggguggucccucaagaccuuccccuaccuuuuguggaaccagugaugccucaaagacaguguccccuccacagcugggugccaggggcaggggauccucaguauagccggugaacccugauaccaggagccugggccucccugaaccccuggcuuccagccaucucaucgccagccuccuccuggaccucuuggcccccagccccuuccccacacagccccagaagggucccagagcugaccccacuccaggaccuaggcccagccccucagccucaucuggagccccugaagaccagucccacccaccuuucuggccucaucugacacugcuccgcauccugcuguguguccuguuccauguuccgguuccauccaaauacacuuucuggaacaaa

SEQ ID NO: 48 - RNA, 110 nt, Artificial Sequence
  Synthetic Downstream UTR; alpha-globin-1
gcuggagccucgguggccaugcuucuugccccuugggccuccccccagccccuccuccccuuccugcacccguacccccguggucuuugaauaaagucugagugggcggc

SEQ ID NO: 49 - RNA, 116 nt, Artificial Sequence
  Synthetic Downstream UTR; 3UTR-018
uaauaggcuggagccucgguggccaugcuucuugccccuugggccuccccccagccccuccuccccuuccugcacccguacccccguggucuuugaauaaagucugagugggcggc

SEQ ID NO: 50 - RNA, 118 nt, Artificial Sequence
  Synthetic Downstream UTR; 3UTR-019
ugauaauaggcuggagccucgguggccaugcuucuugccccuugggccuccccccagccccuccuccccuuccugcacccguacccccuggucuuugaauaaagucugagugggcggc

SEQ ID NO: 51 - RNA, 119 nt, Artificial Sequence
  Synthetic Downstream UTR; 3UTR-020
ugauaauaggcuggagccucgguggccaugcuucuugccccuugggccuccccccagccccuccuccccuuccugcacccguacccccguggucuuugaauaaagucugagugggcggc

SEQ ID NO: 52 - RNA, 138 nt, Artificial Sequence
  Synthetic Downstream UTR; 3′ UTR-018 + miR-122-5p binding site
uaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 53 - RNA, 138 nt, Artificial Sequence
  Synthetic Downstream UTR; 3′ UTR-018 + miR-122-3p binding site
uaauaggcuggagccucgguggccaugcuucuugccccuugggccuccccccagccccuccuccccuuccugcacccguacccccuauuuagugugauaauggcguuguggucuuugaauaaagucugagugggcggc

SEQ ID NO: 54 - RNA, 141 nt, Artificial Sequence
  Synthetic Downstream UTR; 3′ UTR-019 + miR-122-5p binding site
ugauaauaggcuggagccucgguggccaugcuucuugccccuugggccuccccccagccccuccuccccuuccugcacccguaccccccaaacaccauugucacacuccaguggucuuugaauaaagucugagugggcggc

SEQ ID NO: 55 - RNA, 133 nt, Artificial Sequence
  Synthetic Downstream UTR; 3′ UTR + miR-142-3p binding site
gcuggagccucgguggccaugcuucuugccccuugggccuccccccagccccuccuccccuuccugcacccguacccccuccauaaaguaggaaacacuacaguggucuuugaauaaagucugagugggcggc

SEQ ID NO: 56 - RNA, 138 nt, Artificial Sequence
  Synthetic Downstream UTR; Altered 3′ UTR-019 + miR-122-5p binding site
uaauaggcuggagccucgguggccaugcuucuugccccuugggccuccccccagccccuccuccccuuccugcacccguaccccccaaacaccauugucacacuccaguggucuuucaauaaagucucagugggcggc

SEQ ID NO: 57 - RNA, 133 nt, Artificial Sequence
  Synthetic Downstream UTR; Altered 3′ UTR + miR-142-3p binding site endonuclease resistant
gcuggagccucgguggccaugcuucuugccccuugggccuccccccagccccuccuccccuuccugcacccguacccccuccauaaaguaggaaacacuacaguggucuuucaauaaagucucagugggcggc

SEQ ID NO: 58 - RNA, 192 nt, Artificial Sequence
  Synthetic Upstream UTR; combo3_S065 - A (S065 ExtKozak)
aggagaccucauauccaggcucaagaauagagcucaguguuuuguuguuuaaucauuccgacguguuuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 59 - RNA, 23 nt, Artificial Sequence
  Synthetic miR-142-3p
uguaguguuuccuacuuuaugga

SEQ ID NO: 60 - RNA, 21 nt, Artificial Sequence
  Synthetic miR-142-5p
cauaaaguagaaagcacuacu

SEQ ID NO: 61 - RNA, 87 nt, Artificial Sequence
  Synthetic miR-142
gacagugcagucacccauaaaguagaaagcacuacuaacagcacuggaggguguaguguuuccuacuuuauggaugaguguacugug

SEQ ID NO: 62 - RNA, 23 nt, Artificial Sequence
  Synthetic miR-142-3p binding site
uccauaaaguaggaaacacuaca

SEQ ID NO: 63 - RNA, 21 nt, Artificial Sequence
  Synthetic miR-142-5p binding site
aguagugcuuucuacuuuaug

SEQ ID NO: 64 - RNA, 852 nt, Artificial Sequence
  Synthetic ORF; GFP (with endonuclease sensitive sites)
augguguccaagggugaggaauuguuuaccgggguggugccuauucucgucgaacuugacggggaugugaauggacacaaguuuucgguauccggagaaggagagggugacgccacauacggaaagcuuacacucaaauucaucuguacgacggggaaacugcccguacccuggccuacgcucguaaccacgcugacuuauggagugcagugcuuuagcagauaccccgaccauaugaagcagcacgacuucuucaagucggcgaugcccgagggguacgugcaagagaggaccauuuucuucaaagacgauggcaauuacaaaacacgcgcagaagucaaguuugagggcgauacucuggucaaucggaucgaauugaagggaaucgauuucaaagaagauggaaacauccuuggccauaagcucgaguacaacuauaacucgcauaaugucuauaucauggcugacaagcagaaaaacgguaucaaagucaacuuuaagauccgacacaauauugaggacgguucggugcagcuugcggaccacuaucaacagaauacgccgauuggggaugguccgguccuuuugccggauaaccauuaucucucaacccagucagcccugagcaaagauccaaacgagaagagggaccacauggucuugcucgaauucgugacagcggcagggaucacucugggaauggacgaguuguacaagagaucucgagauaucagccauggcuucccgccggcgguggcggcgcaggaugauggcacgcugcccaugucuugugcccaggagagcgggauggaccgucacccugcagccugugcuucugcuaggaucaaugug

SEQ ID NO: 65 - RNA, 85 nt, Artificial Sequence
  Synthetic miR-122
ccuuagcagagcuguggagugugacaaugguguuugugucuaaacuaucaaacgccauuaucacacuaaauagcuacugcuaggc

SEQ ID NO: 66 - RNA, 22 nt, Artificial Sequence
  Synthetic miR-122-3p
aacgccauuaucacacuaaaua

SEQ ID NO: 67 - RNA, 22 nt, Artificial Sequence
  Synthetic miR-122-5p
uggagugugacaaugguguuug

SEQ ID NO: 68 - RNA, 22 nt, Artificial Sequence
  Synthetic miR-122-3p binding site
uauuuagugugauaauggcguu

SEQ ID NO: 69 - RNA, 22 nt, Artificial Sequence
  Synthetic miR-122-5p binding site
caaacaccauugucacacucca

SEQ ID NO: 70 - RNA, 852 nt, Artificial Sequence
  Synthetic ORF; GFP variant 1
augguguccaagggcgaggaauuguuuaccgggguggugccuauucucgucgaacucgacggggaugucaauggacacaaguuuucgguauccggcgaaggcgagggcgacgccacauacggaaagcuuacacucaaauucaucuguacgacggggaaacugcccguacccuggccuacgcucguaaccacgcucacuuauggagugcagugcuuuagcagguaccccgaccauaugaagcagcacgacuucuucaagucggcgaugcccgagggguacgugcaggaaaggaccauuuucuucaaggacgauggcaauuacaaaacacgcgccgaagucaaguucgagggcgauacucuggucaaucggaucgaauuaaagggaaucgauuucaaggaggauggaaacauccuuggccauaagcucgaguacaacuauaacucgcauaaugucuauauuauggccgacaagcagaaaaacgguaucaaagucaacuuuaaaauccgacacaauaucgaggacgguucggugcagcuugcggaccacuaucaacaaaauacgccgauuggggaugguccgguccuuuugccggauaaccauuaucucucaacccagucagcccucagcaaggauccaaacgaaaaaagggaccacauggucuugcucgaauucgucacagcggcagggaucacucugggaauggacgaguuguacaaaaggucucgcgauaucagccauggcuucccgccggcgguggcggcgcaggacgauggcacgcugcccaugucuugugcccaggaaagcgggauggaccgucacccugcagccugugcuucugcuaggaucaaugug

SEQ ID NO: 71 - RNA, 852 nt, Artificial Sequence
  Synthetic ORF ; GFP Variant 2
augguguccaagggcgaggaauuguuuaccgggguggugccuauucucgucgaacucgacggggaugucaauggacacaaguuuucgguauccggcgaaggcgagggcgacgccacauacggaaagcuuacacucaaauucaucuguacgacggggaaacugcccguacccuggccuacgcucguaaccacgcucacuuauggagugcagugcuuuagcagguaccccgaccauaugaagcagcacgacuucuucaagucggcgaugcccgagggguacgugcaggaaaggaccauuuucuucaaggacgauggcaauuacaaaacacgcgccgaagucaaguucgagggcgauacucuggucaaucggaucgaauuaaagggaaucgauuucaaggaggauggaaacauccuuggccauaagcucgaguacaacuauaacucgcauaaugucuauaucauggccgacaagcagaaaaacgguaucaaagucaacuuuaaaauccgacacaauaucgaggacgguucggugcagcuugcggaccacuaucaacaaaauacgccgauuggggaugguccgguccuuuugccggauaaccauuaucucucaacccagucagcccucagcaaggauccaaacgaaaaaagggaccacauggucuugcucgaauucgucacagcggcagggaucacucugggaauggacgaguuguacaaaaggucucgcgauaucagccauggcuucccgccggcgguggcggcgcaggacgauggcacgcugcccaugucuugugcccaggaaagcgggauggaccgucacccugcagccugugcuucugcuaggaucaaugug

SEQ ID NO: 72 - RNA, 852 nt, Artificial Sequence
  Synthetic ORF ; GFP Variant 3
augguguccaagggugaggaauuguuuaccgggguggugccuauucucgucgaacuugacggggaugugaauggacacaaguuuucgguauccggagaaggagagggugacgccacauacggaaagcuuacacucaaauucaucuguacgacggggaaacugcccguacccuggccuacgcucguaaccacgcugacuuauggagugcagugcuuuagcagauaccccgaccauaugaagcagcacgacuucuucaagucggcgaugcccgagggguacgugcaagagaggaccauuuucuucaaggacgauggcaauuacaaaacacgcgcagaagucaaguuugagggcgauacucuggucaaucggaucgaauuaaagggaaucgauuucaaagaagauggaaacauccuuggccauaagcucgaguacaacuauaacucgcauaaugucuauauuauggcugacaagcagaaaaacgguaucaaagucaacuuuaagauccgacacaauauugaggacgguucggugcagcuugcggaccacuaucaacagaauacgccgauuggggaugguccgguccuuuugccggauaaccauuaucucucaacccagucagcccugagcaaagauccaaacgagaagagggaccacauggucuugcucgaauucgugacagcggcagggaucacucugggaauggacgaguuguacaagagaucucgagauaucagccauggcuucccgccggcgguggcggcgcaggaugauggcacgcugcccaugucuugugcccaggagagcgggauggaccgucacccugcagccugugcuucugcuaggaucaaugug

SEQ ID NO: 73 - RNA, 9 nt, Artificial Sequence
  Synthetic Stop Codon ; 3xstop
ugauaauag

SEQ ID NO: 74 - RNA, 186 nt, Artificial Sequence
  Synthetic Upstream UTR; combo3_S065 (S065 ExtKozak)  minus leader
aggagaccucauauccaggcucaagaauagagcucaguguuuuguuguuuaaucauuccguuugcgauauucgcgcaaagcagccagucgcgcgcuugcuuuuaaguagaguuguuuuuccacccguuugccaggcaucuuuaauuuaacauauuuuuauuuuucaggcuaaccuacgccgccacc

SEQ ID NO: 75 - RNA, 35 nt, Artificial Sequence
  Synthetic Upstream UTR; 5′UTR Core
uaagagagaaaagaagaguaagaagaaauauaaga

SEQ ID NO: 76 - DNA, 29 bp, Artificial Sequence
  Synthetic T7 promoter
  misc_feature (21)..(29): n is a, c, g, or t
taatacgactcactatagggnnnnnnnnn

SEQ ID NO: 77 - DNA, 18 bp, Artificial Sequence
  Synthetic T7 promoter
taatacgactcactatag

SEQ ID NO: 78 - DNA, 29 bp, Artificial Sequence
  Synthetic T7 promoter
  misc_feature (20)..(29): n is a, c, g, or t
taatacgactcactataagnnnnnnnnnn

SEQ ID NO: 79 - DNA, 29 bp, Artificial Sequence
  Synthetic T7 promoter
  misc_feature (20)..(29): n is a, c, g, or t
attatgctgagtgatattcnnnnnnnnnn

SEQ ID NO: 80 - RNA, 47 nt, Artificial Sequence
  Synthetic Upstream UTR; Variant 2, 3, 4, 5 5′UTR
gggaaauaagagacaaaacaacaguaacaacaaauauaacagccacc

SEQ ID NO: 81 - RNA, 12 nt, Artificial Sequence
  Synthetic oligonucleotide
aaaaaaaaaaaa